SEQUENCE LISTING (39 sequences)

SEQ ID NO: 1 - DNA, 159 bp, Artificial Sequence
  AAV2 ITR “Flop” conformer nucleic acid sequence
aggaacccctagatggagttggccactccctctctgcgcgctcgctcgctcactgaggccgggcccgaaacgggcccgtcgggcgacctttggtcgcccggcctcagtgagcgagcgagcgcgcagagagggagtggccaactccatctaggggttcct

SEQ ID NO: 2 - DNA, 141 bp, Artificial Sequence
  AAV2 ITR “Flip” conformer nucleic acid sequence
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 3 - DNA, 141 bp, Artificial Sequence
  AAV2 ITR “Flop” conformer nucleic acid sequence
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 4 - protein, 727 aa, Artificial Sequence
  Canine Parvovirus (CTG-VP1-3-Leu-VP2)
LAPPAKRARRGLVPPGYKYLGPGNSLDQGEPTNPSDAAAKEHDEAYAAYLRSGKNPYLYFSPADQRFIDQTKDAKDWGGKIGHYFFRAKKAIAPVLTDTPDHPSTSRPTKPTKRSKPPPHIFINLAKKKKAGAGQVKRDNLAPMSDGAVQPDGGQPAVRNERATGSGNGSGGGGGGGSGGVGISTGTFNNQTEFKFLENGWVEITANSSRLVHLNMPESENYRRVVVNNMDKTAVNGNMALDDIHAQIVTPWSLVDANAWGVWFNPGDWQLIVNTMSELHLVSFEQEIFNVVLKTVSESATQPPTKVYNNDLTASLMVALDSNNTMPFTPAAMRSETLGFYPWKPTIPTPWRYYFQWDRTLIPSHTGTSGTPTNIYHGTDPDDVQFYTIENSVPVHLLRTGDEFATGTFFFDCKPCRLTHTWQTNRALGLPPFLNSLPQSEGATNFGDIGVQQDKRRGVTQMGNTNYITEATIMRPAEVGYSAPYYSFEASTQGPFKTPIAAGRGGAQTYENQAADGDPRYAFGRQHGQKTTTTGETPERFTYIAHQDTGRYPEGDWIQNINFNLPVTNDNVLLPTDPIGGKTGINYTNIFNTYGPLTALNNVPPVYPNGQIWDKEFDTDLKPRLHVNAPFVCQNNCPGQLFVKVAPNLTNEYDPDASANMSRIVTYSDFWWKGKLVFKAKLRASHTWNPIQQMSINVDNQFNYVPSNIGGMKIVYEKSQLAPRKLY

SEQ ID NO: 5 - protein, 727 aa, canine parvovirus
MAPPAKRARRGLVPPGYKYLGPGNSLDQGEPTNPSDAAAKEHDEAYAAYLRSGKNPYLYFSPADQRFIDQTKDAKDWGGKIGHYFFRAKKAIAPVLTDTPDHPSTSRPTKPTKRSKPPPHIFINLAKKKKAGAGQVKRDNLAPMSDGGVQPDGGQPAVRNERATGSGNGSGGGGGGGSGGVGISTGTFNNQTEFKFLENGWVEITANSSRLVHLNMPESENYRRVVVNNLDKTAVNGNMALDDTHAQIVTPWSLVDANAWGVWFNPGDWQLIVNTMSELHLVSFEQEIFNVVLKTVSESATQPPTKVYNNDLTASLMVALDSNNTMPFTPAAMRSETLGFYPWKPTIPTPWRYYFQWDRTLIPSHTGTSGTPTNIYHGTDPDDVQFYTIENSVPVHLLRTGDEFATGTFYFDCKPCRLTHTWQTNRALGLPPFLNSLPQAEGGTNFGYIGVQQDKRRGVTQMGNTNIITEATIMRPAEVGYSAPYYSFEASTQGPFKTPIAAGRGGAQTDENRAADGDPRYAFGRQHGQKTTTTGETPERFTYIAHQDTGRYPEGDWIQNINFNLPVTEDNVLLPTDPIGGKTGINYTNIFNTYGPLTALNNVPPVYPNGQIWDKEFDTDLKPRLHVNAPFVCQNNCPGQLFVKVAPNLTNEYDPDASANMSRIVTYSDFWWKGKLVFKAKLRASHTWNPIQQMSINVDNQFNYVPSNIGGMKIVYEKSQLAPRKLY

SEQ ID NO: 6 - protein, 584 aa, canine parvovirus
MSDGAVQPDGGQPAVRNERATGSGNGSGGGGGGGSGGVGISTGTFNNQTEFKFLENGWVEITANSSRLVHLNMPESENYRRVVVNNLDKTAVNGNMALDDTHAQIVTPWSLVDANAWGVWFNPGDWQLIVNTMSELHLVSFEQEIFNVVLKTVSESATQPPTKVYNNDLTASLMVALDSNNTMPFTPAAMRSETLGFYPWKPTIPTPWRYYFQWDRTLIPSHTGTSGTPTNIYHGTDPDDVQFYTIENSVPVHLLRTGDEFATGTFFFDCKPCRLTHTWQTNRALGLPPFLNSLPQSEGGTNFGYIGVQQDKRRGVTQMGNTNYITEATIMRPAEVGYSAPYYSFEASTQGPFKTPIAAGRGGAQTDENQAADGDPRYAFGRQHGQKTTTTGETPERFTYIAHQDTGRYPEGDWIQNINFNLPVTDDNVLLPTDPIGGKTGINYTNIFNTYGPLTALNNVPPVYPNGQIWDKEFDTDLKPRLHVNAPFVCQNNCPGQLFVKVAPNLTNEYDPDASANMSRIVTYSDFWWKGKLVFKAKLRASHTWNPIQQMSINVDNQFNYVPSNIGGMKIVYEKSQLAPRKLY

SEQ ID NO: 7 - protein, 727 aa, Artificial Sequence
  Feline Panelukepenia Virus (CTG-VP1-3-Leu-VP2)
LAPPAKRARRGLVPPGYKYLGPGNSLDQGEPTNPSDAAAKEHDEAYAAYLRSGKNPYLYFSPADQRFIDQTKDAKDWGGKIGHYFFRAKKAIAPVLTDTPDHPSTSRPTKPTKRSKPPPHIFINLAKKKKAGAGQVKRDNLAPMSDGAVQPDGGQPAVRNERATGSGNGSGGGGGGGSGGVGISTGTFNNQTEFKFLENGWVEITANSSRLVHLNMPESENYKRVVVNNMDKTAVKGNMALDDIHVQIVTPWSLVDANAWGVWFNPGDWQLIVNTMSELHLVSFEQEIFNVVLKTVSESATQPPTKVYNNDLTASLMVALDSNNTMPFTPAAMRSETLGFYPWKPTIPTPWRYYFQWDRTLIPSHTGTSGTPTNVYHGTDPDDVQFYTIENSVPVHLLRTGDEFATGTFFFDCKPCRLTHTWQTNRALGLPPFLNSLPQSEGATNFGDIGVQQDKRRGVTQMGNTDYITEATIMRPAEVGYSAPYYSFEASTQGPFKTPIAAGRGGAQTDENQAADGDPRYAFGRQHGQKTTTTGETPERFTYIAHQDTGRYPEGDWIQNINFNLPVTNDNVLLPTDPIGGKTGINYTNIFNTYGPLTALNNVPPVYPNGQIWDKEFDTDLKPRLHVNAPFVCQNNCPGQLFVKVAPNLTNEYDPDASANMSRIVTYSDFWWKGKLVFKAKLRASHTWNPIQQMSINVDNQFNYVPNNIGAMKIVYEKSQLAPRKLY

SEQ ID NO: 8 - protein, 727 aa, Unknown
  Feline Panelukepenia Virus VP1 amino acid sequence
MAPPAKRARRGLVPPGYKYLGPGNSLDQGEPTNPSDAAAKEHDEAYAAYLRSGKNPYLYFSPADQRFIDQTKDAKDWGGKIGHYFFRAKKAIAPVLTDTPDHPSTSRPTKPTKRSKPPPHIFINLAKKKKAGAGQVKRDNLAPMSDGAVQPDGGQPAVRNERATGSGNGSGGGGGGGSGGVGISTGTFNNQTEFKFLENGWVEITANSSRLVHLNMPESENYKRVVVNNMDKTAVKGNMALDDTHVQIVTPWSLVDANAWGVWFNPGDWQLIVNTMSELHLVSFEQEIFNVVLKTVSESATQPPTKVYNNDLTASLMVALDSNNTMPFTPAAMRSETLGFYPWKPTIPTPWRYYFQWDRTLIPSHTGTSGTPTNVYHGTDPDDVQFYTIENSVPVHLLRTGDEFATGTFFFDCKPCRLTHTWQTNRALGLPPFLNSLPQSEGATNFGDIGVQQDKRRGVTQMGNTDYITEATIMRPAEVGYSAPYYSFEASTQGPFKTPIAAGRGGAQTDENQAADGDPRYAFGRQHGQKTTTTGETPERFTYIAHQDTGRYPEGDWIQNINFNLPVTNDNVLLPTDPIGGKTGINYTNIFNTYGPLTALNNVPPVYPNGQIWDKEFDTDLKPRLHVNAPFVCQNNCPGQLFVKVAPNLTNEYDPDASANMSRIVTYSDFWWKGKLVFKAKLRASHTWNPIQQMSINVDNQFNYVPNNIGAMKIVYEKSQLAPRKLY

SEQ ID NO: 9 - protein, 584 aa, Unknown
  Feline Panelukepenia Virus VP2 amino acid sequence
MSDGAVQPDGGQPAVRNERATGSGNGSGGGGGGGSGGVGISTGTFNNQTEFKFLENGWVEITANSSRLVHLNMPESENYKRVVVNNMDKTAVKGNMALDDIHVQIVTPWSLVDANAWGVWFNPGDWQLIVNTMSELHLVSFEQEIFNVVLKTVSESATQPPTKVYNNDLTASLMVALDSNNTMPFTPAAMRSETLGFYPWKPTIPTPWRYYFQWDRTLIPSHTGTSGTPTNIYHGTDPDDVQFYTIENSVPVHLLRTGDEFATGTFFFDCKPCRLTHTWQTNRALGLPPFLNSLPQSEGATNFGDIGVQQDKRRGVTQMGNTNYITEATIMRPAEVGYSAPYYSFEASTQGPFKTPIAAGRGGAQTDENQAADGDPRYAFGRQHGQKTTTTGETPERFTYIAHQDTGRYPEGDWIQNINFNLPVTNDNVLLPTDPIGGKTGINYTNIFNTYGPLTALNNVPPVYPNGQIWDKEFDTDLKPRLHVNAPFVCQNNCPGQLFVKVAPNLTNEYDPDASANMSRIVTYSDFWWKGKLVFKAKLRASHTWNPIQQMSINVDNQFNYVPSNIGAMKIVYEKSQLAPRKLY

SEQ ID NO: 10 - protein, 707 aa, Artificial Sequence
  Bufavirus (CTG-VP1-3-Leu-VP2)
LPAIRKARGWVPPGYNFLGPFNQDFNKEPTNPSDNAAKQHDLEYNKLINQGHNPYWYYNKADEDFIKATDQAPDWGGKFGNFIFRAKKHIAPELAPPAKKKSKTKHSEPEFSHKHIKPGTKRGKPFHIFVNLARKRARMSEPANDTNEQPDNSPVEQGAGQIGGGGGGGGSGVGHSTGDYNNRTEFIYHGDEVTIICHSTRLVHINMSDREDYIIYETDRGPLFPTTQDLQGRDTLNDSYHAKVETPWKLLHANSWGCWFSPADFQQMITTCRDIAPIKMHQKIENIVIKTVSKTGTGETETTNYNNDLTALLQIAQDNSNLLPWAADNFYIDSVGYVPWRACKLPTYCYHVDTWNTIDINQADTPNQWREIKKGIQWDNIQFTPLETMINIDLLRTGDAWESGNYNFHTKPTNLAYHWQSQRHTGSCHPTVAPLVERGQGTNIQSVNCWQWGDRNNPSSASTRVSNIHIGYSFPEWQIHYSTGGPVINPGSAFSQAPWGSTTEGTRLTQGASEKAIYDWSHGDDQPGARETWWQNNQHVTGQTDWAPKNAHTSELNNNVPAATHFWKNSYHNTFSPFTAVDDHGPQYPWGAIWGKYPDTTHKPMMSAHAPFLLHGPPGQLFVKLAPNYTDTLDNGGVTHPRIVTYGTFWWSGQLIFKGKLRTPRQWNTYNLPSLDKRETMKNTVPNEVGHFELPYMPGRCLPNYTL

SEQ ID NO: 11 - protein, 707 aa, Unknown
  Bufavirus VP1 amino acid sequence
MPAIRKARGWVPPGYNYLGPFNQDFSKKPTNPSDNAARKHDLEYNKLIKQGHNPYWNYNHADEDFIKETDQATDWGGKFGNFVFRAKRALAPELAPPAKKKTKTKHTEPEYSHKHIKAGTKRGKPFYLFVNLARKKARMTDTQDVSEQQSDQPSVASTSAKAGGGGGGGGSGVGHSTGNYNNRTEFYYHGDEVTIVCHSSRHIHLNMSESEEYKIYDTDRGPTFPTDQTLQGRDTINDSYHAQVETPWFLINPNSWGTWMNPADFQQLTTTCREVTLEHLDQTLDNIVIKTVSKQGSGAEETTQYNNDLTALLQVALDKSNQLPWVADNMYLDSLGYIPWRPCKLKQYSYHVNFWNTIDIISGPQQNQWQQVKKEIKWDDLQFTPIETTTEIDLLRTGDSWTSGPYKFNTKPTQLSYHWQSTRHTGSVHPTEPPNAIGQQGRNIIDINGWQWGDRSNPMSAATRVSNFHIGYSWPEWRIHYGSGGPAINPGAPFSQAPWSTDPQVRLTQGASEKAIFDYNHGDDDPAHRDQWWQNNLPMTGQTDWAPKNAHQTNVSNNIPSRQEFWTQDYHNTFGPFTAVDDVGIQYPWGAIWTKTPDTTHKPMMSAHAPFICKDGPPGQLLVKLAPNYTENLQTDGLGNNRIVTYATFWWTGKLVLKGKLRLPRQFNLYNLPGRPRGTEAKKFLPNEIGHFELPFMPGRCMPNYTI

SEQ ID NO: 12 - protein, 569 aa, Unknown
  Bufavirus VP2 amino acid sequence
MTDTQDVSEQQSDQPSVASTSAKAGGGGGGGGSGVGHSTGNYNNRTEFYYHGDEVTIVCHSSRHIHLNMSESEEYKIYDTDRGPTFPTDQTLQGRDTINDSYHAQVETPWFLINPNSWGTWMNPADFQQLTTTCREVTLEHLDQTLDNIVIKTVSKQGSGAEETTQYNNDLTALLQVALDKSNQLPWVADNMYLDSLGYIPWRPCKLKQYSYHVNFWNTIDIISGPQQNQWQQVKKEIKWDDLQFTPIETTTEIDLLRTGDSWTSGPYKFNTKPTQLSYHWQSTRHTGSVHPTEPPNAIGQQGRNIIDINGWQWGDRSNPMSAATRVSNFHIGYSWPEWRIHYGSGGPAINPGAPFSQAPWSTDPQVRLTQGASEKAIFDYNHGDDDPAHRDQWWQNNLPMTGQTDWAPKNAHQTNVSNNIPSRQEFWTQDYHNTFGPFTAVDDVGIQYPWGAIWTKTPDTTHKPMMSAHAPFICKDGPPGQLLVKLAPNYTENLQTDGLGNNRIVTYATFWWTGKLVLKGKLRLPRQFNLYNLPGRPRGTEAKKFLPNEIGHFELPFMPGRCMPNYTI

SEQ ID NO: 13 - protein, 727 aa, Artificial Sequence; identical to SEQ ID NO: 4
  Tusavirus (CTG-VP1-3-Leu-VP2)

SEQ ID NO: 14 - protein, 715 aa, Unknown
  Tusavirus VP1 amino acid sequence
MAPAARPRKGWVPPGYNYLGPGNDLDAGEPTNKSDAAARKHDFAYSAYLKQGLDPYWNFNKADEKFIRDTEGATDWGGRLGHWIFRAKKHILPHLKEPTLAGRKRPAPAHIFVNLANKRKKGLPTRKDQQKDTLDSNAQQPVREADQPDGMAASSSDSGPSSSGGGARAGGVGVSTGDFDNTTLWDFHEDGTATITCNSTRLVHLTRPDSLDYKIIPTQNNTAVQTVGHMMDDDNHTQVLTPWSLVDCNAWGVWLSPHDWQHIMNIGEELELLSLEQEVFNVTLKTATETGPPESRITMYNNDLTAVMMITTDTNNQLPYTPAAIRSETLGFYPWRPTVVPRWRYYFDWDRFLSVTSSSDQSTSIINHSSTQSAIGQFFVIETQLPIALLRTGDSYATGGYKFDCNKVNLGRHWQTTRSLGLPPKIEPPTSESALGTINQNARLGWRWGINDVHETNVVRPCTAGYNHPEWFYTHTLEGPAIDPAPPTSIPSNWGGGTPPDTRASSHNQQRITYNYNHGNKDENLNNFSLNPNIELGSIINQGNFLSYEGNGQQINTTAGVGKNGETATSDPNLVRYMPNTYGVYTAVDHQGPVYPHGQIWDKQIHTDKKPELHCLAPFTCKNNPPGQMFVRIAPNLTDTFNATPTFSEIITYADFWWKGTLKMKIKLRPPHQWNIATVLGAAVNIGDAARFVPNRLGQLEFPVINGRIVPSTVY

SEQ ID NO: 15 - protein, 565 aa, Unknown
  Tusavirus VP2 amino acid sequence
MAASSSDSGPSSSGGGARAGGVGVSTGDFDNTTLWDFHEDGTATITCNSTRLVHLTRPDSLDYKIIPTQNNTAVQTVGHMMDDDNHTQVLTPWSLVDCNAWGVWLSPHDWQHIMNIGEELELLSLEQEVFNVTLKTATETGPPESRITMYNNDLTAVMMITTDTNNQLPYTPAAIRSETLGFYPWRPTVVPRWRYYFDWDRFLSVTSSSDQSTSIINHSSTQSAIGQFFVIETQLPIALLRTGDSYATGGYKFDCNKVNLGRHWQTTRSLGLPPKIEPPTSESALGTINQNARLGWRWGINDVHETNVVRPCTAGYNHPEWFYTHTLEGPAIDPAPPTSIPSNWGGGTPPDTRASSHNQQRITYNYNHGNKDENLNNFSLNPNIELGSIINQGNFLSYEGNGQQINTTAGVGKNGETATSDPNLVRYMPNTYGVYTAVDHQGPVYPHGQIWDKQIHTDKKPELHCLAPFTCKNNPPGQMFVRIAPNLTDTFNATPTFSEIITYADFWWKGTLKMKIKLRPPHQWNIATVLGAAVNIGDAARFVPNRLGQLEFPVINGRIVPSTVY

SEQ ID NO: 16 - protein, 707 aa, Artificial Sequence; identical to SEQ ID NO: 10
  Cutavirus (CTG-VP1-3-Leu-VP2)

SEQ ID NO: 17 - protein, 707 aa, Unknown
  Cutavirus VP1 amino acid sequence
MPAIRKARGWVPPGYNFLGPFNQDFNKEPTNPSDNAAKQHDLEYNKLINQGHNPYWYYNKADEDFIKATDQAPDWGGKFGNFIFRAKKHIAPELAPPAKKKSKTKHSEPEFSHKHIKPGTKRGKPFHIFVNLARKRARMSEPANDTNEQPDNSPVEQGAGQIGGGGGGGGSGVGHSTGDYNNRTEFIYHGDEVTIICHSTRLVHINMSDREDYIIYETDRGPLFPTTQDLQGRDTLNDSYHAKVETPWKLLHANSWGCWFSPADFQQMITTCRDIAPIKMHQKIENIVIKTVSKTGTGETETTNYNNDLTALLQIAQDNSNLLPWAADNFYIDSVGYVPWRACKLPTYCYHVDTWNTIDINQADTPNQWREIKKGIQWDNIQFTPLETMINIDLLRTGDAWESGNYNFHTKPTNLAYHWQSQRHTGSCHPTVAPLVERGQGTNIQSVNCWQWGDRNNPSSASTRVSNIHIGYSFPEWQIHYSTGGPVINPGSAFSQAPWGSTTEGTRLTQGASEKAIYDWSHGDDQPGARETWWQNNQHVTGQTDWAPKNAHTSELNNNVPAATHFWKNSYHNTFSPFTAVDDHGPQYPWGAIWGKYPDTTHKPMMSAHAPFLLHGPPGQLFVKLAPNYTDTLDNGGVTHPRIVTYGTFWWSGQLIFKGKLRTPRQWNTYNLPSLDKRETMKNTVPNEVGHFELPYMPGRCLPNYTL

SEQ ID NO: 18 - protein, 569 aa, Unknown
  Cutavirus VP2 amino acid sequence
MSEPANDTNEQPDNSPVEQGAGQIGGGGGGGGSGVGHSTGDYNNRTEFIYHGDEVTIICHSTRLVHINMSDREDYIIYETDRGPLFPTTQDLQGRDTLNDSYHAKVETPWKLLHANSWGCWFSPADFQQMITTCRDIAPIKMHQKIENIVIKTVSKTGTGETETTNYNNDLTALLQIAQDNSNLLPWAADNFYIDSVGYVPWRACKLPTYCYHVDTWNTIDINQADTPNQWREIKKGIQWDNIQFTPLETMINIDLLRTGDAWESGNYNFHTKPTNLAYHWQSQRHTGSCHPTVAPLVERGQGTNIQSVNCWQWGDRNNPSSASTRVSNIHIGYSFPEWQIHYSTGGPVINPGSAFSQAPWGSTTEGTRLTQGASEKAIYDWSHGDDQPGARETWWQNNQHVTGQTDWAPKNAHTSELNNNVPAATHFWKNSYHNTFSPFTAVDDHGPQYPWGAIWGKYPDTTHKPMMSAHAPFLLHGPPGQLFVKLAPNYTDTLDNGGVTHPRIVTYGTFWWSGQLIFKGKLRTPRQWNTYNLPSLDKRETMKNTVPNEVGHFELPYMPGRCLPNYTL

SEQ ID NO: 19 - protein, 737 aa, Artificial Sequence
  Wuharv Parvovirus (CTG-VP1-3-Leu-VP2)
LLIWALFIWLFYFHEGTIEQLNIYHNGNSLQLLHHSSSLTGWVPPGYNYLGPKKFKLQKKPTNPSDAAARKHDLEYNKLIQQGHNPYFYYNHADEDFIKETNQAKDWGGKFGNYIFRAKRALAPELAPPKKKSKTEKGEPSYSYKNIKPGTKRGKPFYLFVNLAKKKRMSAPAAENNDQQPDAAEPSVAGAKATGGGGGGGSGVGHSTGSFNNRTEFIYHGSEVTIICHATRHIHLNMPDSEEYKIYETNRGPRFPTDQTLQGRDTINDSYHAKVQTPWQLLHANCWGCWFSPADWQQMITTCREFKVEHLEQAIDNIVIKTVTKQGTGAEEITQYNNDLTALLQVAEDKSNLMPWAADNMYIDSIGYVPWRPSKLPTYCYHVNFWNTIDINAGAQQNQWAEVKKGIQWDNIQFTPIENMVDIELLRTGDRWDSGPYTFYCKPSSLEYHWQSTRHTGSCHPSTTPQAIGSVGNNLETLNAWQWGDRNNPSSASTRVSNFHIGYSWPEWQFHYSTGGPVVNPGQPFSQAPWGSEVAGTRLTQGASEKAIYDWNHGDEEHQFRETWWQNNENMTGQTNWAPKNIHQKELTNNVAAQTAFWAQGYHNTFGPFTAFDDHGAQYPWGAIWGKEPNTTHKPLASSHAPFMTANPPGQIFVKLAPNYTDTVDNNGNSSRIVTFGTFWWTGKLVIKAKLRTPRQWNTYQLPSLAARETMKETVPNEIGNFEIPYMPGRAMPNYTL

SEQ ID NO: 20 - protein, 737 aa, Unknown
  Wuharv Parvovirus VP1 amino acid sequence
MLIWALFIWLFYFHEGTIEQLNIYHNGNSLQLLHHSSSLTGWVPPGYNYLGPKKFKLQKKPTNPSDAAARKHDLEYNKLIQQGHNPYFYYNHADEDFIKETNQAKDWGGKFGNYIFRAKRALAPELAPPKKKSKTEKGEPSYSYKNIKPGTKRGKPFYLFVNLAKKKRMSAPAAENNDQQPDAAEPSVAGAKATGGGGGGGSGVGHSTGSFNNRTEFIYHGSEVTIICHATRHIHLNMPDSEEYKIYETNRGPRFPTDQTLQGRDTINDSYHAKVQTPWQLLHANCWGCWFSPADWQQMITTCREFKVEHLEQAIDNIVIKTVTKQGTGAEEITQYNNDLTALLQVAEDKSNLMPWAADNMYIDSIGYVPWRPSKLPTYCYHVNFWNTIDINAGAQQNQWAEVKKGIQWDNIQFTPIENMVDIELLRTGDRWDSGPYTFYCKPSSLEYHWQSTRHTGSCHPSTTPQAIGSVGNNLETLNAWQWGDRNNPSSASTRVSNFHIGYSWPEWQFHYSTGGPVVNPGQPFSQAPWGSEVAGTRLTQGASEKAIYDWNHGDEEHQFRETWWQNNENMTGQTNWAPKNIHQKELTNNVAAQTAFWAQGYHNTFGPFTAFDDHGAQYPWGAIWGKEPNTTHKPLASSHAPFMTANPPGQIFVKLAPNYTDTVDNNGNSSRIVTFGTFWWTGKLVIKAKLRTPRQWNTYQLPSLAARETMKETVPNEIGNFEIPYMPGRAMPNYTL

SEQ ID NO: 21 - protein, 569 aa, Unknown
  Wuharv Parvovirus VP2 amino acid sequence
MSAPAAENNDQQPDAAEPSVAGAKATGGGGGGGSGVGHSTGSFNNRTEFIYHGSEVTIICHATRHIHLNMPDSEEYKIYETNRGPRFPTDQTLQGRDTINDSYHAKVQTPWQLLHANCWGCWFSPADWQQMITTCREFKVEHLEQAIDNIVIKTVTKQGTGAEEITQYNNDLTALLQVAEDKSNLMPWAADNMYIDSIGYVPWRPSKLPTYCYHVNFWNTIDINAGAQQNQWAEVKKGIQWDNIQFTPIENMVDIELLRTGDRWDSGPYTFYCKPSSLEYHWQSTRHTGSCHPSTTPQAIGSVGNNLETLNAWQWGDRNNPSSASTRVSNFHIGYSWPEWQFHYSTGGPVVNPGQPFSQAPWGSEVAGTRLTQGASEKAIYDWNHGDEEHQFRETWWQNNENMTGQTNWAPKNIHQKELTNNVAAQTAFWAQGYHNTFGPFTAFDDHGAQYPWGAIWGKEPNTTHKPLASSHAPFMTANPPGQIFVKLAPNYTDTVDNNGNSSRIVTFGTFWWTGKLVIKAKLRTPRQWNTYQLPSLAARETMKETVPNEIGNFEIPYMPGRAMPNYTL

SEQ ID NO: 22 - protein, 914 aa, Unknown
  Human Parvovirus 4 VP1 amino acid sequence
MSAADAYRPGGKLPLDELMQRMNRAIPVGPEPSSPANRGGGPYQTHFAIGIMYSKAFQGLLKFANALPAELSPVKQLVNQLEHYRRKTSDTRVWYRVYLDMTRLLISVAPPGAANKLRQAAAGITHSKAPNAETLRGIVRFAAAAFVPTIENIDRFFEDSLTNFAKEDLDTWKQLHEQFIKLFHPPDVGVHLVSDSRDEGADSIVEPDLERPAGGGLTLPGYNYVGPGNPLDSGPPQGPVDEAAKHHDERYAEMIEHGDIPYLHGHGADRLMNKELEEKKRRGDITHLTDVVVGNAIRGLWQAKETVGDIADVQLSQVLPPAPPSSDQQPAYSAGEPSAKKARIGTPEESEPTLPVQSDTNTMSVEPAGGGGGVKVKAQWIGGTSFSDSVVITSHTRTSMLADRGGYVPVYKPGSHVDSSQPVMGMKTPYSYIDVNALSAHFTPRDFQQLLDEYDEIKPKSLTIAISAIVIKDVATNQTGTNVSDSASGGITVFADDSYDYPYVLGHNQDTLPGHLPGENYVLPQYGYITRGREIDQQNSIVAISDHKTELFFLEHHDAECLGTGDHWSHHYEFPDDLPWRKLSTPNQTLYARHNPIPSSRLAIMTGVDNDGTAVWKRPEGMDVGRLPLNYVPGPALMMPTDTQIRNTTFRDPVAIGNPATSDRYSVAPLVHQPWSVRTEEWLANKTDYSVHNYLGGVAYTRRKHEESYDKHEEDRDGRVSNPSRVVQIDGDLAAPHVGHTFFVPGHTRVTSGGTDTVYSPKLYQEPVFPLFPGAVWNPNPLSYDCQIWTKIPNTECHFFAQYPLLGGWGVMTPPPMIFVKLRSQPGPPSPGAHTVPQSNLNQYAIFHLHYSMQFLVKRRKRSRRHNPEKPAPFPTTDSGRMPFTLANSLQDPSTPVYEVPSDQWIARNYSHLL

SEQ ID NO: 23 - protein, 552 aa, Unknown
  Human Parvovirus 4 VP2 amino acid sequence
MSVEPAGGGGGVKVKAQWIGGTSFSDSVVITSHTRTSMLADRGGYVPVYKPGSHVDSSQPVMGMKTPYSYIDVNALSAHFTPRDFQQLLDEYDEIKPKSLTIAISAIVIKDVATNQTGTNVSDSASGGITVFADDSYDYPYVLGHNQDTLPGHLPGENYVLPQYGYITRGREIDQQNSIVAISDHKTELFFLEHHDAECLGTGDHWSHHYEFPDDLPWRKLSTPNQTLYARHNPIPSSRLAIMTGVDNDGTAVWKRPEGMDVGRLPLNYVPGPALMMPTDTQIRNTTFRDPVAIGNPATSDRYSVAPLVHQPWSVRTEEWLANKTDYSVHNYLGGVAYTRRKHEESYDKHEEDRDGRVSNPSRVVQIDGDLAAPHVGHTFFVPGHTRVTSGGTDTVYSPKLYQEPVFPLFPGAVWNPNPLSYDCQIWTKIPNTECHFFAQYPLLGGWGVMTPPPMIFVKLRSQPGPPSPGAHTVPQSNLNQYAIFHLHYSMQFLVKRRKRSRRHNPEKPAPFPTTDSGRMPFTLANSLQDPSTPVYEVPSDQWIARNYSHLL

SEQ ID NO: 24 - protein, 663 aa, Unknown
  Human Parvovirus 4 NS1 amino acid sequence
MDAPAWIAVLQIPTGFLSNPANWRDWDGLQRPRNLLADDWPVQELRESVPLFDHAVNLGYCILQQLFASHAVTLPCRVKPSMFLQLEPSSGEENEMHYHLVVNQADMVGRECSNWLRTWKVFMAGYLVAPTWTLSWNIRKTRQGRLYQADMSFVKNYLLPKLPLNDCYYAWTNIDRFEAAVLSVRNRQLSGPQGAIALPFTDAQPQAPAAEGVPPTMAGKGTQRFMDLIDWLVENGIATEKRWLSVNKLSYRSFLGSSGGVLQAKNALQIAKREMVLAHPLLGYLTKNASTFEESNKVAQLFSLNGYNPVDAAWYFAAWARGVWPKRRAIWLWGPASTGKTLLAAAIANLSPSYGCVNWTNQNFPFNDCHCQSLVWWEEGRMTENIVEVAKAVLGGAPVRLDVKNKGSEDYIPTCVIITSNGDLTVTVDGPVVSTQHQEALQTRITMFQFQRMVPDGLAPLPEEEVRSFFKLGEQELNMKGTPPEEFRVPRNFDKQPMASTSNLPKALCAPIDEDQVQWDSEDDWFPPPTQKRRRAVQETPPRTPSEIIEFSSPSPVADAPPTTPDSPGELSLTPTSVSQIVSAPFPEETAERYGAGDIEAFWSEHVFDADWATRLHICPPGGPRPYGLFWTYLWSREFWRFKQSVSRSEAHLVNRRFIWAWW

SEQ ID NO: 25 - DNA, 1667 bp, Homo sapiens
cccagcaatggatcgatgcacggctgtcggggccgacaggctgacctttactgagctcaggttttcatctccctgttgggagcccaggaaggtcttgctgtggagagaggaacggtgagaagccttggcctgcgagggggagaggcttggcgtgggtgcagtgaagacagcttctgagagctgaaagcccttggaggtcacttatttcaatttcttccaaacacaccacttttacagatgagaaaactgagacttggtgagaaatgacttgtccaaggtcactctaagaggctttgacacagctccagaatccagtgtgtgtgtatgtgtgtgtgtacacatccaacatacatacatctgtatattataaatattatacatattattacgtatacatacacagatacattatatgcatgtgtacgtgtatatcgggagtgtctatacatgtgtattatgaaagcctggctgtggctacgtgtgatgccgtgcctgcgctcactctggtcgtcaacagtttggtcccgcaacatcccgggtagccgccgatccctgagccaccaggcatttcatgcagttctgcaaagccatggagaggagctgaagaaacctcatggtccttttcaaatcgtttcttcctcctcctcctctgcaaagattttctctaagcccagtttgaatccttcagaaacagaacttggctgcgaagtcactttgaaagactttccatatgttaattgcagccggccaaggtctggagcagaggtgggagcccaccatctgcagacggggtcggcccccagtgcgctctgcaaatccccgtcatctggcaggtgtcgttttgggttaattaagagctatactgagcccgtttacctgtcacttctgagaattttaggaaactttgactttcttgccatctctgagctttgagcgaaggggaagctgaaaacacctctgaatctggtgatgtttctgcctctgggatctccaggacagctgcattaagtgcatcttatcataacccctttttaaactttttattttaatcagtgttctctagttagtgcattggtttttacagtcacgtcttctatattggaagacagtactgtttgggggaaacccaccatttgtctgaaatttcttaaggctctgctttctctctgtgtctttgaggaaacagcatacattcctctagctttgttctgtgtaatggctttggagaaactttgaatttgcaggtcaggggctctttcacccattggggtttggggctgtcagtgctaacctcagagctctatgttcatggagggatgactcagttacatccccagatagctgggttctcggttggtcaataggcccccttcttcagtatgagagaattttctctctgtgctgttgacaatgttctattaatatatcttggtaggggtttgggtcacacagatctatgcatttgtcaaaacacagcaatagcacatttaagatttgtgtgtttcattatgtgtaaattttgtatccaaagaaaaaactagtaaacaagtaatgaacttcagttaattgtatgcatgctgaagtacttaggggaaagtgtactgatgtttgcatttacttggaaatgaaatacacattaaggtgaaagaaaggctagagggatgaag

SEQ ID NO: 26 - DNA, 1637 bp, Homo sapiens
agtggtgtgatcatggcttactgcagcctcaacctcccagattcaagtgatcctcctgcctcagcctaccgagtagctgggactacagatgcatgccatcacgcctgactaattttaccttttgtagagatgaggtccctctgtgttgccaaaggtggtctagaactcctgggctcaaatgatcctcccccctccctgggccttccaaagtactgggattacaggtgtaagccaatgcactcagccccatgttacttaatagaaaggtttttcttcccctttttcctgcaccctttgctgctctcacggggaatttctagcatctctaagctctggtctccagtctgaggaagttgtgctgcctgtatgtgacaagagaaataagatgttggcacatgaataggatgttcgccctttggtgaactagagcatgtgagccaattcttaagccagatttttcagcagagaacaattgcaattcacaatcacattttccaggcatgactcatccctatagtatacaataatatgaagagaggctggaaaccccatgcttggcaaataccagtgcccaggcactgcaagctttcttttgtggcagatttttcatacaaactgagtccatcagtctcagagtcccattcaataacaaaagaagaaataaatggggagattaactgctattggaaatgaaggtgttgaaaatgtaaactaaacaaagcaaagcaccccttcactcagttggatccttctaacatagaatcaaacagccatctaaaaccaacaggaaaaccggaccgagggtggagagaaaccgtgtggcaccatcaggaggtaactcccatggtgaggaggagtttcctggctcccattagactttcattttaacccttatgatttttccttttttttttttttttgtatttacatcttttctgctgaactaaaatgataaactctgagagcagagattagattagaatcttctcagcctctccacaattttaaaagcagtgctggccacaggaaaaaaaaaaaaaggtactcaaaaaacactttttttgtttgtgaatgacaatttgaaattgactttgagaaatcttggcagccaagaaaatggctggagaagactttacagcttccgagaagtaggaggatgcagcaggcttctggagggtcaggggaggagctgatcaactggaggcgggagagggaggccatagtgggagagatgaaacgggaaaggaatactagcattttttaaaaagcataaggggaacaaagggtggatctttattacaataaagtggaggcagccagggtacaaggtacaagtttatggaggaaaaaaatggcaaaatataggcccagtcttctgtcctcctctctgacagggaagggtattggatgttcactctatgaaaaagcaacatattaagttagttgttctagacaagaaaagtaggaaagatattgtaggaaccctttgccctcaaacacatattggcccaccattctcagaaggcaatctcagctggcatgacagagcatctggttgcagaggctcttggggactgagtggctgctgaacgaacaccagcccctctctttggcccatgggtaaaagcagccactgc

SEQ ID NO: 27 - protein, 584 aa, Artificial Sequence
  mutations with respect to canine parvovirus strain N
MSDGAVQPDGGQPAVRNERATGSGNGSGGGGGGGSGGVGISTGTFNNQTEFKFLENGWVEITANSSRLVHLNMPESENYRRVVVNNMDKTAVNGNMALDDIHAQIVTPWSLVDANAWGVWFNPGDWQLIVNTMSELHLVSFEQEIFNVVLKTVSESATQPPTKVYNNDLTASLMVALDSNNTMPFTPAAMRSETLGFYPWKPTIPTPWRYYFQWDRTLIPSHTGTSGTPTNIYHGTDPDDVQFYTIENSVPVHLLRTGDEFATGTFFFDCKPCRLTHTWQTNRALGLPPFLNSLPQSEGATNFGDIGVQQDKRRGVTQMGNTNYITEATIMRPAEVGYSAPYYSFEASTQGPFKTPIAAGRGGAQTYENQAADGDPRYAFGRQHGQKTTTTGETPERFTYIAHQDTGRYPEGDWIQNINFNLPVTNDNVLLPTDPIGGKTGINYTNIFNTYGPLTALNNVPPVYPNGQIWDKEFDTDLKPRLHVNAPFVCQNNCPGQLFVKVAPNLTNEYDPDASANMSRIVTYSDFWWKGKLVFKAKLRASHTWNPIQQMSINVDNQFNYVPSNIGGMKIVYEKSQLAPRKLY

SEQ ID NO: 28 - DNA, 130 bp, Artificial Sequence
  AAV2 ITR “Flip” conformer nucleic acid sequence
aggaacccctagtgatggagttggccactccctctctgcgcgctcgctcgctcactgaggccgcccgggcaaagcccgggcgtcgggcgacctttggtcgcccggcctcagtgagcgagcgagcgcgcag

SEQ ID NO: 29 - DNA, 130 bp, Artificial Sequence
  AAV2 ITR “Flip” conformer nucleic acid sequence
aggaacccctagtgatggagttggccactccctctctgcgcgctcgctcgctcactgaggcgcgggcccgaaacgggcccgctgcccgctggtttccagcgggcgcctcagtgagcgagcgagcgcgcag

SEQ ID NO: 30 - DNA, 130 bp, Artificial Sequence
  AAV2 ITR “Flop” conformer nucleic acid sequence
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 31 - DNA, 144 bp, Artificial Sequence
  AAV2 ITR “Flop” conformer nucleic acid sequence
aggaacccctagtgatggagttggccactccctctctgcgcgctcgctcgctcactgaggcgcccgctggaaaccacgggctgcgggcccgtttcgggcccgcgcctcagtgagcgagcgagcgcgcagagagggagtgggcaa

SEQ ID NO: 32 - DNA, 42 bp, Artificial Sequence
  B and C stems
gcccgggcaaagcccgggcgtcgggcgacctttggtcgcccg

SEQ ID NO: 33 - DNA, 146 bp, Artificial Sequence
  ITR that belongs to different serotypes of AAV
gccatacctctagtgatggagttggccactccctctatgcgcactcgctcgctcggtggggccggacgtgcaaagcacgtccgtctggcgacctttggtcgccaggccccaccgagcgagcgagtgcgcatagagggagtggccaa

SEQ ID NO: 34 - DNA, 146 bp, Artificial Sequence
  ITR that belongs to different serotypes of AAV
gggcaaacctagatgatggagttggccactccctctatgcgcgctcgctcactcactcggccctggagaccaaaggtctccagactgccggcctctggccggcagggccgagtgagtgagcgagcgcgcatagagggagtggccaa

SEQ ID NO: 35 - DNA, 130 bp, Artificial Sequence; identical to SEQ ID NO: 30
  ITR that belongs to different serotypes of AAV

SEQ ID NO: 36 - DNA, 165 bp, Artificial Sequence
  ITR that belongs to different serotypes of AAV
cggtacccctagtgagggagttggccactccctctatgcgcgctcgctcgctcggtggggcctgcggaccaaaggtccgcagacggcagagctctgctctgccggccccaccgagcgagcgagcgcgcatagagggagtggccaactccatcactaggggtaccg

SEQ ID NO: 37 - DNA, 143 bp, Artificial Sequence
  ITR that belongs to different serotypes of AAV
ttgcccactccctctctgcgcgctcgctcgctcggtggggcctgcggaccaaaggtccgcagacggcagagctctgctctgccggccccaccgagcgagcgagcgcgcagagagggagtgggcaactccatcactaggggtaa

SEQ ID NO: 38 - DNA, 143 bp, Artificial Sequence
  ITR that belongs to different serotypes of AAV
ttgcccactccctctatgcgcgctcgctcgctcggtggggcctgcggaccaaaggtccgcagacggcagagctctgctctgccggccccaccgagcgagcgagcgcgcatagagggagtgggcaactccatcactaggggtaa

SEQ ID NO: 39 - DNA, 469 bp, Artificial Sequence
  ITR that belongs to different serotypes of AAV
cgaaagtcacacccggaccccgacgccctggtaccttactctccctctcctactgttttgacgaccagaatagattccctctgtctcttctcttttccccgtgtgggtcatccggtgggacaggggtgtcttagggagggggtcttgccggacgagagacgggagtagaggaccgtaaaggagagtaggaaaaaaggactatttaaaagttaggtaagtatgatagaccagtaggtgcacttatctataaaaaaaaaaccggtcagtataccggggtaaaagaaacatgaaatgacttcaatcgagatcacttaggtccctcgtccccgacatcccaccccgacctcggacttctttctgttttccctagtgacactattataccacccccctcccaatgggtcaagactggtgaaaaaaagagacagagttggttctttacgtctcacggaagtggtgagacattgga

(19) United States
(12) Patent Application Publication
(10) Pub. No.: US 2024/0066080 A1
(43) Pub. Date: Feb. 29, 2024

(54) PROTOPARVOVIRUS AND TETRAPARVOVIRUS COMPOSITIONS AND METHODS FOR GENE THERAPY

(71) Applicant: Synteny Therapeutics, Inc., Cambridge, MA (US)

(72) Inventors: Robert Kotin, Cambridge, MA (US); Sebastian Aguirre, Cambridge, MA (US)

(21) Appl. No.: 18/268,733

(22) PCT Filed: Dec. 23, 2021
(86) PCT No.: PCT/US21/65108

§ 371 Date: Jun. 21, 2023

Related U.S. Application Data
(60) Provisional application No. 63/129,848, filed on Dec. 23, 2020.

(51) Int. Cl. A61K 35/76; C07K 14/005; C12N 9/22; C12N 15/113; C12N 15/86

(52) U.S. Cl. CPC A61K 35/76; C07K 14/005; C12N 9/22; C12N 15/113; C12N 15/86; C12N 2310/11; C12N 2310/141; C12N 2310/20; C12N 2750/14033; C12N 2750/14043

(57) ABSTRACT

Disclosed are recombinant virions that have a capsid protein, or a variant thereof, of a protoparvovirus or tetraparvovirus, and a nucleic acid that includes a heterologous nucleic acid.

### CROSS-REFERENCE TO RELATED APPLICATIONS

This application claims the benefit of priority to U.S. Provisional Application No. 63/129,848, filed on Dec. 23, 2020; the entire contents of which are incorporated herein by reference.

### BACKGROUND

Efficient delivery of a therapeutic transgene is a prerequisite for successful gene therapy. When gene therapy was conceptualized in the early 1970s, mammalian viruses were proposed as an effective vehicle to deliver a gene ‘drug.’ Since then, viral vectors have been intensively investigated and broadly used in gene transfer applications. In recent years, adeno-associated virus (AAV) has emerged as a preferred viral vector for gene therapy due to its ability to transduce a wide range of cell types, cross the blood-brain-barrier, and maintain long-term stable expression predominantly as an episomal element. AAV vectors, derived from the non-pathogenic dependoparvovirus genus of the Parvovirus family, retain no virus genes and have been developed for human applications with relatively few reports of vector related serious adverse events.

However, certain characteristics of AAV impose limitations to its application to gene therapy. In particular, AAV is only capable of packaging less than 5 kb of therapeutic DNA, excluding many therapeutic genes and approaches from development. For example, a therapeutic gene for treating the serious genetic diseases with the greatest incidence, namely Duchenne muscular dystrophy, hemophilia A, and cystic fibrosis, exceeds the size limitation of AAV, thus excluding AAV-mediated gene therapy as a treatment option for these diseases. Moreover, many AAV serotypes appear to be endemic, which results in extensive anti-viral immunity in human populations, complicating AAV gene transfer in many subjects. The prevalence of seroconversion to AAVs has been estimated as ≥70% in adults. Seroconversion typically occurs in childhood due to a productive (co-)infection with a wild-type AAV and helper virus, often adenovirus, generating antibodies that cross-react with epitopes common to most primate AAV capsids. Currently, prospective gene therapy patients are screened for neutralizing antibodies (nAbs) and may be ineligible for AAV gene therapy if nAbs exceed an arbitrarily selected threshold titer. Thus, a large portion of patient population is excluded from gene therapy by AAV. Furthermore, although the natural diversity of AAVs is vast, and host tropism differs among AAV species, several important cell types and tissues for gene therapy remain to be unlocked for targeting.

Accordingly, there is a great need for viral compositions and methods for gene therapy that incorporate the utility of AAV vectors while overcoming the limitations.

### SUMMARY OF INVENTION

The present invention is based, at least in part, on the discovery that a recombinant virion comprising at least one capsid protein or a variant thereof of a protoparvovirus or tetraparvovirus is particularly advantageous as a vehicle for gene therapy.

First, due to a larger virion genome size, a protoparvovirus (˜5.3 kb (e.g., canine parvovirus) compared with ˜4.7 kb of AAV) or a tetraparvovirus (˜5.3 kb) can package a nucleic acid at least 0.6 kb greater than AAV, thereby allowing delivery of a therapeutic gene(s) whose size exceeds the capacity of AAV. The larger virion genome size also allows delivery of a therapeutic transgene(s) together with genomic safe harbor (GSH) sequences that accommodate site-specific recombination of the transgene(s) at a desired genomic location. Such site-specific recombination allows integration of the transgene at an inert location in the genome, as opposed to random integration that could disrupt an essential gene and its expression. Second, unlike AAV, protoparvovirus or tetraparvovirus is not as prevalent as AAV. Thus, administration of a recombinant virion comprising a capsid protein of a protoparvovirus or a tetrapavovirus, e.g., comprising a therapeutic gene, would not trigger an extensive anti-viral immune reaction that precludes efficient gene delivery. Accordingly, a recombinant virion comprising a capsid protein of a protoparvovirus or a tetrapavovirus can achieve gene delivery with the efficiency unparalleled to AAV. Third, protoparvovirus and tetrapavovirus have an extraordinary tropism for specific tissues. For example, protoparvovirus has a tropism for hematopoietic stem cells and is particularly useful for the treatment or prevention of hematologic diseases such as hemoglobinopathies, anemia, myeloproliferative disorders, coagulopathies, and cancer. In addition, protoparvovirus can efficiently transcytose across the cells via its interaction with a transferrin receptor. Thus, protoparvovirus can cross the blood-brain barrier (BBB) and deliver therapeutic genes to nerve cells that are hidden behind an endothelial barrier. Accordingly, a recombinant virion comprising a capsid protein of protoparvovirus provides a novel means of gene therapy for the patients afflicted with e.g., neurodegenerative or neuromuscular diseases. Similarly, tetraparvovirus has a tropism for cells/organs including stem cells (CD34+ stem cells; mesenchymal stem cells), bone marrow, lung, small intestine, and liver. Accordingly, a recombinant virion comprising either protoparvovirus or tetraparvovirus capsid protein(s) provides a new modality for gene therapy that can target specific cells/tissues/organs for the treatment or prevention of a wide range of human diseases.

In certain aspects, provided herein are recombinant virions comprising at least one capsid protein (or a variant thereof) of a protoparvovirus or tetraparvovirus, or a pharmaceutical composition comprising said recombinant virions. Also provided herein are the recombinant virions having homology arms (e.g., sequences with homology to the genomic DNA of a target cell) that can facilitate integration of a heterologous nucleic acid into a specific site within a target genome, and methods of integrating said nucleic acid within the target genome. In some embodiments, the integration is mediated by cellular processes, such as homologous recombination or non-homologous end joining. In some embodiments, the integration is initiated and facilitated by an exogenously introduced nuclease (e.g., ZFN, TALEN, CRISPR/Cas9-gRNA). In some embodiments, the variant of the at least one capsid protein alters the affinity and/or specificity of the recombinant virion to at least one cellular receptor involved in internalization of the recombinant virion, and/or allows affinity purification.

In certain aspects, also provided herein are methods of preventing or treating a disease in a subject using the recombinant virions described herein. In some embodiments, the recombinant virion is administered to the subject, thereby preventing or treating the disease in vivo. In some embodiments, the method comprises obtaining a plurality of cells from a subject, transducing the recombinant virions described herein, and administering an effective amount of the transduced cells to the subject. A high affinity and specificity of the protoparvovirus or tetraparvovirus capsid protein(s) for different cell types make these recombinant virions particularly useful in gene therapy for a wide range of human diseases. In some embodiments, the methods further include re-administering an additional amount of the virion, pharmaceutical composition, or transduced cells (e.g., for repeat dosing after an attenuation or for calibration).

In some embodiments, the nucleic acid of the recombinant virions and/or pharmaceutical compositions encodes a protein, e.g., a therapeutic protein. In some embodiments, the nucleic acid decreases or eliminates the expression of an endogenous gene (e.g., via RNAi, CRISPR).

In certain aspects, provided herein are methods of treating a disease, further comprising administering to the subject or contacting the cells with an agent that modulates the expression of the nucleic acid. In some embodiments, the agent is selected from a small molecule, a metabolite, an oligonucleotide, a riboswitch, a peptide, a peptidomimetic, a hormone, a hormone analog, and light. In some embodiments, the agent is selected from tetracycline, cumate, tamoxifen, estrogen, and an antisense oligonucleotide (ASO). In some embodiments, the method further comprises re-administering the agent one or more times at intervals. In some embodiments, the re-administration of the agent results in pulsatile expression of the nucleic acid. In some embodiments, the time between the intervals and/or the amount of the agent is increased or decreased based on the serum concentration and/or half-life of the protein expressed from the nucleic acid.

In certain aspects, the disclosure provides use of the recombinant virions and/or pharmaceutical compositions for the treatment or prevention of a disease of a subject. In certain aspects, the disclosure provides use of the recombinant virions and/or pharmaceutical compositions described herein for the preparation of a medicament for treating a subject (e.g., human) in need thereof.

In certain aspects, provided herein are methods of modulating gene expression in a cell or a subject, comprising transducing the recombinant virions and/or pharmaceutical compositions described herein. Such modulation may involve increasing or restoring the expression of an endogenous gene whose expression is aberrantly lower than the expression in a healthy subject. Alternatively, the modulation may involve decreasing or eliminating the expression of an endogenous gene whose expression is aberrantly higher than the expression in a healthy subject.

In certain aspects, provided herein are methods of modulating a function and/or structure of a protein in a target cell, whose function and/or structure is different from the wild-type protein (e.g., due to a mutation or aberrant gene expression). In certain embodiments, the said modulation may improve and/or restore the function and/or structure of a defective protein in a cell of a subject afflicted with a disease.

In certain aspects, further provided herein are methods and compositions for producing the recombinant virions described herein. In some embodiments, the recombinant virions are produced in mammalian cells by introducing a set of genes that express the virus structural and non-structural proteins and the virion genome. In preferred embodiments, the recombinant virions are produced by infecting insect cells. In certain embodiments, a nucleic acid comprising a sequence necessary for producing virions (e.g., a nucleic acid comprising at least one ITR sequence or origin of virion DNA replication, a nucleic acid encoding at least one viral replication protein, a nucleic acid encoding at least one capsid protein) is introduced to the mammalian cells or insect cells transiently. In some embodiments, said nucleic acid is integrated within the mammalian or insect cell genome.

### BRIEF DESCRIPTION OF FIGURES

FIG. 1A-FIG. 1C show a secondary structure of AAV ITR and a schematic diagram of a rolling hairpin replication model. FIG. 1A shows the structure of AAV ITR that forms an extensive secondary structure. The ITR can acquire two configurations (flip and flop). FIG. 1B shows a schematic diagram showing the rolling hairpin replication model by which a viral nucleic acid replicates. FIG. 1C shows alignment of exemplary sequences of ITR that belongs to different serotypes of AAV.

FIG. 2 shows schematic diagrams representing a heterologous nucleic acid/a transgene construct containing a β-globin gene operably linked to a β-globin promoter flanked at the 5′ terminus by one or more HS sequences. Mammalian β-globin gene is regulated by a regulatory region called the locus control region (LCR) containing a series of 5 DNase I hypersensitive sites (HS1-HS5). The HSs is required for efficient expression of the β-globin gene. Each transgene construct is placed between two homology arms (a 5′ homology arm and a 3′ homology arm), which facilitates site-specific integration at a target cell genome by homologous recombination.

FIG. 3 shows schematic diagrams representing a heterologous nucleic acid/a transgene construct containing various promoters. Each promoter (e.g., CAG promoter, AHSP promoter, MND promoter, W-A promoter, PKLR promoter) is operably linked to a transgene of interest, and the entire construct is placed between two homology arms (a 5′ homology arm and a 3′ homology arm), which facilitates site-specific integration at a target cell genome by homologous recombination.

FIG. 4 shows partial DNA sequence of the erythroid-specific promoter of PKLR. A 469-bp region comprising the upstream regulatory domain. Conserved elements between the human and rat PK-R promoter are depicted by dotted lines. The cytosine of the PK-R transcriptional start site is underlined. GATA-1, CAC/SpI motifs, and the regulatory element PKR-RE 1 in the upstream 270-bp region are shown in boxes (orientation indicated by arrows).

FIG. 5A and FIG. 5B show exemplary miRNAs that can be targeted by the recombinant virions described herein. The erythroparvoviral recombinant virions may comprise the miRNA sequences. Alternatively, the recombinant virions may comprise a nucleic acid sequence that inactivates the miRNAs.

FIG. 6 shows pulsatile transgene expression systems. The schematic diagrams show both negative and positive regulation of expression. Example I (upper panel) shows that an ASO (an antisense oligonucleotides ASO or AON) can negatively regulate gene expression post-transcriptionally. Without ASO, a primary transcript (left) is spliced into a translatable mRNA (top line). The addition of an ASO (red line) complementary to the splice acceptor at the 3′ end of the intron/5′ end of Exon 2 interferes with splicing. Thus, in the presence of ASO, the intron remains in the transcript. The unprocessed RNA is either untranslatable or produces a non-functional protein upon translation. Example II (lower panel) illustrates that an ASO can positively affect gene expression post-transcriptionally. A primary transcript (left) contains 4 exons: exon 1, exon 3, and exon 4 encode the therapeutic protein, and exon 2 contains either a nonsense mutation(s) or an out-of-frame-mutation (OOF). Such exon 2 can be engineered into any transgene. Without the ASO, the transcript is processed into a mature mRNA comprising 4 exons (bottom line), i.e., exon 2 with a nonsense mutation(s) or an OOF mutation remains. Thus, the resulting mRNA translates into a truncated or non-functional protein. By contrast, the addition of ASO interferes with splicing, and the mature mRNA consists of exon 1, exon 3, and exon 4, i.e., exon 2 with a nonsense mutation(s) or an OOF mutation is spliced out. Thus, at the default state (no ASO), the therapeutic protein is not produced. Only upon the addition of ASO, the therapeutic protein is produced, thereby resulting in positive regulation.

FIG. 7 shows a schematic diagram of the Baculovirus Expression Vector (BEV) system for generating and characterizing the components necessary for manufacturing the recombinant virions of the present disclosure.

FIG. 8A and FIG. 8B show the successful overexpression of canine parvovirus (CPV) capsid proteins. FIG. 8A shows the overexpressed CPV capsid proteins (VP1 and VP2) separated by polyacrylamide gel electrophoresis (PAGE) in the presence of sodium dodecyl sulfate (SDS). The gel was stained with Coomassie blue. Lane 1: molecular weight markers. Lane 2: overexpressed CPV capsids (cell fraction). Lane 3: overexpressed CPV capsids (supernatant). FIG. 8B shows the Western blot analysis. The overexpressed CPV VP2 was detected using an anti-CPV VP2 antibody. In silico design was utilized to identify the VP protein start codon. The expression of the CPV capsid proteins was successfully adapted into the BEV expression system. The overexpression achieved the desired CPV VP1/VP2 stoichiometry ratio of 1:25.

FIG. 9A-FIG. 9C show the development and characterization of additional reagents necessary to produce the recombinant virions of the present disclosure. FIG. 9A shows the Western blot analysis. The overexpressed AAV2 Rep proteins (p5 (Rep78 and Rep68) and p19 (Rep 52 and Rep 40) proteins) were detected using an anti-AAV Rep antibody. Rep a, Rep b, and Rep c refer to independent clones of cells that express the Rep proteins. FIG. 9B shows a schematic diagram of a reporter GFP gene construct flanked by the AAV2 ITRs (AAV2 ITR-GFP transgene). A functional recombinant virion requires efficient amplification of a transgene enclosed by viral ITRs, e.g., AAV2 ITRs. FIG. 9C shows successful amplification of AAV2 ITR-GFP transgene and demonstrates the functionality of AAV2 ITR and Rep system components.

### DETAILED DESCRIPTION OF THE INVENTION

In certain aspects, provided herein are recombinant virions, pharmaceutical compositions, and methods that allow efficient gene therapy.

### Definitions

The articles “a” and “an” are used herein to refer to one or to more than one (i.e. to at least one) of the grammatical object of the article. By way of example, “an element” means one element or more than one element.

The term “administering” is intended to include routes of administration which allow a therapy to perform its intended function. Examples of routes of administration include injection (intramuscular, subcutaneous, intravenous, parenterally, intraperitoneally, intrathecal, intranasal, intracranial, intravitreal, subretinal, etc.) routes. The routes of administration also include direct injection to the bone marrow. The injection can be a bolus injection or can be a continuous infusion. Depending on the route of administration, the agent can be coated with or disposed in a selected material to protect it from natural conditions which may detrimentally affect its ability to perform its intended function.

The term “gene” is used broadly to refer to any nucleic acid associated with a biological function. The term “gene” applies to a specific genomic sequence, as well as to a cDNA or an mRNA encoded by that genomic sequence. Genes can be associated with regulatory elements, such as enhancers, promoters, and locus control regions, untranslated regions (UTRs), introns, polyadenylation signals, Kozak motifs, TATA-boxes or TATA-less promoters, and post-transcriptional elements, e.g., WPRE.

The term “heterologous” is art-recognized, and when used in relation to a nucleic acid in a recombinant virion, the heterologous nucleic acid is heterologous to the virus from which the at least one capsid protein originates.

The term “homologous recombination” is art-recognized, and when used in relation to a nucleic acid insertion in a target genome, it is intended to include homology-dependent repair.

“Identity” as between nucleic acid sequences of two nucleic acid molecules can be determined as a percentage of identity using known computer algorithms such as the “FASTA” program, using for example, the default parameters as in Pearson et al. (1988) Proc. Natl. Acad. Sci. USA 85:2444 (other programs include the GCG program package (Devereux, J., et al., Nucleic Acids Research 12(I):387 (1984)), BLASTP, BLASTN, FASTA Atschul, S. F., et al., J Molec Biol 215:403 (1990); Guide to Huge Computers, Martin J. Bishop, ed., Academic Press, San Diego, 1994, and Carillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.)).

The term “subject” or “patient” refers to any healthy or diseased animal, mammal or human, or any animal, mammal or human. In some embodiments, the subject is afflicted with a hematologic disease. In various embodiments of the methods of the present invention, the subject has not undergone treatment. In other embodiments, the subject has undergone treatment.

A “therapeutically effective amount” of a substance or cells or virions is an amount capable of producing a medically desirable result (e.g., clinical improvement) in a treated patient with an acceptable benefit: risk ratio, preferably in a human or non-human mammal.

The term “treating” includes prophylactic and/or therapeutic treatments. The term “prophylactic or therapeutic” treatment is art-recognized and includes administration to the subject one or more of the compositions described herein. If it is administered prior to clinical manifestation of the unwanted condition (e.g., disease or other unwanted state of the subject), then the treatment is prophylactic (i.e., it protects the subject against developing the unwanted condition); whereas, if it is administered after manifestation of the unwanted condition, the treatment is therapeutic (i.e., it is intended to diminish, ameliorate, or stabilize the existing unwanted condition or side effects thereof).

### Recombinant Virion

In certain aspects, provided herein is a recombinant virion comprising (1) at least one capsid protein or a variant thereof, of a protoparvovirus or a genotypic variant thereof, and (2) a nucleic acid, wherein the nucleic acid comprises a heterologous nucleic acid.

In some embodiments, the protoparvovirus or a genotypic variant thereof is of a species selected from Carnivore protoparvovirus, Carnivore protoparvovirus 1, Chiropteran protoparvovirus 1, Eulipotyphla protoparvovirus 1, Primate protoparvovirus 1, Primate protoparvovirus 2, Primate protoparvovirus 3, Primate protoparvovirus 4, Rodent protoparvovirus 1, Rodent protoparvovirus 2, Rodent protoparvovirus 3, Ungulate protoparvovirus 1, and Ungulate protoparvovirus 2.

In some embodiments, the protoparvovirus or a genotypic variant thereof is selected from canine parvovirus, feline panelukepenia virus, human bufavirus 1, human bufavirus 2, human bufavirus 3, human tusavirus, human cutavirus, Wuharv parvovirus, porcine parvovirus, minute virus of mice, megabat bufavirus, and a genotypic variant thereof.

In certain aspects, provided herein is a recombinant virion comprising (1) at least one capsid protein or a variant thereof, of a tetraparvovirus or a genotypic variant thereof, and (2) a nucleic acid, wherein the nucleic acid comprises a heterologous nucleic acid.

In some embodiments, the tetraparvovirus or a genotypic variant thereof is of a species selected from Chiropteran tetraparvovirus 1, Primate tetraparvovirus 1, Ungulate tetraparvovirus 1, Ungulate tetraparvovirus 2, Ungulate tetraparvovirus 3, and Ungulate tetraparvovirus 4.

In some embodiments, the tetraparvovirus or a genotypic variant thereof is selected from human parvovirus 4, human parvovirus 4 genotype 1, human parvovirus 4 genotype 2, human parvovirus 4 genotype 3, chimpanzee parvovirus 4, eidolon helvum parvovirus, bovine hokovirus 1, bovine hokovirus 2, porcine hokovirus, porcine cnvirus, yak parvovirus, ovine hokovirus 1, opossum tetraparvovirus, rodent tetraparvovirus, tetraparvovirus sp., and a genotypic variant thereof.

In some embodiments, the tetraparvovirus is human parvovirus 4, human parvovirus 4 genotype 1, human parvovirus 4 genotype 2, human parvovirus 4 genotype 3, or a genotypic variant thereof.

The recombinant virion may be icosahedral. In some embodiments, the capsid protein or a variant thereof comprises structural proteins VP1 and/or VP2.

VP2 may be present in excess of VP1. For example, VP2 may be present in excess of VP1 by at least about 1%, 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 250%, 300%, 350%, 400%, 450%, 500%, 550%, 600%, 650%, 700%, 750%, 800%, 850%, 900%, 950%, 1000%, 1500%, 2000%, 2500%, 3000%, 3500%, 4000%, 4500%, 5000%, 5500%, 6000%, 6500%, 7000%, 7500%, 8000%, 9000%, or 10000%.

In some embodiments, VP1 comprises an amino acid sequence that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to a sequence selected from SEQ ID NOs: 4, 5, 7, 8, 10, 11, 13, 14, 16, 17, 19, 20, and 22.

In some embodiments, VP2 comprises an amino acid sequence that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to a sequence selected from SEQ ID NOs: 4, 6, 7, 9, 10, 12, 13, 15, 16, 18, 19, 21, and 23.

In certain aspects, provided herein are recombinant virions comprising a heterologous nucleic acid, wherein the heterologous nucleic acid comprises a nucleic acid sequence that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to a nucleic acid sequence of a target cell.

In some embodiments, the heterologous nucleic acid is at least about 30%, 35%, 40%, 45%, 50%, 51%, 52%, 53%, 54%, 55%, 6%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to the nucleic acid of a mammal, preferably wherein the mammal is a human.

In some embodiments, the heterologous nucleic acid is not operably linked to a protoparvovirus or tetraparvovirus promoter.

In some embodiments, the recombinant virion of the present disclosure comprises at least one inverted terminal repeat (ITR). In some embodiments, the at least one ITR comprises: (a) a dependoparvovirus ITR, (b) an AAV ITR, optionally an AAV2 ITR, (c) a protoparvovirus ITR, or (d) a tetraparvovirus ITR.

In some embodiments, the protoparvovirus ITR is selected from the ITRs of canine parvovirus, feline panelukepenia virus, human bufavirus 1, human bufavirus 2, human bufavirus 3, human tusavirus, human cutavirus, Wuharv parvovirus, porcine parvovirus, minute virus of mice, megabat bufavirus, and a genotypic variant thereof.

In some embodiments, the tetraparvovirus ITR is selected from the ITRs of human parvovirus 4, human parvovirus 4 genotype 1, human parvovirus 4 genotype 2, human parvovirus 4 genotype 3, chimpanzee parvovirus 4, eidolon helvum parvovirus, bovine hokovirus 1, bovine hokovirus 2, porcine hokovirus, porcine cnvirus, yak parvovirus, ovine hokovirus 1, opossum tetraparvovirus, rodent tetraparvovirus, tetraparvovirus sp., and a genotypic variant thereof.

In some embodiments, the tetraparvovirus ITR is selected from the ITRs of human parvovirus 4, human parvovirus 4 genotype 1, human parvovirus 4 genotype 2, human parvovirus 4 genotype 3, and a genotypic variant thereof.

In certain aspects, provided herein are recombinant virions comprising a nucleic acid, wherein the nucleic acid is deoxyribonucleic acid (DNA). In some embodiments, the DNA is single-stranded or self-complementary duplex.

In some embodiments, the nucleic acid comprises a Rep protein-dependent origin of replication (ori), thereby allowing replication of said nucleic acid (e.g., for vector production). In some embodiments, the nucleic acid comprises a nucleic acid operably linked to a promoter, optionally placed between two ITRs.

In some embodiments, the promoter is selected from: (a) a promoter heterologous to the nucleic acid to which it is operably linked; (b) a promoter that facilitates the tissue-specific expression of the nucleic acid, preferably wherein the promoter facilitates hematopoietic cell-specific expression or erythroid lineage-specific expression; (c) a promoter that facilitates the constitutive expression of the nucleic acid; and (d) a promoter that is inducibly expressed, optionally in response to a metabolite or small molecule or chemical entity.

In some embodiments, the promoter is selected from the CMV promoter, β-globin promoter, CAG promoter, AHSP promoter, MND promoter, Wiskott-Aldrich promoter, and PKLR promoter.

In some embodiments, the nucleic acid is not operably linked to a promoter in the vectors, and is instead dependent on homology-dependent repair (HDR) for incorporation into a genomic region for expression, either into a heterologous locus—for example, utilizing HDR into an albumin exon to produce a fusion protein, or into the homologous genetic locus to restore the open reading frame. In either of these cases, the vector DNA remains “silent” unless integrated into the cellular genome at a site that enables transcriptional activity.

The recombinant virion of the present disclosure may comprise a heterologous nucleic acid encoding a coding RNA and/or a non-coding RNA.

For example, a coding RNA may comprise: (a) a gene encoding a protein or a fragment thereof, preferably a human protein or a fragment thereof; (b) a nucleic acid encoding a nuclease, optionally a Transcription Activator-Like Effector Nuclease (TALEN), a zinc-finger nuclease (ZFN), a meganuclease, a megaTAL, or a CRISPR endonuclease, (e.g., a Cas9 endonuclease or a variant thereof); (c) a nucleic acid encoding a reporter, e.g., luciferase or GFP; and/or (d) a nucleic acid encoding a drug resistance protein, e.g., neomycin resistance.

In some embodiments, a coding RNA is codon-optimized for expression in a target cell.

In some embodiments, the recombinant virion comprises a heterologous nucleic acid comprising a gene encoding a polypeptide, or a fragment thereof, selected from (HBA1, HBA2, HBB, HBG1, HBG2, HBD, HBE1, and/or HBZ), alpha-hemoglobin stabilizing protein (AHSP), coagulation factor VIII, coagulation factor IX, von Willebrand factor, dystrophin or truncated dystrophin, micro-dystrophin, utrophin or truncated utrophin, micro-utrophin, usherin (USH2A), CEP290, INS, F8 or a fragment thereof (e.g., fragment encoding B-domain deleted polypeptide (e.g., VIII SQ, p-VIII)), and cystic fibrosis transmembrane conductance regulator (CFTR).

In some embodiments, a non-coding RNA comprises lncRNA, piRNA, miRNA, shRNA, siRNA, antisense RNA, and/or guide RNA.

In some embodiments, the coding RNA, the protein, or the non-coding RNA increases or restores the expression of an endogenous gene of a target cell. Alternatively, in some embodiments, the coding RNA, the protein, or the non-coding RNA decreases or eliminates the expression of an endogenous gene of a target cell.

In certain aspects, provided herein are recombinant virions comprising a nucleic acid encoding (a) hepcidin or a fragment thereof, and/or homeostatic iron regulator (HFE) or a fragment thereof, (b) at least one non-coding RNA (e.g., piRNA, miRNA, shRNA, siRNA, antisense RNA) that targets DMT-1, ferroportin, and/or an endogenous mutant form of HFE; (c) a CRISPR/Cas system that targets DMT-1, ferroportin, and/or an endogenous mutant form of HFE; and/or (d) any combination of any one of the nucleic acids listed in (a) to (c).

In some embodiments, the recombinant virion (a) increases the expression of HFE and/or hepcidin in the transduced cell; and/or (b) decreases the expression of DMT-1, ferroportin, and/or an endogenous mutant form of HFE in the transduced cell.

In some embodiments, the recombinant virion prevents or treats hemochromatosis, hereditary hemochromatosis, juvenile hemochromatosis, and/or Wilson's disease.

In certain aspects, provided herein are recombinant virions comprising a nucleic acid encoding (a) a soluble form of the TNFα receptor, a soluble form of the IL-6 receptor, a soluble form of the IL-12 receptor, and/or a soluble form of the IL-1β receptor; (b) at least one non-coding RNA (e.g., piRNA, miRNA, shRNA, siRNA, antisense RNA) that targets the TNFα receptor, IL-6 receptor, IL-12 receptor, and/or IL-1β receptor; (c) a CRISPR/Cas system that targets the TNFα receptor, IL-6 receptor, IL-12 receptor, and/or IL-1β receptor; and/or (d) any combination of any one of the nucleic acids listed in (a) to (c).

In some embodiments, the recombinant virion (a) increases the expression of a soluble form of the TNFα receptor, a soluble form of the IL-6 receptor, a soluble form of the IL-12 receptor, or a soluble form of the IL-1β receptor in the transduced cell; and/or (b) decreases the expression of the TNFα receptor, IL-6 receptor, IL-12 receptor, or IL-1β receptor in the transduced cell.

In some embodiments, the recombinant virion prevents or treats rheumatoid arthritis, inflammatory bowel disease, psoriatic arthritis, juvenile chronic arthritis, psoriasis, and/or ankylosing spondylitis.

In certain aspects, provided herein are recombinant virions comprising a nucleic acid encoding a protein or a fragment thereof selected from IRGM, NOD2, ATG2B, ATG9, ATG5, ATG7, ATG16L1, BECN1, EI24/PIG8, TECPR2, WDR45/WIP14, CHMP2B, CHMP4B, Dynein, EPG5, HspB8, LAMP2, LC3b UVRAG, VCP/p97, ZFYVE26, PARK2/Parkin, PARK6/PINK1, SQSTM1/p62, SMURF, AMPK, and ULK1.

In some embodiments, the recombinant virion increases the expression of said protein or a fragment thereof in the transduced cells. In some embodiments, the recombinant virion modulates autophagy.

In some embodiments, the recombinant virion prevents or treats an autophagy-related disease. In some embodiments, the autophagy-related disease is selected from cancer, neurodegenerative disease (e.g., Alzheimer's disease, Parkinson's disease, Huntington's disease, ataxias), inflammatory disease, inflammatory bowel disease, Crohn's disease, rheumatoid arthritis, lupus, multiple sclerosis, chronic obstructive pulmony disease/COPD, pulmonary fibrosis, cystic fibrosis, Sjogren's disease, hyperglycemic disorders, type I diabetes, type II diabetes, insulin resistance, hyperinsulinemia, insulin-resistant diabetes (e.g. Mendenhall's Syndrome, Werner Syndrome, leprechaunism, and lipoatrophic diabetes), dyslipidemia, hyperlipidemia, elevated low-density lipoprotein (LDL), depressed highdensity lipoprotein (HDL), elevated triglycerides, metabolic syndrome, liver disease, renal disease, cardiovascular disease, ischemia, stroke, complications during reperfusion, muscle degeneration, atrophy, symptoms of aging (e.g., muscle atrophy, frailty, metabolic disorders, low grade inflammation, atherosclerosis, stroke, age-associated dementia and sporadic form of Alzheimer's disease, pre-cancerous states, and psychiatric conditions including depression), spinal cord injury, arteriosclerosis, infectious diseases (e.g., bacterial, fungal, viral), AIDS, tuberculosis, defects in embryogenesis, infertility, lysosomal storage diseases, activator deficiency/GM2 gangliosidosis, alpha-mannosidosis, aspartylglucoaminuria, cholesteryl ester storage disease, chronic hexosaminidase A deficiency, cystinosis, Danon disease, Fabry disease, Farber disease, fucosidosis, galactosialidosis, Gaucher Disease (Types I, II and III), GM1 Gangliosidosis, (infantile, late infantile/juvenile and adult/chronic), Hunter syndrome (MPS II), I-Cell disease/Mucolipidosis II, Infantile Free Sialic Acid Storage Disease (ISSD), Juvenile Hexosaminidase A Deficiency, Krabbe disease, Lysosomal acid lipase deficiency, Metachromatic Leukodystrophy, Hurler syndrome, Scheie syndrome, Hurler-Scheie syndrome, Sanfilippo syndrome, Morquio Type A and B, Maroteaux-Lamy, Sly syndrome, mucolipidosis, multiple sulfate deficiency, Niemann-Pick disease, Neuronal ceroid lipofuscinoses, CLN6 disease, Jansky-Bielschowsky disease, Pompe disease, pycnodysostosis, Sandhoff disease, Schindler disease, Tay-Sachs, and Wolman disease.

In certain aspects, provided herein are recombinant virions comprising a nucleic acid encoding (a) CFTR or a fragment thereof, (b) at least one non-coding RNA (e.g., piRNA, miRNA, shRNA, siRNA, antisense RNA) that targets an endogenous mutant form of CFTR, (c) a CRISPR/Cas system that targets an endogenous mutant form of CFTR; and/or (d) any combination of any one of the nucleic acids listed in (a) to (c).

In some embodiments, the recombinant virion (a) increases the expression of CFTR or a fragment thereof; and/or (b) decreases the expression of the endogenous mutant form of CFTR in the transduced cell. In some embodiments, the recombinant virion prevents or treats cystic fibrosis.

In certain aspects, provided herein are recombinant virions comprising a nucleic acid encoding ATPB1, ATPB11, or ABCB4, or a fragment thereof, related to different forms of progresive familial intrahepatic cholastesis.

In certain aspects, provided herein are recombinant virions comprising nucleic acids encoding CPS1 or a fragment thereof, related to lysosomal storage disorder.

In certain aspect provided herein are recombinant virions comprising a nucleic acid encoding ATPB7 or a fragment thereof, a gene related to Wilson disease, a pathology driven by the excessive accumulation of copper in the liver.

In certain aspects, provided herein are recombinant virions comprising a nucleic acid encoding KRT5, KRT14, PLEC1, Col7A1, ITGB4, ITGA6, LAMA3, LAMB3, LAMC2, or KIND1, or a fragment thereof, which are genes related to Epidermolysis Bullosa (EB).

In certain aspects, provided herein are recombinant virions comprising a non-coding DNA. In some embodiments, the non-coding DNA comprises: (a) a transcription regulatory element (e.g., an enhancer, a transcription termination sequence, an untranslated region (5′ or 3′ UTR), a proximal promoter element, a locus control region, a polyadenylation signal sequence), and/or (b) a translation regulatory element (e.g., Kozak sequence, woodchuck hepatitis virus post-transcriptional regulatory element). In some embodiments, the transcription regulatory element is a locus control region, optionally a β-globin LCR or a DNase hypersensitive site (HS) of β-globin LCR.

In certain aspects, provided herein are recombinant virions comprising a nucleic acid sequence that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to the nucleic acid sequence of a genomic safe harbor (GSH) of the target cell.

In some embodiments, the nucleic acid that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to a GSH is placed 5′ and 3′ to the nucleic acid to be integrated, thereby allowing integration to a specific locus in the target genome by homologous recombination.

In certain embodiments, the GSH is AAVS1, ROSA26, CCR5, Kif6, Pax5, an intergenic region of NUPL2, collagen, HTRP, HI 1 (a thymidine kinase encoding nucleic acid at HI 1 locus), beta-2 microglobulin, GAPDH, TCR, RUNX1, KLHL7, mir684, KCNH2, GPNMB, MIR4540, MIR4475, MIR4476, PRL32P21, LOC105376031, LOC105376032, LOC105376030, MELK, EBLN3P, ZCCHC7, or RNF38. In some embodiments, the GSH is AAVS1, ROSA26, CCR5, Kif6, Pax5, or an intergenic region of NUPL2.

In some embodiments, the nucleic acid is integrated into the genome of a target cell upon transduction. In preferred embodiments, the the nucleic acid is integrated into a GSH of the genome of a target cell upon transduction. In some embodiments, the GSH is AAVS1, ROSA26, CCR5, Kif6, Pax5, an intergenic region of NUPL2, collagen, HTRP, HI 1 (a thymidine kinase encoding nucleic acid at HI 1 locus), beta-2 microglobulin, GAPDH, TCR, RUNX1, KLHL7, mir684, KCNH2, GPNMB, MIR4540, MIR4475, MIR4476, PRL32P21, LOC105376031, LOC105376032, LOC105376030, MELK, EBLN3P, ZCCHC7, or RNF38. In some embodiments, the GSH is AAVS1, ROSA26, CCR5, Kif6, Pax5, or an intergenic region of NUPL2.

In some embodiments, the nucleic acid is integrated into the target genome by homologous recombination followed by a DNA break formation induced by an exogenous nuclease. In some embodiments, the nuclease is TALEN, ZFN, a meganuclease, a megaTAL, or a CRISPR endonuclease (e.g., a Cas9 endonuclease or a variant thereof).

The recombinant virion of the present disclosure may comprise a nucleic acid comprising a nucleic acid sequence encoding at least one replication protein and capsid protein or a variant thereof. In some embodiments, the recombinant virion is autonomously replicating.

In some embodiments, the recombinant virion binds and/or transduces a cancer cell or non-cancerous cell. In some embodiments, the recombinant virion binds and/or transduces a stem cell (e.g., hematopoietic stem cell, CD34+ stem cell, CD36+ stem cell, mesenchymal stem cell, cancer stem cell). In some embodiments, the recombinant virion binds and/or transduces a cell expressing the transferrin receptor (CD71).

In some embodiments, the recombinant virion binds and/or transduces a hematopoietic cell, hematopoietic progenitor cell, hematopoietic stem cell, erythroid lineage cell, megakaryocyte, erythroid progenitor cell (EPC), CD34+ cell, CD36+ cell, mesenchymal stem cell, nerve cell, intestinal cell, intestinal stem cell, gut epithelial cell, endothelial cell, lung cell, enterocyte, liver cell (e.g., hepatocyte, hepatic stellate cells (HSCs), Kupffer cells (KCs), liver sinusoidal endothelial cells (LSECs)), brain microvascular endothelial cell (BMVECs), erythroid progenitor cell, lymphoid progenitor cell, B lymphoblast cell, B cell, T cell, basophilic Endemic Burkitt Lymphoma (EBL), polychromatic erythroblast, epidermal stem cell, P63-positive keratinocyte-derived stem cell, keratinocyte, pancreatic 3-cell, K cell, L cell, and/or orthochromatic erythroblast.

### Rational Modification of the Protoparvovirus Capsid Protein

Protoparvovirus transduces cells via its interaction with transferrin receptors (TfR) that are expressed on the target cells. The ability of the protoparvovirus (e.g., canine parvovirus) to interact with TfR from various species relies on the specific amino acids residues located in the VP2 proteins. For canine parvovirus, these interactions map to amino acid residues 93, 300 and 323 of VP2 (Hueffer, Govindasamy et al. 2003, Hueffer, Parker et al. 2003). Furthermore, VP2 position 300 is the most variable amino acid in the genome of the carnivore parvovirus species, suggesting a relevant role in determining a host range (Allison, Organtini et al. 2016). Transferrin receptors undergo post-translational modifications, notably N-linked glycosylation, resulting an antennary structure with terminal sialic acid moieties. These residues in VP2 interact with sialic acids in the transferrin receptor. The interaction with the transferrin receptor sialic acids are critical for the viral capsid to transcytose from the apical to basolateral side of the cell. Thus, the use of a parvovirus capsid harboring specific mutations in VP2 (CPV aa 90 to 95, aa 299 to 301 and/or 320 to 325) are used herein to rationally modulate the host range and its ability for transcytosis.

Accordingly, in certain aspects, the recombinant virions described herein encompass the recombinant viral particles comprising a wild-type VP2 and/or VP2 comprising one or more mutations. In preferred embodiments, such recombinant virion shows altered tropism, biodistribution, altered interaction with TfR and/or cells expressing TfR, altered ability to transduce cells expressing TfR, and/or altered ability to transcytose from the apical to basolateral side of the cell.

In some embodiments, the recombinant virion comprising one or more VP2 mutations specifically transduces the first target cells (e.g., enterocytes) but has the reduced capability to cross the epithelial barriers by transcytosis and reach other tissues, thereby accumulating in the first target cells. This provides enhanced targeting and gene delivery of the first target cells. In some embodiments, the orally administered recombinant virions comprising one or more VP2 mutations provide preferential targeting and gene delivery of the cells in gut epithelia that express TfR, e.g., enterocytes, thereby making it an ideal viral vector for diseases such as hemochromatosis (also described later).

In some embodiments, the recombinant virion comprising one or more VP2 mutations specifically transduces the first target cells and can cross the epithelial barriers by transcytosis and reach other tissues. In preferred embodiments, the recombinant virion comprising one or more VP2 mutations shows enhanced efficiency in transcytosis across the epithelial barriers. This provides enhanced targeting and gene delivery to cells of nervous system by crossing the blood-brain barrier.

In some embodiments, the recombinant virion comprises one or more mutations in canine parvovirus VP2.

In some embodiments, the recombinant virion comprises a variant capsid protein, wherein the variant capsid comprises a VP2 sequence having one or more mutations with respect to canine parvovirus strain N (UniProtKB—P12930) or the amino acid sequence SEQ ID NO: 27. In some embodiments, the one or more mutations are at a region of VP2 having the amino acid residues (i) 91-95, (ii) 297-301, and/or (iii) 320-324 of SEQ ID NO: 27 or the corresponding amino acid residues of VP2 of other protoparvovirus. In some embodiments, the one or more mutations comprise a substitution, deletion, and/or insertion.

In some embodiments, the one or more mutations alter the affinity and/or specificity of the recombinant virion to at least one cellular receptor involved in internalization of the recombinant virion, optionally wherein the at least one cellular receptor is the transferrin receptor. In some embodiments, the one or more mutations alter: a) the tropism or affinity of the recombinant virion to a cell; b) the ability of the recombinant virion to transduce a cell; and/or

c) the ability of the recombinant virion to transcytose across the cell.

In certain aspects, further provided herein are recombinant virions comprising at least one capsid protein or a variant thereof comprising a heterologous peptide tag. In some embodiments, the heterologous peptide tag allows affinity purification using an antibody, an antigen-binding fragment of an antibody, or a nanobody. In some embodiments, the heterologous peptide tag comprises an epitope/tag selected from hemagglutinin, His (e.g., 6×-His), FLAG, E-tag, TK15, Strep-tag II, AU1, AU5, Myc, Glu-Glu, KT3, and IRS.

### Pharmaceutical Compositions

In certain aspects, provided herein are pharmaceutical compositions comprising the recombinant virion described herein and a carrier and/or a diluent. As used herein the pharmaceutically acceptable carrier is intended to include any and all solvents, dispersion media, antibacterial and antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. For determining compatibility, various relevant factors, such as osmolarity, viscosity, and/or baricity can be considered. Supplementary active compounds can also be incorporated into the compositions.

A pharmaceutical composition of the present invention is formulated to be compatible with its intended route of administration. Examples of routes of administration include parenteral, e.g., intravenous, intradermal, subcutaneous, oral, intranasal (e.g., inhalation), transdermal, transmucosal, intravascular, intracerebral, parenteral, intraperitoneal, epidural, intraspinal, intrasternal, intra-articular, intra-synovial, intrathecal, intra-arterial, intracardiac, intramuscular, intrapulmonary, and rectal administration. In certain embodiments, a direct injection into the bone marrow is contemplated. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For example, Ringer's solution and lactated Ringer's solution are USP approved for formulating IV therapeutics, and those solutions are used in some embodiments. In certain embodiments, the excipient and vector compatibility to retain biological activity is established according to suitable methods. For intravenous administration or injection to the bone marrow, suitable carriers include physiological saline, bacteriostatic water, Cremophor EL™ (BASF, Parsippany, NJ) or phosphate buffered saline (PBS). In all cases, the composition should be sterile and should be fluid to the extent that easy syringeability exists. It must be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Inhibition of the action of microorganisms can be achieved by various antibacterial and antifungal agents, for example, parabens, chlorobutanol, phenol, ascorbic acid, thimerosal, and the like, to the extent that they do not affect the integrity/activity of the viral compositions described herein. In many cases, it is preferable to include isotonic agents, for example, sugars, polyalcohols such as manitol, sorbitol, sodium chloride in the composition.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above.

For administration by inhalation, the viral particles described herein are delivered in the form of an aerosol spray from pressured container or dispenser which contains a suitable propellant, e.g., a gas such as carbon dioxide, or a nebulizer.

Systemic administration can also be by transmucosal means. For transmucosal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories.

### Protoparvovirus

Protoparvovirus species include human bufavirus genotypes 1, 2 and 3, human tusavirus, human cutavirus, canine parvovirus, porcine parvovirus, minute virus of mice and megabat bufavirus (see also Table 1 for the nomenclature designated by International Committee on Taxonomy of Viruses (ICTV); world wide web at talk.ictvonline.org/taxonomy/).

Protoparvovirus is of particular interest as a gene therapy vector, given the following intrinsic characteristics. First, neutralizing antibodies against human protoparvovirus, bufavirus, tusavirus, and cutavirus have low prevalence in many Western countries (Vaisanen, Mohanraj et al. 2018). While the circulation of human protoparvovirus, inferred by the prevalence of virus-specific antibodies, have shown to be greater than 50% in the Middle East or Africa, the circulation in European countries and in the United States is strikingly low, varying between 0% and 5% (Vaisanen, Mohanraj et al. 2018). This aspect makes protoparvovirus particularly attractive for gene therapy as compared to AAV-derived vectors, which has a human IgG prevalence of 40-70%.

Second, protoparvovirus has capacity to encapsulate and deliver a larger nucleic acid molecule. Bufavirus can incorporate DNA molecules of ˜5.1 Kb, allowing the design and delivery of genomes that encode larger proteins or contain cis-acting regulatory elements in these vectors (when compared to AAV), while tusavirus and cutavirus can incorporate a genome similar to the size of AAV (˜4.6 Kb).

Third, protoparvovirus can target certain cell types/tissues/organs. Human bufavirus and tusavirus have been isolated from the respiratory and gastrointestinal (GI) tracks (or stool) in humans, and studies performed in non-human primates suggest that bufavirus can elicit a systemic infection (Vaisanen, Mohanraj et al. 2018). Accordingly, bufavirus can be used for gene therapy targeting different human organs including but not limited to the small intestine, liver, heart, lung, brain, and muscle. In addition, parvovirus-derived capsids can tolerate harsh environmental conditions such as low pH levels or physiological conditions found in the stomach. Such tolerance makes a recombinant virion comprising a capsid protein(s) of a protoparvovirus suitable for transducing cells of the gastrointestinal track, including the intestinal stem cells. The small intestine epithelium is organized into two fundamental structures: villi and crypts. Villi form functional absorptive units populated by a diverse group of differentiated cells, including enterocytes, goblet, enteroendocrine, tuft, and microfold cells. Each villus is supported by at least six invaginations, or crypts of Lieberkuhn (Clevers 2013). The crypt is occupied mainly by undifferentiated cells, including transit-amplifying cells; however, differentiated enteroendocrine and Paneth cells also reside in the crypt. Wedged between Paneth cells are the crypt base columnar cells, which maintain homeostasis through both self-renewal and continuous replacement of the differentiated cells that are constantly turned-over. Targeting the intestinal stem cells with the recombinant virion comprising a protoparvovirus capsid(s) of the present disclosure, therefore, opens the possibility to prevent or treat different GI related complications including hereditary hemochromatosis, or inflammatory bowel disease. The use of validated genomic safe harbors for targeting a transgene in intestinal stem cells is substantially beneficial for providing a long-term expression and avoiding any differentiation effect that is often associated with random genomic insertion.

### Features

Members of genus Protoparvovirus are monophyletic and share standard NS1 sequence identity criteria. The genus is split into two branches, one occupied by founder members of the family that have been studied in great detail, whereas the second branch is occupied exclusively by predicted viruses whose coding sequences were identified recently in the wild using virus discovery approaches, but whose biology remains minimally explored. Genomes of the founder protoparvoviruses are distinctive because they contain many reiterations of the tetranucleotide sequence 5′-TGGT-3′ (or its complement 5′-ACCA-3′), which is the modular binding motif of the NS1 duplex DNA recognition site, generally depicted as (TGGT)2-3 (Cotmore et al., 1995). Minute virus of mice NS1 recognizes variably spaced, tandem and inverted, clusters of the TGGT motif, allowing it to bind to a wide variety of sequences distributed throughout replicative-form viral DNA. TGGT/ACCA tetranucleotide clusters are also dispersed throughout the genomes of the new viruses, suggesting significant biological similarities with founder members. For example, in the 4822 nt sequence of bufavirus 1a (human) (JX027296) there are 95 copies of ACCA or TGGT, while in the 4452 nt sequence of a melanoma-associated human cutavirus (KX685945) there are 105 separate copies.

### Virion

X-ray reconstructions indicate that the first ordered VP residues in protoparvovirus capsids are located inside the particle at the base of the 5-fold pore, leaving unresolved VP1 and VP2 N-termini of ˜180 and 37 residues, respectively (Halder et al., 2013, Agbandje-McKenna et al., 1998, Xie and Chapman 1996). The C-terminal region of this unresolved sequence forms a slender glycine-rich chain, present in both VP1 and VP2, which in minute virus of mice (MVM) variant VLPs can be modeled into claw-like densities positioned inside the capsid below the 5-fold channels in some cryoEM reconstructions (Subramanian et al., 2017). However, in X-ray structures of MVM virions, but not empty particles, the first 10 amino acids from a single copy of this sequence (VP2 G37-G28) can be modeled into submolar density that occupies the central pore of most 5-fold cylinders. Although all VP1 and VP2 N-terminal peptides are sequestered in empty particles, a subset of MVM VP2 N-termini become exposed at the virion surface early during genome encapsidation (Cotmore and Tattersall 2005a), presumably via a poorly understood conformational shift that involves expansion of the 5-fold cylinders. These externalized VP2 N-termini contain a nuclear export signal (Maroto et al., 2004) that in some cells effectively converts the trafficking-neutral capsid into a nuclear export-competent particle. Virions are released from infected cells in this form (Cotmore and Tattersall 2005a), but both in the extracellular environment and during cell entry, exposed N-termini undergo proteolytic cleavage, which removes ˜25 amino acids and converts VP2 to a form called VP3. Because X-ray structures show slightly less than one polyglycine tract threaded through each cylinder, it is significant that ˜90% of the ˜50 MVM VP2 termini eventually become surface exposed and cleaved. X-ray structures of cleaved, predominantly VP3, virions indicate that this proteolysis allows the polyglycine tract of cleaved proteins to be retracted into the capsid interior, where it folds back and assumes additional icosahedral ordering extending to residue G30, while being replaced in the cylinders by a new cluster of VP2 N-termini (Govindasamy L, Gurda BL, Halder S, Van Vliet K, McKenna R, Cotmore S F, Tattersall P, Agbandje-McKenna M. 2010, unpublished observations). Externalized VP2 N-termini also serve an important structural role, stabilizing the cylinders prior to cell entry and preventing premature exposure of VP1 N-termini and ultimately the genome (Cotmore and Tattersall 2012). Thus, in members of the genus Protoparvovirus, the 5-fold cylinders serve as portals for three different forms of cargo, mediating 1) genome translocation into and out of the intact particle, 2) VP1SR extrusion prior to bilayer transit, and 3) early externalization of some VP2 N-termini concomitant with genome encapsidation. This is in sharp contrast to viruses in many other parvovirus genera, which rely on just one or two of these portal functions.

A second distinctive feature of protoparvovirus virions is that in X-ray structures not only is the capsid icosahedrally ordered, but so is ˜11-34% of the single-stranded DNA genome, forming patches in each asymmetric unit that are positioned below a cavity on the interior capsid surface. This ordered DNA comprises 2-3 short (8-11 nt) single-strands, which adopt an inverted-loop configuration with phosphates chelated in the interior by two Mg++ ions while the bases point outwards towards the capsid shell where they establish non-covalent interactions with specific amino acid side chains (Halder et al., 2013, Agbandje-McKenna et al., 1998, Chapman and Rossmann 1995). Atomic force microscopy has been used to probe the rigidity of individual MVM particles along their 5-fold, 3-fold and 2-fold symmetry axes, which showed that in empty particles, but not in DNA-containing virions, the two-fold axes can be easily distorted by nanoindentation, suggesting that the genome has a major influence on capsid rigidity of this region (Carrasco et al., 2006). Single alanine mutations that did not compromise intracapsid interactions but did disrupt major interactions between the capsid and bound DNA patches, had no effect on empty particles but abrogated the genome-enhanced 2-fold rigidity seen in full particles, indicating that it derives predominantly from these ordered DNA:capsid interactions (Carrasco et al., 2008). This perhaps indicates the importance of a full-length, 5 kb genome in establishing wild-type capsid dynamics, as also suggested by in vitro uncoating studies (Cotmore et al., 2010).

### Genome Organization and Replication

Protoparvoviruses have heterotelomeric genomes of around 5 kb, flanked by hairpin telomeres of ˜120 nt at their left-end, generally in a single sequence orientation, while the right-end hairpin is ˜250 nt and can be present as either of two inverted-complementary sequences dubbed “flip” and “flop.” The right-end of protoparvovirus genomes can be excised from replication intermediates in the hairpin configuration by hairpin transfer, which in MVM involves the binding of NS1 complexes to two separate clusters of (TGGT)2-3 binding sites, one that positions NS1 over the cleavage site (5′-CTATCA-3′) and a second that is ˜120 bp away, at the hairpin axis. For cleavage to occur, NS1 complexes at these two sites must be coordinated, and the origin refolded, by recruiting DNA bending proteins from the host HMGB family, which bind to NS1 and create an essential ˜30 bp double-helical loop in the intervening G-rich origin DNA (Cotmore et al., 2000). In contrast, origin sequences generated from the left end of this virus are not cleaved in the hairpin configuration because there is a critical TC/GAA mismatch in the hairpin stem. To create an active origin, the left hairpin must be unfolded and copied to form a base-paired junction region that spans adjacent genomes in dimer RF, in which the two arms of the hairpin are effectively segregated on either side of the symmetry axis. However, only the TC arm gives rise to an active origin because the dinucleotide serves as a spacer element that is positioned between the NS1 binding site and the binding site for an essential co-factor, called parvovirus initiation factor (PIF, also known as glucocorticoid modulatory element binding protein GMEB). PIF is a heterodimeric host complex that binds to two spaced 5′-ACGT-3′ half sites positioned near the axis of the DNA palindrome. In the active origin, PIF is able to interact with NS1 across the TC dinucleotide, stabilizing its binding to the relatively weak NS1 binding site, but it cannot stabilize NS1 binding to an identical binding site across the GAA trinucleotide in the inactive (GAA) arm (Christensen et al., 2001). In consequence, sequences in the hairpin configuration or perfectly-duplex hairpin arms carrying the GAA sequence are not cleaved, making them potentially available for alternative roles such as driving transcription from the adjacent P4 promoter (Gu et al., 1995). Due to major disparities in cleavage efficiency between the left- and right-end origins, progeny negative-sense single-strands are preferentially displaced from the right end of the genome, with the result that protoparvoviruses typically displace and package predominantly (˜99%) negative-sense progeny ssDNA.

Viruses in this genus use two transcriptional promoters at map units (mu) 4 and 38, and a single polyadenylation site corresponding to mu 95, to create 3 major size classes of mRNAs, all of which have a short intron sequence between 46-48 mu removed (Pintel et al., 1983). In MVM this splice has alternative donors (D1 and D2) and acceptors (A1 and A2) of different strengths, which are positioned within a region of 120 nt so that a potential D2:A1 splice is eliminated by minimal intron size constraints. Splicing therefore creates 3 forms of each mRNA size class that are expressed with different stoichiometry (Haut and Pintel 1999). Transcripts arising from P4 that have just this central intron removed encode a single form of NS1, translation of which terminates upstream of D1. In some P4 transcripts however, a second, long intron between 10-40 mu is also excised, creating mRNAs that encode NS2 proteins of ˜25 kDa. These share 85 amino acids of N-terminal sequence with NS1, but are then spliced into a different reading frame and finally reach the short central intron where 2 disparate C-terminal hexapeptides can be added. This generates variants called NS2P and NS2Y that are expressed in a ˜5:1 ratio. P38 transcription is strongly transactivated by the C-terminal domain of NS1, mediated by NS1 binding to upstream 5′-TGGT-3′ repeat sequences (Christensen et al., 1995, Lorson et al., 1996). Alternative splicing at the short intron also causes two size variants of the capsid protein to be expressed with ˜1:5 stoichiometry, with VP1 (˜83 kDa) initiating at an ATG codon positioned between the two acceptor sites while VP2 (˜64 kDa) initiates downstream of the splice.

During infection, newly synthesized capsid proteins assemble as two types of trimers (VP2-only and 1×VP1+2×VP2) in the cytoplasm, and are transported into the nucleus for capsid-assembly using a non-conventional, structure-dependent trafficking motif (Lombardo et al., 2000). However, this translocation is restricted to S-phase (Gil-Ranedo et al., 2015), and is dependent upon trimer phosphorylation by the cellular Raf-1 kinase (Riolobos et al., 2010).

Ancillary proteins encoded by protoparvoviruses include the NS2 variants, which appear to have multiple functions that are mostly mediated by interactions with host proteins, and a small alternatively translated (SAT) protein (Zidori et al., 2005). MVM NS2 is not essential in transformed human cell lines, but its absence in murine cells leads to rapid cessation of duplex DNA amplification early in the infectious cycle by an unknown mechanism (Naeger et al., 1990, Ruiz et al., 2006). This early defect can be abrogated by relatively low levels of NS2 expression, but much higher levels of NS2 are required later in the cycle to enable efficient capsid assembly (Cotmore et al., 1997), which is a pre-requisite for the subsequent accumulation of progeny DNA single-strands, and for virion release. In the late capsid defect, VP proteins are expressed, but most fail to assemble into capsids and are rapidly degraded, perhaps reflecting inadequacies in the nuclear translocation of precursor subunits linked to a severe dislocation in normal nuclear/cytoplasmic protein trafficking, as discussed below. During MVM infection NS2 associates with proteins from the cellular 14-3-3 family (Brockhaus et al., 1996) and with the nuclear export factor CRM1 (Bodendorf et al., 1999). Significantly, the NS2 nuclear export signal (NES) engages CRM1 with “supraphysiological” affinity, which is independent of the presence of RanGTP and thus can potentially resist cytoplasmic release (Engelsma et al., 2008). During wildtype MVM infection CRM1 can be detected in the perinuclear cytoplasm, but this redistribution is exacerbated in infections with mutant viruses that carry point mutations close to the NS2 NES that cause CRM1 to bind at even higher affinity (López-Bueno et al., 2004). These mutations also accelerate the onset of a late step in infection, which is characterized by the cytoplasmic accumulation of large, typically nuclear structures including NS1 and empty capsids, again suggesting major disruptions in normal nuclear/cytoplasmic trafficking pathways. Following transfection into A9 fibroblasts, wildtype MVMi genomes express low levels of NS2, but when these genomes were engineered to express one of the NS2-NES mutations, the resulting low levels of mutant NS2 were able to drive wildtype levels of virus progeny accumulation, confirming that the cumulative late infection blocks seen in cells expressing insufficient NS2 result from the stoichiometric limitation of NS2:CRM1 interactions (Choi et al., 2005). Studies with mutant viruses in which NS2:CRM1 binding was impaired, rather than enhanced, similarly indicate that during infection this interaction is required for the efficient release of virions (Eichwald et al., 2002, Miller and Pintel 2002).

The second protoparvovirus ancillary protein, SAT, is encoded within the capsid gene and is expressed late, from the same mRNA as VP2. SAT accumulates in the endoplasmic reticulum (ER) of the infected cell (Zidori et al., 2005). Like NS2, it enhances the rate at which virus spreads through cultures but it acts via a different mechanism that involves induction of irreversible ER-stress and is linked to enhanced cell necrosis (Mészáros et al., 2017b). Although both SAT and the dependoparvovirus ancillary protein, AAP, occupy similar positions in the capsid gene and contain essential N-terminal hydrophobic domains, these proteins are not known to exhibit functional homology. Thus, in protoparvoviruses early virion export is a distinctive feature that can be driven by multiple mechanisms, either occurring prior to cell lysis and mediated by VP2 signals or Crm1 interactions that vary with cell type, or linked to enhanced cell necrosis and driven by SAT. During export, some virions are known to be internalized in COPII vesicles in the endoplasmic reticulum and undergo gelsolin-dependent trafficking to the Golgi, where they undergo tyrosine phosphorylation, and perhaps by other modifications that enhance their subsequent particle-to-infectivity ratios (Bar et al., 2008, Bar et al., 2013). Release at early times in the cycle allows infection to spread rapidly, potentially enhancing overall progeny production from infected tissues and prior to the accumulation of neutralizing antibodies.

### Biology

Kilham rat virus (KRV), one of the original viruses used to establish family Parvoviridae, was isolated in 1959 from lysates of an experimental rat tumor (Kilham and Olivier 1959). Over the next decade, a succession of similar single-stranded DNA viruses were discovered in transplantable tumors, tissue culture cell lines, or laboratory stocks of other viruses. Some of these, such as MVM, closely resemble viruses now known to infect wild rodents, while other members of the same species (Rodent protoparvovirus 1), such as LuIII (M81888), appear to be distant recombinants of viruses found in nature. Studied extensively in the intervening years, these viruses have served as important model systems for defining the basic characteristics and underlying biology of the family. In rodents, viruses from species Rodent protoparvovirus 1 exhibit a range of pathologies, from asymptomatic viremia to teratogenesis and fetal or neonatal cell death. While these viruses fail to infect normal human cells, host restrictions are often relaxed when human cells undergo oncogenic transformation, allowing the viruses to become preferentially oncolytic, and suggesting their potential for use in clinical cancer virotherapy. To this end, Phase I/IIa clinical trials were recently completed using virus H-1 (X01457) to target advanced glioblastoma, which provided evidence that the virus was well tolerated and could partially disrupt the local immune suppression commonly associated with this cancer (Geletneky et al., 2017, Angelova et al., 2017).

In some cells parvovirus infection results in delayed but significant type 1 IFN release, whereas pretreatment with exogenous IFN-beta strongly inhibits the viral life cycle (Grekova et al., 2010, Mattei et al., 2013). During MVMp infection of mouse embryonic fibroblasts (MEFs) the IFN response did not involve mitochondrial antiviral signaling protein (MAVS) and RIG-I sensing and did not conspicuously inhibit viral DNA replication (Mattei et al., 2013), although pretreatment of cells with IFN-beta-neutralizing antibody did enhance infection in another study (Grekova et al., 2010). However, infected MEFs become unresponsive to Poly (I:C) stimulation, suggesting that the virus is able to inactivate antiviral immune mechanisms elicited by type I IFNs.

Important pathogens in this genus include feline parvovirus (FPV), also known as feline panleukopenia virus, and closely related mink and raccoon parvoviruses, which have existed for over 100 years, and canine parvovirus (CPV), which arose as a variant in the mid-1970s and in 1978 spread worldwide, causing a disease pandemic among dogs, wolves and coyotes. These variants all belong to a single species, Carnivore protoparvovirus 1. In adult animals, viruses in this species predominantly infect lymphoid tissues, leading to leukopenia or lymphopenia, and intestinal epithelia, resulting in severe diarrhea, dehydration and fever. In contrast, infection of neonates is characterized by cerebellar lesions in kittens or ferrets, potentially leading to ataxia, or by myocarditis in puppies. Disease is well controlled by vaccination, but mortality in affected litters varies between 20 and 100 percent (reviewed in (Kailasan et al., 2015a)).

Porcine parvovirus (PPV), a member of the species Ungulate protoparvovirus 1, is a major cause of fetal death and infertility in pigs worldwide, although PPV infection alone rarely causes disease in non-pregnant pigs or piglets. However, when seronegative pregnant sows are exposed to a virulent PPV strain during the first 70 days of gestation, transplacental infection can lead to a syndrome called SMEDI (stillbirths, mummification, embryonic death, and infertility) (Mészáros et al., 2017a). Weakly pathogenic and vaccine strains of PPV exist (e.g., NADL-2), which are lethal if injected into the amniotic fluid but they do not cross the placental barrier as efficiently as pathogenic strains (e.g., Kresse), so disease is rare. Widespread vaccination programs are in place to prevent SMEDI, but some newly emerging virulent PPV variants cannot be neutralized by antibodies raised by exposure to current vaccine strains (Mészáros et al., 2017a). Co-infection with PPV can also potentiate the effect of porcine circovirus type 2 (PCV-2, Porcine circovirus 2, family Circoviridae) in the development of post-weaning multisystemic wasting syndrome (PMWS).

Most of the newly discovered viruses segregate to species in a new branch of the Protoparvovirus tree, established for bufavirus 1a (human). Two genotypes of this virus, BuV1 and BuV2, were identified in 2012 in viral metagenomic analysis of fecal samples from diarrheic children in Burkina Faso and Tunisia (hence the name “bufavirus”) (Phan et al., 2012), while a third genotype, BuV3, was later discovered in the diarrheal feces of Bhutanese children (Yahiro et al., 2014). To date, BuV DNA has been detected in the diarrhea of children from Burkina Faso, Tunisia, Bhutan, Thailand, Turkey, China, and Finland, and of adults from Finland, the Netherlands, Thailand, and China, but has not been found in non-diarrheal feces, suggesting a causal relationship (Vaisanen et al., 2017). When analyzed for the presence of anti-BuV1 capsid IgG, the seroprevalences of adults from Finland and the USA were low (˜2-4%), but much higher rates were found for adults in Iraq (˜85%), Iran (˜56%) and Kenya (˜72%) (Vaisanen et al., 2018).

A second human protoparvovirus in the bufavirus branch, called cutavirus (CuV), was detected in a small number of diarrheal samples from Brazilian and Botswanan children, and in four French skin biopsies of cutaneous T-cell lymphomas, from which the virus derives its name (Phan et al., 2016), and in malignant skin lesions from a Danish melanoma patient (Mollerup et al., 2017). The etiological significance of CuV in human disease has yet to be determined.

Prevalence rates for IgG against CuV were evenly low (0-˜6%) in the same sample series mentioned above for bufavirus, confirming that CuV is widely distributed through human populations (Vaisanen et al., 2018). In contrast, IgG directed against a third new, as yet unclassified protoparvovirus that was detected in a Tunisian human fecal sample (hence tusavirus, TuV) (Phan et al., 2014) was not present in the same panels of sera, and its DNA has yet to be detected in other fecal samples (Vaisanen et al., 2017, Vaisanen et al., 2018), so evidence for TuV being a human virus is thus, so far, insufficient. It segregates phylogenetically with viruses occupying the original branch of the protoparvovirus phylogenetic tree, discussed previously.

TABLE 1
Exemplar Isolates of Protoparvovirus

| Species of Protoparvovirus | Carnivore protoparvovirus | |
|---|---|---|
| | Carnivore protoparvovirus 1 | |
| | Chiropteran protoparvovirus 1 | |
| | Eulipotyphla protoparvovirus 1 | |
| | Primate protoparvovirus 1 | |
| | Primate protoparvovirus 2 | |
| | Primate protoparvovirus 3 | |
| | Primate protoparvovirus 4 | |
| | Rodent protoparvovirus 1 | |
| | Rodent protoparvovirus 2 | |
| | Rodent protoparvovirus 3 | |
| | Ungulate protoparvovirus 1 | |
| | Ungulate protoparvovirus 2 | |
| Exemplary Viruses | Accession No. | Ref Seq No. |
| Sea otter parvovirus | KU561552 | NC_030837 |
| Canine parvovirus | M19296 | NC_001539 |
| Megabat bufavirus 1 | LC085675 | NC_029797 |
| Mpulungu (shrew) bufavirus | AB937988 | NC_026815 |
| Bufavirus 1a (human) | JX027296 | NC_038544 |
| Wuharv (rhesus) parvovirus 1 | JX627576 | NC_039049 |
| Cutavirus (human); | KT868811 | NC_039050 |
| Human Cutavirus 1 | | |
| Tusavirus; | KJ495710 | |
| Human tusavirus | | |
| Minute virus of mice | J02275 | NC_001510 |
| Rat parvovirus 1 | AF036710 | NC_038545 |
| Rat bufavirus SY-2015 | KT716186 | NC_028650 |
| Porcine parvovirus; | L23427 | NC_001718 |
| Porcine parvovirus 5 | | |
| Porcine bufavirus; | KT965075 | NC_043446 |
| Protoparvovirus (porcine) | | |
| Porcine parvovirus 2 | | NC_025965 |
| Porcine parvovirus 6 | | NC_023860 |
| Feline panelukepenia virus | FJ231389; | |
| | KP769859 | |
| Human bufavirus 1 | JQ918261 | |
| Human bufavirus 2 | JX027297 | |
| Human bufavirus 3 | AB847989 | |

### Tetraparvovirus

Tetraparvovirus genus includes the human parvovirus 4 (PARV4), porcine parvovirus 2, eidolon elvum parvovirus, yak parvovirus, porcine hokovirus and ovine hokovirus. PARV4 was originally detected in plasma from a person at risk for infection with HIV through injection drug use (Jones, Kapoor et al. 2005). It has a genome of ˜5.3 Kb, 600 nt larger than AAV and its capsid is highly resistant to temperature which makes it a remarkably versatile and stable viral vector. PARV4 is endemic in certain geographic areas, but elsewhere is found confined only to certain high-risk groups such as patients with HIV, HBV or HCV infections, in the setting of persons who inject drugs and those with a history of multiple transfusions. It remains uncertain whether PARV4 actually causes the observed disease, or is a non-pathogenic, opportunistic virus that was detected in a highly exposed, at-risk (for viral infection) population. Seroprevalence of PARV4 in the general population varies in different parts of the world ranging from 0% to 25% (Sharp, Lail et al. 2009). Although, the anti-PARV4 antibody prevalence in patients with chronic viral infections is reported to be between 15-35% in different regions of the world, the prevalence in healthy individuals from other Western countries has been shown to be lower than 1%. A PARV4 IgG screening of UK blood donors identified a seropositivity rate of 4.8%, which is significantly lower than the observed seropositivity rate for primate AAV antigens (Matthews, Sharp et al. 2017). This highlights one of the main benefits of the use of PARV4 as a gene therapy vector. The reduced seroprevalence of neutralizing antibodies in the population of several Western countries, including the United States, together with the tropism for specific target tissues makes it a versatile gene therapy vector. PARV4 tropism and sites of latency are not fully understood, but compelling data suggest that bone marrow, the respiratory tract, liver and gut represent potential sites of viral replication and may be reservoirs for the virus in latent or persistent infected individuals. Therefore, PARV4 has utility to deliver a gene of interest to prevent or treat a broad range of human diseases.

### Genome Organization and Replication

PARV4 is the exemplar virus of the type species, Primate tetraparvovirus 1. This species includes three distinct PARV4 genotypes (G1 AY622943, G2 DQ873390, and G3 EU874248) that have <3% amino acid sequence divergence, plus a virus that infects chimpanzees (HQ 113143). Current knowledge of PARV4 has been reviewed in detail elsewhere (Qiu et al., 2017, Matthews et al., 2017). G1 is the predominant form circulating in Europe and North America at the moment, but G2 (sometimes known as PARV5) also circulates in these areas and is most common in those who were likely infected in the 1980s, suggesting that it may have been the predominant form at that time. G2 and G2-like forms also circulate in Asia and Brazil, whereas G3 is the major strain found in Africa. Since no complete genomes are available, the PARV4 gene expression profile has been explored provisionally by transfecting its coding sequences into tissue culture cells. Because DNA replication commonly affects parvovirus transcription patterns, these sequences were first inserted between adeno-associated virus 5 (AAV5) terminal hairpins and the resulting hybrid induced to replicate by co-transfection into 293 cells with constructs encoding AAV5Rep78 and adenovirus helper factors (Lou et al., 2012). Two promoters, P6 and P38, were identified, which generate transcripts encoding NS and VP proteins respectively. Some P6 transcripts predicted to initiate in the same frame as NS1 are then spliced into an alternate frame, potentially giving rise to a small NS2 protein, although this product was not detected. Messenger RNAs encoding VP2 and NS1 were observed, generating proteins with molecular masses of ˜80 and ˜65 kDa respectively, but it remains uncertain how the predicted VP1 coding sequences, which include a VP1 specific region of at least 261 amino acids containing a putative PLA2 domain, are accessed.

### Biology

Little is known about the biology or clinical significance of any members of this genus. PARV4 genomes have been detected in plasma during acute infection but often with low viral loads (<3×104 genome copies/ml) and viremia appears protracted, typically lasting from one to several months. Viral genomes have also been detected in some liver and bone marrow samples, although where the virus replicates remains unknown. PARV4 DNA-positive or PARV4 IgG-positive plasmas are rare in the general population of North America and Europe, but occur more frequently in individuals carrying other blood-borne viruses, most notably human immunodeficiency virus, hepatitis B virus or hepatitis C virus, or who have behavioural risk factors for parenteral infection such as intravenous drug use or reliance on hemoderivatives from pooled human plasma (Lahtinen et al., 2011, Sharp et al., 2009). However, in Africa PARV4 is endemic and its epidemiology appears very different, with around 30-50% of the general population in sub-Saharan and South Africa testing seropositive for PARV4 IgG. Viremia is also detected frequently, for example in one study 8.6% of young asymptomatic children in Ghana were DNA-positive (Panning et al., 2010), and viral DNA has been found in nasal and stool samples from African children, suggesting that in this locale transmission is likely by foodborne, respiratory, or contact-mediated routes.

PARV4 genotypes 1 and 2 predominate in Europe and North Africa, whereas G3 is the major form in Africa; thus genetic differences that affect virus biology may contribute to these extreme epidemiological disparities. Alternatively, characteristics of the host population may be critical. Accordingly, much current research is directed at clarifying virus susceptibility and transmission routes. For example, a recent study from Brazil, where the circulating G2 virus might be predicted to follow a parenteral route/high-risk group pattern, revealed that around 6% of individuals infected with human T-cell lymphotropic virus (HTLV) were also positive for PARV4 DNA, but the majority of these co-infected individuals had no history suggesting a parenteral transmission route, indicating that additional factors or routes are likely involved in this locale (Slavov et al., 2017).

Tetraparvoviruses that infect non-human hosts also appear endemic. For example, 63% of chimpanzees and 18% of gorillas from a group of 73 wild-caught apes sampled in Cameroon tested seropositive for antibodies against the chimpanzee virus in species Primate tetraparvovirus 1 (Beierwaltes 1991), while viruses in the 4 ungulate species, Ungulate tetraparvovirus 1, Ungulate tetraparvovirus 2, Ungulate tetraparvovirus 3, and Ungulate tetraparvovirus 4, were commonly detected in the serum and tissues of livestock (Lau et al., 2008, Tse et al., 2011). Similarly, a bat virus (in species Chiropteran tetraparvovirus 1) was detected at high concentration in blood samples and tissues from Eidolon helvum bats in Ghana. This virus was particularly abundant in samples from spleen and kidney, suggesting these organs as likely sites for viral replication (Canuti et al., 2011).

TABLE 2
Exemplar Isolates of Tetraparvovirus

| Species of Tetraparvovirus | Chiropteran tetraparvovirus 1 | |
|---|---|---|
| | Primate tetraparvovirus 1 | |
| | Ungulate tetraparvovirus 1 | |
| | Ungulate tetraparvovirus 2 | |
| | Ungulate tetraparvovirus 3 | |
| | Ungulate tetraparvovirus 4 | |
| | Accession | |
| Exemplary Viruses | No. | Ref Seq No. |
| Eidolon helvum parvovirus | JQ037753 | NC_016744 |
| Human parvovirus 4 | AY622943 | NC_007018 |
| (Genotype 1) | | |
| Human parvovirus 4 | DQ873389.1 | |
| (Taxonomy ID: 289365) | | |
| Human parvovirus 4 | DQ873391.1 | |
| (Genotype 2) | | |
| (Taxonomy ID: 1511920) | | |
| Human parvovirus 4 | EU874248.1 | |
| (Genotype 3) | | |
| Bovine hokovirus 1 | EU200669 | NC_038898 |
| Bovine hokovirus 1 | EU200670.1 | |
| Bovine hokovirus 2 | KU172423.1 | |
| Porcine hokovirus; | EU200677 | NC_038546 |
| Porcine parvovirus 3 | | |
| Porcine parvovirus 2; | GU938300 | NC_038883 |
| Parvovirus YX-2010/CHN | | |
| Porcine Cnvirus | GU938301.1 | |
| (Taxonomy ID: 754189) | | |
| Ovine hokovirus | JF504699 | NC_038547 |
| Chimpanzee Parvovirus 4 | MH215556.1 | |
| (Taxonomy ID: 1511922) | | |
| Yak Parvovirus | | NC_028136 |
| Opossum tetraparvovirus | MG745671 | |
| Rodent tetraparvovirus | MG745670.1 | |
| Tetraparvovirus sp. | | NC_031670.1 |

### Genotypic Variants of Viruses

An ordinarily skilled artisan appreciates that a species of virus comprises clusters of genetic variants (Van Regenmortel MHV (2000) Virus Taxonomy—Seventh Report of the International Committee on Taxonomy of Viruses). Genetic variants may comprise mutations (that encompasses point mutations and insertions-deletions of different lengths), hypermutations, several types of recombination, and genome segment reassortments. Mutation is observed in all viruses, with no known exceptions (Domingo (2019) Virus as Populations 2020:35-71). Recombination is also widespread, and its occurrence was soon accepted for DNA viruses as well as RNA viruses. Genome segment reassortment, a type of variation close to chromosomal exchanges in sexual reproduction, is an adaptive asset of segmented viral genomes, as continuously evidenced by the ongoing evolution of the influenza viruses. The three modes of virus genome variation are compatible, and reassortant-recombinant-mutant genomes are continuously arising in present-day viruses.

Accordingly, the genetic variant of the viruses described herein may comprise a polypeptide described herein or those belonging to a virus described herein (e.g., a capsid protein (VP1, VP2), NS protein, etc.) with an amino acid sequence that is at least, about, or no more than 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to the amino acid sequence of the exemplary sequences presented herein or the amino acid sequence of the polypeptide of the exemplary viruses referenced herein.

### Genomic Safe Harbors (GSHs)

Genomic safe harbors (GSH) are intragenic, intergenic, or extragenic regions of the human and model species genomes that are able to accommodate the predictable expression of newly integrated DNA without significant adverse effects on the host cell or organism. GSHs may comprise intronic or exonic gene sequences as well as intergenic or extragenic sequences. While not being limited to theory, a useful safe harbor must permit sufficient transgene expression to yield desired levels of the transgene-encoded protein or non-coding RNA. A GSH also should not predispose cells to malignant transformation, nor interfere with progenitor cell differentiation, nor significantly alter normal cellular functions. What distinguishes a GSH from a fortuitous good integration event is the predictability of outcome, which is based on prior knowledge and validation of the GSH.

The larger genome size of the recombinant virion described herein allows delivery of a therapeutic transgene(s) together with GSH sequences, which is otherwise not possible with virions having a limited genome size, e.g., AAV. Accordingly, the recombinant virions of the present disclosure not only facilitates delivery of a larger transgene compared with e.g., AAV, but also facilitates a safe delivery of a transgene by allowing codelivery of the GSH sequences that ensures predictable expression of the transgene without adverse effects on the host cells. Exemplary GSHs that have been targeted for transgene addition include (i) the adeno-associated virus site 1 (AAVS1), a naturally occurring, non-germline, site of integration of AAV virus DNA on chromosome 19; (ii) the chemokine (C-C motif) receptor 5 (CCR5) gene, a chemokine receptor gene known as an HIV-1 coreceptor; (iii) the human ortholog of the mouse Rosa26 locus, a locus extensively validated in the murine setting for the insertion of ubiquitously expressed transgenes; and (iv) albumin in murine cells (see, e.g., U.S. Pat. Nos. 7,951,925; 8,771,985; 8,110,379; and 7,951,925; U.S. Patent Publication Nos. 2010/0218264; 2011/0265198; 2013/0137104; 2013/0122591; 2013/0177983; 2013/0177960; 2015/0056705 and 2015/0159172; all of which are incorporated by reference). Additional GSHs include Kif6, Pax5, collagen, HTRP, HI 1 (a thymidine kinase encoding nucleic acid at HI 1 locus), beta-2 microglobulin, GAPDH, TCR, RUNX1, KLHL7, NUPL2 or an intergenic region thereof, mir684, KCNH2, GPNMB, MIR4540, MIR4475, MIR4476, PRL32P21, LOC105376031, LOC105376032, LOC105376030, MELK, EBLN3P, ZCCHC7, RNF38, or loci meeting the criteria of a genome safe harbor as described herein (see e.g., WO 2019/169233 A1, WO 2017/079673 A1; incorporated by reference). These GSHs provide a non-limiting representation of the GSHs that can be used with the recombinant virions described herein. The present disclosure contemplates the use of any GSHs that are known in the art.

In some embodiments, GSH allows safe and targeted gene delivery that has limited off-target activity and minimal risk of genotoxicity, or causing insertional oncogenesis upon integration of foreign DNA, while being accessible to highly specific nucleases with minimal off-target activity.

In some embodiments, GSH has any one or more of the following properties: (i) outside a gene transcription unit; (ii) located between 5-50 kilobases (kb) away from the 5′ end of any gene; (iii) located between 5-300 kb away from cancer-related genes; (iv) located 5-300 kb away from any identified microRNA; and (v) outside ultra-conserved regions and long noncoding RNAs. In some embodiments, a GSH locus has any or more of the following properties: (i) outside a gene transcription unit; (ii) located >50 kilobases (kb) from the 5′ end of any gene; (iii) located >300 kb from cancer-related genes; (iv) located >300 kb from any identified microRNA; and (v) outside ultra-conserved regions and long noncoding RNAs. In studies of lentiviral vector integrations in transduced induced pluripotent stem cells, analysis of over 5,000 integration sites revealed that ˜17% of integrations occurred in safe harbors. The vectors that integrated into these safe harbors were able to express therapeutic levels of β-globin from their transgene without perturbing endogenous gene expression.

In some embodiments, GSH is AAVS1. AAVS1 was identified as the adeno-associated virus common integration site on chromosome 19 and is located in chromosome 19 (position 19ql3.42) and was primarily identified as a repeatedly recovered site of integration of wild-type AAV in the genome of cultured human cell lines that have been infected with AAV in vitro. Integration in the AAVS1 locus interrupts the gene phosphatase 1 regulatory subunit 12C (PPP1R12C; also known as MBS85), which encodes a protein with a function that is not clearly delineated. The organismal consequences of disrupting one or both alleles of PPP1R12C are currently unknown. No gross abnormalities or differentiation deficits were observed in human and mouse pluripotent stem cells harboring transgenes targeted in AAVS1. Originally, AAV DNA integration into AAVS1 site was Rep-dependent, however, there are commercially available CRISPR/Cas9 reagents available for targeting which preserved the functionality of the targeted allele and maintained the expression of PPP1R12C at levels that are comparable to those in non-targeted cells. AAVS1 was also assessed using ZFN-mediated recombination into iPSCs or CD34+ cells.

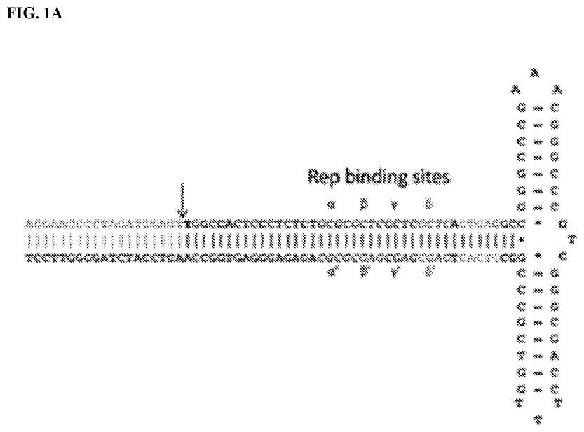

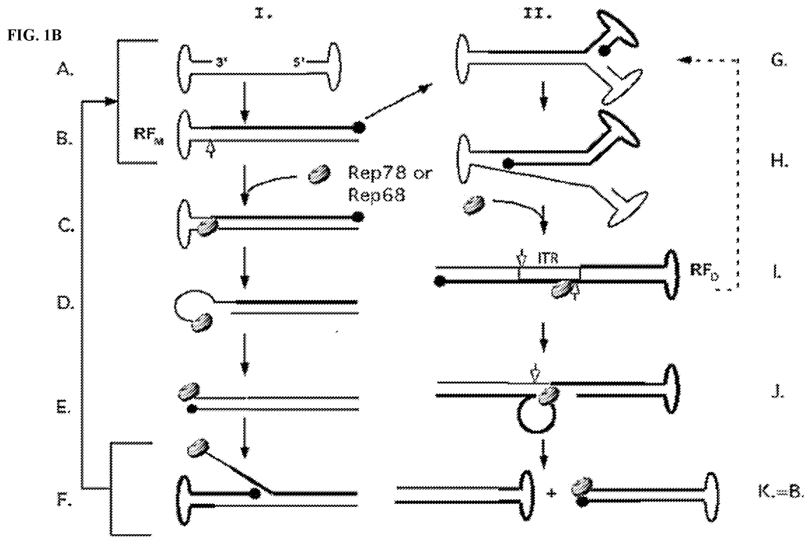

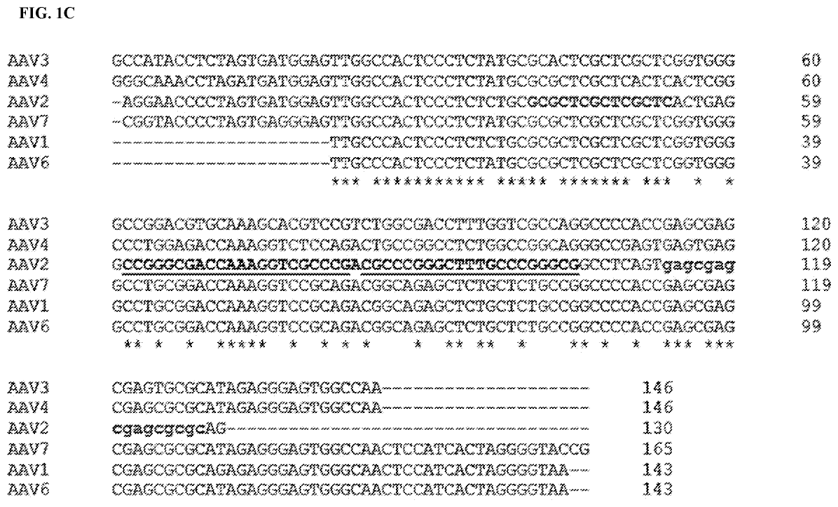

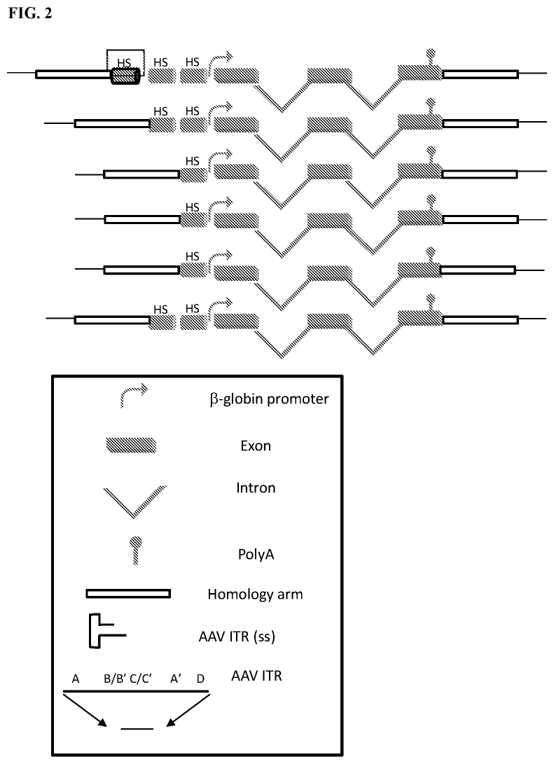

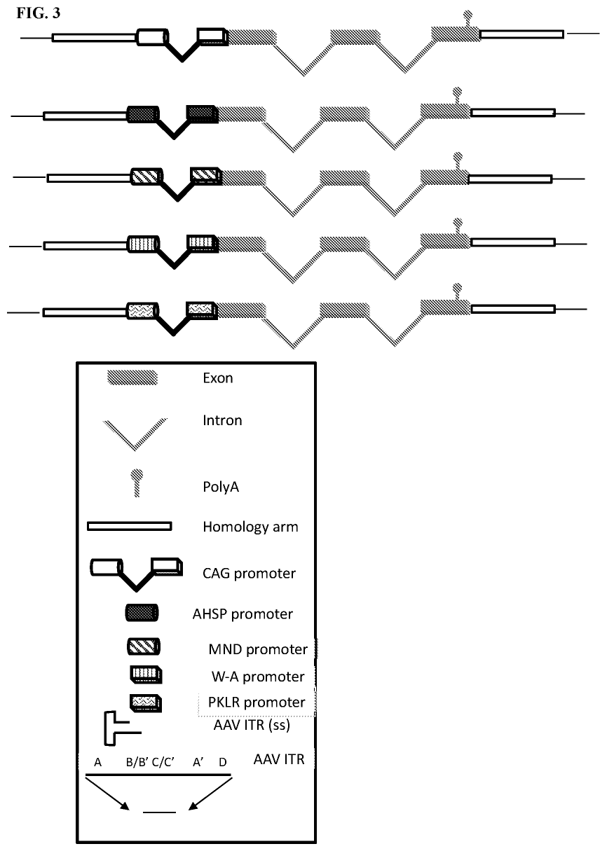

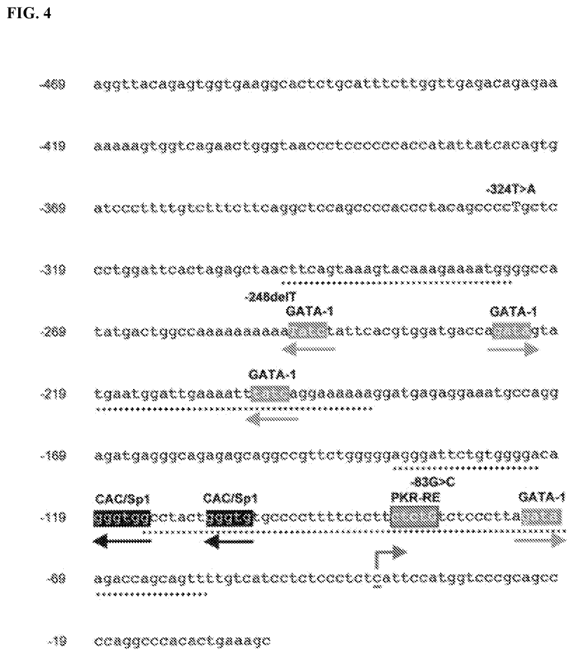

As originally characterized, the AAVS1 locus is >4 kb and is identified as chromosome 19 nucleotides 55,1 13,873-55,1 17,983 (human genome assembly GRCh38/hg38) and overlaps with exon 1 of the PPP1R12C gene that encodes protein phosphatase 1 regulatory subunit 12C. This >4 kb region is extremely G+C nucleotide content rich and is a gene-rich region of particularly gene-rich chromosome 19 (see FIG. 1A of Sadelain et al, Nature Revs Cancer, 2012; 12; 51-58), and some integrated promoters can indeed activate or cis-activate neighboring genes, the consequence of which in different tissues is presently unknown. PPP1R12C exon 1 5′untranslated region contains a functional AAV origin of DNA synthesis indicated within a known sequence (Urcelay et al. 1995).

AAVS1 GSH was identified by characterizing the AAV provirus structure in latently infected human cell lines with recombinant bacteriophage genomic libraries generated from latently infected clonal cell lines (Detroit 6 clone 7374 IIID5) (Kotin and Berns 1989), Kotin et al, isolated non-viral, cellular DNA flanking the provirus and used a subset of “left” and “right” flanking DNA fragments as probes to screen panels of independently derived latently infected clonal cell lines. In approximately 70% of the clonal isolates, AAV DNA was detected with the cell-specific probe (Kotin et al. 1991; Kotin et al. 1990). Sequence analysis of the pre-integration site identified near homology to a portion of the AAV inverted terminal repeat (Kotin, Linden, and Beems 1992). Although lacking the characteristic interrupted palindrome, the AAVS1 locus retained the Rep binding elements and terminal resolution sites homologous to the AAV ITR (FIG. 1A).

The selection of the exonic integration site is non-obvious, and perhaps counter-intuitive, since insertion and expression of foreign DNA likely disrupts the expression of the endogenous genes. Apparently, insertion of the AAV genome into this locus does not adversely affect cell viability or iPSC differentiation (DeKelver et al. 2010; Wang et al. 2012; Zou et al. 2011). The AAVS1 locus is within the 5′ UTR of the highly conserved PPP1R12C gene. The Rep-dependent minimal origin of DNA synthesis is conserved in the 5′UTR of the human, chimapanzee, and gorilla PPP1R12C gene. However, the commercially available CRISPR/Cas9 reagents used for integrating DNA into AAVS1 target PPP1R12C intron 1 rather than the exon.

In some embodiments, GSH is any one of Kif6, Pax5, collagen, HTRP, HI 1, beta-2 microglobulin, GAPDH, TCR, RUNX1, KLHL7, an intergenic region of NUPL2, mir684, KCNH2, GPNMB, MIR4540, MIR4475, MIR4476, PRL32P21, LOC105376031, LOC105376032, LOC105376030, MELK, EBLN3P, ZCCHC7, and RNF38.

In some embodiments, GSH is the Pax 5 gene gene (also known as Paired Box 5, or “B-cell lineage specific activator protein,” or BSAP). In humans PAX5 is located on chromosome 9 at 9p 13.2 and has orthologues across many vertebrate species, including, human, chimp, macaque, mouse, rat, dog, horse, cow, pig, opossum, platypus, chicken, lizard, xenopus, C. elegans, drosophila and zebrafish. PAX5 gene is located at Chromosome 9: 36,833,275-37,034,185 reverse strand (GRCh38:CM000671.2) or 36,833,272-37,034,182 in GRCh37 coordinates.

Additional exemplary GSHs are listed in Table 3A and Table 3B.

TABLE 3A
Exemplary GSH loci in Homo Sapiens (see, e.g., WO 2019/169232;
incorporated by reference)

| Gene | Chromosomal location | Accession number/location |
|---|---|---|
| PAX5 | Chromosome 9: | NC_000009.12 (36833274 ... 37035949, complement) |
| | 36,833,275-37,034,185 | |
| | reverse strand | |
| MIR4540 | | NC_000009.12 (36864254 ... 36864308, complement) |
| MIR4475 | GRCh38.p7 | NC_000009.12 (36823539 ... 36823599, complement) |
| | (GCF_000001405.33) | |
| MIR4476 | GRCh38.p7 | NC_000009.12 (36893462 ... 36893531, complement) |
| | (GCF_000001405.33) | |
| PRL32P21 | GRCh38.p7 | NC_000009.12 (37046835 ... 37047242) |
| | (GCF_000001405.33) | |
| LOC105376031 | GRCh38.p7 | NC_000009.12 (37027763 ... 37031333) |
| | (GCF_000001405.33) | |
| LOC105376032 | GRCh38.p7 | NC_000009.12 (37002697 ... 37007774) |
| | (GCF_000001405.33) | |
| LOC105376030 | GRCh38.p7 | NC_000009.12 (36779475 ... 36830456) |
| | (GCF_000001405.33) | |
| MELK | GRCh38.p7 | NC_000009.12 (36572862 ... 36677683) |
| | (GCF_000001405.33) | |
| EBLN3P | GRCh38.p7 | NC_000009.12 (37079896 ... 37090401) |
| | (GCF_000001405.33) | |
| ZCCHC7 | GRCh38.p7 | NC_000009.12 (37120169 ... 37358149) |
| | (GCF_000001405.33) | |
| RNF38 | GRCh38.p7 | NC_000009.12 (36336398 ... 36487384, complement) |
| | (GCF_000001405.33) | |

TABLE 3B
Exemplary GSH loci (see, e.g., WO 2019/169232; incorporated by reference)

| | | | |
|---|---|---|---|
| Taxonomic | | | |
| Rank | Brief description | Species | Chromosomal location |

Intergenic Loci

| | | | |
|---|---|---|---|
| Macropoddae | - mAAV_eve integration between | M. domestica | chromosome 1: |
| (lanonomic | cadherin (cdh) 8 and cdh 16. | | cdh 8:674,639,xxxi675.163,xxx |
| rank: Family) | Because the macropod genome is | | cdh 10: 680,370,7xx-680,581,xxx |
| | poorly annotated, another | | Intergenic distance = 5.2 Mb |
| | marsupial Mondelphis domestica | | Empty EVE locus in M. domestica |
| | with more completely assemble | | 674,422,470-675-422,729 |
| | genome is used as a substitute | | |
| | genome. | | |
| | | Mouse | ch 9 |
| | | | cdh 8: 99,028,769-99,416,471 |
| | | | cdh11: 192,632,055-102,785,111 |
| | | | Intergenic distance = 3.2 Mb |
| | | Homo sapiens | chromosome 18 |
| | | | cdh 8: 61,647,242-62,036,835 |
| | | | cdh 11: 64,943,753-65,122,198 |
| | | | Intergenic distance = 2.9 Mb |
| Lepondae | Lepondae EVE located between | H. Sapiens | Chromosome 7: |
| (Family)-the | NUPL2 and GPNMB | | -KLH7->NUPL2-)min684-KCNH2 |
| Family | The gene order is: | M. mus | -KLH7->NUPL2-)min684-KCNH2 |
| Lepondae are | -Fam126A-KLH7---NUPL2--- | | |
| rabbits and | -EVE---GPNMB---IGF28P3- | | |
| hares species | MALSU1 | | |
| of the | | | |
| Lagomorph | | | |
| Order. | | | |

Intragenic loci

| | | | |
|---|---|---|---|
| Cetauea | EVE integrated into an intron of | H. sapiens | chromosome 9: |
| (Order) | PAX5 | | (Pax5)36,833.275-37,034,185 |
| | | M. mus | Chromosome 4: |
| | | | (Pax5)44,531,506-44,710,440 |
| (Family- | Myotis EVE integrated int the K38 | H. sapien | Chromosome 6: |
| Vaspetitionidae | gene, intronic or exonic | | (KH6)36,329,990-39,725,405 |
| Order- | | M. mus | Chromosome 17: |
| Chiroptera | | | (KH6)49,754,497-50,049,172 |
| Myotis | | | |
| (Genus) | | | |
| Myotinae | | | |
| (Subfamily) | | | |

Integration to a Target Genome Integration to the target genome may be driven by cellular processes, such as homologous recombination or non-homologous end-joining (NHEJ). The integration may also be initiated and/or facilitated by an exogenously introduced nuclease. In preferred embodiments, the nucleic acid packaged within the recombinant virions described herein is integrated to a specific locus within the genome, e.g., GSH. In some embodiments, the GSH is any locus that permits sufficient transgene expression to yield desired levels of the transgene-encoded protein or non-coding RNA. A GSH also should not predispose cells to malignant transformation nor significantly alter normal cellular functions. The site-specific integration to a GSH may be mediated by the nucleic acid homologous to the GSH that is placed 5′ and 3′ to the nucleic acid to be integrated. Such homologous donor sequences may provide a template for homology-dependent repair that allows integration at the desired locus.

In preferred embodiments, the recombinant virion described herein comprises a nucleic acid comprising a nucleic acid sequence that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to the nucleic acid sequence of a genomic safe harbor (GSH) of the target cell. In some embodiments, the said nucleic acid that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to a GSH is placed 5′ and 3′ (homology arms) to a nucleic acid to be integrated, thereby allowing insertion (of the nucleic acid located between the homology arms) to a specific locus in the target genome by homologous recombination. In some embodiments, the nucleic acid to be integrated is any one of the nucleic acids operably linked to a promoter described herein. In some embodiments, the GSH is AAVS1, ROSA26, CCR5, Kif6, Pax5, an intergenic region of NUPL2, collagen, HTRP, HI 1 (a thymidine kinase encoding nucleic acid at HI 1 locus), beta-2 microglobulin, GAPDH, TCR, RUNX1, KLHL7, mir684, KCNH2, GPNMB, MIR4540, MIR4475, MIR4476, PRL32P21, LOC105376031, LOC105376032, LOC105376030, MELK, EBLN3P, ZCCHC7, or RNF38. In some embodiments, the GSH is AAVS1, ROSA26, CCR5, Kif6, Pax5, or an intergenic region of NUPL2.

In certain embodiments, the nucleic acid of the recombinant virion is integrated into the genome of a target cell upon transduction. In some embodiments, the nucleic acid is integrated into a GSH or EVE. In some embodiments, the GSH is AAVS1, ROSA26, CCR5, Kif6, Pax5, an intergenic region of NUPL2, collagen, HTRP, HI 1 (a thymidine kinase encoding nucleic acid at HI 1 locus), beta-2 microglobulin, GAPDH, TCR, RUNX1, KLHL7, mir684, KCNH2, GPNMB, MIR4540, MIR4475, MIR4476, PRL32P21, LOC105376031, LOC105376032, LOC105376030, MELK, EBLN3P, ZCCHC7, or RNF38. In some embodiments, the GSH is AAVS1, ROSA26, CCR5, Kif6, Pax5, or an intergenic region of NUPL2. In some embodiments, the nucleic acid is integrated into the target genome by homologous recombination followed by a DNA break formation induced by an exogenously-introduced nuclease. In some embodiments, the nuclease is TALEN, ZFN, a meganuclease, a megaTAL, or a CRISPR endonuclease (e.g., a Cas9 endonuclease or a variant thereof). In some embodiments, the CRISPR endonuclease is in a complex with a guide RNA.

In certain aspects, provided herein are methods of integrating a heterologous nucleic acid into a GSH in a cell, comprising: (a) transducing the cell with one or more virions described herein comprising a heterologous nucleic acid flanked at the 5′ end and 3′ end by a donor nucleic acid sequence that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to the target GSH nucleic acid; or (b) transducing the cell with one or more virions described herein comprising (i) a heterologous nucleic acid flanked at the 5′ end and 3′ end by a donor nucleic acid sequence that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to the target GSH nucleic acid, and (ii) a nucleic acid encoding a nuclease (e.g., Cas9 or a variant thereof, ZFN, TALEN) and/or a guide RNA, wherein the nuclease or the nuclease/gRNA complex makes a DNA break at the GSH, which is repaired using the donor nucleic acid, thereby integrating a heterologous nucleic acid at GSH. In some embodiments, (i) the heterologous nucleic acid flanked by a donor nucleic acid that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to the target GSH nucleic acid and (ii) the nucleic acid encoding a nuclease and/or the gRNA are transduced in separate virions. In some embodiments, the GSH is AAVS1, ROSA26, CCR5, Kif6, Pax5, an intergenic region of NUPL2, collagen, HTRP, HI 1 (a thymidine kinase encoding nucleic acid at HI 1 locus), beta-2 microglobulin, GAPDH, TCR, RUNX1, KLHL7, mir684, KCNH2, GPNMB, MIR4540, MIR4475, MIR4476, PRL32P21, LOC105376031, LOC105376032, LOC105376030, MELK, EBLN3P, ZCCHC7, or RNF38. In some embodiments, the GSH is AAVS1, ROSA26, CCR5, Kif6, Pax5, or an intergenic region of NUPL2.

For integration of the nucleic acid located between the 5′ and 3′ homology arms, the 5′ and 3′ homology arms should be long enough for targeting to the GSH and allow (e.g., guide) integration into the genome by homologous recombination. To increase the likelihood of integration at a precise location and enhance the probability of homologous recombination, the 5′ and 3′ homology arms may include a sufficient number of nucleic acids. In some embodiments, the 5′ and 3′ homology arms may include at least 10 base pairs but no more than 5,000 base pairs, at least 50 base pairs but no more than 5,000 base pairs, at least 100 base pairs but no more than 5,000 base pairs, at least 200 base pairs but no more than 5,000 base pairs, at least 250 base pairs but no more than 5,000 base pairs, or at least 300 base pairs but no more than 5,000 base pairs. In some embodiments, the 5′ and 3′ homology arms include about 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, or 500 base pairs. Detailed information regarding the length of homology arms and recombination frequency is art-known, see e.g., Zhang et al. “Efficient precise knock in with a double cut HDR donor after CRISPR/Cas9-mediated double-stranded DNA cleavage.” Genome biology 18.1 (2017): 35, which is incorporated herein in its entirety by reference.

The 5′ and 3′ homology arms may be any sequence that is homologous with the GSH target sequence in the genome of the host cell. In some embodiments, the 5′ and 3′ homology arms may be homologous to portions of the GSH described herein. Furthermore, the 5′ and 3′ homology arms may be non-coding or coding nucleotide sequences.

In some embodiments, the 5′ and/or 3′ homology arms can be homologous to a sequence immediately upstream and/or downstream of the integration or DNA cleavage site on the chromosome. Alternatively, the 5′ and/or 3′ homology arms can be homologous to a sequence that is distant from the integration or DNA cleavage site, such as at least 1, 2, 5, 10, 15, 20, 25, 30, 50, 75, 100, 125, 150, 175, 200, 225, 250, 275, 300, 325, 350, 375, 400, 425, 450, 475, 500 or more base pairs away from the integration or DNA cleavage site, or partially or completely overlapping with the DNA cleavage site (e.g., can be a DNA break induced by an exogenously-introduced nuclease). In some embodiments, the 3′ homology arm of the nucleotide sequence is proximal to an ITR.

### Gene-Editing Systems

In some embodiments, the methods and compositions described herein are used to integrate a nucleic acid delivered by a recombinant virion described herein into any specific locus (e.g., GSH) within the target genome. In some embodiments, the integration is initiated and/or facilitated by an exogenously introduced nuclease, and the DNA break induced by the nuclease is repaired using the homology arms as a guide for homologous recombination, thereby inserting the nucleic acid flanked by the said homology arms into the target genome.

For example, a double-strand break (DSB) for can be created by a site-specific nuclease such as a zinc-finger nuclease (ZFN) or TAL effector domain nuclease (TALEN). See, for example, Urnov et al. (2010) Nature 435(7042):646-51; U.S. Pat. Nos. 8,586,526; 6,534,261; 6,599,692; 6,503,717; 6,689,558; 7,067,317; 7,262,054, the disclosures of which are incorporated by reference.

Another nuclease system involves the use of a so-called acquired immunity system found in bacteria and archaea known as the CRISPR/Cas system. CRISPR/Cas systems are found in 40% of bacteria and 90% of archaea and differ in the complexities of their systems. See, e.g., U.S. Pat. No. 8,697,359. The CRISPR loci (clustered regularly interspaced short palindromic repeat) are regions within the organism's genome where short segments of foreign DNA are integrated between short repeat palindromic sequences. These loci are transcribed and the RNA transcripts (“pre-crRNA”) are processed into short CRISPR RNAs (crRNAs). There are three types of CRISPR/Cas systems which all incorporate these RNAs and proteins known as “Cas” proteins (CRISPR associated). Types I and III both have Cas endonucleases that process the pre-crRNAs, that, when fully processed into crRNAs, assemble a multi-Cas protein complex that is capable of cleaving nucleic acids that are complementary to the crRNA.

In type II systems, crRNAs are produced using a different mechanism where a trans-activating RNA (tracrRNA) complementary to repeat sequences in the pre-crRNA, triggers processing by a double strand-specific RNase III in the presence of the Cas9 protein or a variant thereof Cas9 is then able to cleave a target DNA that is complementary to the mature crRNA however cleavage by Cas9 is dependent both upon base-pairing between the crRNA and the target DNA, and on the presence of a short motif in the crRNA referred to as the PAM sequence (protospacer adjacent motif) (see Qi et al (2013) Cell 152: 1173). In addition, the tracrRNA must also be present as it base pairs with the crRNA at its 3′ end, and this association triggers Cas9 activity.

The Cas9 protein has at least two nuclease domains: one nuclease domain is similar to a HNH endonuclease, while the other resembles a Ruv endonuclease domain. The HNH-type domain appears to be responsible for cleaving the DNA strand that is complementary to the crRNA while the Ruv domain cleaves the non-complementary strand. The variants of Cas9 are art-recognized, e.g., Cas9 nickase mutant that reduces off-target activity (see e.g., Ran et al. (2014) Cell 154(6): 1380-1389), nCas, Cas9-D10A.

The requirement of the crRNA-tracrRNA complex can be avoided by use of an engineered “single-guide RNA” (sgRNA) that comprises the hairpin normally formed by the annealing of the crRNA and the tracrRNA (see Jinek et al (2012) Science 337:816 and Cong et al (2013) Sciencexpress/10.1126/science.1231143). Thus, exogenously introduced CRISPR endonuclease (e.g., Cas9 or a variant thereof) and a guide RNA (e.g., sgRNA or gRNA) can induce a DNA break at a specific locus within the genome of a target cell. Non-limiting examples of single-guide RNA or guide RNA (sgRNA or gRNA) sequences suitable for targeting are shown in Table 1 in US Application 2015/0056705, which is incorporated herein in its entirety by reference. In addition, a sgRNA or gRNA may comprise a sequence of GSH loci described herein, including those in Table 3A and Table 3B.

In some embodiments, the gene editing nucleic acid sequence encodes a gene editing nucleic acid molecule selected from the group consisting of: a sequence specific nuclease, one or more guide RNA (gRNA), CRISPR/Cas, a ribonucleoprotein (RNP) or any combination thereof. In some embodiments, the sequence-specific nuclease comprises: a TAL-nuclease, a zinc-finger nuclease (ZFN), a meganuclease, a megaTAL, or an RNA guide endonuclease of a CRISPR/Cas system (e.g., Cas proteins e.g. CAS 1-9, Csy, Cse, Cpfl, Cmr, Csx, Csf, cpfl, nCAS, or others). These gene editing systems are known to those of skill in the art, See for example, TALENS described in International Patent Application No. PCT/US2013/038536, and U.S. Patent Publication No. 2017-0191078-A9 which are incorporated by reference in their entirety. CRISPR cas9 systems are known in the art and described in U.S. patent application Ser. No. 13/842,859 filed on March 2013, and U.S. Pat. Nos. 8,697,359, 8,771,945, 8,795,965, 8,865,406, 8,871,445. The recombinant virion described herein is also useful for deactivated nuclease systems, such as CRISPRi or CRISPRa dCas systems, nCas, or Cas13 systems.

Guide RNAs (gRNAS)

In general, a guide sequence is any polynucleotide sequence having sufficient complementarity with a target polynucleotide sequence to hybridize with the target sequence and direct sequence-specific targeting of an RNA-guided endonuclease complex to the selected genomic target sequence. In some embodiments, a guide RNA binds to a target sequence and e.g., a CRISPR associated protein that can form a ribonucleoprotein (RNP), for example, a CRISPR/Cas complex.

In some embodiments, the guide RNA (gRNA) sequence comprises a targeting sequence that directs the gRNA sequence to a desired site in the genome, is fused to a crRNA and/or tracrRNA sequence that permit association of the guide sequence with the RNA-guided endonuclease. In some embodiments, the degree of complementarity between a guide sequence and its corresponding target sequence, when optimally aligned using a suitable alignment algorithm, is at least 50%, 60%, 75%, 80%, 85%, 90%, 95%, 97.5%, 99%, or more. Optimal alignment can be determined with the use of any suitable algorithm for aligning sequences, such as the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif), SOAP, and Maq.

A guide sequence can be selected to target any target sequence. In some embodiments, the target sequence is a sequence within a genome of a cell or within a GSH as disclosed herein. In some embodiments, the guide RNA can be complementary to either strand of the targeted DNA sequence. It is appreciated by one of skill in the art that for the purposes of targeted cleavage by an RNA-guided endonuclease, target sequences that are unique in the genome are preferred over target sequences that occur more than once in the genome. Bioinformatics software can be used to predict and minimize off-target effects of a guide RNA (see e.g., Naito et al. “CRISPRdirect: software for designing CRISPR/Cas guide RNA with reduced off-target sites” Bioinformatics (2014), epub; Heigwer et al. “E-CRISP: fast CRISPR target site identification” Nat. Methods 11:122-123 (2014); Bae et al. “Cas-OFFinder: a fast and versatile algorithm that searches for potential off-target sites of Cas9 RNA-guided endonucleases” Bioinformatics 30(10): 1473-1475 (2014); Aach et al. “CasFinder: Flexible algorithm for identifying specific Cas9 targets in genomes” BioRxiv (2014)).

In general, a “crRNA/tracrRNA fusion sequence,” as that term is used herein refers to a nucleic acid sequence that is fused to a unique targeting sequence and that functions to permit formation of a complex comprising the guide RNA and the RNA-guided endonuclease. Such sequences can be modeled after CRISPR RNA (crRNA) sequences in prokaryotes, which comprise (i) a variable sequence termed a “protospacer” that corresponds to the target sequence as described herein, and (ii) a CRISPR repeat. Similarly, the tracrRNA (“transactivating CRISPR RNA”) portion of the fusion can be designed to comprise a secondary structure similar to the tracrRNA sequences in prokaryotes (e.g., a hairpin), to permit formation of the endonuclease complex. In some embodiments, the single transcript further includes a transcription termination sequence, such as a polyT sequence, for example six T nucleotides. In some embodiments, a guide RNA can comprise two RNA molecules and is referred to herein as a “dual guide RNA” or “dgRNA.” In some embodiments, the dgRNA may comprise a first RNA molecule comprising a crRNA, and a second RNA molecule comprising a tracrRNA. The first and second RNA molecules may form a RNA duplex via the base pairing between the flagpole on the crRNA and the tracrRNA. When using a dgRNA, the flagpole need not have an upper limit with respect to length.

In other embodiments, a guide RNA can comprise a single RNA molecule and is referred to herein as a “single guide RNA” or “sgRNA.” In some embodiments, the sgRNA can comprise a crRNA covalently linked to a tracrRNA. In some embodiments, the crRNA and tracrRNA can be covalently linked via a linker. In some embodiments, the sgRNA can comprise a stem-loop structure via the base-pairing between the flagpole on the crRNA and the tracrRNA. In some embodiments, a single-guide RNA is at least 50, at least 60, at least 70, at least 80, at least 90, at least 100, at least 110, at least 120 or more nucleotides in length (e.g., 75-120, 75-110, 75-100, 75-90, 75-80, 80-120, 80-110, 80-100, 80-90, 85-120, 85-110, 85-100, 85-90, 90-120, 90-110, 90-100, 100-120, 100-120 nucleotides in length). In some embodiments, a nucleic acid vector as described herein for integration of a nucleic acid of interest into a GSH loci, or composition thereof comprises a nucleic acid that encodes at least 1 gRNA. For example, the second polynucleotide sequence may encode between 1 gRNA and 50 gRNAs, or any integer from 1-50. Each of the polynucleotide sequences encoding the different gRNAs can be operably linked to a promoter. In some embodiments, the promoters that are operably linked to the different gRNAs may be the same promoter. The promoters that are operably linked to the different gRNAs may be different promoters. The promoter may be a constitutive promoter, an inducible promoter, a repressible promoter, or a regulatable promoter.

In some embodiments, a nucleic acid for integration into a GSH locus encodes or the recombinant virion comprising the said nucleic acid is administered in conjunction with another virion comprising a nucleic acid that encodes a Cas nickase (nCas; e.g., Cas9 nickase or Cas9-D10A). It is contemplated herein that such an nCas enzyme is used in conjunction with a guide RNA that comprises homology to a GSH as described herein and can be used, for example, to release physically constrained sequences or to provide torsional release. Releasing physically constrained sequences can, for example, “unwind” the vector such that a homology directed repair (HDR) template homology arm(s) are exposed for interaction with the genomic sequence.

In some embodiments, zinc finger nuclease is used to induce a DNA break that facilitates integration of the desired nucleic acid. “Zinc finger nuclease” or “ZFN” as used interchangeably herein refers to a chimeric protein molecule comprising at least one zinc finger DNA binding domain effectively linked to at least one nuclease or part of a nuclease capable of cleaving DNA when fully assembled. “Zinc finger” as used herein refers to a protein structure that recognizes and binds to DNA sequences. The zinc finger domain is the most common DNA-binding motif in the human proteome. A single zinc finger contains approximately 30 amino acids and the domain typically functions by binding 3 consecutive base pairs of DNA via interactions of a single amino acid side chain per base pair.

In some embodiments, a nucleic acid for integration described herein is integrated into a target genome in a nuclease-free homology-dependent repair systems, e.g., as described in Porro et al., Promoterless gene targeting without nucleases rescues lethality of a Crigler-Najjar syndrome mouse model, EMBO Molecular Medicine, (2017). In some embodiments, the in vivo gene targeting approaches are suitable for the insertion of a donor sequence, without the use of nucleases. In some embodiments, the donor sequence may be promoterless.

In some embodiments, the nuclease located between the restriction sites can be a RNA-guided endonuclease. As used herein, the term “RNA-guided endonuclease” refers to an endonuclease that forms a complex with an RNA molecule that comprises a region complementary to a selected target DNA sequence, such that the RNA molecule binds to the selected sequence to direct endonuclease activity to a selected target DNA sequence in a GSH identified herein.

### CRISPR CAS Systems

As art-recognized and described above, a CRISPR-CAS9 system includes a combination of protein and ribonucleic acid (“RNA”) that can alter the genetic sequence of an organism (see, e.g., US publication 2014/0170753). CRISPR-Cas9 provides a set of tools for Cas9-mediated genome editing via nonhomologous end joining (NHEJ) or homologous recombination in mammalian cells. One of ordinary skill in the art may select between a number of known CRISPR systems such as Type I, Type II, and Type III. In some embodiments, a nucleic acid described herein for integration of a nucleic acid of interest into a GSH loci can be designed to include the sequences encoding one or more components of these systems such as the guide RNA, tracrRNA, or Cas (e.g., Cas9 or a variant thereof). In certain embodiments, a single promoter drives expression of a guide sequence and tracrRNA, and a separate promoter drives Cas (e.g., Cas9 or a variant thereof) expression. One of skill in the art will appreciate that certain Cas nucleases require the presence of a protospacer adjacent motif (PAM) adjacent to a target nucleic acid sequence.

RNA-guided nucleases including Cas (e.g., Cas9 or a variant thereof) are suitable for initiating and/or facilitating the integration of a nucleic acid delivered by a recombinant virion described herein. The guide RNAs can be directed to the same strand of DNA or the complementary strand.

In some embodiments, the methods and compositions described herein can comprise and/or be used to deliver CRISPRi (CRISPR interference) and/or CRISPRa (CRISPR activation) systems to a host cell. CRISPRi and CRISPRa systems comprise a deactivated RNA-guided endonuclease (e.g., Cas9 or a variant thereof) that cannot generate a double strand break (DSB). This permits the endonuclease, in combination with the guide RNAs, to bind specifically to a target sequence in the genome and provide RNA-directed reversible transcriptional control.

Accordingly, in some embodiments, the nucleic acid compositions and methods described herein for integration of a nucleic acid of interest into a GSH locus can comprise a deactivated endonuclease, e.g., RNA-guided endonuclease and/or Cas9 or a variant thereof, wherein the deactivated endonuclease lacks endonuclease activity, but retains the ability to bind DNA in a site-specific manner, e.g., in combination with one or more guide RNAs and/or sgRNAs. In some embodiments, the vector can further comprise one or more tracrRNAs, guide RNAs, or sgRNAs. In some embodiments, the de-activated endonuclease can further comprise a transcriptional activation domain.

In some embodiments, the nucleic acid compositions and methods described herein for integration of a nucleic acid of interest into a GSH locus can comprise a hybrid recombinase. For example, Hybrid recombinases based on activated catalytic domains derived from the resolvase/invertase family of serine recombinases fused to Cys2-His2 zinc-finger or TAL effector DNA-binding domains are a class of reagents capable improved targeting specificity in mammalian cells and achieve excellent rates of site-specific integration. Suitable hybrid recombinases include those described in Gaj et al. Enhancing the Specificity of Recombinase-Mediated Genome Engineering through Dimer Interface Redesign, Journal of the American Chemical Society, (2014).

The nucleases described herein can be altered, e.g., engineered to design sequence specific nuclease (see, e.g., U.S. Pat. No. 8,021,867). Nucleases can be designed using the methods described in e.g., Certo et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8,163,514, the contents of each are incorporated herein by reference in their entirety. Alternatively, nuclease with site specific cutting characteristics can be obtained using commercially available technologies e.g., Precision BioSciences' Directed Nuclease Editor™ genome editing technology.

### MegaTALs

In some embodiments, the nuclease described herein can be a megaTAL. MegaTALs are engineered fusion proteins which comprise a transcription activator-like (TAL) effector domain and a meganuclease domain. MegaTALs retain the ease of target specificity engineering of TALs while reducing off-target effects and overall enzyme size and increasing activity. MegaTAL construction and use is described in more detail in, e.g., Boissel et al. 2014 Nucleic Acids Research 42(4):2591-601 and Boissel 2015 Methods Mol Biol 1239: 171-196. Protocols for megaTAL-mediated gene knockout and gene editing are known in the art, see, e.g., Sather et al. Science Translational Medicine 2015 7(307):ral56 and Boissel et al. 2014 Nucleic Acids Research 42(4):2591-601. MegaTALs can be used as an alternative endonuclease in any of the methods and compositions described herein.

### Marker/Reporter Genes

Exemplary marker genes include but not limited to any of fluorescent reporter genes, e.g., GFP, RFP and the like, as well as bioluminescence reporter genes. Exemplary marker genes include, but are not limited to, glutathione-S-transferase (GST), horseradish peroxidase (HRP), chloramphenicol acetyltransferase (CAT) beta-galactosidase, beta-glucuronidase, luciferase, green fluorescent proteins (e.g., GFP, GFP-2, tagGFP, turboGFP, sfGFP, EGFP, Emerald, Azami Green, Monomeric Azami Green, CopGFP, AceGFP, ZsGreenl), HcRed, DsRed, cyan fluo-rescent protein (CFP), yellow fluorescent proteins (e.g., YFP, EYFP, Citrine, Venus YPet, PhiYFP, ZsYellowl), cyan fluorescent proteins (e.g., ECFP, Cerulean, CyPet AmCyanl, Midoriishi-Cyan) red fluorescent proteins (e.g., mKate, mKate2, mPlum, DsRed monomer, mCherry, mRFPl, DsRed-Express, DsRed2, HcRed-Tandem, HcRed 1, AsRed2, eqFP6l 1, mRaspberry, mStrawberry, Jred), orange fluorescent proteins (e.g., mOrange, mKO, Kusabira-Orange, monomeric Kusabira-Orange, mTangerine, tdTomato) and autofluorescent proteins including blue fluorescent protein (BFP).

Marker genes may also include, without limitation, DNA sequences encoding β-lactamase, β-galactosidase (LacZ), alkaline phosphatase, thymidine kinase, green fluorescent protein (GFP), chloramphenicol acetyltransferase (CAT), luciferase, and others well known in the art. When associated with regulatory elements which drive their expression, the reporter sequences, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. For example, where the marker sequence is the LacZ gene, the presence of the vector carrying the signal is detected by assays for β-galactosidase activity. In some embodiments, where the marker gene is green fluorescent protein or luciferase, the vector carrying the signal may be measured colorimetrically based on visible light absorbance or light production in a luminometer, respectively. Such reporters can, for example, be useful in verifying the tissue-specific targeting capabilities and tissue specific promoter regulatory activity of a nucleic acid.

Marker genes include, but are not limited to, sequences encoding proteins that mediate antibiotic resistance (e.g., ampicillin resistance, neomycin resistance, G418 resistance, puromycin resistance), sequences encoding colored or fluorescent or luminescent proteins (e.g., green fluorescent protein, enhanced green fluorescent protein, red fluorescent protein, luciferase), and proteins which mediate cellular metabolism resulting in enhanced cell growth rates and/or gene amplification (e.g., dihydrofolate reductase).

### Nucleic Acids

### Non-Coding RNA & Coding RNA

In some embodiments, the nucleic acid of interest encodes a receptor, toxin, a hormone, an enzyme, a marker protein encoded by a marker gene (see above), or a cell surface protein or a therapeutic protein, peptide or antibody or fragment thereof. In some embodiments, a nucleic acid of interest for use in the vector compositions as disclosed herein encodes any polypeptide of which expression in the cell is desired, including, but not limited to antibodies, antigens, enzymes, receptors (cell surface or nuclear), hormones, lymphokines, cytokines, reporter polypeptides, growth factors, and functional fragments of any of the above.

In some embodiments, a nucleic acid of interest for use in the recombinant virion as disclosed herein encodes a polypeptide that is lacking or non-functional in the subject having a disease, including but not limited to any of the diseases described herein. In some embodiments, the disease is a genetic disease.

In some aspects, a nucleic acid of interest as defined herein encodes a nucleic acid for use in methods of preventing or treating one or more genetic deficiencies or dysfunctions in a mammal, such as for example, a polypeptide deficiency or polypeptide excess in a mammal, and particularly for preventing, treating or reducing the severity or extent of deficiency in a human manifesting one or more of the disorders linked to a deficiency in such polypeptides in cells and tissues. The method involves administration of the nucleic acid of interest (e.g., a nucleic acid as described by the disclosure) that encodes one or more therapeutic peptides, polypeptides, siRNAs, microRNAs, antisense nucleotides, etc. packaged in the recombinant virion described herein, preferably in a pharmaceutically acceptable composition, to the subject in an amount and for a period of time sufficient to prevent or treat the deficiency or disorder in the subject suffering from such a disorder.

Thus, in some embodiments, nucleic acids of interest for use in the vector compositions as disclosed herein can encode one or more peptides, polypeptides, or proteins, which are useful for the treatment or prevention of a disease in a mammalian subject.

Exemplary nucleic acids of interest for use in the compositions and methods as disclosed herein include but not limited to: BDNF, CNTF, CSF, EGF, FGF, G-SCF, GM-CSF, gonadotropin, IFN, IFG-1, M-CSF, NGF, PDGF, PEDF, TGF, VEGF, TGF-B2, TNF, prolactin, somatotropin, XIAP1, IL-1, IL-2, IL-3, IL-4, IL-5, IL-6, IL-7, IL-8, IL-9, IL-10, IL-10(187A), viral IL-10, IL-11, IL-12, IL-13, IL-14, IL-15, IL-16, IL-17, IL-18, VEGF, FGF, SDF-1, connexin 40, connexin 43, SCN4a, HIFia, SERCa2a, ADCY1, and ADCY6.

In some embodiments, the nucleic acid may comprise a coding sequence or a fragment thereof selected from the group consisting of a mammalian R globin gene (e.g., HBA1, HBA2, HBB, HBG1, HBG2, HBD, HBE1, and/or HBZ), alpha-hemoglobin stabilizing protein (AHSP), a B-cell lymphoma/leukemia 11 A (BCL11A) gene, a Kruppel-like factor 1 (KLF1) gene, a CCR5 gene, a CXCR4 gene, a PPP1R12C (AAVS1) gene, an hypoxanthine phosphoribosyltransferase (HPRT) gene, an albumin gene, a Factor VIII gene, a Factor IX gene, a Leucine-rich repeat kinase 2 (LRRK2) gene, a Huntingtin (HTT) gene, a rhodopsin (RHO) gene, a Cystic Fibrosis Transmembrane Conductance Regulator (CFTR) gene, F8 or a fragment thereof (e.g., fragment encoding B-domain deleted polypeptide (e.g., VIII SQ, p-VIII)), a surfactant protein B gene (SFTPB), a T-cell receptor alpha (TRAC) gene, a T-cell receptor beta (TRBC) gene, a programmed cell death 1 (PD1) gene, a Cytotoxic T-Lymphocyte Antigen 4 (CTLA-4) gene, an human leukocyte antigen (HLA) A gene, an HLA B gene, an HLA C gene, an HLA-DPA gene, an HLA-DQ gene, an HLA-DRA gene, a LMP7 gene, a Transporter associated with Antigen Processing (TAP) 1 gene, a TAP2 gene, a tapasin gene (TAPBP), a class II major histocompatibility complex transactivator (CUT A) gene, a dystrophin gene (DMD), a glucocorticoid receptor gene (GR), an IL2RG gene, an RFX5 gene, a FAD2 gene, a FAD3 gene, a ZP15 gene, a KASII gene, a MDH gene, and/or an EPSPS gene.

In some embodiments, a nucleic acid of interest for use in the recombinant virion disclosed herein can be used to restore the expression of genes that are reduced in expression, silenced, or otherwise dysfunctional in a subject. Similarly, in some embodiments, a nucleic acid of interest for use in the recombinant virion disclosed herein can also be used to knockdown the expression of genes that are aberrantly expressed in a subject.

In some embodiments, the dysfunctional gene is a tumor suppressor that has been silenced in a subject having cancer. In some embodiments, the dysfunctional gene is an oncogene that is aberrantly expressed in a subject having a cancer. Exemplary genes associated with cancer (oncogenes and tumor suppressors) include but not limited to: AARS, ABCB 1, ABCC4, ABI2, ABL1, ABL2, ACK1, ACP2, ACY1, ADSL, AK1, AKR1C2, AKT1, ALB, ANPEP, ANXAS, ANXA7, AP2M1, APC, ARHGAPS, ARHGEFS, ARID4A, ASNS, ATF4, ATM, ATPSB, ATPSO, AXL, BARD1, BAX, BCL2, BHLHB2, BLMH, BRAF, BRCA1, BRCA2, BTK, CANX, CAP1, CAPN1, CAPNS1, CAV1, CBFB, CBLB, CCL2, CCND1, CCND2, CCND3, CCNE1, CCTS, CCYR61, CD24, CD44, CD59, CDC20, CDC25, CDC25A, CDC25B, CDC2LS, CDK10, CDK4, CDK5, CDK9, CDKL1, CDKN1A, CDKN1B, CDKN1C, CDKN2A, CDKN2B, CDKN2D, CEBPG, CENPC1, CGRRF1, CHAFlA, CIB1, CKMT1, CLK1, CLK2, CLK3, CLNS1A, CLTC, COL1A1, COL6A3, COX6C, COX7A2, CRAT, CRHR1, CSF1R, CSK, CSNK1G2, CTNNA1, CTNNB1, CTPS, CTSC, CTSD, CUL1, CYR61, DCC, DCN, DDX10, DEK, DHCR7, DHRS2, DHX8, DLG3, DVL1, DVL3, E2F1, E2F3, E2F5, EGFR, EGR1, EIF5, EPHA2, ERBB2, ERBB3, ERBB4, ERCC3, ETV1, ETV3, ETV6, F2R, FASTK, FBN1, FBN2, FES, FGFR1, FGR, FKBP8, FN1, FOS, FOSL1, FOSL2, FOXG1A, FOXO1A, FRAP1, FRZB, FTL, FZD2, FZDS, FZD9, G22P1, GAS6, GCNSL2, GDF1S, GNA13, GNAS, GNB2, GNB2L1, GPR39, GRB2, GSK3A, GSPT1, GTF21, HDAC1, HDGF, HMMR, HPRT1, HRB, HSPA4, HSPA5, HSPA8, HSPB1, HSPH1, HYAL1, HYOU1, ICAM1, ID1, ID2, IDUA, IER3, IFITMI, IGF1R, IGF2R, IGFBP3, IGFBP4, IGFBPS, IL1B, ILK, ING1, IRF3, ITGA3, ITGA6, ITGB4, JAK1, JARID1A, JUN, JUNB, JUND, K-ALPHA-1, KIT, KITLG, KLK10, KPNA2, KRAS2, KRT18, KRT2A, KRT9, LAMB1, LAMP2, LCK, LCN2, LEP, LITAF, LRPAP1, LTF, LYN, LZTR1, MADH1, MAP2K2, MAP3K8, MAPK12, MAPK13, MAPKAPK3, MAPREl, MARS, MAS1, MCC, MCM2, MCM4, MDM2, MDM4, MET, MGST1, MICB, MLLT3, MME, MMP1, MMP14, MMP17, MMP2, MNDA, MSH2, MSH6, MT3, MYB, MYBL1, MYBL2, MYC, MYCLI, MYCN, MYD88, MYL9, MYLK, NEO1, NF1, NF2, NFKB I, NFKB2, NFSF7, NID, NINJ1, NMBR, NME1, NME2, NME3, NOTCH 1, NOTCH2, NOTCH4, NPM1, NQO1, NR1D1, NR2F1, NR2F6, NRAS, NRG1, NSEP1, OSM, PA2G4, PABPC1, PCNA, PCTK1, PCTK2, PCTK3, PDGFA, PDGFB, PDGFRA, PDPK1, PEA15, PFDN4, PFDN5, PGAM1, PHB, PIK3CA, PIK3CB, PIK3CG, PIM1, PKM2, PKMYT1, PLK2, PPARD, PPARG, PPIH, PPP1CA, PPP2RSA, PRDX2, PRDX4, PRKAR1A, PRKCBP1, PRNP, PRSS15, PSMA1, PTCH, PTEN, PTGS1, PTMA, PTN, PTPRN, RABSA, RAC1, RADSO, RAF1, RALBP1, RAPlA, RARA, RARB, RASGRF1, RB1, RBBP4, RBL2, REA, REL, RELA, RELB, RET, RFC2, RGS19, RHOA, RHOB, RHOC, RHOD, RIPK1, RPN2, RPS6 KB 1, RRM1, SARS, SELENBP1, SEMA3C, SEMA4D, SEPP1, SERPINH1, SFN, SFPQ, SFRS7, SHB, SHH, SIAH2, SIVA, SIVA TP53, SKI, SKIL, SLC16A1, SLC1A4, SLC20A1, SMO, SMPD1, SNAI2, SND1, SNRPB2, SOCS1, SOCS3, SOD1, SORT1, SPINT2, SPRY2, SRC, SRPX, STAT1, STAT2, STAT3, STAT5B, STC1, TAF1, TBL3, TBRG4, TCF1, TCF7L2, TFAP2C, TFDP1, TFDP2, TGFA, TGFB1, TGFBR1, TGFBR2, TGFBR3, THBS1, TIE, TIMP1, TIMP3, TJP1, TK1, TLE1, TNF, TNFRSF10A, TNFRSF10B, TNFRSFlA, TNFRSFlB, TNFRSF6, TNFSF7, TNK1, TOB1, TP53, TP53BP2, TP5313, TP73, TPBG, TPT1, TRADD, TRAM1, TRRAP, TSG101, TUFM, TXNRD1, TYR03, UBC, UBE2L6, UCHL1, USP7, VDAC1, VEGF, VHL, VIL2, WEE1, WNT1, WNT2, WNT2B, WNT3, WNTSA, WT1, XRCC 1, YES 1, YWHAB, YWHAZ, ZAP70, and ZNF9.

In some embodiments, the dysfunctional gene is HBB. In some embodiments, the HBB comprises at least one nonsense, frameshift, or splicing mutation that reduces or eliminates the β-globin production. In some embodiments, HBB comprises at least one mutation in the promoter region or polyadenylation signal of HBB. In some embodiments, the HBB mutation is at least one of c.17A>T, c.-1360G, c.92+1G>A, c.92+6T>C, c.93-21G>A, c.1180T, c.316-106OG, c.25_26delAA, c.27_28insG, c.92+5G>C, c.1180T, c. 135delC, c.315+1G>A, c.-78A>G, c.52A>T, c.59A>G, c.92+5G>C, c. 124_127delTTCT, c.316-1970T, c.-78A>G, c.52A>T, c. 124_127delTTCT, c.316-197C>T, C.-1380T, c.-79A>G, c.92+5G>C, c.75T>A, c.316-2A>G, and c.316-2A>C.

In certain embodiments, the sickle cell disease is improved by gene therapy (e.g., stem cell gene therapy) that introduces an HBB variant that comprises one or more mutations comprising anti-sickling activity. In some embodiments, the HBB variant may be a double mutant (βAS2; T87Q and E22A). In other embodiments, the HBB variant may be a triple-mutant β-globin variant (βAS3; T87Q, E22A, and G16D). A modification at 316, glycine to aspartic acid, serves a competitive advantage over sickle globin (PS, HbS) for binding to a chain. A modification at 322, glutamic acid to alanine, partially enhances axial interaction with α20 histidine. These modifications result in anti-sickling properties greater than those of the single T87Q-modified variant and comparable to fetal globin. In a SCD murine model, transplantation of bone marrow stem cells transduced with SIN lentivirus carrying βAS3 reversed the red blood cell physiology and SCD clinical symptoms. Accordingly, this variant is being tested in a clinical trial (Identifier no: NCT02247843), Cytotherapy (2018) 20(7): 899-910.

In some embodiments, the dysfunctional gene is CFTR. In some embodiments, CFTR comprises a mutation selected from AF508, R553X, R74W, R668C, S977F, L997F, K1060T, A1067T, R1070Q, R1066H, T3381, R334W, G85E, A46D, 1336K, H1054D, M1V, E92K, V520F, H1085R, R560T, L927P, R560S, N1303K, M1101K, L1077P, R1066M, R1066C, L1065P, Y569D, A561E, A559T, S492F, L467P, R347P, S341P, I507del, G1061R, G542X, W1282X, and 2184InsA.

In some embodiments, a nucleic acid of interest as defined herein encodes a small interfering nucleic acid (e.g., shRNAs, miRNAs) that inhibits the expression of a gene product associated with cancer (e.g., oncogenes) may be used to prevent or treat the cancer. In some embodiments, a nucleic acid of interest as defined herein encodes a gene product associated with cancer (or a functional RNA that inhibits the expression of a gene associated with cancer) for use, e.g., for research purposes, e.g., to study the cancer or to identify therapeutics that prevent or treat the cancer.

An ordinarily skilled artisan also appreciates that the nucleic acids of interest can comprise one or more mutations that result in conservative amino acid substitutions which may provide functionally equivalent variants, or homologs of a protein or polypeptide. Additionally contemplated in this disclosure is a nucleic acid of interest in a recombinant virion described herein, having a dominant negative mutation. For example, a nucleic acid of interest can encode a mutant protein that interacts with the same elements as a wild-type protein, and thereby blocks some aspects of the function of the wild-type protein.

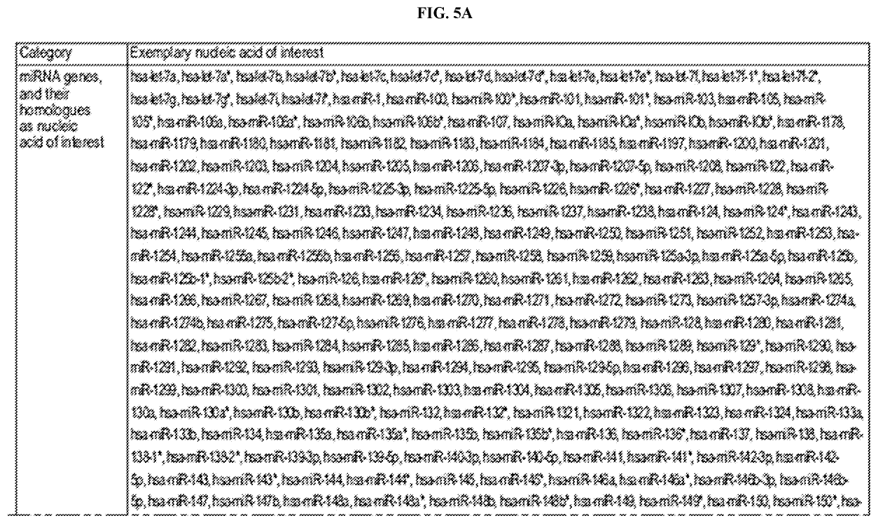

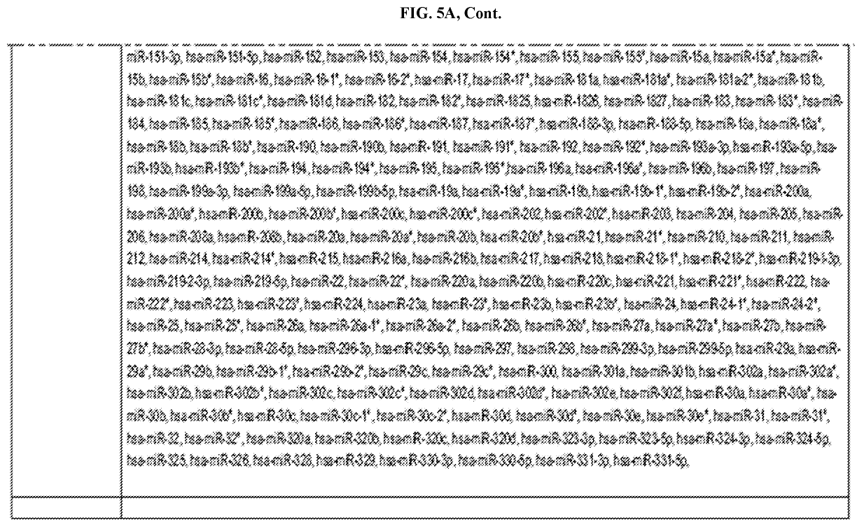

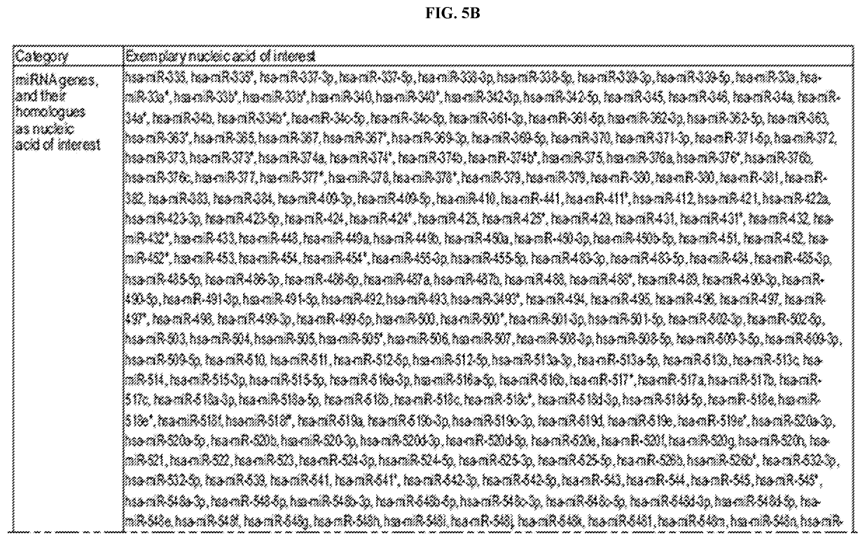

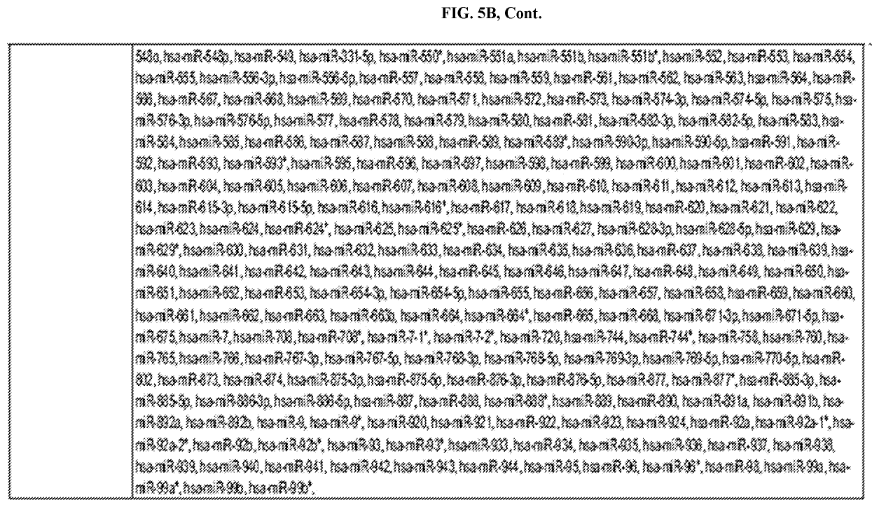

In some embodiments, the nucleic acid of interest in a recombinant virion disclosed herein includes miRNAs. miRNAs and other small interfering nucleic acids regulate gene expression via target RNA transcript cleavage/degradation or translational repression of the target messenger RNA (mRNA). miRNAs are natively expressed, typically as final 19-25 non-translated RNA products. miRNAs exhibit their activity through sequence-specific interactions with the 3′ untranslated regions (UTR) of target mRNAs. These endogenously expressed miRNAs form hairpin precursors which are subsequently processed into a miRNA duplex, and further into a “mature” single stranded miRNA molecule. This mature miRNA guides a multiprotein complex, miRISC, which identifies target site, e.g., in the 3′ UTR regions, of target mRNAs based upon their complementarity to the mature miRNA. FIG. 5A and FIG. 5B disclose a non-limiting list of miRNA genes, and their homologues, or as targets for small interfering nucleic acids encoded by the nucleic acid described herein (e.g., miRNA sponges, antisense oligonucleotides, TuD RNAs).

A miRNA inhibits the function of the mRNAs it targets and, as a result, inhibits expression of the polypeptides encoded by the mRNAs. Thus, blocking (partially or totally) the activity of the miRNA (e.g., silencing the miRNA) can effectively induce, or restore, expression of a polypeptide whose expression is inhibited (de-repress the polypeptide). In some embodiments, de-repression of polypeptides encoded by mRNA targets of a miRNA is accomplished by inhibiting the miRNA activity in cells through any one of a variety of methods. For example, blocking the activity of a miRNA can be accomplished by hybridization with a small interfering nucleic acid (e.g., antisense oligonucleotide, miRNA sponge, TuD RNA) that is complementary, or substantially complementary to, the miRNA, thereby blocking interaction of the miRNA with its target mRNA. As used herein, a small interfering nucleic acid that is substantially complementary to a miRNA is one that is capable of hybridizing with a miRNA, and blocking the miRNA's activity. In some embodiments, a small interfering nucleic acid that is substantially complementary to a miRNA is a small interfering nucleic acid that is complementary with the miRNA at all but 1, 2, 3, 4, 5, 6, 7, 8, 9, 10, 11, 12, 13, 14, 15, 16, 17, or 18 bases. In some embodiments, a small interfering nucleic acid sequence that is substantially complementary to a miRNA, is an small interfering nucleic acid sequence that is complementary with the miRNA at, at least, one base.

### Regulatory Sequences

A nucleic acid of a recombinant virion disclosed herein may also comprise transcriptional or translational regulatory sequences, for example, promoters, enhancers, insulators, internal ribosome entry sites, sequences encoding 2A peptides and/or polyadenylation signals.

In some embodiments, the regulatory sequence includes a suitable promoter sequence, being able to direct transcription of a gene operably linked to the promoter sequence, such as a nucleic acid of interest as described herein. In embodiments, an enhancer sequence is provided upstream of the promoter to increase the efficacy of the promoter. In some embodiments, the regulatory sequence includes an enhancer and a promoter, wherein the second nucleotide sequence includes an intron sequence upstream of the nucleotide sequence encoding a nuclease, wherein the intron includes one or more nuclease cleavage site(s), and wherein the promoter is operably linked to the nucleotide sequence encoding the nuclease.

Suitable promoters, including those described herein, can be derived from viruses and can therefore be referred to as viral promoters, or they can be derived from any organism, including prokaryotic or eukaryotic organisms. In some embodiments, promoters are derived from insect cells or mammalian cells. Suitable promoters can be used to drive expression by any RNA polymerase (e.g., pol l, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (Miyagishi et al., Nature Biotechnology 20, 497-500 (2002)), an enhanced U6 promoter (e.g., Xia et al.,

Nucleic Acids Res. 2003 Sep. 1; 31(17)), a human H 1 promoter (Hl), and the like. In some embodiments, these promoters are altered to include one or more nuclease cleavage sites.

A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter, as well as the promoters listed below. Such promoters and/or enhancers can be used for expression of any gene of interest, e.g., the gene editing molecules, donor sequence, therapeutic proteins etc.). For example, the nucleic acid may comprise a promoter that is operably linked to the DNA endonuclease or CRISPR/Cas9-based system. The promoter operably linked to the CRISPR/Cas9-based system or the site-specific nuclease coding sequence may be a promoter from simian virus 40 (SV40), a CAG promoter, a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human ubiquitin C (hUbC), human actin, human myosin, human hemoglobin, human muscle creatine, or human metalothionein. The promoter may also be a tissue specific promoter, such as a liver specific promoter, natural or synthetic. In one embodiment, delivery to the liver can be achieved using endogenous ApoE specific targeting of the composition comprising a vector to hepatocytes via the low density lipoprotein (LDL) receptor present on the surface of the hepatocyte. In some embodiments, use is made of in silico designed synthetic promoters having an assembly of regulatory elements. These synthetic promoters are not naturally occurring and are designed either for optimal expression in the target tissue, regulated expression, or for accommodation in a virus capsid.

In some embodiments, the promoter may be selected from: (a) a promoter heterologous to the nucleic acid, (b) a promoter that facilitates the tissue-specific expression of the nucleic acid, preferably wherein the promoter facilitates hematopoietic cell-specific expression or erythroid lineage-specific expression, (c) a promoter that facilitates the constitutive expression of the nucleic acid, and (d) a promoter that is inducibly expressed, optionally in response to a metabolite or small molecule or chemical entity. Examples of inducible promoters include those regulated by tetracycline, cumate, rapamycin, FKCsA, ABA, tamoxifen, blue light, and riboswitch. Additional details are provided in e.g., Kallunki et al. (2019) Cells 8:E796, which is incorporated by reference. In some embodiments, the promoter is selected from the CMV promoter, β-globin promoter, CAG promoter, AHSP promoter, MND promoter, Wiskott-Aldrich promoter, and PKLR promoter.

### Sequences

As used herein, coding region refers to regions of a nucleotide sequence comprising codons which are translated into amino acid residues, whereas noncoding region refers to regions of a nucleotide sequence that are not translated into amino acids. Transcribed non-coding sequences may be upstream (5′-UTR), downstream (3′-UTR), or intronic. Non-transcribed non-coding sequences may have cis-acting. regulatory functions, e.g., enhancer and promoter, or act as “spacers,” non-transcribed DNA used to separate functional groups in the DNA, e.g., polylinkers or “stuffer” DNA used to increase the size of the vector genome.

Complement or complementary to refers to the broad concept of sequence complementarity between regions of two nucleic acid strands or between two regions of the same nucleic acid strand. It is known that an adenine residue of a first nucleic acid region is capable of forming specific hydrogen bonds (base pairing) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some embodiments, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. In other embodiments, all nucleotide residues of the first portion are capable of base pairing with nucleotide residues in the second portion.

A nucleic acid is operably linked when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well-known feature of the genetic code is its degeneracy, whereby, for most of the amino acids used to make proteins, more than one coding nucleotide triplet may be employed (illustrated above). Therefore, a number of different nucleotide sequences may code for a given amino acid sequence. The universality of the genetic code provides that such nucleotide sequences are considered functionally equivalent since they result in the production of the same amino acid sequence in all organisms, although mitochondria and plastids and similar symbiotic organelles have a slightly different genetic code. Although not all codons are utilized with similar translation efficiency, rare codons may lower the protein production due to limiting tRNA pools. Moreover, occasionally, a methylated variant of a purine or pyrimidine may be found in a given nucleotide sequence. Such methylations do not affect the coding relationship between the trinucleotide codon and the corresponding amino acid.

In making the changes in the amino sequences of polypeptide, the hydropathic index of amino acids may be considered. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (<RTI 3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well-known to those of skill in the art and include: arginine and lysine; glutamate and aspartate; serine and threonine; glutamine and asparagine; and valine, leucine and isoleucine.

It is also known in the art that a nucleic acid encoding a polypeptide can be codon-optimized for certain host cells, without altering the amino acid sequence. Codon-optimization describes gene engineering approaches that use synonymous codon changes to increase protein production. This is possible because most amino acids are encoded by more than one codon. Replacing rare codons with frequently used ones have shown to increase protein expression.

In view of the foregoing, the nucleotide sequence of a DNA or RNA encoding a nucleic acid (or any portion thereof) described herein (e.g., a therapeutic nucleic acid) can be used to derive the polypeptide amino acid sequence, using the genetic code to translate the DNA or RNA into an amino acid sequence. Likewise, for polypeptide amino acid sequence, corresponding nucleotide sequences that can encode the polypeptide can be deduced from the genetic code (which, because of its redundancy, will produce multiple nucleic acid sequences for any given amino acid sequence). Thus, description and/or disclosure herein of a nucleotide sequence which encodes a polypeptide should be considered to also include description and/or disclosure of the amino acid sequence encoded by the nucleotide sequence. Similarly, description and/or disclosure of a polypeptide amino acid sequence herein should be considered to also include description and/or disclosure of all possible nucleotide sequences that can encode the amino acid sequence.

Finally, nucleic acid and amino acid sequence information for nucleic acid and polypeptide molecules useful in the present invention are well-known in the art and readily available on publicly available databases, such as the National Center for Biotechnology Information (NCBI).

TABLE 4
Sequences

```
As described above, an ordinarily skilled artisan appreciates the
genotypic variation of the viral sequences. Accordingly, the sequences
presented herein represent sequences of exemplar isolates.
SEQ ID NO: 1, 2, and 3 - represent examples of AAV ITRs. Wave underline
- terminal resolution site (trs); dotted underline - A and A' stem;
solid underline - B/B' and C/C' stems.
SEQ ID NO: 1
AAV2 ITR “Flop” conformer nucleic acid sequence

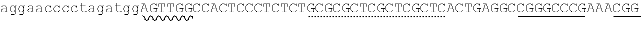

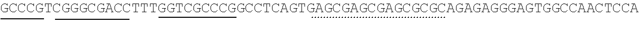


TCTAGGGGTTCCT
SEQ ID NO: 2
AAV2 ITR “Flip” conformer nucleic acid sequence
cctgcaggcagCTGCGCGCTCGCTCGCTCACTGAGGCCGCCCGGGCAAAGCCCGGGCGTCGGGCGACCTTTGG
TCGCCCGGCCTCAGTGAGCGAGCGAGCGCGCAGAGAGGGAGTGGCCAACTCCATCACTAGGGGTTCCT
SEQ ID NO: 3
AAV2 ITR “Flop” conformer nucleic acid sequence

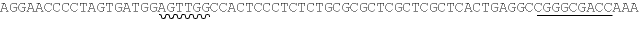

GGTCGCCCGACGCCCGGGCTTTGCCCGGGCGGCCTCAGTGAGCGAGCGAGCGCGCAGctgcctgcagg
SEQ ID NO: 4
Canine Parvovirus (CTG-VP1-3-Leu-VP2) (UniProtKB - P12930 (CAPSD_PAVCN))
LAPPAKRARRGLVPPGYKYLGPGNSLDQGEPTNPSDAAAKEHDEAYAAYLRSGKNPYLYFSPADQRFIDQTKD
AKDWGGKIGHYFFRAKKAIAPVLTDTPDHPSTSRPTKPTKRSKPPPHIFINLAKKKKAGAGQVKRDNLAPMSD
GAVQPDGGQPAVRNERATGSGNGSGGGGGGGSGGVGISTGTENNOTEFKFLENGWVEITANSSRLVHLNMPES
ENYRRVVVNNMDKTAVNGNMALDDIHAQIVTPWSLVDANAWGVWFNPGDWQLIVNTMSELHLVSFEQEIFNVV
LKTVSESATQPPTKVYNNDLTASLMVALDSNNTMPFTPAAMRSETLGFYPWKPTIPTPWRYYFQWDRTLIPSH
TGTSGTPTNIYHGTDPDDVQFYTIENSVPVHLLRTGDEFATGTFFFDCKPCRLTHTWOTNRALGLPPELNSLP
QSEGATNFGDIGVQQDKRRGVTOMGNTNYITEATIMRPAEVGYSAPYYSFEASTQGPFKTPIAAGRGGAQTYE
NQAADGDPRYAFGRQHGQKTTTTGETPERFTYIAHQDTGRYPEGDWIQNINFNLPVTNDNVLLPTDPIGGKTG
INYTNIFNTYGPLTALNNVPPVYPNGQIWDKEFDTDLKPRLHVNAPFVCONNCPGOLFVKVAPNLTNEYDPDA
SANMSRIVTYSDFWWKGKLVFKAKLRASHTWNPIQQMSINVDNQFNYVPSNIGGMKIVYEKSQLAPRKLY
*SEQ ID NO: 4 comprises the sequences of both VP1 and VP2 of the CPV-N
strain. The entire sequence corresponds to VP1, whereas the VP2 sequence
starts at the bolded “M”, which marks the first methione of VP2.
Underlined residues define the receptor binding domains that influence
internalization and transcytosis. Mutations within these regions
alter the receptor interaction and modulate transcytosis.
Amino acid 93 of VP2 (numbered starting from the bolded “M”) and the
adjacent residues are involved in TfR interaction and host range.
Amino acid 300 and the adjacent residues of VP2 (starting from the
bolded “M”) influence host range (interaction with TfR).
Amino acid 323 and the adjacent residues of VP2 (starting from the
bolded “M”) are involved in TfR interaction and host range.
SEQ ID NO: 5
Canine Parvovirus VP1 amino acid sequence (GenBank AXQ00350)
    1 mappakrarr glvppgykyl gpgnsldqge ptnpsdaaak ehdeayaayl rsgknpylyf
  61 spadqrfidq tkdakdwggk ighyffrakk aiapvltdtp dhpstsrptk ptkrskppph
121 ifinlakkkk agagqvkrdn lapmsdggvq pdggqpavrn eratgsgngs gggggggsgg
181 vgistgtinn qtefkfleng wveitanssr lvhlnmpese nyrrvvvnnl dktavngnma
241 lddthaqivt pwslvdanaw gvwfnpgdwq livntmselh lvsfeqeifn vvlktvsesa
301 tqpptkvynn dltaslmval dsnntmpftp aamrsetlgf ypwkptiptp wryyfqwdrt
361 lipshtgtsg tptniyhgtd pddvqfytie nsvpvhllrt gdefatgtfy fdckpcrlth
421 twqtnralgl ppflnslpqa eggtnfgyig vqqdkrrgvt qmgntniite atimrpaevg
481 ysapyysfea stqgpfktpi aagrggaqtd enraadgdpr yafgrqhgqk ttttgetper
541 ftyiahqdtg rypegdwiqn infnlpvted nvllptdpig gktginytni fntygpltal
601 nnvppvypng qiwdkefdtd lkprlhvnap fvcqnncpgq lfvkvapnlt neydpdasan
661 msrivtysdf wwkgklvfka klrashtwnp iqqmsinvdn qfnyvpsnig gmkivyeksq
721 laprkly
SEQ ID NO: 6
Canine Parvovirus VP2 amino acid sequence (Genbank CAA86612)
    1 msdgavqpdg gqpavrnera tgsgngsggg ggggsggvgi stgtfnnqte fkflengwve
  61 itanssrlvh Inmpesenyr rvvvnnldkt avngnmaldd thaqivtpws lvdanawgvw
121 fnpgdwqliv ntmselhlvs feqeifnvvl ktvsesatqp ptkvynndlt aslmvaldsn
181 ntmpftpaam rsetlgfypw kptiptpwry yfqwdrtlip shtgtsgtpt niyhgtdpdd
241 vqfytiensv pvhllrtgde fatgtfffdc kpcrlthtwq tnralglppf Inslpqsegg
301 tnfgyigvqq dkrrgvtqmg ntnyiteati mrpaevgysa pyysfeastq gpfktpiaag
361 rggaqtdenq aadgdpryaf grqhgqkttt tgetperfty iahqdtgryp egdwiqninf
421 nlpvtddnvl lptdpiggkt ginytnifnt ygpltalnnv ppvypngqiw dkefdtdlkp
481 rlhvnapfvc qnncpgqlfv kvapnltney dpdasanmsr ivtysdfwwk gklvfkaklr
541 ashtwnpiqq msinvdnqfn yvpsniggmk ivyeksqlap rkly
SEQ ID NO: 7
Feline Panelukepenia Virus (CTG-VP1-3-Leu-VP2)
LAPPAKRARRGLVPPGYKYLGPGNSLDQGEPTNPSDAAAKEHDEAYAAYLRSGKNPYLYFSPADQRFIDQTKD
AKDWGGKIGHYFFRAKKAIAPVLTDTPDHPSTSRPTKPTKRSKPPPHIFINLAKKKKAGAGQVKRDNLAPMSD
GAVQPDGGQPAVRNERATGSGNGSGGGGGGGSGGVGISTGTENNQTEFKFLENGWVEITANSSRLVHLNMPES
ENYKRVVVNNMDKTAVKGNMALDDIHVQIVTPWSLVDANAWGVWFNPGDWQLIVNTMSELHLVSFEQEIFNVV
LKTVSESATQPPTKVYNNDLTASLMVALDSNNTMPFTPAAMRSETLGFYPWKPTIPTPWRYYFQWDRTLIPSH
TGTSGTPTNVYHGTDPDDVQFYTIENSVPVHLLRTGDEFATGTFFFDCKPCRLTHTWQTNRALGLPPFLNSLP
QSEGATNFGDIGVQQDKRRGVTQMGNTDYITEATIMRPAEVGYSAPYYSFEASTQGPFKTPIAAGRGGAQTDE
NOAADGDPRYAFGRQHGQKTTTTGETPERFTYIAHQDTGRYPEGDWIQNINFNLPVTNDNVLLPTDPIGGKTG
INYTNIFNTYGPLTALNNVPPVYPNGQIWDKEFDTDLKPRLHVNAPFVCONNCPGOLFVKVAPNLTNEYDPDA
SANMSRIVTYSDFWWKGKLVFKAKLRASHTWNPIQQMSINVDNQFNYVPNNIGAMKIVYEKSQLAPRKLY
SEQ ID NO: 7 comprises the sequences of both VP1 and VP2 of this strain
of feline panelukepenia virus. The entire sequence corresponds to VP1,
whereas the VP2 sequence starts at the bolded “M”, which marks the first
methione of VP2.
SEQ ID NO: 8
Feline Panelukepenia Virus VP1 amino acid sequence (GenBank AK188071)
    1 mappakrarr glvppgykyl gpgnsldqge ptnpsdaaak ehdeayaayl rsgknpylyf
  61 spadqrfidq tkdakdwggk ighyffrakk aiapvltdtp dhpstsrptk ptkrskppph
121 ifinlakkkk agagqvkrdn lapmsdgavq pdggqpavrn eratgsgngs gggggggsgg
181 vgistgtfnn qtefkfleng wveitanssr lvhlnmpese nykrvvvnnm dktavkgnma
241 lddthvqivt pwslvdanaw gvwfnpgdwq livntmselh lvsfeqeifn vvlktvsesa
301 tqpptkvynn dltaslmval dsnntmpftp aamrsetlgf ypwkptiptp wryyfqwdrt
361 lipshtgtsg tptnvyhgtd pddvqfytie nsvpvhllrt gdefatgtff fdckpcrlth
421 twqtnralgl ppflnslpqs egatnfgdig vqqdkrrgvt qmgntdyite atimrpaevg
481 ysapyysfea stqgpfktpi aagrggaqtd enqaadgdpr yafgrqhgqk ttttgetper
541 ftyiahqdtg rypegdwiqn infnlpvtnd nvllptdpig gktginytni fntygpltal
601 nnvppvypng qiwdkefdtd lkprlhvnap fvcqnncpgq lfvkvapnlt neydpdasan
661 msrivtysdf wwkgklvfka klrashtwnp iqqmsinvdn qfnyvpnnig amkivyeksq
721 laprkly
SEQ ID NO: 9
Feline Panelukepenia Virus VP2 amino acid sequence (GenBank ACI31203)
    1 msdgavqpdg gqpavrnera tgsgngsggg ggggsggvgi stgtfnnqte fkflengwve
  61 itanssrlvh Inmpesenyk rvvvnnmdkt avkgnmaldd ihvqivtpws lvdanawgvw
121 fnpgdwqliv ntmselhlvs feqeifnvvl ktvsesatqp ptkvynndlt aslmvaldsn
181 ntmpftpaam rsetlgfypw kptiptpwry yfqwdrtlip shtgtsgtpt niyhgtdpdd
241 vqfytiensv pvhllrtgde fatgtfffdc kpcrlthtwq tnralglppf Inslpqsega
301 tnfgdigvqq dkrrgvtqmg ntnyiteati mrpaevgysa pyysfeastq gpfktpiaag
361 rggaqtdenq aadgdpryaf grqhgqkttt tgetperfty iahqdtgryp egdwiqninf
421 nlpvtndnvl lptdpiggkt ginytnifnt ygpltalnnv ppvypngqiw dkefdtdlkp
481 rlhvnapfvc qnncpgqlfv kvapnltney dpdasanmsr ivtysdfwwk gklvfkaklr
541 ashtwnpiqq msinvdnqfn yvpsnigamk ivyeksqlap rkly
SEQ ID NO: 10
Bufavirus (CTG-VP1-3-Leu-VP2)
LPAIRKARGWVPPGYNFLGPFNQDENKEPTNPSDNAAKQHDLEYNKLINQGHNPYWYYNKADEDFIKATDQAP
DWGGKFGNFIFRAKKHIAPELAPPAKKKSKTKHSEPEFSHKHIKPGTKRGKPFHIFVNLARKRARMSEPANDT
NEQPDNSPVEQGAGQIGGGGGGGGSGVGHSTGDYNNRTEFIYHGDEVTIICHSTRLVHINMSDREDYIIYETD
RGPLFPTTQDLQGRDTLNDSYHAKVETPWKLLHANSWGCWFSPADFQQMITTCRDIAPIKMHQKIENIVIKTV
SKTGTGETETTNYNNDLTALLQIAQDNSNLLPWAADNFYIDSVGYVPWRACKLPTYCYHVDTWNTIDINQADT
PNQWREIKKGIQWDNIQFTPLETMINIDLLRTGDAWESGNYNFHTKPTNLAYHWQSQRHTGSCHPTVAPLVER
GQGTNIQSVNCWOWGDRNNPSSASTRVSNIHIGYSFPEWQIHYSTGGPVINPGSAFSQAPWGSTTEGTRLTQG
ASEKAIYDWSHGDDQPGARETWWQNNQHVTGQTDWAPKNAHTSELNNNVPAATHEWKNSYHNTFSPFTAVDDH
GPQYPWGAIWGKYPDTTHKPMMSAHAPFLLHGPPGOLFVKLAPNYTDTLDNGGVTHPRIVTYGTFWWSGQLIF
KGKLRTPRQWNTYNLPSLDKRETMKNTVPNEVGHFELPYMPGRCLPNYTL
SEQ ID NO: 10 comprises the sequences of both VP1 and VP2 of this strain
of bufavirus. The entire sequence corresponds to VP1, whereas the VP2
sequence starts at the bolded “M”, which marks the first methione of VP2.
SEQ ID NO: 11
Bufavirus VP1 amino acid sequence (GenBank AFN44271)
    1 mpairkargw vppgynylgp fnqdfskkpt npsdnaarkh dleynklikq ghnpywnynh
  61 adedfiketd qatdwggkfg nfvfrakral apelappakk ktktkhtepe yshkhikagt
121 krgkpfylfv nlarkkarmt dtqdvseqqs dqpsvastsa kagggggggg sgvghstgny
181 nnrtefyyhg devtivchss rhihlnmses eeykiydtdr gptfptdqtl qgrdtindsy
241 haqvetpwfl inpnswgtwm npadfqqltt tcrevtlehl dqtldnivik tvskqgsgae
301 ettqynndlt allqvaldks nqlpwvadnm yldslgyipw rpcklkqysy hvnfwntidi
361 isgpqqnqwq qvkkeikwdd lqftpiettt eidllrtgds wtsgpykfnt kptqlsyhwq
421 strhtgsvhp teppnaigqq grniidingw qwgdrsnpms aatrvsnfhi gyswpewrih
481 ygsggpainp gapfsqapws tdpqvrltqg asekaifdyn hgdddpahrd qwwqnnlpmt
541 gqtdwapkna hqtnvsnnip srqefwtqdy hntfgpftav ddvgiqypwg aiwtktpdtt
601 hkpmmsahap fickdgppgq llvklapnyt enlqtdglgn nrivtyatfw wtgklvlkgk
661 lrlprqfnly nlpgrprgte akkflpneig hfelpfmpgr cmpnyti
SEQ ID NO: 12
Bufavirus VP2 amino acid sequence (GenBank AFN44272)
    1 mtdtqdvseq qsdqpsvast sakagggggg ggsgvghstg nynnrtefyy hgdevtivch
  61 ssrhihlnms eseeykiydt drgptfptdq tlqgrdtind syhaqvetpw flinpnswgt
121 wmnpadfqql tttcrevtle hldqtldniv iktvskqgsg aeettqynnd ltallqvald
181 ksnqlpwvad nmyldslgyi pwrpcklkqy syhvnfwnti diisgpqqnq wqqvkkeikw
241 ddlqftpiet tteidllrtg dswtsgpykf ntkptqlsyh wqstrhtgsv hpteppnaig
301 qqgrniidin gwqwgdrsnp msaatrvsnf higyswpewr ihygsggpai npgapfsqap
361 wstdpqvrlt qgasekaifd ynhgdddpah rdqwwqnnlp mtgqtdwapk nahqtnvsnn
421 ipsrqefwtq dyhntfgpft avddvgiqyp wgaiwtktpd tthkpmmsah apfickdgpp
481 gqllvklapn ytenlqtdgl gnnrivtyat fwwtgklvlk gklrlprqfn lynlpgrprg
541 teakkflpne ighfelpfmp grcmpnyti
SEQ ID NO: 13
Tusavirus (CTG-VP1-3-Leu-VP2)
LAPPAKRARRGLVPPGYKYLGPGNSLDQGEPTNPSDAAAKEHDEAYAAYLRSGKNPYLYFSPADQRFIDQTKD
AKDWGGKIGHYFFRAKKAIAPVLTDTPDHPSTSRPTKPTKRSKPPPHIFINLAKKKKAGAGQVKRDNLAPMSD
GAVQPDGGQPAVRNERATGSGNGSGGGGGGGSGGVGISTGTENNQTEFKFLENGWVEITANSSRLVHLNMPES
ENYRRVVVNNMDKTAVNGNMALDDIHAQIVTPWSLVDANAWGVWFNPGDWQLIVNTMSELHLVSFEQEIFNVV
LKTVSESATQPPTKVYNNDLTASLMVALDSNNTMPFTPAAMRSETLGFYPWKPTIPTPWRYYFQWDRTLIPSH
TGTSGTPTNIYHGTDPDDVQFYTIENSVPVHLLRTGDEFATGTFFFDCKPCRLTHTWOTNRALGLPPELNSLP
QSEGATNFGDIGVQQDKRRGVTQMGNTNYITEATIMRPAEVGYSAPYYSFEASTQGPFKTPIAAGRGGAQTYE
NQAADGDPRYAFGRQHGQKTTTTGETPERFTYIAHQDTGRYPEGDWIQNINFNLPVTNDNVLLPTDPIGGKTG
INYTNIFNTYGPLTALNNVPPVYPNGQIWDKEFDTDLKPRLHVNAPFVCONNCPGOLFVKVAPNLTNEYDPDA
SANMSRIVTYSDFWWKGKLVFKAKLRASHTWNPIQQMSINVDNQFNYVPSNIGGMKIVYEKSQLAPRKLY
SEQ ID NO: 13 comprises the sequences of both VP1 and VP2 of this strain
of tusavirus. The entire sequence corresponds to VP1, whereas the VP2
sequence starts at the bolded “M”, which marks the first methione of VP2.
SEQ ID NO: 14
Tusavirus VP1 amino acid sequence (GenBank AIT18930)
    1 mapaarprkg wvppgynylg pgndldagep tnksdaaark hdfaysaylk qgldpywnfn
  61 kadekfirdt egatdwggrl ghwifrakkh ilphlkeptl agrkrpapah ifvnlankrk
121 kglptrkdqq kdtldsnaqq pvreadqpdg maasssdsgp sssgggarag gvgvstgdfd
181 nttlwdfhed gtatitonst rlvhltrpds ldykiiptqn ntavqtvghm mdddnhtqvl
241 tpwslvdcna wgvwlsphdw qhimnigeel ellsleqevf nvtlktatet gppesritmy
301 nndltavmmi ttdtnnqlpy tpaairsetl gfypwrptvv prwryyfdwd rflsvtsssd
361 qstsiinhss tqsaigqffv ietqlpiall rtgdsyatgg ykfdcnkvnl grhwqttrsl
421 glppkieppt sesalgting narlgwrwgi ndvhetnvvr pctagynhpe wfythtlegp
481 aidpapptsi psnwgggtpp dtrasshnqq ritynynhgn kdenlnnfsl npnielgsii
541 nqgnflsyeg ngqqinttag vgkngetats dpnlvrympn tygvytavdh qgpvyphgqi
601 wdkqihtdkk pelhclapft cknnppgqmf vriapnltdt fnatptfsei ityadfwwkg
661 tlkmkiklrp phqwniatvl gaavnigdaa rfvpnrlgql efpvingriv pstvy
SEQ ID NO: 15
Tusavirus VP2 amino acid sequence (GenBank AIT18929)
    1 maasssdsgp sssgggarag gvgvstgdfd nttlwdfhed gtatitonst rlvhltrpds
  61 ldykiiptqn ntavqtvghm mdddnhtqvl tpwslvdcna wgvwlsphdw qhimnigeel
121 ellsleqevf nvtlktatet gppesritmy nndltavmmi ttdtnnqlpy tpaairsetl
181 gfypwrptvv prwryyfdwd rflsvtsssd qstsiinhss tqsaigqffv ietqlpiall
241 rtgdsyatgg ykfdcnkvnl grhwqttrsl glppkieppt sesalgting narlgwrwgi
301 ndvhetnvvr pctagynhpe wfythtlegp aidpapptsi psnwgggtpp dtrasshnqq
361 ritynynhgn kdenlnnfsl npnielgsii nqgnflsyeg ngqqinttag vgkngetats
421 dpnlvrympn tygvytavdh qgpvyphgqi wdkqihtdkk pelhclapft cknnppgqmf
481 vriapnltdt fnatptfsei ityadfwwkg tlkmkiklrp phqwniatvl gaavnigdaa
541 rfvpnrlgql efpvingriv pstvy
SEQ ID NO: 16
Cutavirus (CTG-VP1-3-Leu-VP2)
LPAIRKARGWVPPGYNFLGPFNQDENKEPTNPSDNAAKQHDLEYNKLINQGHNPYWYYNKADEDFIKATDQAP
DWGGKFGNFIFRAKKHIAPELAPPAKKKSKTKHSEPEFSHKHIKPGTKRGKPFHIFVNLARKRARMSEPANDT
NEQPDNSPVEQGAGQIGGGGGGGGSGVGHSTGDYNNRTEFIYHGDEVTIICHSTRLVHINMSDREDYIIYETD
RGPLFPTTQDLQGRDTLNDSYHAKVETPWKLLHANSWGCWFSPADFQQMITTCRDIAPIKMHQKIENIVIKTV
SKTGTGETETTNYNNDLTALLQIAQDNSNLLPWAADNFYIDSVGYVPWRACKLPTYCYHVDTWNTIDINQADT
PNQWREIKKGIQWDNIQFTPLETMINIDLLRTGDAWESGNYNFHTKPTNLAYHWQSQRHTGSCHPTVAPLVER
GQGTNIQSVNCWQWGDRNNPSSASTRVSNIHIGYSFPEWQIHYSTGGPVINPGSAFSQAPWGSTTEGTRLTQG
ASEKAIYDWSHGDDQPGARETWWONNQHVTGQTDWAPKNAHTSELNNNVPAATHEWKNSYHNTFSPFTAVDDH
GPQYPWGAIWGKYPDTTHKPMMSAHAPFLLHGPPGOLFVKLAPNYTDTLDNGGVTHPRIVTYGTFWWSGQLIF
KGKLRTPROWNTYNLPSLDKRETMKNTVPNEVGHFELPYMPGRCLPNYTL
SEQ ID NO: 16 comprises the sequences of both VP1 and VP2 of this strain
of cutavirus The entire sequence corresponds to VP1, whereas the VP2
sequence starts at the bolded “M”, which marks the first methione of VP2.
SEQ ID NO: 17
Cutavirus VP1 amino acid sequence (GenBank YP_009508805)
    1 mpairkargw vppgynflgp fnqdfnkept npsdnaakqh dleynkling ghnpywyynk
  61 adedfikatd qapdwggkfg nfifrakkhi apelappakk ksktkhsepe fshkhikpgt
121 krgkpfhifv nlarkrarms epandtneqp dnspveqgag qigggggggg sgvghstgdy
181 nnrtefiyhg devtiichst rlvhinmsdr edyiiyetdr gplfpttqdl qgrdtIndsy
241 hakvetpwkl lhanswgcwf spadfqqmit tordiapikm hqkienivik tvsktgtget
301 ettnynndlt allqiaqdns nllpwaadnf yidsvgyvpw racklptycy hvdtwntidi
361 nqadtpnqwr eikkgiqwdn iqftpletmi nidllrtgda wesgnynfht kptnlayhwq
421 sqrhtgschp tvaplvergq gtniqsvncw qwgdrnnpss astrvsnihi gysfpewqih
481 ystggpvinp gsafsqapwg sttegtrltq gasekaiydw shgddqpgar etwwqnnqhv
541 tgqtdwapkn ahtselnnnv paathfwkns yhntfspfta vddhgpqypw gaiwgkypdt
601 thkpmmsaha pfllhgppgq lfvklapnyt dtldnggvth privtygtfw wsgqlifkgk
661 lrtprqwnty nlpsldkret mkntvpnevg hfelpympgr clpnytl
SEQ ID NO: 18
Cutavirus VP2 amino acid sequence (GenBank YP_009508807)
    1 msepandtne qpdnspveqg agqigggggg ggsgvghstg dynnrtefiy hgdevtiich
  61 strlvhinms dredyiiyet drgplfpttq dlqgrdtInd syhakvetpw kllhanswgc
121 wfspadfqqm ittcrdiapi kmhqkieniv iktvsktgtg etettnynnd ltallqiaqd
181 nsnllpwaad nfyidsvgyv pwracklpty cyhvdtwnti dinqadtpnq wreikkgiqw
241 dniqftplet minidllrtg dawesgnynf htkptnlayh wqsqrhtgsc hptvaplver
301 gqgtniqsvn cwqwgdrnnp ssastrvsni higysfpewq ihystggpvi npgsafsqap
361 wgsttegtrl tqgasekaiy dwshgddqpg aretwwqnnq hvtgqtdwap knahtselnn
421 nvpaathfwk nsyhntfspf tavddhgpqy pwgaiwgkyp dtthkpmmsa hapfllhgpp
481 gqlfvklapn ytdtldnggv thprivtygt fwwsgqlifk gklrtprqwn tynlpsldkr
541 etmkntvpne vghfelpymp grclpnytl
SEQ ID NO: 19
Wuharv Parvovirus (CTG-VP1-3-Leu-VP2)
LLIWALFIWLFYFHEGTIEQLNIYHNGNSLQLLHHSSSLTGWVPPGYNYLGPKKFKLOKKPTNPSDAAARKHD
LEYNKLIQQGHNPYFYYNHADEDFIKETNQAKDWGGKFGNYIFRAKRALAPELAPPKKKSKTEKGEPSYSYKN
IKPGTKRGKPFYLFVNLAKKKRMSAPAAENNDQQPDAAEPSVAGAKATGGGGGGGSGVGHSTGSENNRTEFIY
HGSEVTIICHATRHIHLNMPDSEEYKIYETNRGPRFPTDOTLOGRDTINDSYHAKVOTPWQLLHANCWGCWFS
PADWQQMITTCREFKVEHLEQAIDNIVIKTVTKOGTGAEEITQYNNDLTALLQVAEDKSNLMPWAADNMYIDS
IGYVPWRPSKLPTYCYHVNFWNTIDINAGAQQNQWAEVKKGIQWDNIQFTPIENMVDIELLRTGDRWDSGPYT
FYCKPSSLEYHWQSTRHTGSCHPSTTPQAIGSVGNNLETLNAWOWGDRNNPSSASTRVSNFHIGYSWPEWQFH
YSTGGPVVNPGQPFSQAPWGSEVAGTRLTQGASEKAIYDWNHGDEEHQFRETWWONNENMTGQTNWAPKNIHQ
KELTNNVAAQTAFWAQGYHNTFGPFTAFDDHGAQYPWGAIWGKEPNTTHKPLASSHAPFMTANPPGQIFVKLA
PNYTDTVDNNGNSSRIVTFGTFWWTGKLVIKAKLRTPRQWNTYQLPSLAARETMKETVPNEIGNFEIPYMPGR
AMPNYTL
SEQ ID NO: 19 comprises the sequences of both VP1 and VP2 of this strain
of Wuharv parvovirus. The entire sequence corresponds to VP1, whereas
the VP2 sequence starts at the bolded “M”, which marks the first
methione of VP2.
  SEQ ID NO: 20
Wuharv Parvovirus VP1 amino acid sequence (GenBank YP_009508803)
    1 mliwalfiwl fyfhegtieq lniyhngnsl qllhhssslt gwvppgynyl gpkkfklqkk
  61 ptnpsdaaar khdleynkli qqghnpyfyy nhadedfike tnqakdwggk fgnyifrakr
121 alapelappk kksktekgep sysyknikpg tkrgkpfylf vnlakkkrms apaaenndqq
181 pdaaepsvag akatgggggg gsgvghstgs fnnrtefiyh gsevtiicha trhihlnmpd
241 seeyliyetn rgprfptdqt lqgrdtinds yhakvqtpwq llhancwgcw fspadwqqmi
301 ttcrefkveh leqaidnivi ktvtkqgtga eeitqynndl tallqvaedk snlmpwaadn
361 myidsigyvp wrpsklptyc yhvnfwntid inagaqqnqw aevkkgiqwd niqftpienm
421 vduekkrtgd rwdsgpytfy ckpssleyhw qstrhtgsch psttpqaigs vgnnletlna
481 wqwfdrnnps sastrvsnfh igyswpewqf hystggpvvn pgqpfsqapw gsevagtrlt
541 qgasekaiyd wnhgdeehqf retwwqnnen mtgqtnwapk nihqkeltnn vaaqtafwaq
601 gyhntfgpft afddhgaqyp wgaiwgkepn tthkplassh apfmtanppg qifvklapny
661 tdtvdnngns srivtfgtfw wtgklvikak lrtprqwnty qlpslaaret mketvpneig
721 nfeipympgr ampnytl
SEQ ID NO: 21
Wuhary Parvovirus VP2 amino acid sequence (GenBank YP_009508803.1)
    1 nsaoaaebbd qqodaaeosv agakatgggg gggsgvghst gsfnnrtefi yhgsevtiic
  61 hatrhihlnm pdseeykiye tnrgprfptd qtlqgrdtin dsyhakvqtp wqllhancwg
121 cwfspadwqq mittcrefkv ehleqaidni viktvtkqgt gaeeitqynn ditallqvae
181 dksnlmpwaa dnmyidsigy vpwrpsklpt ycyhvnfwnt idinagaqqn qwaevkkgiq
241 wdniqftpie nmvdiellrt gdrwdsgpyt fyckpssley hwqstrhtgs chpsttpqai
301 gsvgnnletl nawqwgdmn pssastrvsn fhigyswpew qfhystggpv vnpgqpfsqa
361 pwgsevagtr ltqgasekai ydwnhgdeeh qfretwwqnn enmtgqtnwa pknihqkelt
421 nnvaaqtafw aqgyhntfgp ftafddhgaq ypwgaiwgke pntthkplas shapfmtanp
481 pgqifyklap nytdtvdnng nssrivtfgt fwwtgklvik aklrtprqwn tyqlpslaar
541 etmketvpne ignfeipymp grampnytl
SEQ ID NO: 22
Human Parvovirus 4 VP1 amino acid sequence (GenBank AEE69129)
    1 msaadayrpg gklpldelmq rmnraipvgp epsspanrgg gpyqthfaig imyskafqgl
  61 lkfanalpae lspvkqlvnq lehyrrktsd trvwyrvyld mtrllisvap pgaanklrqa
121 aagithskap naetlrgivr faaaafvpti enidrffeds ltnfakedld twkqlheqfi
181 klfhppdvgv hlvsdsrdeg adsivepdle rpagggltlp gynyvgpgnp ldsgppqgpv
214 deaakhhder yaemiehgdi pylhghgadr lmnkeleekk rrgdithltd vvvgnairgl
301 wqaketvgdi advqlsqvlp pappssdqqp aysagepsak karigtpees eptlpvqsdt
361 ntmsvepagg gggvkvkaqw iggtsfsdsv vitshtrtsm ladrggyvpv ykpgshvdss
421 qpvmgmktpy syidvnalsa hftprdfqql ldeydeikpk sltiaisaiv ikdvatnqtg
481 tnvsdsasgg itvfaddsyd ypyvlghnqd tlpghlpgen yvlpqygyit rgreidqqns
541 ivaisdhkte lfflehhdae clgtgdhwsh hyefpddlpw rklstpnqtl parhnpipss
601 rlaimtgvdn dgtavwkrpe gmdvgrlpln yvpgpalmmp tdtqirnttf rdpvaignpa
661 tsdrysvapl vhqpwsvrte ewlanktdys vhnylggvay trrkheesyd kheedrdgrv
721 snpsrvvqid gdlaaphvgh tffvpghtrv tsggtdtvys pklyqepvfp 1fpgavwnpn
781 plsydcqiwt kipntechff aqypllggwg vmtpppmifv klrsqpgpps pgahtvpqsn
841 lnqyaifhlh ysmqflvkrr krsrrhnpek papfpttdsg rmpftlansl qdpstpvyev
901 psdqwiarny shll
SEQ ID NO: 23
Human Parvovirus 4 VP2 amino acid sequence (GenBank AEE69130)
    1 msvepagggg gvkvkaqwig gtsfsdsvvi tshtrtsmla drggyvpvyk pgshvdssqp
  61 vmgmktpysy idvnalsahf tprdfqqlld eydeikpksl tiaisaivik dvatnqtgtn
121 vsdsasggit vfaddsydyp yvlghnqdtl pqhlpgenyv lpqygyitrg reidqqnsiv
181 aisdnktelf flehhdaecl gtgdhwshhy efpddlpwrk lstpnqtlya rhnpipssrl
241 aimtgvdndg tavwkrpegm dvgrlplnyv pgpalmmptd tqirnttfrd pvaignpats
301 drysvaplvh qpwsvrteew lanktdysvh nylggvaytr rkheesydkh eedrdgrvsn
361 psrvvqidgd laaphvghtf fvpghtrvts ggtdtvyspk lyqepvfplf pgavwnpnpl
421 sydcqiwtki pntechffaq ypllggwgvm tpppmifvkl rsqpgppspg ahtvpqsnln
481 qyaifhlhys mqflvkrrkr srrhnpekpa pfpttdsgrm pftlanslqd pstpvyevps
541 dqwiarnysh ll
SEQ ID NO: 24
Human Parvovirus 4 NS1 amino acid sequence (GenBank AEE69128.1)
    1 mdapawiavl qiptgflsnp anwrdwdglq rprnlladdw pvqelresvp lfdhavnlgy
  61 cilqqlfash avtlpcrvkp smflqlepss geenemhyhl vvnqadmvgr ecsnwlrtwk
121 vfmagylvap twtlswnirk trqgrlyqad msfvknyllp klplndcyya wtnidrfeaa
181 vlsvrnrqls gpqgaialpf tdaqpqapaa egvpptmagk gtqrfmdlid wlvengiate
241 krwlsvnkls yrsflgssgg vlqaknalqi akremvlahp llgyltknas tfeesnkvaq
301 lfslngynpv daawyfaawa rgvwpkrrai wlwgpastgk tllaaaianl spsygcvnwt
361 nqnfpfndch cqslvwweeg rmteniveva kavlggapvr ldvknkgsed yiptcviits
421 ngdltvtvdg pvvstqhqea lqtritmfqf grmvpdglap lpeeevrsff klgeqelnmk
481 gtppeefrvp rnfdkqpmas tsnlpkalca pidedqvqwd seddwfpppt qkrrravqet
541 pprtpseiie fsspspvada ppttpdspge lsltptsvsq ivsapfpeet aerygagdie
601 afwsehvfda dwatrlhicp pggprpyglf wtylwsrefw rfkqsvsrse ahlvnrrfiw
661 aww
Reprsentative GSH wequences
SEQ ID NO: 25
PAX5 Genomic safe harbor sequence >NG_033894.1:184716-186382 Homo
sapiens paired box 5 (PAX5), RefSeqGene (LRG_1384) on chromosome 9
CCCAGCAATGGATCGATGCACGGCTGTCGGGGCCGACAGGCTGACCTTTACTGAGCTCAGGTTTTCATCT
CCCTGTTGGGAGCCCAGGAAGGTCTTGCTGTGGAGAGAGGAACGGTGAGAAGCCTTGGCCTGCGAGGGGG
AGAGGCTTGGCGTGGGTGCAGTGAAGACAGCTTCTGAGAGCTGAAAGCCCTTGGAGGTCACTTATTTCAA
TTTCTTCCAAACACACCACTTTTACAGATGAGAAAACTGAGACTTGGTGAGAAATGACTTGTCCAAGGTC
ACTCTAAGAGGCTTTGACACAGCTCCAGAATCCAGTGTGTGTGTATGTGTGTGTGTACACATCCAACATA
CATACATCTGTATATTATAAATATTATACATATATTACGTATACATACACACGATACATTATATGCATGT
GTACGTGTATATCGGGAGTGTCTATACATGTGTATTATGAAAGCCTGGCTGTGGCTACGTGTGATGCCGT
GCCTGCGCTCACTCTGGTCGTCAACAGTTTGGTCCCGCAACATCCCGGGTAGCCGCCGATCCCTGAGCCA
CCAGGCATTTCATGCAGTTCTGCAAAGCCATGGAGAGGAGCTGAAGAAACCTCATGGTCCTTTTCAAATC
GTTTCTTCCTCCTCCTCCTCTGCAAAGATTTTCTCTAAGCCCAGTTTGAATCCTTCAGAAACAGAACTTG
GCTGCGAAGTCACTTTGAAAGACTTTCCATATGTTAATTGCAGCCGGCCAAGGTCTGGAGCAGAGGTGGG
AGCCCACCATCTGCAGACGGGGTCGGCCCCCAGTGCGCTCTGCAAATCCCCGTCATCTGGCAGGTGTCGT
TTTGGGTTAATTAAGAGCTATACTGAGCCCGTTTACCTGTCACTTCTGAGAATTTTAGGAAACTTTGACT
TTCTTGCCATCTCTGAGCTTTGAGCGAAGGGGAAGCTGAAAACACCTCTGAATCTGGTGATGTTTCTGCC
TCTGGGATCTCCAGGACAGCTGCATTAAGTGCATCTTATCATAACCCCTTTTTAAACTTTTTATTTTAAT
CAGTGTTCTCTAGTTAGTGCATTGGTTTTTACAGTCACGTCTTCTATATTGGAAGACAGTACTGTTTGGG
GGAAACCCACCATTTGTCTGAAATTTCTTAAGGCTCTGCTTTCTCTCTGTGTCTTTGAGGAAACAGCATA
CATTCCTCTAGCTTTGTTCTGTGTAATGGCTTTGGAGAAACTTTGAATTTGCAGGTCAGGGGCTCTTTCA
CCCATTGGGGTTTGGGGCTGTCAGTGCTAACCTCAGAGCTCTATGTTCATGGAGGGATGACTCAGTTACA
TCCCCAGATAGCTGGGTTCTCGGTTGGTCAATAGGCCCCCTTCTTCAGTATGAGAGAATTTTCTCTCTGT
GCTGTTGACAATGTTCTATTAATATATCTTGGTAGGGGTTTGGGTCACACAGATCTATGCATTTGTCAAA
ACACAGCAATAGCACATTTAAGATTTGTGTGTTTCATTATGTGTAAATTTTGTATCCAAAGAAAAAACTA
GTAAACAAGTAATGAACTTCAGTTAATTGTATGCATGCTGAAGTACTTAGGGGAAAGTGTACTGATGTTT
GCATTTACTTGGAAATGAAATACACATTAAGGTGAAAGAAAGGCTAGAGGGATGAAG
SEQ ID NO: 26
KIF6 Genomic Safe Harbor Sequence
>NG 054928.1:303712-305348 Homo sapiens kinesin family member 6 (KIF6),
RefSeqGene on chromosome 6
AGTGGTGTGATCATGGCTTACTGCAGCCTCAACCTCCCAGATTCAAGTGATCCTCCTGCCTCAGCCTACC
GAGTAGCTGGGACTACAGATGCATGCCATCACGCCTGACTAATTTTACCTTTTGTAGAGATGAGGTCCCT
CTGTGTTGCCAAAGGTGGTCTAGAACTCCTGGGCTCAAATGATCCTCCCCCCTCCCTGGGCCTTCCAAAG
TACTGGGATTACAGGTGTAAGCCAATGCACTCAGCCCCATGTTACTTAATAGAAAGGTTTTTCTTCCCCT
TTTTCCTGCACCCTTTGCTGCTCTCACGGGGAATTTCTAGCATCTCTAAGCTCTGGTCTCCAGTCTGAGG
AAGTTGTGCTGCCTGTATGTGACAAGAGAAATAAGATGTTGGCACATGAATAGGATGTTCGCCCTTTGGT
GAACTAGAGCATGTGAGCCAATTCTTAAGCCAGATTTTTCAGCAGAGAACAATTGCAATTCACAATCACA
TTTTCCAGGCATGACTCATCCCTATAGTATACAATAATATGAAGAGAGGCTGGAAACCCCATGCTTGGCA
AATACCAGTGCCCAGGCACTGCAAGCTTTCTTTTGTGGCAGATTTTTCATACAAACTGAGTCCATCAGTC
TCAGAGTCCCATTCAATAACAAAAGAAGAAATAAATGGGGAGATTAACTGCTATTGGAAATGAAGGTGTT
GAAAATGTAAACTAAACAAAGCAAAGCACCCCTTCACTCAGTTGGATCCTTCTAACATAGAATCAAACAG
CCATCTAAAACCAACAGGAAAACCGGACCGAGGGTGGAGAGAAACCGTGTGGCACCATCAGGAGGTAACT
CCCATGGTGAGGAGGAGTTTCCTGGCTCCCATTAGACTTTCATTTTAACCCTTATGATTTTTCCTTTTTT
TTTTTTTTTTGTATTTACATCTTTTCTGCTGAACTAAAATGATAAACTCTGAGAGCAGAGATTAGATTAG
AATCTTCTCAGCCTCTCCACAATTTTAAAAGCAGTGCTGGCCACAGGAAAAAAAAAAAAAGGTACTCAAA
AAACACTTTTTTTGTTTGTGAATGACAATTTGAAATTGACTTTGAGAAATCTTGGCAGCCAAGAAAATGG
CTGGAGAAGACTTTACAGCTTCCGAGAAGTAGGAGGATGCAGCAGGCTTCTGGAGGGTCAGGGGAGGAGC
TGATCAACTGGAGGCGGGAGAGGGAGGCCATAGTGGGAGAGATGAAACGGGAAAGGAATACTAGCATTTT
TTAAAAAGCATAAGGGGAACAAAGGGTGGATCTTTATTACAATAAAGTGGAGGCAGCCAGGGTACAAGGT
ACAAGTTTATGGAGGAAAAAAATGGCAAAATATAGGCCCAGTCTTCTGTCCTCCTCTCTGACAGGGAAGG
GTATTGGATGTTCACTCTATGAAAAAGCAACATATTAAGTTAGTTGTTCTAGACAAGAAAAGTAGGAAAG
ATATTGTAGGAACCCTTTGCCCTCAAACACATATTGGCCCACCATTCTCAGAAGGCAATCTCAGCTGGCA
TGACAGAGCATCTGGTTGCAGAGGCTCTTGGGGACTGAGTGGCTGCTGAACGAACACCAGCCCCTCTCTT
TGGCCCATGGGTAAAAGCAGCCACTGC
Additional Representative AAV ITR sequences
As with SEQ ID NOs: 1, 2, and 3, further provided herein are
representative sequences of AAV2 ITRs. Wave underline - terminal
resolution site (trs); solid underline - B/B' and C/C' stems.
SEQ ID NO: 28
AAV2 ITR “Flip” conformer nucleic acid sequence

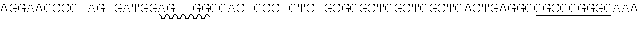

GCCCGGGCGTCGGGCGACCTTTGGTCGCCCGGCCTCAGTGAGCGAGCGAGCGCGCAG
The orientation of B/B' and C/C' stems from 5' to 3': B -> B'-> C -> C'
SEQ ID NO: 29
AAV2 ITR “Flip” conformer nucleic acid sequence

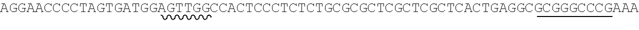

CGGGCCCGCTGCCCGCTGGTTTCCAGCGGGCGCCTCAGTGAGCGAGCGAGCGCGCAG
The orientation of B/B' and C/C' stems from 5' to 3': rB' -> rB -> rC'
-> rC “r” represents the reverse sequence of the B/B' and C/C' stems.
SEQ ID NO: 30
AAV2 ITR “Flop” conformer nucleic acid sequence

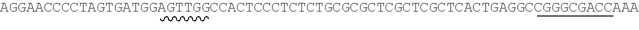

GGTCGCCCGACGCCCGGGCTTTGCCCGGGCGGCCTCAGTGAGCGAGCGAGCGCGCAG
The orientation of B/B' and C/C' stems from 5' to 3': C -> C' -> B -> B'
SEQ ID NO: 31
AAV2 ITR “Flop” conformer nucleic acid sequence

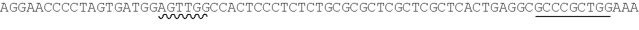

CCACGGGCTGCGGGCCCGTTTCGGGCCCGCGCCTCAGTGAGCGAGCGAGCGCGCAGAGAGGGAGTGGGCAA
The orientation of B/B' and C/C' stems from 5' to 3': rC' -> rC -> rB'
-> rB. “r” represents the reverse sequence of the B/B' and C/C' stems.
```

Additional representative ITR sequences of AAV2 and other AAV serotypes are known in the art. Examples of such sequences can be found at least in International Publication No. WO 2017/152149; Lusby et al. (1980) J Virol 34:402-409; Berns (2020) Human Gene Therapy, 31:518-523; Samulski et al. (2020) Human Gene Therapy, 31:151-162; and Tyson et al. (1990) J theor Biol 144:155-169; each of which is incorporated herein by reference.

A functional AAV ITR contains nucleic acid sequences comprising (i) binding sites for the p5 Rep proteins, (ii) nicking site (trs) sequences, and (iii) energetically stable secondary structure formed by the interrupted palindromic sequences as represented in FIG. 1A.

An ordinarily skilled artisan appreciates that AAV ITR sequences can be variable yet functionally equivalent because the sequences preserve the functional aspects: binding sites for p5 Rep proteins, trs sequences, and secondary structure formed by the interrupted palindromic sequences. Notably, for the reasons stated, AAV ITR sequences comprising the functional sequences that are complementary, reverse, or reverse complementary are equally functional.

### Protoparvovirus and Tetraparvovirus ITR Sequences

Similar to AAV ITRs, a functional protoparvovirus or tetraparvovirus ITR contains nucleic acid sequences comprising (i) binding sites for the NS proteins (Rep), (ii) nicking site (trs) sequences, and (iii) energetically stable secondary structure formed by the interrupted palindromic sequences. Representative sequences are known in the art.

An ordinarily skilled artisan appreciates that protoparvovirus or tetraparvovirus ITR sequences can be variable yet functionally equivalent because the sequences preserve the functional aspects: binding sites for NS proteins, trs sequences, and secondary structure formed by the interrupted palindromic sequences. Notably, for the reasons stated, protoparvovirus or tetraparvovirus ITR sequences comprising the functional sequences that are complementary, reverse, or reverse complementary are equally functional.

Included above are cDNA, ssDNA, and RNA nucleic acid molecules (e.g., thymidines replaced with uridines), nucleic acid molecules encoding orthologs or variants of the encoded proteins, as well as nucleic acid sequences comprising a nucleic acid sequence having at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the nucleic acid sequence of any SEQ ID NO listed above, or a portion thereof. Such nucleic acid molecules can have a function of the full-length nucleic acid as described further herein.

Included above are orthologs or variants of the proteins, as well as polypeptide molecules comprising an amino acid sequence having at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of any SEQ ID NO listed above, or a portion thereof. Such polypeptides can have a function of the full-length polypeptide as described further herein.

### Pulsatile Gene Expression and Tunable Gene Expression

In certain aspects, the recombinant virions, pharmaceutical compositions, and/or methods of the present disclosure utilize a pulsatile and/or tunable gene expression. As used herein, tunable gene expression allows regulation of the transgene expression at will, e.g., using a small molecule or an oligonucleotide (e.g., tetracycline or antisense oligonucleotides (ASO or AON), respectively) to turn on or turn off the expression of the transgene. While tunable gene expression is often achieved using an inducible promoter or a repressible promoter, the tunable regulation is intended to include the regulation of gene expression beyond transcription.

Accordingly, tunable gene expression is intended to encompass temporal regulation at transcriptional, post-transcriptional, translational, and/or post-translational levels. Tunable expression is compatible with spatial control of the gene expression. For example, spatial control of a transgene may be facilitated by placing a transgene under a tissue-specific promoter, which is then combined with an expression-modulating agent (e.g., tetracycline or ASO) that mediates temporal control.

Pulsatile gene expression refers to turning on and off the production of the transgene at regular intervals. Any tunable gene expression system may be utilized for pulsatile gene expression. In addition, it is contemplated herein that modulation of any gene expression described herein may be used in combination with pulsatile gene expression.

Pulsatile gene expression is important for the success of gene therapy. Obtaining physiological and long-term protein expression levels remains a major challenge in gene therapy applications. High-level expression of a transgene can induce ER stress and unfolded protein response months after treatment, leading to a pro-inflammatory state and cell death, jeopardizing the therapy's benefit. The pulsatile transgene expression strategy (PTES) can spare the target cell from overexpression stress, and allow long-term expression of the transgene without gradual reduction in expression over time. In addition, the pulsatile and/or tunable expression may improve, e.g., the efficiency of the production and/or stability of the protein encoded by the transgene.

In some embodiments, PTES described herein is a tunable expression system where the default state is off until a reagent turns-on or disinhibits expression, allowing calibration of dose to meet patients' specific needs, providing greater safety and long-term benefits. The timing of the pulses can be determined from the initial serum levels (t0) and the half-life (t½) of protein of interest (see Example 11).

### Exemplary Tunable Expression System

### Tetracycline-Controlled Operator System

A bacterial regulatory element, the Tn10-specified tetracycline-resistance operon of E. coli, can be used to regulate gene expression. For example, there are three exemplary configurations of this system: (1) The repression-based configuration, in which a Tet operator (TetO) is inserted between the constitutive promoter and gene of interest and where the binding of the tet repressor (TetR) to the operator suppresses downstream gene expression. In this system, the addition of tetracycline results in the disruption of the association between TetR and TetO, thereby triggering TetO-dependent gene expression. (2) Tet-off configuration, where tandem TetO sequences are positioned upstream of the minimal constitutive promoter followed by cDNA of gene of interest. Here, a chimeric protein consisting of TetR and VP16 (tTA), a eukaryotic transactivator derived from herpes simplex virus type 1, is converted into a transcriptional activator, and the expression plasmid is transfected together with the operator plasmid. Thus, culturing cells with tetracycline switches off the exogenous gene expression, while removing tetracycline switches it on. (3) Tet-on configuration, where the exogenous gene is expressed when tetracycline is added to the growth medium. Even though tetracycline is nontoxic to mammalian cells at the low concentration required to regulate TetO-dependent gene expression, its continuous presence may not be desired. Thus, a mutant tTA with four amino acid substitutions, termed rtTA, was developed by random mutagenesis of tTA. Unlike tTA, rtTA binds to TetO sequences in the presence of tetracycline, thereby activating the silent minimal promoter.

### Cumate-Controlled Operator System

The cumate-controlled operator originates from the p-cmt and p-cym operons in Pseudomonas putida. The corresponding repressor contains an N-terminal DNA-binding domain recognizing the imperfect repeat between the promoter and the beginning of the first gene in the p-cymene degradative pathway. Similar to a tetracycline-controlled operator system, the cumate operator (CuO) and its repressor (CymR) can be engineered into three configurations: (1) The repressor configuration, which is realized by placing CuO downstream of a constitutive promoter, where the binding of CymR to CuO efficiently suppresses downstream gene expression. The addition of cumate releases CymR, thereby triggering downstream gene expression. (2) Activator configuration, where chimeric molecular (cTA) is formed via the fusion of CymR and VP16. In this configuration, a minimal promoter was placed downstream of the multimerized operator binding sites (6×CuO). (3) Reverse activator configuration, for which after the random mutagenesis and screening, cTA mutant (rcTA) that binds to CuO upon addition of cumate was generated. In this configuration, the addition of cumate triggered downstream gene expression.

### Protein-Protein Interaction-Based Chimeric System

1. Induction of Target Gene by Control of the Interaction Between FKBP12 and mTOR

Rapamycin and its analog FK506 bind to a cytosolic protein FKBP12. This complex further binds to mTOR, forming a tripartite complex. Therefore, fusing FKBP12 and mTOR with a DNA-binding domain of ZFHD1 and the activation domain of NF-κB p65 protein, respectively, bridges both domains to drive expression of the gene of interest in a rapamycin-dependent fashion. Due to the immunosuppressive and the cell cycle inhibitory effect of FK506 and rapamycin, a new synthetic compound, FKCsA, which is a heterodimer of FK506 and cyclosporin A (an immunosuppressant complexed with protein cyclophilin), was developed and was shown to exhibit neither toxicity nor immunosuppressive effects. To trigger gene expression, the addition of FKCsA to cells hinges FKBP12 fused with the Gal4 DNA-binding domain (Gal4DBD) and cyclophilin fused with VP16, thereby activating expression of the gene of interest downstream of upstream activation sequence (UAS, Gal4DBD binding site).

### 2. Induction of Target Gene by Control of the Interaction Between PYL1 and ABI1

Abscisic acid (ABA)-regulated interaction between two plant proteins is used to regulate gene expression in a temporal and quantitive manner in mammalian cells. The two proteins are PYL1 (abscisic acid receptor) and ABI1 (protein phosphatase 2C56), which are important players of the ABA signaling pathway required for stress responses and developmental decisions in plants. According to the crystal structure of PYL1-ABA-ABI1 complex, interacting complementary surfaces of PYL1 (amino acids 33 to 209) and ABI1 (amino acids 126 to 423) were chosen for chimeric protein construction. Similarly, Gal4DBD was fused with ABI1 and VP16 with PYL1. Thus after transfecting this ABA-activator cassette and UAS-driven reporter into mammalian cells, ABA significantly induced the reporter's production. Compared to the rapamycin system, the ABA system has two compelling advantages: first, ABA is present in many foods containing plant extracts and oils-its lack of toxicity is supported by an extensive evaluation by the Environmental Protection Agency (EPA), secondly, since the ABA signaling pathway does not exist in mammalian cells, there should be no competing endogenous binding proteins as in the rapamycin systems. To further avoid any catalysis of possible unexpected substrates by ABI1, a mutation critical for its phosphatase activity was introduced into the chimeric protein.

### 3. Induction of Target Gene by Light Sensitive Protein-Protein Interactions

Two light-switchable transgene systems were developed by taking advantage of light-induced protein-protein interactions. The first one got inspiration from the molecular basis of the circadian rhythm of fungi. Vivid (VVD), a photoreceptor and light-oxygen-voltage (LOV) domain-containing protein from Neurospora crassa, forms a rapidly exchanging dimer upon blue-light activation. Thus, the chimeric protein consisting of VVD and Gal4 residues 1-65 dimerizes and becomes a transcriptional activator under blue light-illumination, while the active dimer disassociates in the absence of blue light. This means that the expression of the reporter downstream of UAS can be switched on and off in a spatiotemporal manner utilizing blue light. Moreover, mutagenesis optimization of VVD further reduced the background expression to a minimal level, making the system even more feasible. Another light-switchable transgene system (photoactivatable (PA)-Tet-OFF/ON) exploits the Arabidopsis thaliana-derived blue light-responsive heterodimer formation, consisting of the cryptochrome 2 (Cry2) photoreceptor and cryptochrome-interacting basic helix-loop-helix 1 (CIB1). Photolyase homology region (PHR) at Cry2's N-terminal part is the chromophore-binding domain that binds to Flavin adenine dinucleotide (FAD) by a noncovalent bond. CIB1 interacts with Cry2 in blue light-dependent manner. Thus, to make an inducible expression system, PHR was fused with the transcription activation domain of p65, and CIB1 was fused with the DNA binding, dimerization and Tetracycline-binding domains of TetR (residues 1-206). Accordingly, the reporter gene can be switched on with blue light illumination, while switching off can be achieved in two ways, either by the absence of the blue light or tetracycline addition. Meanwhile, a tetracycline insensitive mutation, H100Y, was established to make it purely dependent on illumination. Applying the same chimeric structure, but replacing TetR with rtTA, the reporter gene can be switched on with either blue light illumination or tetracycline, and switched off either by absence of the blue light or removal of tetracycline. Generally, two advantages of light-switchable transgene systems overwhelm all other systems. One is their rapid on and off cycle. Due to the nature of circadian rhythm, the two above-mentioned protein-protein interactions are dynamic, leading to a fast response and turnover. Even short pulses of light for 1-2 min are sufficient to induce luciferase expression, which has been shown to peak 1.1 h later and decline to the background level 3 h later. The other advantage is its precise spatial induction. Illumination within restricted areas or cell populations can be realized with advanced illumination sources, by which the reporter expression can be selectively induced in certain cells or subcellular regions of interest. These unique features will not only greatly facilitate the future cell-cell behavior studies, but also provide vast potential for clinical gene therapy.

### 4. Tamoxifen Controlled System

The tamoxifen inducible system, one of the best-characterized “reversible switch” models, has a number of beneficial features (e.g., reviewed by Whitfield et al. (2015) Cold Spring Harb Protoc. 2015(3):227-234). In this system, the hormone-binding domain of the mammalian estrogen receptor is used as a heterologous regulatory domain. Upon ligand binding, the receptor is released from its inhibitory complex and the fusion protein becomes functional. For example, a ligand-binding domain (LBD) of the estrogen receptor (ER) can be fused with a transgene, the product of which is a chimeric protein that can be activated by anti-estrogen tamoxifen or its derivative 4-OH tamoxifen (4-OH-TAM).

This system has been used in combination with a recombinase to generate a regulatable recombinase that modifies the genome. For example, either single or two plasmid systems can be used to achieve inducible gene expression. The first successful case was done in mouse embryonic cells. Two plasmids were transfected together. One was Cre-ER constitutive expressing plasmid, the other contained gene trap sequence flanked by LoxP, followed by β-galactosidase (LacZ) open reading frame. As a consequence, expression of LacZ could only be restored when Cre-loxP-mediated recombination was triggered and the gene trap sequence was excised. By these means, the reporter gene could be induced not only in undifferentiated embryonic stem cells and embryoid bodies, but also in all tissues of a 10-day-old chimeric fetus or specific differentiated adult tissues. In another example, to induce enhanced green fluorescent protein (EGFP) expression in baby hamster kidney (BHK) cells and to simplify the plasmid construction, Cre-ER cDNA flanked by LoxP sites were inserted between phosphoglycerate kinase (PGK) promoter and EGFP encoding sequence. In this system, Cre-ER functions as a gene trap to block the transcription of EGFP without 4-OH-TAM. Ignition of recombinase activity by 4-OH-TAM melts off the Cre-ER cassette and restores EGFP expression driven by PGK promoter. To exclude the effect exerted by endogenous steroids, three distinct ERs are mostly exploited: (1) mouse ER™ with a G525R mutation, (2) human ERT with G521R mutation and (3) human ERT2 containing three mutations G400V/M543/L544A.

### 5. Riboswitch-Regulatable Expression System

A riboswitch-regulatable expression system takes advantage of bacteria-derived RNA aptamers linked with hammerhead ribozymes (aptazymes). Aptamer acts as a molecular sensor and transducer for the whole apparatus, while ribozyme responds to the signal with conformation change and mRNA cleavage. For example, Gram-positive bacteria's aptazyme can directly sense excessive glucosamine-6-phosphate (GlcN6P) and cleave mRNA of the glms gene, whose protein product is an exzyme that converts fructose-6-phosphate (Fru6P) and glutamine to GlcN6P. These aptazymes, responding to tetracycline, theophylline, guanine, etc. were engineered to both knock down and overexpress the gene of interest (as reviewed by e.g., Yokobayashi et al. (2019) Curr Opin Chem Biol 52:72-78).

### 6. ASO (Antisense Oligonucleotides) Regulated Expression System

ASO can bind to DNA or RNA. ASO has demonstrated effective gene regulation acting at the RNA level to either activate the RISC complex and degrade the mRNA, or interfering with recognition of cis-acting elements. ASO are routinely formulated in lipid nanoparticles that efficiently transfect cells. The ASO are used for “knock-down” applications, either gain-of-function (i.e., dominant negative), transcripts, or homozygous recessive diseases. In diseases caused by dominant negative mutations where the ASO is not specific to the transcript from the mutant allele, e.g., Huntington's disease and other poly-glutamine expansion diseases, restoration of normal cell function may be accomplished using gene replacement using a vector—delivered transgene with alternative synonymouse codons that reduce sequence complementarity to exogenous ASO. Thus, the ASO depletes the transcripts from the endogenous alleles but the vector-driven transcripts are unaffected.

As illustrated in FIG. 11, ASO can modulate splicing to either negatively or positively regulate gene expression (see also Havens and Hastings (2016) Nucleic Acids Research 44:6549-6563). Example I of FIG. 11 shows that an ASO (an antisense oligonucleotides ASO or AON) can negatively regulate gene expression post-transcriptionally. Without ASO, a primary transcript is spliced into a translatable mRNA. The addition of an ASO (red line) complementary to the splice acceptor at the 3′ end of the intron/5′ end of Exon 2 interferes with splicing. Thus, in the presence of ASO, the intron remains in the transcript. This unprocessed RNA comprising the intron is either untranslatable or produces a non-functional protein upon translation.

Example II of FIG. 11 also illustrates that an ASO can positively affect gene expression post-transcriptionally. A primary transcript (left) contains 4 exons: exon 1, exon 3, and exon 4 encode the therapeutic protein, and exon 2 contains either a nonsense mutation(s) or an out-of-frame-mutation (OOF). Such exon 2 can be engineered into any transgene. Without the ASO, the transcript is processed into a mature mRNA comprising 4 exons, i.e., exon 2 with a nonsense mutation(s) or an OOF mutation remains. Thus, the resulting mRNA translates into a truncated or non-functional protein. By contrast, the addition of ASO interferes with splicing, and the mature mRNA consists of exon 1, exon 3, and exon 4, i.e., exon 2 with a nonsense mutation(s) or an OOF mutation is spliced out. Thus, at the default state (no ASO), the therapeutic protein is not produced. Only upon the addition of ASO, the therapeutic protein is produced, thereby resulting in positive regulation.

These approaches allow for knock-down of constitutively active transgene expression, i.e., default on. In some embodiments, the default on state is preferred. In other embodiments, a default off condition is preferred.

### Exemplary Pulsatile Gene Expression for Hemophilia A

In certain aspects, the recombinant virions, pharmaceutical compositions, and methods provided herein use the pulsatile gene expression for gene therapy for a subject afflicted with hemophilia A. In some embodiments, an ASO regulated expression system is used to transduce a gene encoding human coagulation Factor VIII (FVIII) to hepatocytes in a subject afflicted with hemophilia A. In some embodiments, a pulsatile gene expression (the transgene encoding FVIII is turned on and off at certain intervals) is used to regulate the amount of FVIII produced (see Example 11). The delivery and regulation of the transgene encoding FVIII or an active fragment thereof (e.g., with its B-domain deletion), the compositions and methods described herein address a long-felt medical need for which there is still no solution.

In 2020, the FDA did not approve the Biomarin biologics license application (BLA) for Valoctocogene Roxaparvovec (or BMN270) as a treatment for hemophilia A (HemA). A recombinant adeno-associated virus type 5 (rAAV5) delivered a derivative of the gene for human coagulation factor VIII (FVIII) to the liver of HemA patients. At higher doses, FVIII was expressed and secreted into the circulation of patients at levels equal to or greater than physiological levels effectively “curing” the treated patients. However, long-term expression levels decreased 0.5 to 0.33 each year during the three-year follow-up. Although the FVIII expression remained at levels that are clinically beneficial, the FDA expressed concern that if expression continued to decline at the same rate, the patients would revert to their hemophiliac phenotype. There are no definitive explanations for the decremental expression pattern: previous clinical studies for hemophilia B established that loss of FIX expression was primarily attributed to acute inflammation elicited by processed AAV capsid antigens. However, prophylactic steroid treatment attenuated or eliminated the capsid immune response and is now routine for liver directed rAAV treatments. Several possible explanations that account for the loss of FVIII expression are contemplated herein.

FVIII has been a difficult recombinant protein to produce in either microbial or eukaryotic expression systems. The development of the “B-domain” deleted version of FVIII reduced the size of the open-reading frame and improved the expression level. However, the FVIII expression levels were still substantially lower than other proteins. To overcome these low levels, Biomarin increased the vector dose in the clinical studies. Patients were treated with 6E+13 vector particles (referred to as vector genomes, or vg) per kg. Based on large animal models, a small minority of hepatocytes take-up (transduced) with rAAV5-FVIII and as a result of the large number of vg per cell, then express relatively large quantities of FVIII. The metabolic demand for FVIII expression likely disrupts the normal requirements for hepatocyte protein expression. The hepatocyte cellular compartments normally involved in protein folding and secretion may become congested with the FVIII. Endothelial cells that produce FVIII production are likely specialized for this activity and produce FVIII from the allele on the single X chromosome under the transcriptional control of the highly regulated native FVIII promoter.

Accordingly, in order to prevent gradual reduction in expression of the transgene encoding FVIII, the transgene is turned on and off at regular intervals to achieve a long-term efficacy. The timing of the pulses is determined based on the serum level and half-life of the FVIII protein (see Example 11 for details). For FVIII for hemophila A prevention or treatment, the ideal state is off until transiently activated. ASO can be used to elicit either a negative or a positive effect by interfering with cis—acting elements in the primary transcript, thereby providing flexibility in regulation of the pulsatile gene expression.

### Methods of Preventing or Treating Diseases

In certain aspects, provided herein are methods of preventing or treating a disease using the recombinant virion or pharmaceutical compositions described herein. In some embodiments, the recombinant virions disclosed herein provide to the subject a nucleic acid of interest (e.g., those encoding a therapeutic protein or a fragment thereof) transiently, e.g., the nucleic acid transduced by the recombinant virions is eventually lost after a certain period of expression. In preferred embodiments, the nucleic acid transduced by the recombinant virions integrates stably inside the cells.

In some embodiments, provided herein are methods of preventing or treating a disease, comprising administering to a subject in need thereof an effective amount of the recombinant virion or pharmaceutical composition of the present disclosure. In some embodiments, the nucleic acid encodes a protein. In some embodiments, the nucleic acid decreases or eliminates the expression of an endogenous gene. In some embodiments, provided herein are methods of preventing or treating a disease, comprising: (a) administering to a subject in need thereof an effective amount of the recombinant virion described herein comprising a nucleic acid that increases or restores the expression of a gene whose endogenous expression is aberrantly lower than the expression in a healthy subject; or (b) administering to a subject in need thereof an effective amount of the virion described herein comprising a nucleic acid that decreases or eliminates the expression of a gene whose endogenous expression is aberrantly higher than the expression in a healthy subject.

In some embodiments, provided herein are methods of preventing or treating a disease, comprising: (a) obtaining a plurality of cells from a subject with the disease, (b) transducing the cells with the virion described herein, optionally further selecting or screening for the transduced cells, and (c) administering an effective amount of the transduced cells to the subject. In some embodiments, the cells are autologous to the subject. In other embodiments, the cell are allogeneic to the subject. There are advantages of preparing transduced cells in vitro or ex vivo. First, the existence and location of the transgene in the target cell genome can be verified before administering them to the patient, thereby avoiding interfering with cell functions or off target effects. This improves safety, even without the use of GSH. Second, the transduced cells can be administered to a subject in need thereof without the recombinant virions. This eliminate any concern for triggering immune response or inducing neutralizing antibodies that inactivate recombinant virions. Accordingly, the transduced cells can be safely redosed or the dose can be titrated without any adverse effect.

In some embodiments, the at least one recombinant virion, pharmaceutical composition, or transduced cells of the present disclosure are administered via intravascular, intracerebral, parenteral, intraperitoneal, intravenous, epidural, intraspinal, intrasternal, intra-articular, intra-synovial, intrathecal, intra-arterial, intracardiac, intramuscular, intranasal, intrapulmonary, skin graft, or oral administration.

In some embodiments, the disease is selected from endothelial dysfunction, cystic fibrosis, cardiovascular disease, renal disease, cancer, hemoglobinopathy, anemia, hemophilia, myeloproliferative disorder, coagulopathy, sickle cell disease, alpha-thalassemia, beta-thalassemia, hemophilia (e.g., hemophilia A), Fanconi anemia, familial intrahepatic cholestasis, epidermolysis bullosa, Fabry, Gaucher, Nieman-Pick A, Nieman-Pick B, GM1 Gangliosidosis, Mucopolysaccharidosis (MPS) I (Hurler, Scheie, Hurler/Scheie), MPS II (Hunter), MPS VI (Maroteaux-Lamy), hematologic cancer, hemochromatosis, hereditary hemochromatosis, juvenile hemochromatosis, cirrhosis, hepatocellular carcinoma, pancreatitis, diabetes mellitus, cardiomyopathy, arthritis, hypogonadism, heart disease, heart attack, hypothyroidism, glucose intolerance, arthropathy, liver fibrosis, Wilson's disease, ulcerative colitis, Crohn's disease, Tay-Sachs disease, neurodegenerative disorder, Spinal muscular atrophy type 1, Huntington's disease, Canavan's disease, lysosomal storage diseases, rheumatoid arthritis, inflammatory bowel disease, psoriatic arthritis, juvenile chronic arthritis, psoriasis, and ankylosing spondylitis, and autoimmune disease, neurodegenerative disease (e.g., Alzheimer's disease, Parkinson's disease, Huntington's disease, ataxias), inflammatory disease, inflammatory bowel disease, Crohn's disease, rheumatoid arthritis, lupus, multiple sclerosis, chronic obstructive pulmony disease/COPD, pulmonary fibrosis, Sjogren's disease, hyperglycemic disorders, type I diabetes, type II diabetes, insulin resistance, hyperinsulinemia, insulin-resistant diabetes (e.g. Mendenhall's Syndrome, Werner Syndrome, leprechaunism, and lipoatrophic diabetes), dyslipidemia, hyperlipidemia, elevated low-density lipoprotein (LDL), depressed highdensity lipoprotein (HDL), elevated triglycerides, metabolic syndrome, liver disease, renal disease, cardiovascular disease, ischemia, stroke, complications during reperfusion, muscle degeneration, atrophy, symptoms of aging (e.g., muscle atrophy, frailty, metabolic disorders, low grade inflammation, atherosclerosis, stroke, age-associated dementia and sporadic form of Alzheimer's disease, pre-cancerous states, and psychiatric conditions including depression), spinal cord injury, arteriosclerosis, infectious diseases (e.g., bacterial, fungal, viral), AIDS, tuberculosis, defects in embryogenesis, infertility, lysosomal storage diseases, activator deficiency/GM2 gangliosidosis, alpha-mannosidosis, aspartylglucoaminuria, cholesteryl ester storage disease, chronic hexosaminidase A deficiency, cystinosis, Danon disease, Farber disease, fucosidosis, galactosialidosis, Gaucher Disease (Types I, II and III), GM1 Gangliosidosis, (infantile, late infantile/juvenile and adult/chronic), Hunter syndrome (MPS II), I-Cell disease/Mucolipidosis II, Infantile Free Sialic Acid Storage Disease (ISSD), Juvenile Hexosaminidase A Deficiency, Krabbe disease, Lysosomal acid lipase deficiency, Metachromatic Leukodystrophy, Hurler syndrome, Scheie syndrome, Hurler-Scheie syndrome, Sanfilippo syndrome, Morquio Type A and B, Maroteaux-Lamy, Sly syndrome, mucolipidosis, multiple sulfate deficiency, Neuronal ceroid lipofuscinoses, CLN6 disease, Jansky-Bielschowsky disease, Pompe disease, pycnodysostosis, Sandhoff disease, Schindler disease, and Wolman disease.

In some embodiments, provided herein are methods of preventing or treating a hemoglobinopathy, comprising: (a) administering to a subject in need thereof an effective amount of the virion described herein, comprising a nucleic acid that encodes a hemoglobin subunit, or (b) obtaining erythroid-lineage cells or bone marrow cells from a subject in need thereof, transducing the cells with the virion described herein, comprising a nucleic acid that encodes a hemoglobin subunit, optionally further selecting or screening for the transduced cells; and administering an effective amount of the cells to the subject. In some embodiments, the hemoglobinopathy is beta-thalassemia or sickle cell disease.

In certain aspects, provided herein are methods of preventing or treating a disease using at least one recombinant virion or pharmaceutical composition comprises at least one capsid protein or variant thereof of a protoparvovirus or a genotypic variant thereof.

Canine parvovirus (CPV) is one of the most studied species of protoparvovirus. CPV infects wild and domestic dogs. CPV has a genome size of ˜5.3 kb, 600 bp larger than AAV. The large genome makes CPV particularly attractive for the transfer of genes in human cells that cannot be accommodated in AAV derived vectors. Because CPV does not normally infect humans, there is no humoral immunity pre-existing against CPV in the human population, i.e., humans are seronegative for CPV capsid antigens. This is in stark contrast to AAV; humans are seropositive for AAV capsid antigen such that the presence of neutralizing AAV antibodies excludes a large percentage of patients eligible for AAV gene therapy. Therefore, the lack of neutralizing antibodies against CPV antigen in humans makes the CPV viral particles, or a recombinant virion comprising at least one capsid protein of CPV or a variant thereof, particularly useful for highly potent gene therapy applications to prevent or treat different human genetic diseases that cannot be treated efficiently with AAV-derived vectors. CPV uses the canine transferrin receptor (TfR or CD71) as a cellular receptor to enter the cell, a protein expressed in the external membrane of the canine host cells (Goodman, Lyi et al. 2010). CPV also can interact with the human TfR counterpart and therefore internalize and transduce human cells. In addition, as described above, the VP2 capsid protein of CPV can be engineered to comprise one or more mutations that alter tropism and the specificity/affinity of target cell interaction and eventually the efficiency of target cell transduction.

As described herein, protoparvovirus transduces cells via its interaction with transferrin receptors (TfR) that are expressed on the target cells. TfR or CD71 is expressed in brain microvascular endothelial cells (BMVECs) the major element of the blood-brain barrier (BBB) (Navone, Marfia et al. 2013). The BBB constitute the primary limitation for passage of substances, both soluble and cellular, from the blood into the brain. CD71 has become an alternative to drive receptor specific transcystosis and deliver macromolecules such as antibodies to the brain parenchyma. Thus, protoparvovirus (e.g., CPV) can exploit the use of CD71 to translocate to the brain via systemic administration and transduce brain cells to prevent or treat different neurodegenerative disorders and neuromuscular disorders including but not limited to spinal muscular atrophy type 1, Huntington's disease, Canavan's disease, and lysosomal storage diseases. TfR or CD71 is also highly expressed in erythroid progenitor cells at early stage during differentiation and B lymphoblast cells. CD71 expression transiently overlap with CD34 expression in progenitor cells, before differentiation to the lymphoid or erythroid lineages. Thus, protoparvovirus (e.g., CPV) can transduce the stem cells and be used for T cells, B cells or NK cells derived therapies after differentiation from stem cells. Some of these uses are in cancer therapy, antimicrobial or autoimmunity related therapies. After HSC differentiation to the myeloid progenitors lineage, CD71/TfR is highly expressed in basophilic Endemic Burkitt lymphoma (EBL), polychromatic erythroblast and orthochromatic erythroblasts during erythropoiesis, before the final step to produce non-nucleated erythrocytes, therefore protoparvovirus (e.g., CPV) vectors can be used for the treatment or prevention of non-malignant hemoglobinopathies such as sickle cell disease by expressing anti-sickling versions of the hemoglobin genes.

In certain aspects, provided herein are methods of preventing or treating a disease using at least one recombinant virion comprising at least one capsid protein or a variant thereof of a bufavirus, cutavirus, or tusavirus. Bufavirus, cutavirus, tusavirus, or a recombinant virion comprising at least one capsid protein or variant thereof of any one of said viruses, has broad applications for gastrointestinal disorders and other target tissues. For instance, cutavirus has been isolated from skin samples in patients with cutaneous T cells lymphomas and melanomas, showing a tropism for T and B cells. Such tropism makes cutavirus attractive for gene transfer applications in lymphoid progenitor cells and subsequent applications (i) in differentiated T cells such as CAR-T and related cancer therapies, or (ii) in differentiated B cells and their applications to express therapeutic human antibodies against invading pathogens, tumor cells (e.g., tumor antigens or neoantigens), or chronic autoimmune disease.

In some embodiments, the at least one recombinant virion comprises a capsid protein(s) or variant thereof of a cutavirus. In some embodiments, the at least one recombinant virion or pharmaceutical composition targets a T cell, B cell, and/or a lymphoid progenitor cell. In some embodiments, the at least one recombinant virion, pharmaceutical composition, or transduced cells prevent or treat cancer.

In certain aspects, provided herein are methods of preventing or treating a disease using at least one recombinant virion comprising at least one capsid protein or a variant thereof of a tetraparvovirus, e.g., human parvovirus 4 (PARV4). Tetraparvovirus genus contains the human Parvovirus 4 (PARV4), porcine Parvovirus 2, Eidolon elvum parvovirus, Yak parvovirus, Porcine Hokovirus and Ovine Hokovirus. Human parvovirus 4 (PARV4) was originally detected in plasma from a person at risk for infection with HIV through injection drug use (Jones, Kapoor et al. 2005). PARV4 has a genome of ˜5.3 Kb, 900 nucleotides larger than AAV. PARV4's capsid is highly resistant to temperature, which makes it a remarkably versatile and stable viral vector. PARV4 is endemic in certain geographic areas, but elsewhere is found confined only to certain high-risk groups such as patients with HIV, HBV or HCV infections, in the setting of persons who inject drugs (PWIDs) and those with a history of multiple transfusions. It remains uncertain whether PARV4 actually causes the observed disease, or is a non-pathogenic, opportunistic virus that was detected in a highly exposed, at-risk (for viral infection) population. Seroprevalence of PARV4 in the general population varies in different parts of the world ranging from 0% to 25% (Sharp, Lail et al. 2009). Although, the anti-PARV4 antibody prevalence in patients with chronic viral infections is reported to be between 15-35% in different regions of the world, the prevalence in healthy individuals from other Western countries has been shown to be lower than 1%. A PARV4 IgG screening of UK blood donors identified a seropositivity rate of 4.8%, which is significantly lower than the observed seropositivity rate for primate AAV antigen (Matthews, Sharp et al. 2017). This highlights one of the main benefits of the use of PARV4 as a gene therapy vector. The reduced seroprevalence of neutralizing antibodies in the population of several Western countries, including the U.S., together with tropism for specific target tissues makes it a versatile gene therapy vector. PARV4 tropism and sites of latency are not fully understood, but compelling data suggest that bone marrow, respiratory tract, liver, and gut represent potential sites of viral replication and may be reservoirs for the virus in latent or persistent infected individuals. Therefore, a PARV4 vector is particularly useful for delivering a therapeutic gene to prevent or treat a broad range of human diseases, including hematologic diseases.

In some embodiments, the at least one recombinant virion or pharmaceutical composition comprises a nucleic acid encoding a protein or a fragment thereof selected from a hemoglobin gene (HBA1, HBA2, HBB, HBG1, HBG2, HBD, HBE1, and/or HBZ), alpha-hemoglobin stabilizing protein (AHSP), coagulation factor VIII, coagulation factor IX, von Willebrand factor, dystrophin or truncated dystrophin, micro-dystrophin, utrophin or truncated utrophin, micro-utrophin, usherin (USH2A), CEP290, INS, F8 or a fragment thereof (e.g., fragment encoding B-domain deleted polypeptide (e.g., VIII SQ, p-VIII)), and cystic fibrosis transmembrane conductance regulator (CFTR).

In some embodiments, the at least one recombinant virion or pharmaceutical composition transduces (a) a CD34+ stem cell, optionally transduces ex vivo; (b) a mesenchymal stem cell, optionally transduces ex vivo; (c) a liver cell, (d) a small intestinal cell, and/or (e) a lung cell.

For example, in some embodiments, a recombinant virion comprising at least one capsid protein of a tetraparvovirus (e.g., PARV4) is used for ex vivo modification (e.g., delivering a therapeutic gene and/or agents that downregulate a disease-associated mutant gene) in CD34+ stem cells. In some embodiments, a recombinant virion comprising at least one capsid protein of a tetraparvovirus (e.g., PARV4) is used for ex vivo modification (e.g., delivering a therapeutic gene and/or agents that downregulate a disease-associated mutant gene) in mesenchymal stem cells. In some embodiments, a recombinant virion comprising at least one capsid protein of a tetraparvovirus (e.g., PARV4) is delivered to the small intestine preferably via oral administration. In some embodiments, a recombinant virion is administered to the liver cells via systemic intravenous administration. In some embodiments, a recombinant virion is administered to the liver cells preferably via hepatic artery or portal vein.

In some embodiments, the at least one recombinant virion or pharmaceutical composition comprises a nucleic encoding (a) CFTR or a fragment thereof, (b) at least one non-coding RNA (e.g., piRNA, miRNA, shRNA, siRNA, antisense RNA) that targets an endogenous mutant form of CFTR, (c) a CRISPR/Cas system that targets an endogenous mutant form of CFTR; and/or (d) any combination of any one of the nucleic acids listed in (a) to (c). In some embodiments, the at least one recombinant virion or pharmaceutical composition is delivered to the lung via an intranasal or intrapulmonary administration. In some embodiments, the at least one recombinant virion or pharmaceutical composition (a) increases the expression of CFTR or fragment thereof, and/or (b) decreases the expression of an endogenous mutant form of CFTR in the transduced cell. In some embodiments, the at least one recombinant virion or pharmaceutical composition prevents or treats cystic fibrosis.

In some embodiments, the methods of preventing or treating a disease further include re-administering at least one additional amount of the virion, pharmaceutical composition, or transduced cells. In some embodiments, the re-administering the at least one additional amount is performed after an attenuation in the treatment subsequent to administering the initial effective amount of the virion, pharmaceutical composition, or transduced cells. In some embodiments, the at least one additional amount is the same as the initial effective amount. In some embodiments, the at least one additional amount is more than the initial effective amount. In some embodiments, the at least one additional amount is less than the initial effective amount. In certain embodiments, the at least one additional amount is increased or decreased based on the expression of an endogenous gene and/or the nucleic acid of the recombinant virion. The endogenous gene includes a biomarker gene whose expression is, e.g., indicative of or relevant to diagnosis and/or prognosis of the disease.

In certain aspects, the methods of preventing or treating a disease further comprise administering to the subject or contacting the cells with an agent that modulates the expression of the nucleic acid. In some embodiments, the agent is selected from a small molecule, a metabolite, an oligonucleotide, a riboswitch, a peptide, a peptidomimetic, a hormone, a hormone analog, and light. In some embodiments, the agent is selected from tetracycline, cumate, tamoxifen, estrogen, and an antisense oligonucleotide (ASO). In some embodiments, the methods further comprise re-administering the agent one or more times at intervals. In some embodiments, the re-administration of the agent results in pulsatile expression of the nucleic acid. In some embodiments, the time between the intervals and/or the amount of the agent is increased or decreased based on the serum concentration and/or half-life of the protein expressed from the nucleic acid.

In certain aspects, further provided herein are methods of modulating (i) gene expression, or (ii) function and/or structure of a protein in a cell, the method comprising transducing the cell with the virion or pharmaceutical composition described herein comprising a nucleic acid that modulates the gene expression, or the function and/or structure of the protein in the cell. In some embodiments, such nucleic acid comprises the sequence encoding CRISPRi or CRISPRa agents. In some embodiments, the gene expression, or the function and/or structure of the protein is increased or restored. In some embodiments, the gene expression, or the function and/or structure of the protein is decreased or eliminated.

### Exemplary Diseases

In certain aspects, the methods, recombinant virions, and/or pharmaceutical compositions described herein may be used for prevention and/or treatment of various diseases.

The recombinant virions and/or pharmaceutical compositions comprising at least one capsid protein of protoparvovirus or tetraparvovirus are useful for transducing a hematopoietic cells, hematopoietic progenitor cell, hematopoietic stem cells, erythroid lineage cell, megakaryocyte, erythroid progenitor cell (EPC), CD34+ cell, CD36+ cell, mesenchymal stem cell, nerve cell, intestinal cells, intestinal stem cell, gut epithelial cell, endothelial cells, lung cells, enterocyte, liver cell (e.g., hepatocyte, hepatic stellate cells (HSCs), Kupffer cells (KCs), liver sinusoidal endothelial cells (LSECs)), brain microvascular endothelial cell (BMVECs), erythroid progenitor cell, lymphoid progenitor cells, B lymphoblast cell, T cells, B cells, basophilic Endemic Burkitt Lymphoma (EBL), polychromatic erythroblast, and/or orthochromatic erythroblast.

In addition, the methods, recombinant virions, and/or pharmaceutical compositions described herein are particularly useful in delivering a nucleic acid (e.g., a therapeutic nucleic acid) in vivo (e.g., administering directly to a subject, e.g., targeting a specific tissue via viral tropism), as well as in vitro or ex vivo (obtaining a plurality of cells from a subject, transducing the said cells using the recombinant virions, and administering the subject an effective number of transduced cells).

### Hemochromatosis

The biodistribution of an orally administered protoparvovirus gene therapy vector is dictated by TfR-mediated transcytosis in the intestinal epithelia. Translocation of gut epithelial barrier by the gene therapy vector can expand its biodistribution. By a rational modification of the protoparvovirus capsid (e.g., VP2) as described herein, the present invention encompasses the use of such engineered viral vector with tropism restricted to or predominantly targeting enterocytes due to an inability to transcytose from the lumen to the lamina propia in the intestinal epithelia. Gene therapy using such engineered virions provides an ideal means to prevent or treat genetic diseases in the gut epithelia such as hereditary hemochromatosis.

Hereditary hemochromatosis (HH) is an autosomal recessive genetic disorder and the most prevalent genetic disease in Caucasians (Centers for Disease Control and Preventions; World Wide Web at cdc.gov). An estimated one million people in the United States have hereditary hemochromatosis, surpassing the prevalence of cystic fibrosis and muscular dystrophy combined (Bacon, Powell et al. 1999). HH is characterized by dysregulation in iron absorption. In HH patients, iron absorption is defective and the body absorbs iron in excess. High levels of intracellular iron deposition induce the formation of genotoxic oxygen radicals and lipoperoxidation, which establishes a pro-inflammatory response that result in chronic damage to a number of organs. The clinical features of the disease arise as result of decades of continuous accumulation of iron in parenchymal cells of the liver, heart and pancreas. In the most advanced form, HH is manifested as cirrhosis, hepatocellular cancer, diabetes mellitus, hypogonadism, cardiomyopathy, arthritis, and skin pigmentation. Enterocytes in the intestinal villi mediate the apical uptake of iron from the intestinal lumen; iron is then exported from the cells into the circulation. The apical divalent metal transporter-1 (DMT1) transports iron from the lumen into the cells, while ferroportin, a basolateral membrane bound transporter, export iron from the enterocytes into the circulation (Ezquer, Nunez et al. 2006). HH patients show an increased transepithelial iron uptake, which leads to body iron accumulation and the subsequent chronic complications (cirrhosis, hepatocellular carcinoma, pancreatitis, cardiomyopathy, arthritis and diabetes).

The most common cause of hereditary hemochromatosis is a mutation of the human homeostatic iron regulator (HFE) gene, identified on chromosome 6. Mutations in HFE are responsible for almost 90% of HH cases. The HFE gene encodes a major histocompatibility complex MHC class I-like molecule. HFE binds to 02-microglobulin, which determines its localization to the plasma membrane (Waheed, Parkkila et al. 1997). The main mutation described for HFE in association with HH is a single nucleotide change in exon 4 that results in a tyrosine for cysteine amino acid substitution at position 282 (C282Y) of the unprocessed HFE protein (Feder, Gnirke et al. 1996). This mutation affects its proper post-translational processing in the Golgi apparatus, disrupting its interaction with p2-microglobulin, and its subsequent localization in the cellular membrane. (Feder, Tsuchihashi et al. 1997, Waheed, Parkkila et al. 1997). A second mutation in the HFE gene, in which an aspartic acid moiety replaces histidine at position 63 (H63D) of the HFE protein, has also been reported (Gochee, Powell et al. 2002). The mutated and unfolded HFE protein is then accumulated in the ER-Golgi network, inducing the activation of the unfolded protein response (UPR), thus, exacerbating the pro-inflammatory program and subsequent outcome of the disease (de Almeida and de Sousa 2008, Liu, Lee et al. 2011). HFE coordinates the activity of both the iron import and iron export machinery in intestinal cells and is part of a multi-protein complex involved in transcriptional regulation of the hepcidin gene in the liver. Loss of HFE function is also associated with a drastic reduction in hepcidin expression, a negative regulator of iron uptake. Lack of HFE or hepcidin consequently results in an elevated incorporation of dietary iron and accumulation in different organs.

Another more severe form of the disease is Juvenile hemochromatosis (JH). This type of hemochromatosis is inherited and described as type II hemochromatosis. Type II hemochromatosis is categorized as type IIa or type IIb depending on the affected genes. In types IIa and IIb, the early iron overload onset occurs before 30 years of age. The consequences are severe heart disease or heart attack, hypothyroidism, little to no menstruation or hypogonadism. Hemochromatosis type IIa, results from an autosomal recessive mutation in the hepcidin gene, in chromosome 19.

Juvenile hemochromatosis is characterized by onset of severe iron overload occurring typically in the first to third decade of life. Males and females are equally affected. Prominent clinical features include hypogonadotropic hypogonadism, cardiomyopathy, glucose intolerance and diabetes, arthropathy, and liver fibrosis or cirrhosis. Hepatocellular cancer has been reported occasionally, while cardiac involvement is the main cause of morbidity and mortality.

Interestingly, the only accepted treatment for this disease is medieval, and involves periodic bleeding (phlebotomy) to reduce the iron load that is borne primarily through non-covalent coordination with heme molecules in red blood cells. At present, initially one or two units of blood (500-1000 ml) each containing 200-250 mg of iron are removed weekly until serum ferritin levels are reduced below 50 ng/ml and transferrin saturation drops to a value below 30% (requiring 2 to 3 years). Less aggressive bleeding, but life-long maintenance therapy, is then mandatory to keep the transferrin saturation value below 50% and the serum ferritin levels below 100 ng/ml (Wojcik, Speechley et al. 2002).

One therapy for hemochromatosis of different etiologies is the inhibition of DMT1 protein synthesis by the use of a siRNA in the enterocyte, which markedly inhibit apical iron uptake by intestinal epithelial cells (Ezquer, Nunez et al. 2006). The divalent metal transporter DMT-1 recently has been shown to also transport copper ions (Arredondo et al., 2003), thus inhibition of DMT-1 gene expression is of value in reducing liver injury in Wilson's disease, a condition in which copper export from cells is diminished. Decreasing the uncontrolled iron uptake in the enterocytes of HH patients will restrict the iron accumulation in several affected organs.

Another approach to control the iron load is through inhibition of ferroportin gene expression in enterocytes, to reduce the basolateral iron export. In this case, absorbed iron would only accumulate inside the enterocyte. Additionally, the accumulation of iron should lead to a reduction in the expression of the apical DMT-1 transporter gene by the IRE/IRP mechanism, producing a dual inhibitory effect. Further, any accumulated iron would be lost into the intestinal lumen by the normal slough of enterocytes.

The use of a recombinant virion comprising at least one capsid protein or variant thereof of protoparvovirus or tetraparvovirus to express wild-type HFE in enterocytes can restore the HFE activity and also positively modulate the expression of DMT-1 and ferroportin, thereby having a broad therapeutic effect. A combinatorial strategy using one or more recombinant virions described herein that co-express and/or co-administer wild-type HFE and an siRNA to silence DMT-1 can also enhance the clinical benefit.

The peptide hepcidin is a key regulator of iron metabolism. It is synthesized predominantly in the liver and secreted as a 20-25 amino acid peptide. Mutations of the hepcidin gene are responsible forjuvenile hemochromatosis (Roetto, Papanikolaou et al. 2003). HFE modulates the expression of hepcidin in the liver. Hepcidin negatively regulates iron release from reticuloendothelial macrophages and from the enterocytes that mediate intestinal absorption of iron (Nemeth, Tuttle et al. 2004, Nemeth, Roetto et al. 2005, Rivera, Liu et al. 2005). The use of a recombinant virion described herein to deliver and express hepcidin in the liver can reduce the uptake of iron by the body and reduce the toxicity associated with iron overload, thereby preventing all form of hemochromatosis.

One of the main characteristics of the hereditary hemochromatosis (e.g., due to a lack of functional HFE protein) is an upregulation of factors involved in iron homeostasis in enterocytes, such as DMT-1, ferroportin, and/or Transferrin receptors (TfR). As described above, TfR or CD71 is the cellular receptor for protoparvovirus (e.g., canine parvovirus) that facilitates transduction of protoparvovirus or a recombinant virion comprising at least one of its capsid protein. Accordingly, the upregulation of TfR in subjects afflicted with hereditary hemochromatosis provides an excellent opportunity to target and enrich the therapeutic genes and/or agents, which modulate the expression of various proteins involved in iron homeostasis in enterocytes, using a recombinant virion comprising at least one capsid protein or variant thereof of protoparvovirus (e.g., canine parvovirus). The larger genome size of a protoparvovirus or a recombinant virion comprising at least one of its capsid proteins additionally provides flexibility in packaging a larger gene and/or agents for gene therapy to prevent or treat hemochromatosis.

In certain aspects, provided herein are methods of preventing or treating a disease using at least one recombinant virion, pharmaceutical composition, or transduced cells comprising a nucleic acid encoding (a) hepcidin or a fragment thereof, and/or homeostatic iron regulator (HFE) or a fragment thereof, (b) at least one non-coding RNA (e.g., piRNA, miRNA, shRNA, siRNA, antisense RNA) that targets DMT-1, ferroportin, and/or an endogenous mutant form of HFE; (c) a CRISPR/Cas system that targets DMT-1, ferroportin, and/or an endogenous mutant form of HFE; and/or (d) any combination of any one of the nucleic acids listed in (a) to (c).

In some embodiments, the fragment is a biologically active fragment.

In some embodiments, the subject is administered with the at least one recombinant virion or pharmaceutical composition comprising a nucleic acid encoding:
a) hepcidin or a fragment thereof, wherein the at least one recombinant virion or pharmaceutical composition is administered to the subject intravenously, optionally wherein (i) said nucleic acid is operably linked to a promotor, and/or (ii) the at least one recombinant virion or pharmaceutical composition transduces a hepatocyte;
b) HFE or a fragment thereof, wherein the at least one recombinant virion or pharmaceutical composition is administered to the subject intravenously, optionally wherein (i) said nucleic acid is operably linked to a promotor, and/or (ii) the at least one recombinant virion or pharmaceutical composition transduces a hepatocyte;
c) HFE or a fragment thereof, wherein the at least one recombinant virion or pharmaceutical composition is administered to the subject orally, optionally wherein (i) said nucleic acid is operably linked to a promotor, and/or (ii) the at least one recombinant virion or pharmaceutical composition transduces an enterocyte;
d) at least one non-coding RNA (e.g., piRNA, miRNA, shRNA, siRNA, antisense RNA) that targets an endogenous mutant form of HFE, wherein the at least one recombinant virion or pharmaceutical composition is administered to the subject intravenously, optionally wherein (i) said nucleic acid is operably linked to a promotor, and/or (ii) the at least one recombinant virion or pharmaceutical composition transduces a hepatocyte;
e) at least one non-coding RNA (e.g., piRNA, miRNA, shRNA, siRNA, antisense RNA) that targets an endogenous mutant form of HFE, wherein the at least one recombinant virion or pharmaceutical composition is administered to the subject orally, optionally wherein (i) said nucleic acid is operably linked to a promotor, and/or (ii) the at least one recombinant virion or pharmaceutical composition transduces an enterocyte;
f) at least one non-coding RNA (e.g., piRNA, miRNA, shRNA, siRNA, antisense RNA) that targets DMT-1, wherein the at least one recombinant virion or pharmaceutical composition is administered to the subject orally, optionally wherein (i) said nucleic acid is operably linked to a promotor, and/or (ii) the at least one recombinant virion or pharmaceutical composition transduces an enterocyte;
g) at least one non-coding RNA (e.g., piRNA, miRNA, shRNA, siRNA, antisense RNA) that targets ferroportin, wherein the at least one recombinant virion or pharmaceutical composition is administered to the subject orally, optionally wherein (i) said nucleic acid is operably linked to a promotor, and/or (ii) the recombinant virion or pharmaceutical composition transduces an enterocyte; or
h) a combination of two or more of any one of a) to g).

In some embodiments, the method comprises a combination of two or more of any one of b) to e).

In some embodiments, the recombinant virion or pharmaceutical composition a) increases the expression of HFE or a fragment thereof, and/or hepcidin or a fragment thereof in the transduced cell; and/or b) decreases the expression of DMT-1, ferroportin, and/or an endogenous mutant form of HFE in the transduced cell. In some embodiments, the at least one recombinant virion, pharmaceutical composition, or transduced cells prevent or treat hemochromatosis, hereditary hemochromatosis, juvenile hemochromatosis, and/or Wilson's disease.

### Inflamatory Bowel Disease (IBD)

Inflammatory Bowel Diseases (IBD) include a series of disorders that involve chronic inflammation of the human digestive tract. The most common forms of IBDs are ulcerative colitis and Crohn's disease. These are complex, multifactorial disorders characterized by chronic relapsing intestinal inflammation. Although etiology remains largely unknown, recent research has suggested that genetic factors, environment, microbiota, and autoimmune responses are contributory factors in the pathogenesis (Hendrickson, Gokhale et al. 2002). An estimated 3 million people in the U.S. have been diagnosed with IBD (World Wide Web at cdc.gov/ibd/data-statistics.htm), with 70,000 new cases of Crohn's disease or ulcerative colitis diagnosed each year. There is currently no cure for these painful disorders and the treatments represent an estimated annual financial healthcare burden of 6.3 billion dollar (Limanskiy, Vyas et al. 2019). The multifactorial components associated with IBD converge in the activation of a pro-inflammatory program, fundamentally mediated by genes activated by the NFkB pathway. The main pro-inflammatory cytokines induced during IBD that mediate the IBD pathobiology are TNFα, IL-1β, IL-12 and IL-6. Given the high resistance of different protoparvovirus- and tetraparvovirus-derived virions to low pH and the ability of these viral vectors to transduce human enterocytes, the recombinant virions of the present disclosure constitute a novel therapeutic approach to modulate expression and/or function of various cytokines as well as subsequent maintenance of the gastrointestinal epithelial barrier integrity.

In some embodiments, a recombinant virion comprising at least one capsid protein of a protoparvovirus or tetraparvovirus, or a virion comprising an engineered capsid protein, is used to express a soluble form of the TNFα receptor, soluble form of the IL-6 receptor, soluble form of IL-12 receptor, and/or the soluble form of IL-1β receptor. These soluble forms of said receptors can be secreted to the small intestine lamina propia where they specifically neutralize the ligands (e.g., pro-inflammatory cytokines).

A soluble form of the membrane-bound receptors can be expressed by delivering a gene encoding a soluble secreted form of the receptor. For example, a 17-kDa soluble moiety of TNFα is known to be released from cells after proteolytic cleavage of the 26-kDa type II transmembrane isoform by TNFα-converting enzyme (TACE; ADAM-17) (Kriegler et al. (1988) Cell 53:45-53). Thus, a recombinant virion of the present disclosure comprising a gene encoding the 17-kDa moiety (or any desired portion of the extracellular domain, e.g., the portion that interacts with the ligand to be antagonized/neutralized) fused to a signal peptide (e.g., IL-2 signal peptide; see e.g., Ardestani et al. (2013) Cancer Res. 73:3938-3950) can be delivered in vivo to a subject in need thereof (e.g., a subject afflicted with IBD or other inflammatory disorders) to express the soluble form of TNFα in said subject. Alternatively, either autologous or allogeneic cells can be transduced in vitro or ex vivo with such a virion comprising a gene encoding a secreted soluble form of a membrane protein, and said cells can be transferred to a subject in need thereof to treat the subject. Similar strategies can be used for any membrane bound protein.

In certain aspects, provided herein is at least one recombinant virion, pharmaceutical composition, or transduced cells comprise a nucleic acid encoding (a) a soluble form of the TNFα receptor, a soluble form of the IL-6 receptor, a soluble form of the IL-12 receptor, and/or a soluble form of the IL-1β receptor; (b) at least one non-coding RNA (e.g., piRNA, miRNA, shRNA, siRNA, antisense RNA) that targets the TNFα receptor, IL-6 receptor, IL-12 receptor, and/or IL-1β receptor; (c) a CRISPR/Cas system that targets the TNFα receptor, IL-6 receptor, IL-12 receptor, and/or IL-1β receptor; and/or (d) any combination of any one of the nucleic acids listed in (a) to (c).

In some embodiments, the at least one recombinant virion or pharmaceutical composition a) increases the expression of a soluble form of the TNFα receptor, a soluble form of the IL-6 receptor, a soluble form of the IL-12 receptor, or a soluble form of the IL-1β receptor in the transduced cell; and/or b) decreases the expression of the TNFα receptor, IL-6 receptor, IL-12 receptor, or IL-1β receptor in the transduced cell.

In some embodiments, the at least one recombinant virion, pharmaceutical composition, or transduced cells prevent or treat rheumatoid arthritis, inflammatory bowel disease, psoriatic arthritis, juvenile chronic arthritis, psoriasis, and/or ankylosing spondylitis.

Accordingly, the recombinant virions of the present disclosure comprising the said therapeutic genes and/or agents modulate chronic inflammation in a subject and provide therapeutic benefit by decreasing the activation of T cells, NK cells, and other effector immune cells, and allow subsequent repair of the damaged epithelial barrier. The therapeutic benefit can be further enhanced by the combination strategies provided herein.

### Autophagy-Related Diseases

The methods, recombinant virions, and/or pharmaceutical compositions of the present disclosure can be used to modulate the critical components of the autophagy-lysosome pathway. Autophagy plays crucial roles in differentiation and development, cellular and tissue homeostasis, protein and organelle quality control, metabolism, immunity, and protection against aging and diverse diseases. The macro-autophagy form of autophagy (hereinafter referred to as autophagy) is an evolutionarily conserved lysosomal degradation pathway that controls cellular bioenergetics (by recycling cytoplasmic components) and cytoplasmic quality (by eliminating protein aggregates, damaged organelles, lipid droplets, and intracellular pathogens) (Levine, Packer et al. 2015). In addition, independently of lysosomal degradation, the autophagic machinery can be deployed in the process of phagocytosis, apoptotic corpse clearance, secretion, exocytosis, antigen presentation, and regulation of inflammatory signaling. As a result of the broad range of cellular functions, the autophagy pathway plays a key role in protection against aging and certain cancers, infections, neurodegenerative disorders, metabolic diseases, inflammatory diseases, and muscle diseases (Levine, Packer et al. 2015).

Numerous diseases are associated with the accumulation of undesired, potentially cytotoxic cellular debris, such as misfolded-protein aggregates, nucleic acids and/or pieces of damaged organelles such as mitochondria. Autophagy also degrades lipids, allowing catabolic utilization of the fatty acids, and exerts a profound impact on fatty acid metabolic diseases such as gangliodosis, e.g., GM1, Tay-Sachs disease. Several rare autosomal disorders such as lysosomal storage disorders, are associated with the failure to degrade accumulated “cellular garbage” which generally results in the initiation of a low level but chronic inflammatory program with multiple devastating consequences such as tissue damage and cancer.

The accumulated cytoplasmic materials, known as damage associated molecular patterns (DAMPs), are considered to be ligands of a myriad of pattern recognition receptors (PRRs) that include TLRs 1-10, cGAS, IFI16, RIG-I, MDA5, NLRP family of the inflammasome proteins. Upon sensing of foreign and self-molecules, PRRs induce multiple signaling cascades with an autocrine and paracrine ability to execute fundamental cellular processes such as activation of the NFkB signaling pathway, IFN-I pathway, IFN-II pathway, IFN-III pathway, and autophagy pathways that include the AMPK, Beclin-I, PI3K pathways. Different events have been proposed to initiate the autophagy program, such as nutrient starvation conditions or exercise. AMPK activators, such as the blood glucose regulatory drug Metformin, are known to activate autophagy and increased the life span of experimental animals. The first molecular events in the activation of autophagy are the formation of an intracellular, cytosolic, double membrane structure (the autophagosome) by different cascade events that trigger congregation of proteins, such as the Atg family of proteins. The autophagosome encloses DAMPs and/or PAMPs present in the cells, the phenomenon known as the membrane nucleation stage. The next step in the autophagy pathway is the elongation and closure of the autophagosome. Finally, this matured and completely formed antophagosomes fuse with lysosomes, which contain broadly acting nucleases and proteases in a low pH environment, forming the autolysosome where the cargo is degraded into soluble and non-toxic, constituent components, thus decreasing the cytoplasmic abundance of DAMPs.

The induction of autophagy in specific tissues including liver, central nervous system (CNS) or gut, can greatly benefit patients suffering a myriad of different chronic disorders. Thus, provided herein is at least one recombinant virion, pharmaceutical composition, or transduced cells comprising a nucleic acid encoding a protein or a fragment thereof selected from IRGM, NOD2, ATG2B, ATG9, ATG5, ATG7, ATG16L1, BECN1, EI24/PIG8, TECPR2, WDR45/WIP14, CHMP2B, CHMP4B, Dynein, EPG5, HspB8, LAMP2, LC3b UVRAG, VCP/p97, ZFYVE26, PARK2/Parkin, PARK6/PINK1, SQSTM1/p62, SMURF, AMPK, and ULK1. In some embodiments, the at least one recombinant virion or pharmaceutical composition increases the expression of said protein or a fragment thereof in the transduced cells. In some embodiments, the at least one recombinant virion, pharmaceutical composition, or transduced cells modulate autophagy. In some embodiments, the at least one recombinant virion, pharmaceutical composition, or transduced cells prevent or treat an autophagy-related disease.

In some embodiments, the autophagy-related disease is selected from selected from cancer, neurodegenerative disease (e.g., Alzheimer's disease, Parkinson's disease, Huntington's disease, ataxias), inflammatory disease, inflammatory bowel disease, Crohn's disease, rheumatoid arthritis, lupus, multiple sclerosis, chronic obstructive pulmony disease/COPD, pulmonary fibrosis, cystic fibrosis, Sjogren's disease, hyperglycemic disorders, type I diabetes, type II diabetes, insulin resistance, hyperinsulinemia, insulin-resistant diabetes (e.g. Mendenhall's Syndrome, Werner Syndrome, leprechaunism, and lipoatrophic diabetes), dyslipidemia, hyperlipidemia, elevated low-density lipoprotein (LDL), depressed highdensity lipoprotein (HDL), elevated triglycerides, metabolic syndrome, liver disease, renal disease, cardiovascular disease, ischemia, stroke, complications during reperfusion, muscle degeneration, atrophy, symptoms of aging (e.g., muscle atrophy, frailty, metabolic disorders, low grade inflammation, atherosclerosis, stroke, age-associated dementia and sporadic form of Alzheimer's disease, pre-cancerous states, and psychiatric conditions including depression), spinal cord injury, arteriosclerosis, infectious diseases (e.g., bacterial, fungal, viral), AIDS, tuberculosis, defects in embryogenesis, infertility, lysosomal storage diseases, activator deficiency/GM2 gangliosidosis, alpha-mannosidosis, aspartylglucoaminuria, cholesteryl ester storage disease, chronic hexosaminidase A deficiency, cystinosis, Danon disease, Fabry disease, Farber disease, fucosidosis, galactosialidosis, Gaucher Disease (Types I, II and III), GM1 Gangliosidosis, (infantile, late infantile/juvenile and adult/chronic), Hunter syndrome (MPS II), I-Cell disease/Mucolipidosis II, Infantile Free Sialic Acid Storage Disease (ISSD), Juvenile Hexosaminidase A Deficiency, Krabbe disease, Lysosomal acid lipase deficiency, Metachromatic Leukodystrophy, Hurler syndrome, Scheie syndrome, Hurler-Scheie syndrome, Sanfilippo syndrome, Morquio Type A and B, Maroteaux-Lamy, Sly syndrome, mucolipidosis, multiple sulfate deficiency, Niemann-Pick disease, Neuronal ceroid lipofuscinoses, CLN6 disease, Jansky-Bielschowsky disease, Pompe disease, pycnodysostosis, Sandhoff disease, Schindler disease, Tay-Sachs, and Wolman disease.

As used herein, the term “autophagy-related diseases” refers to diseases that result from disruption in autophagy or cellular self-digestion. Autophagic dysfunction is associated with cancer, neurodegeneration, microbial infection and aging, among numerous other disease states and/or conditions. Although autophagy plays a principal role as a protective process for the cell, it also plays a role in cell death. Disease states and/or conditions which are mediated through autophagy (which refers to the fact that the disease state or condition may manifest itself as a function of the increase or decrease in autophagy in the patient or subject to be treated and treatment or prevention requires administration of an inhibitor or agonist of autophagy in the patient or subject) include, for example, cancer, including metastasis of cancer, lysosomal storage diseases (discussed hereinbelow), neurodegeneration (including, for example, Alzheimer's disease, Parkinson's disease, Huntington's disease; other ataxias), immune response (T cell maturation, B cell and T cell homeostasis, counters damaging inflammation) and chronic inflammatory diseases (may promote excessive cytokines when autophagy is defective), including, for example, inflammatory bowel disease, including Crohn's disease, rheumatoid arthritis, lupus, multiple sclerosis, chronic obstructive pulmony disease/COPD, pulmonary fibrosis, cystic fibrosis, Sjogren's disease; hyperglycemic disorders, type I diabetes, type II diabetes, affecting lipid metabolism islet function and/or structure, excessive autophpagy may lead to pancreatic b-cell death and related hyperglycemic disorders, including severe insulin resistance, hyperinsulinemia, insulin-resistant diabetes (e.g. Mendenhall's Syndrome, Werner Syndrome, leprechaunism, and lipoatrophic diabetes) and dyslipidemia (e.g. hyperlipidemia as expressed by obese subjects, elevated low-density lipoprotein (LDL), depressed highdensity lipoprotein (HDL), and elevated triglycerides) and metabolic syndrome, liver disease (excessive autophagic removal of cellular entities-endoplasmic reticulum), renal disease (apoptosis in plaques, glomerular disease), cardiovascular disease (especially including ischemia, stroke, pressure overload and complications during reperfusion), muscle degeneration and atrophy, symptoms of aging (including amelioration or the delay in onset or severity or frequency of aging-related symptoms and chronic conditions including muscle atrophy, frailty, metabolic disorders, low grade inflammation, atherosclerosis and associated conditions such as cardiac and neurological both central and peripheral manifestations including stroke, age-associated dementia and sporadic form of Alzheimer's disease, pre-cancerous states, and psychiatric conditions including depression), stroke and spinal cord injury, arteriosclerosis, infectious diseases (microbial infections, removes microbes, provides a protective inflammatory response to microbial products, limits adapation of autophagy of host by microbe for enhancement of microbial growth, regulation of innate immunity) including bacterial, fungal, cellular and viral (including secondary disease states or conditions associated with infectious diseases), including AIDS and tuberculosis, among others, development (including erythrocyte differentiation), embryogenesis/fertility/infertility (embryo implantation and neonate survival after termination of transplacental supply of nutrients, removal of dead cells during programmed cell death) and aging (increased autophagy leads to the removal of damaged organelles or aggregated macromolecules to increase health and prolong life, but increased levels of autophagy in children/young adults may lead to muscle and organ wasting resulting in aging/progeria).

The term “lysosomal storage disorder” refers to a disease state or condition that results from a defect in lysosomomal storage. These disease states or conditions generally occur when the lysosome malfunctions. Lysosomal storage disorders are caused by lysosomal dysfunction usually as a consequence of deficiency of an enzyme required for the metabolism of lipids, glycoproteins or mucopolysaccharides. The incidence of lysosomal storage disorder (collectively) occurs at an incidence of about about 1:5,000-1:10,000. The lysosome is commonly referred to as the cell's recycling center because it processes unwanted material into substances that the cell can utilize. Lysosomes break down this unwanted matter via high specialized enzymes. Lysosomal disorders generally are triggered when a particular enzyme exists in too small an amount or is missing altogether. When this happens, substances accumulate in the cell. In other words, when the lysosome doesn't function normally, excess products destined for breakdown and recycling are stored in the cell. Lysosomal storage disorders are genetic diseases, but these may be treated using autophagy modulators (autostatins) as described herein. All of these diseases share a common biochemical characteristic, i.e., that all lysosomal disorders originate from an abnormal accumulation of substances inside the lysosome. Lysosomal storage diseases mostly affect children who often die as a consequence at an early stage of life, many within a few months or years of birth. Many other children die of this disease following years of suffering from various symptoms of their particular disorder.

Examples of lysosomal storage diseases include, for example, activator deficiency/GM2 gangliosidosis, alpha-mannosidosis, aspartylglucoaminuria, cholesteryl ester storage disease, chronic hexosaminidase A deficiency, cystinosis, Danon disease, Fabry disease, Farber disease, fucosidosis, galactosialidosis, Gaucher Disease (Types I, II and III), GM1 Gangliosidosis, including infantile, late infantile/juvenile and adult/chronic), Hunter syndrome (MPS II), I-Cell disease/Mucolipidosis II, Infantile Free Sialic Acid Storage Disease (ISSD), Juvenile Hexosaminidase A Deficiency, Krabbe disease, Lysosomal acid lipase deficiency, Metachromatic Leukodystrophy, Hurler syndrome, Scheie syndrome, Hurler-Scheie syndrome, Sanfilippo syndrome, Morquio Type A and B, Maroteaux-Lamy, Sly syndrome, mucolipidosis, multiple sulfate deficiency, Niemann-Pick disease, Neuronal ceroid lipofuscinoses, CLN6 disease, Jansky-Bielschowsky disease, Pompe disease, pycnodysostosis, Sandhoff disease, Schindler disease, Tay-Sachs, and Wolman disease, among others.

### Inflammatory Disorders

The methods, recombinant virions, and/or pharmaceutical compositions described herein can be used, for example, for preventing or treating (reducing, partially or completely, the adverse effects of) an autoimmune disease, such as chronic inflammatory bowel disease, systemic lupus erythematosus, psoriasis, muckle-wells syndrome, rheumatoid arthritis, multiple sclerosis, or Hashimoto's disease; an allergic disease, such as a food allergy, pollenosis, or asthma; an infectious disease, e.g., infection with Clostridium difficile; an inflammatory disease such as a TNF-mediated inflammatory disease (e.g., an inflammatory disease of the gastrointestinal tract, such as pouchitis, a cardiovascular inflammatory condition, such as atherosclerosis, or an inflammatory lung disease, such as chronic obstructive pulmonary disease); a pharmaceutical composition for suppressing rejection in organ transplantation or other situations in which tissue rejection might occur; a pharmaceutical composition for improving immune functions; or a pharmaceutical composition for suppressing the proliferation or function of immune cells.

In some embodiments, the methods provided herein are useful for the treatment or prevention of inflammation. In certain embodiments, the inflammation of any tissue and organs of the body, including musculoskeletal inflammation, vascular inflammation, neural inflammation, digestive system inflammation, ocular inflammation, inflammation of the reproductive system, and other inflammation, as discussed below.

Immune disorders of the musculoskeletal system include, but are not limited, to those conditions affecting skeletal joints, including joints of the hand, wrist, elbow, shoulder, jaw, spine, neck, hip, knew, ankle, and foot, and conditions affecting tissues connecting muscles to bones such as tendons. Examples of such immune disorders, which may be treated with the methods and compositions described herein include, but are not limited to, arthritis (including, for example, osteoarthritis, rheumatoid arthritis, psoriatic arthritis, ankylosing spondylitis, acute and chronic infectious arthritis, arthritis associated with gout and pseudogout, and juvenile idiopathic arthritis), tendonitis, synovitis, tenosynovitis, bursitis, fibrositis (fibromyalgia), epicondylitis, myositis, and osteitis (including, for example, Paget's disease, osteitis pubis, and osteitis fibrosa cystic).

Ocular immune disorders refers to an immune disorder that affects any structure of the eye, including the eye lids. Examples of ocular immune disorders which may be treated with the methods and compositions described herein include, but are not limited to, blepharitis, blepharochalasis, conjunctivitis, dacryoadenitis, keratitis, keratoconjunctivitis sicca (dry eye), scleritis, trichiasis, and uveitis

Examples of nervous system immune disorders which may be treated with the methods and compositions described herein include, but are not limited to, encephalitis, Guillain-Barre syndrome, meningitis, neuromyotonia, narcolepsy, multiple sclerosis, myelitis and schizophrenia. Examples of inflammation of the vasculature or lymphatic system which may be treated with the methods and compositions described herein include, but are not limited to, arthrosclerosis, arthritis, phlebitis, vasculitis, and lymphangitis.

Examples of digestive system immune disorders which may be treated with the methods and pharmaceutical compositions described herein include, but are not limited to, cholangitis, cholecystitis, enteritis, enterocolitis, gastritis, gastroenteritis, inflammatory bowel disease, ileitis, and proctitis. Inflammatory bowel diseases include, for example, certain art-recognized forms of a group of related conditions. Several major forms of inflammatory bowel diseases are known, with Crohn's disease (regional bowel disease, e.g., inactive and active forms) and ulcerative colitis (e.g., inactive and active forms) the most common of these disorders. In addition, the inflammatory bowel disease encompasses irritable bowel syndrome, microscopic colitis, lymphocytic-plasmocytic enteritis, coeliac disease, collagenous colitis, lymphocytic colitis and eosinophilic enterocolitis. Other less common forms of IBD include indeterminate colitis, pseudomembranous colitis (necrotizing colitis), ischemic inflammatory bowel disease, Behcet's disease, sarcoidosis, scleroderma, IBD-associated dysplasia, dysplasia associated masses or lesions, and primary sclerosing cholangitis.

Examples of reproductive system immune disorders which may be treated with the methods and pharmaceutical compositions described herein include, but are not limited to, cervicitis, chorioamnionitis, endometritis, epididymitis, omphalitis, oophoritis, orchitis, salpingitis, tubo-ovarian abscess, urethritis, vaginitis, vulvitis, and vulvodynia.

The methods and compositions described herein may be used to prevent or treat autoimmune conditions having an inflammatory component. Such conditions include, but are not limited to, acute disseminated alopecia universalise, Behcet's disease, Chagas' disease, chronic fatigue syndrome, dysautonomia, encephalomyelitis, ankylosing spondylitis, aplastic anemia, hidradenitis suppurativa, autoimmune hepatitis, autoimmune oophoritis, celiac disease, Crohn's disease, diabetes mellitus type 1, type 2 diabetes, giant cell arteritis, goodpasture's syndrome, Grave's disease, Guillain-Barre syndrome, Hashimoto's disease, Henoch-Schonlein purpura, Kawasaki's disease, lupus erythematosus, microscopic colitis, microscopic polyarteritis, mixed connective tissue disease, Muckle-Wells syndrome, multiple sclerosis, myasthenia gravis, opsoclonus myoclonus syndrome, optic neuritis, ord's thyroiditis, pemphigus, polyarteritis nodosa, polymyalgia, rheumatoid arthritis, Reiter's syndrome, Sjogren's syndrome, temporal arteritis, Wegener's granulomatosis, warm autoimmune haemolytic anemia, interstitial cystitis, Lyme disease, morphea, psoriasis, sarcoidosis, scleroderma, ulcerative colitis, and vitiligo.

The methods and compositions described herein may be used to prevent or treat T-cell mediated hypersensitivity diseases having an inflammatory component. Such conditions include, but are not limited to, contact hypersensitivity, contact dermatitis (including that due to poison ivy), uticaria, skin allergies, respiratory allergies (hay fever, allergic rhinitis, house dustmite allergy) and gluten-sensitive enteropathy (Celiac disease).

Other immune disorders which may be treated with the methods and pharmaceutical compositions include, for example, appendicitis, dermatitis, dermatomyositis, endocarditis, fibrositis, gingivitis, glossitis, hepatitis, hidradenitis suppurativa, iritis, laryngitis, mastitis, myocarditis, nephritis, otitis, pancreatitis, parotitis, percarditis, peritonoitis, pharyngitis, pleuritis, pneumonitis, prostatistis, pyelonephritis, and stomatisi, transplant rejection (involving organs such as kidney, liver, heart, lung, pancreas (e.g., islet cells), bone marrow, cornea, small bowel, skin allografts, skin homografts, and heart valve xengrafts, sewrum sickness, and graft vs host disease), acute pancreatitis, chronic pancreatitis, acute respiratory distress syndrome, Sexary's syndrome, congenital adrenal hyperplasis, nonsuppurative thyroiditis, hypercalcemia associated with cancer, pemphigus, bullous dermatitis herpetiformis, severe erythema multiforme, exfoliative dermatitis, seborrheic dermatitis, seasonal or perennial allergic rhinitis, bronchial asthma, contact dermatitis, atopic dermatitis, drug hypersensistivity reactions, allergic conjunctivitis, keratitis, herpes zoster ophthalmicus, iritis and oiridocyclitis, chorioretinitis, optic neuritis, symptomatic sarcoidosis, fulminating or disseminated pulmonary tuberculosis chemotherapy, idiopathic thrombocytopenic purpura in adults, secondary thrombocytopenia in adults, acquired (autoimmune) haemolytic anemia, regional enteritis, autoimmune vasculitis, multiple sclerosis, chronic obstructive pulmonary disease, solid organ transplant rejection, sepsis. Preferred treatments include treatment of transplant rejection, rheumatoid arthritis, psoriatic arthritis, multiple sclerosis, Type 1 diabetes, asthma, inflammatory bowel disease, systemic lupus erythematosus, psoriasis, chronic obstructive pulmonary disease, and inflammation accompanying infectious conditions (e.g., sepsis).

### Neurodegenerative & Neuroinflammatory Disorders

The methods, recombinant virions, and/or pharmaceutical compositions described herein may be used to prevent or treat neurodegenerative and neurological diseases. In certain embodiments, the neurodegenerative and/or neurological disease is Parkinson's disease, Alzheimer's disease, prion disease, Huntington's disease, motor neuron diseases (MND), spinocerebellar ataxia, spinal muscular atrophy, dystonia, idiopathicintracranial hypertension, epilepsy, nervous system disease, central nervous system disease, movement disorders, multiple sclerosis, encephalopathy, peripheral neuropathy, post-operative cognitive dysfunction, frontotemporal dementia, stroke, transient ischemic attack, vascular dementia, Creutzfeldt-Jakob disease, multiple sclerosis, prion disease, Pick's disease, corticobasal degeneration, Parkinson's disease, Lewy body dementia, progressive supranuclear palsy, dementia pugilistica (chronic traumatic encephalopathy), frontotemporal dementia, parkinsonism linked to chromosome 17, Lytico-Bodig disease, Tangle-predominant dementia, ganglioglioma, gangliocytoma, meningioangiomatosis, subacute sclerosing panencephalitis, lead encephalopathy, tuberous sclerosis, Hallervorden-Spatz disease, lipofuscinosis, argyrophilic grain disease, and frontotemporal lobar degeneration.

The methods, recombinant virions, and/or pharmaceutical compositions described herein may be used to prevent or treat neuroinflammation and/or neuroinflammatory diseases, e.g., using a recombinant virion of the present disclosure to deliver a nucleic acid comprising a gene encoding one or more cytokines that alleviate inflammation. Neuroinflammatory diseases include, but not limited to, an autoimmune disease, an inflammatory disease, a neurogenerative disease, a neuromuscular disease, or a psychiatric disease. In some embodiments, the methods and compositions provided herein are useful for treatment or prevention of the inflammation of central nervous system, including brain inflammation, peripheral nerves inflammation, neural inflammation, spinal cord inflammation, ocular inflammation, and/or other inflammation.

Examples of disorders associated with neuroinflammation or neuroinflammatory disorders which may be treated with the methods and compositions described herein include, but are not limited to, encephalitis (inflammation of the brain), encephalomyelitis (inflammation of the brain and spinal cord), meningitis (inflammation of the membranes that surround the brain and spinal cord), Guillain-Barre syndrome, neuromyotonia, narcolepsy, multiple sclerosis, myelitis, schizophrenia, acute disseminated encephalomyelitis (ADEM), accute optic neuritis (AON), transverse myelitis, neuromyelitis optica (NMO), Alzheimer's disease, Parkinson's disease, amyotrophic lateral sclerosis, frontotemporal lobar dementia, optic neuritis, neuromyelitis optica spectrum disorder (NMOSD), auto-immune encephalitis, anti-NMDA receptor encephalitis, Rasmussen's encephalitis, acute necrotizing encephalopathy of childhood (ANEC), opsoclonus-myoclonus ataxia syndrome, traumatic brain injury, Huntington's disease, depression, anxiety, migraine, myasthenia gravis, acute ischemic stroke, epilepsy, synucleinopathies, frontotemporal dementia, progressive nonfluent aphasia, semantic dementia, Nodding syndrome, cerebral ischemia, neuropathic pain, autism spectrum disorder, fibromyalgia syndrome, progressive supranuclear palsy, corticobasal degeneration, systemic lupus erythematosus, prion disease, motor neurone diseases (MND), spinocerebellar ataxia, spinal muscular atrophy, dystonia, idiopathicintracranial hypertension, nervous system disease, central nervous system disease, movement disorders, encephalopathy, peripheral neuropathy, or post-operative cognitive dysfunction.

### Cancer

Cancer, tumor, or hyperproliferative disorder refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells may exist alone within an animal, or may be a non-tumorigenic cancer cell, such as a leukemia cell. Cancers include, but are not limited to, B cell cancer, (e.g., multiple myeloma, Diffuse large B-cell lymphoma (DLBCL), Follicular lymphoma, Chronic lymphocytic leukemia (CLL), small lymphocytic lymphoma (SLL), Mantle cell lymphoma (MCL), Marginal zone lymphomas, Burkitt lymphoma, Waldenström's macroglobulinemia, Hairy cell leukemia, Primary central nervous system (CNS) lymphoma, Primary intraocular lymphoma, the heavy chain diseases, such as, for example, alpha chain disease, gamma chain disease, and mu chain disease, benign monoclonal gammopathy, and immunocytic amyloidosis), T cell cancer (e.g., T-lymphoblastic lymphoma/leukemia, non-Hodgkin lymphomas, Peripheral T-cell lymphomas, Cutaneous T-cell lymphomas (e.g., mycosis fungoides, Sezary syndrome), Adult T-cell leukemia/lymphoma, Angioimmunoblastic T-cell lymphoma, Extranodal natural killer/T-cell lymphoma, Enteropathy-associated intestinal T-cell lymphoma (EATL), Anaplastic large cell lymphoma (ALCL), Hodgkin lymphoma), melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma (SCLC), bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithlelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated.

### Familial Intrahepatic Cholestasis

The methods, recombinant virions, and/or pharmaceutical compositions described herein may be used to prevent or treat familial intrahepatic cholestasis (PFIC), a genetic disease associated with mutations in the ATPB1, ATPB11 and ABCB4 genes which results in PFIC type 1, 2 and 3, respectively. This rare autosomal recessive disease drives the disruption of the bile secretory pathway, characterized by ductular proliferation in the liver and progressive intrahepatic cholestasis with elevated gamma-glutamyltranspeptidase (GGT) activity. ABCB4 mutations are the most prevalent forms of the disease. The ABCB4 gene is located on chromosome 7q21.1 and encodes for the lipid floppase MDR3 protein, involved in causing PFIC3. MDR3 is primarily expressed at the canalicular membrane of the liver and acts as a phospholipid translocator, i.e., phosphatidylcholine (PC). MDR3 protects the hepatocytemembrane from detergent activity of bile salts. The PFIC3 defect is characterized by reduced secretion of phosphatidylcholine (PC) into bile, thus impairing the bile secretory transport system (Davit-Spraul, et al., PMID: 20422496). Reduced PC secretion causes toxicity in the liver which results in the activation of a pro-inflammatory program with a concomitant destruction of hepatocytes that further progresses to intrahepatic liver cirrhosis. Other less prevalent forms of the disease are caused by mutations in ATPB1 and ATPB11 genes which result in similar outcomes. In various embodiments, the recombinant virions described herein are administered in vivo by direct injection to a cell, tissue, or organ of a subject in need of gene therapy.

### Wilson Disease

The methods, recombinant virions, and/or pharmaceutical compositions described herein may be used to prevent or treat Wilson Disease (WD). WD is a monogenic, autosomal recessively inherited condition, associated with mutations in the ATP7B gene, which encode a copper-transporting P-type ATPase. More than 600 pathogenic variants in ATP7B have been identified, with single-nucleotide missense and nonsense mutations being the most common, followed by insertions/deletions, and, rarely, splice site mutations. ATP7B is most highly expressed in the liver, but is also found in the kidney, placenta, mammary glands, brain, and lung. ATPB7 disruption leads to increased intracellular copper levels. Human dietary intake of copper is about 1.5-2.5 mg/day, which is absorbed in the stomach and duodenum, bound to circulating albumin, and transported to the liver for regulation and excretion. The antioxidant protein 1 (ATOX1) delivers copper to ATPB7 by copper-dependent protein-protein interaction. Within hepatocytes, ATP7B performs two important functions in either the trans-Golgi network (TGN) or in cytoplasmic vesicles. In the TGN, ATP7B activates ceruloplasmin by packaging six copper molecules into apoceruloplasmin, which is then secreted into the plasma. In the cytoplasm, ATP7B sequesters excess copper into vesicles and excretes it via exocytosis across the apical canalicular membrane into bile (Bull et al., 1993; Tanzi et al., 1993; Yamaguchi et al., 1999; Cater et al., 2007). Due to the binary role of the ATP7B transporter in both the synthesis and excretion of copper, defects in its function lead to copper accumulation triggering oxidative stress and free radical formation as well as mitochondrial dysfunction arising independently of oxidative stress. The combined effects results in the induction of a pro-inflammatory state and subsequent cell death in hepatic and brain tissue as well as other organs. In various embodiments, the recombinant virions described herein are administered in vivo by direct injection to a cell, tissue, or organ of a subject in need of gene therapy.

### Lysosomal Storage Disorders

The methods, recombinant virions, and/or pharmaceutical compositions described herein may be used to prevent or treat lysosomal storage diseases (LSD). These are inherited metabolic diseases that are characterized by an abnormal build-up of various toxic materials in the body's cells as a result of enzyme deficiencies. The methods and compositions described herein may be used to prevent or treat carbamoyl phosphate synthetase 1 deficiency (CPS1D), a rare autosomal recessive disorder, characterized by a destructive metabolic disease dominated by severe hyperammonemia that affect multiple organs, including in some cases changes in brain white matter. CPS1 plays a paramount role in liver ureagenesis since it catalyzes the first and rate-limiting step of the urea cycle, the major pathway for nitrogen disposal in humans. CPS1 deficiency leads to urea cycle disorder and accumulation of ammonia. Therefore, marked hyperammonemia and decreased downstream production of the urea cycle can be observed in patients with CPS1 deficiency. The superabundant ammonia can enter the central nervous system and exerts its toxic effects on the brain. Accumulation of ammonia induces toxicity and lead to cell death. In various embodiments, the recombinant virions described herein are administered in vivo by direct injection to a cell, tissue, or organ of a subject in need of gene therapy.

### Epidermolysis Bullosa

The methods, recombinant virions, and/or pharmaceutical compositions described herein may be used to prevent or treat genetic diseases of the skin. Human epidermis is mainly composed of keratinocytes organized in distinct stratified cellular layers. The adhesion of basal keratinocytes to the epidermal basement membrane is mediated by the hemidesmosomes (HDs), which are multiprotein complexes linking the epithelial intermediate filament network to the dermal anchoring fibrils. Hemidesmosomes are formed by the clustering of several cytoplasmic and transmembrane proteins. The cytoplasmic HD plaque components, which include HD1/plectin and the bullous pemphigoid antigen 1 (BP230), act as linkers for elements of the cytoskeleton at the cytoplasmic surface of plasma membrane. The transmembrane constituents of HDs, which include the α6β4 integrin and the bullous pemphigoid antigen 2 (BP180), serve as cell receptors connecting the cell interior to extracellular matrix proteins. Hemidesmosome-mediated adhesion relies on the binding of the α6β4 integrin to laminin-5, a major basal lamina component formed by distinct polypeptides, α3, β3, and γ2, encoded by 3 different genes known as LAMA3, LAMB3, and LAMC2, respectively. Laminin-5 interacts physically with α6β4 integrin on the basal surface of epidermal keratinocytes to promote HD formation as well as with the amino-terminal NC-1 domain of type VII collagen in dermal anchoring fibrils to enhance basement membrane zone integrity. The relevance of these proteins in maintaining the integrity of the skin has been proven by the identification of somatic mutations present in patients with epidermolysis bullosa (EB). At least 16 genetic mutations in various genes (e.g., KRT5, KRT14, PLEC1, Col7A1, ITGB4, ITGA6, LAMA3, LAMB3, LAMC2, and KIND1) have been associated with different types of EB. Since keratinocytes are responsible for the synthesis of proteins involved in maintaining the dermal-epidermal junction, a gene therapeutic intervention to prevent or treat this disease requires the genetic modification of these cells.

In certain aspects, the methods, recombinant virions, and/or pharmaceutical compositions described herein can be used to specifically modify stem cells of the skin for different skin disorders such as EB. For example, a cutavirus-derived recombinant virion (e.g., a recombinant virion comprising at least one capsid protein of a cutavirus) can be used to deliver a therapeutic transgene into an epidermal stem cell. In some embodiments, the epidermal stem cell is a holoclone-forming cell. In some embodiments, the holoclone-forming cells are P63-positive keratinocytes-derived stem cells that have the maximum proliferative capacity, and thus are considered as epithelial stem cells. In some embodiments, the therapeutic transgene further comprises the GSH sequences that facilitate stable integration into a known genomic location by homologous recombination. The use of GSH allows stable and persistent transgene expression throughout differentiation of the skin cells. In some embodiments, the epidermal stem cells, P63-positive keratinocyte-derived stem cells, or keratinocytes are modified ex vivo by the recombinant virions and pharmaceutical compositions of the present disclosure. In some such embodiments, the modified epidermal stem cells, P63-positive keratinocyte-derived stem cells, or keratinocytes are applied to the the skin surface as a skin graft.

Accordingly, in certain aspects, provided herein are methods of preventing or treating epidermolysis bullosa, wherein the the at least one recombinant virion, pharmaceutical composition, or transduced cells comprise a nucleic acid encoding KRT5, KRT14, PLEC1, Col7A1, ITGB4, ITGA6, LAMA3, LAMB3, LAMC2, and/or KIND1. In some embodiments, the recombinant virion comprises at least one capsid protein of a protoparvovirus. In some embodiments, the recombinant virion comprises at least one capsid protein of a cutavirus. In some embodiments, the at least one recombinant virion transduces epidermal stem cells, P63-positive keratinocyte-derived stem cells, or keratinocytes. In some embodiments, the transduced epidermal cells are stem cells, P63-positive keratinocyte-derived stem cells, or keratinocytes. In some embodiments, the methods further comprise grafting the transduced cells on the skin of the subject. In some embodiments, the at least one recombinant virion, pharmaceutical composition, or transduced cells of the present disclosure prevent or treat epidermolysis bullosa.

### Hematologic Diseases

In certain aspects, in addition to the hematologic diseases described below, the methods, recombinant virions, and/or pharmaceutical compositions described herein can be used for treatment or prevention of a disease such as endothelial dysfunction, cystic fibrosis, cardiovascular disease, peripheral vascular disease, stroke, heart disease (e.g., including congenital heart disease), diabetes, insulin resistance, chronic kidney failure, atherosclerosis, tumor growth (e.g., including those of endothelial cells), metastasis, hypertension (e.g., pulmonary arterial hypertension, other forms of pulmonary hypertension), atherosclerosis, restenosis, Hepatitis C, liver cirrhosis, hyperlipidemia, hypercholesterolemia, metabolic syndrome, renal disease, inflammation, and venous thrombosis.

In certain aspects, a hematologic disease includes any one of the following: hemoglobinopathy (e.g., sickle cell disease, thalassemia, methemoglobinemia), anemia (iron-deficiency anemia, megaloblastic anemia, hemolytic anemias, myelodysplastic syndrome, myelofibrosis, neutropenia, agranulocytosis, Glanzmann's thrombasthenia, thrombocytopenia, Wiskott-Aldrich syndrome, myeloproliferative disorders (e.g., polycythemia vera, erythrocytosis, leukocytosis, thrombocytosis), coagulopathies, a hematologic cancer, hemochromatosis, asplenia, hypersplenism (e.g., Gaucher's disease), hemophagocytic lymphohistiocytosis, tempi syndrome, and AIDS.

In some embodiments, the exemplary hemolytic anemia includes: Hereditary spherocytosis, Hereditary elliptocytosis, Congenital dyserythropoietic anemia, Glucose-6-phosphate dehydrogenase deficiency (G6PD), pyruvate kinase deficiency, autoimmune hemolytic anemia (e.g., idiopathic anemia, Systemic lupus erythematosus (SLE), Evans syndrome, Cold agglutinin disease, Paroxysmal cold hemoglobinuria, Infectious mononucleosis), alloimmune hemolytic anemia (e.g., hemolytic disease of the newborn, such as Rh disease, ABO hemolytic disease of the newborn, anti-Kell hemolytic disease of the newborn, Rhesus c hemolytic disease of the newborn, Rhesus E hemolytic disease of the newborn), Paroxysmal nocturnal hemoglobinuria, Microangiopathic hemolytic anemia, Fanconi anemia, Diamond-Blackfan anemia, and Acquired pure red cell aplasia.

In some embodiments, the exemplary coagulopathy includes: thrombocytosis, disseminated intravascular coagulation, hemophilia (e.g., hemophilia A, hemophilia B, hemophilia C), von Willebrand disease, and antiphospholipid syndrome.

In some embodiments, the exemplary hematologic cancer includes: Hodgkin's disease, Non-Hodgkin's lymphoma, Burkitt's lymphoma, Anaplastic large cell lymphoma, Splenic marginal zone lymphoma, T-cell lymphoma (e.g., Hepatosplenic T-cell lymphoma, Angioimmunoblastic T-cell lymphoma, Cutaneous T-cell lymphoma), Multiple myeloma, Waldenstrom macroglobulinemia, Plasmacytoma, Acute lymphocytic leukemia (ALL), Chronic lymphocytic leukemia (CLL), Acute myelogenous leukemia (AML), Acute megakaryoblastic leukemia, Chronic Idiopathic Myelofibrosis, Chronic myelogenous leukemia (CML), T-cell prolymphocytic leukemia, B-cell prolymphocytic leukemia, Chronic neutrophilic leukemia, Hairy cell leukemia, T-cell large granular lymphocyte leukemia, AIDS-related lymphoma, Sezary syndrome, Waldenstrom Macroglobulinemia, Chronic Myeloproliferative Neoplasms, Langerhans Cell Histiocytosis, Myelodysplastic Syndromes, and Aggressive NK-cell leukemia.

As used herein, the hemoglobinopathy includes any disorder involving the presence of an abnormal hemoglobin molecule in the blood. Examples of hemoglobinopathies included, but are not limited to, hemoglobin C disease, hemoglobin sickle cell disease (SCD), sickle cell anemia, and thalassemias. Also included are hemoglobinopathies in which a combination of abnormal hemoglobins are present in the blood (e.g., sickle cell/Hb-C disease).

As used herein, thalassemia refers to a hereditary disorder characterized by defective production of hemoglobin. Examples of thalassemias include α- and β-thalassemia. β-thalassemias are caused by a mutation in the beta globin chain, and can occur in a major or minor form. In the major form of β-thalassemia, children are normal at birth, but develop anemia during the first year of life. The mild form of—thalassemia produces small red blood cells and the thalassemias are caused by deletion of a gene or genes from the globin chain, α-thalassemia typically results from deletions involving the HBA1 and HBA2 genes. Both of these genes encode α-globin, which is a component (subunit) of hemoglobin. There are two copies of the HBA1 gene and two copies of the HBA2 gene in each cellular genome. As a result, there are four alleles that produce α-globin. The different types of a thalassemia result from the loss of some or all of these alleles. Hb Bart syndrome, the most severe form of a thalassemia, results from the loss of all four α-globin alleles. HbH disease is caused by a loss of three of the four α-globin alleles. In these two conditions, a shortage of α-globin prevents cells from making normal hemoglobin. Instead, cells produce abnormal forms of hemoglobin called hemoglobin Bart (Hb Bart) or hemoglobin H (HbH). These abnormal hemoglobin molecules cannot effectively carry oxygen to the body's tissues. The substitution of Hb Bart or HbH for normal hemoglobin causes anemia and the other serious health problems associated with a thalassemia.

As used herein, the sickle cell disease refers to a group of autosomal recessive genetic blood disorders, which results from mutations in a globin gene and which is characterized by red blood cells that under hypoxic conditions, convert from the typical biconcave form into an abnormal, rigid, sickle shape that cannot course through capillaries, thereby exacerbating the hypoxia. They are defined by the presence of Ps-gene coding for a β-globin chain variant in which glutamic acid is substituted by valine at amino acid position 6 of the peptide, and second p-gene that has a mutation mat allows for the crystallization of HbS leading to a clinical phenotype. Sickle cell anemia refers to a specific form of sickle cell disease in patients who are homozygous for the mutation that causes HbS. Other common forms of sickle cell disease include HbS/β-thalassemia, HbS/HbC and HbS/HbD.

In certain embodiments, methods and compositions are provided herein to treat, prevent, or ameliorate a hemoglobinopathy that is selected from the group consisting of: hemoglobin C disease, hemoglobin sickle cell disease (SCD), sickle cell anemia, hereditary anemia, thalassemia, β-thalassemia, thalassemia major, thalassemia intermedia, a-thalassemia, and hemoglobin H disease. In some embodiments, the hemoglobinopathy is β-thalassemia. In some embodiments, the hemoglobinopathy is sickle cell anemia. In various embodiments, the recombinant virions described herein are administered in vivo by direct injection to a cell, tissue, or organ of a subject in need of gene therapy. In various other embodiments, cells are transduced in vitro or ex vivo with the recombinant virions described herein. The transduced cells are then administered to a subject in need of gene therapy, e.g., within a pharmaceutical formulation disclosed herein.

As described above, provided herein are methods of preventing or treating a hemoglobinopathy in a subject. In various embodiments, the method comprises administering an effective amount of a cell transduced with the recombinant virions described herein or a population of the said transduced cells (e.g., HSCs, CD34+ or CD36 cells, erythroid lineage cells, embryonic stem cells, or iPSCs) to the subject. For treatment or prevention, the amount administered can be an amount effective in producing the desired clinical benefit. An effective amount can be provided in one or a series of administrations. An effective amount can be provided in a bolus or by continuous perfusion. An effective amount can be administered to a subject in one or more doses. In terms of treatment or prevention, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the ordinary skill of one in the art. Several factors are typically taken into account when determining an appropriate dosage to achieve an effective amount. These factors include age, sex and weight of the subject, the condition being treated, the severity of the condition.

### Type I Diabetes

In certain aspects, the methods, recombinant virions, and/or pharmaceutical compositions described herein can be used for treatment or prevention of type I diabetes. Enteroendocrine cells in the small intestine, especially in the duodenum and jejunum, are attractive targets for an insulin gene transfer strategy to prevent or treat patients with type 1 diabetes mellitus. K cells and L cells of the intestine are innately specialized to respond to nutrients in the lumen, especially glucose. They respond by secreting GIP and GLP-1 into the blood, potentiating the glucose-induced insulin response. In normal individuals, the kinetics and plasma concentrations attained for GIP, GLP-1 and insulin following a meal are highly similar, and so are those of GIP and GLP-1 in patients with type 1 diabetes mellitus (Vilsboll et al., 2003). K cells and L cells synthesize the PC1/3 and PC2 peptidases that allow proinsulin processing into mature insulin. Importantly, K cells and L cells are not destroyed along with the pancreatic β-cells by the immune system of patients with type 1 diabetes mellitus (Vilsboll et al., 2003). Due to their common developmental origin, pancreatic β-cells, K-cells and L-cells show marked similarities, which include: (i) the expression of the PC1/3 and PC2 peptidases needed for the conversion of proinsulin to insulin, (ii) the presence of GLUT-2 glucose transporter, (iii) a glucosedependent mechanism for hormone secretion, with granules that can store and readily secrete their respective hormones (Spooner et al., 1970, Baggio & Drucker 2007). Thus, it is expected that if gastrointestinal enteroendocrine cells of patients with type 1 diabetes mellitus were endowed with the ability to express the preproinsulin gene (e.g., by introducing an insulin gene, INS, encoding a preproinsulin protein or transcript variants thereof, e.g., NP_000198.1, NP_001172026.1, NP_001172027.1, and/or NP_001278826.1, they would contribute to the normalization of postprandrial blood glucose. Intestinal stem cells, are located in the gut epithelium krypt. These cells express the transferrin receptor (TfR) on the cellular membrane, making them susceptible to transduction by canine or feline parvovirus-derived vectors. Thus, the recombinant virions and/or pharmaceutical compositions presented herein provide an attractive tool to incorporate the preproinsulin gene driven by GIP regulatory elements. Furthermore, site directed integration into the described genomic safe harbors, allows safe stem cell differentiation to insulin-producer K and L cells.

### Hemophilia A

Hemophilia A is an inherited bleeding disorder in which the blood does not clot normally. People with hemophilia A bleed more than normal after an injury, surgery, or dental procedure. This disorder can be severe, moderate, or mild. In severe cases, heavy bleeding occurs after minor injury or even when there is no injury (spontaneous bleeding). Bleeding into the joints, muscles, brain, or organs can cause pain and other serious complications. In milder forms, there is no spontaneous bleeding, and the disorder might only be diagnosed after a surgery or serious injury. Hemophilia A is caused by having low levels of a protein called factor VIII. Factor VIII is needed to form blood clots. The disorder is inherited in an X-linked recessive manner and is caused by changes (mutations) in the F8 gene. The diagnosis of hemophilia A is made through clinical symptoms and specific laboratory tests to measure the amount of clotting factors in the blood. The main prevention or treatment is replacement therapy, during which clotting factor VIII is dripped or injected slowly into a vein. Hemophilia A mainly affects males. With prevention or treatment, most people with this disorder do well. Some people with severe hemophilia A may have a shortened lifespan due to the presence of other health conditions and rare complications of the disorder.

Patients afflicted with hemophilia A stands to benefit from gene therapy that introduces the F8 transgene encoding a full length factor VIII (FVIII) or a B-domain-deleted FVIII (e.g., FVIII-SQ, p-VIII, p-VIII-LMW; Sandberg et al. (2001) Thromb Haemost 85:93-100), which retains activity necessary to provide therapeutic benefits in human (Rangarajan et al. (2017) N Engl J Med 377:2519-30). The recombinant virions, pharmaceutical compositions, and methods of the present disclosure provide improved viral vectors and prevention/treatment methods for patients afflicted with hemophilia A, in part due to the ability of the recombinant virions to package larger genes compared with AAV, low immunogenicity, and pulsatile gene regulation (see Example 9 and section “Pulsatile Gene Expression or Inducible Gene Expression”).

In some embodiments, the disease treated includes one selected from those presented in Table 5.

TABLE 5

| | | | | |
|---|---|---|---|---|
| | | | Major Organ | CNS |
| Disease | Defective Protein | Materials Stored | Systems Affected | Patholog |

Sphingolipidoses

| | | | | |
|---|---|---|---|---|
| Fabry | α-Galactoridase A | Globotriasylcetamide | Kidney, heart | + |
| | | and blood Group B sub- | | |
| | | stances | | |
| Gaucher | β-Glucosidase | Glucosylceramide | Spleen, liver, bone | + |
| | | glucosylsphingosine | marrow | (types 2 and 3) |
| Niemann-Pick A | Sphingomyclinase | Sphingomyelin | Spleen, liver, bone | + |
| and B | | | marrow, lung (type B) | (type A) |
| GM1 Gangliosidosis | β-Galactosidase | GM1 ganglioside | Skeleton, heart | + |

Mucopolysaccharidoses

| | | | | |
|---|---|---|---|---|
| MPS1 (Hurler, Scheie, | α-Iduronidase | Dermatan sulphate and | Organomegaly, | + |
| Hurler-Scheie) | | heparin sulphate | skeleton, cye | |
| MPS II (Hunter) | Iduronate-2-sulphatase | Dermatan sulphate and | Organomegaly, heart | + |
| | | heparan sulphate | | |
| MPS VI (Maroteaux- | acetylgalaclosamine-4- | Dermatan sulphate | Spleen, lives, heart, | − |
| Lamy) | sulphatase (arylsulpha- | | macrocephaly, | |
| | tase B) | | | |

In some embodiments, following administration of one or more of the presently disclosed transduced cells, peripheral blood of the subject is collected and hemoglobin level is measured. A therapeutically relevant level of hemoglobin is produced following administration of the recombinant virions or the cells transduced with the recombinant virions. Therapeutically relevant level of hemoglobin is a level of hemoglobin that is sufficient (1) to improve anemia, (2) to improve or restore the ability of the subject to produce red blood cells containing normal hemoglobin, (3) to improve or correct ineffective erythropoiesis in the subject, (4) to improve or correct extra-medullary hematopoiesis (e.g., splenic and hepatic extra-medullary hematopoiesis), and/or (S) to reduce iron accumulation, e.g., in peripheral tissues and organs. Therapeutically relevant level of hemoglobin can be at least about 7 g/dL Hb, at least about 7.5 g/dL Hb, at least about 8 g/dL Hb, at least about 8.5 g/dL Hb, at least about 9 g/dL Hb, at least about 9.5 g/dL Hb, at least about 10 g/dL Hb, at least about 10.5 g/dL Hb, at least about 11 g/dL Hb, at least about 11.5 g/dL Hb, at least about 12 g/dL Hb, at least about 12.5 g/dL Hb, at least about 13 g/dL Hb, at least about 13.5 g/dL Hb, at least about 14 g/dL Hb, at least about 14.5 g/dL Hb, or at least about 15 g/dL Hb. Additionally or alternatively, therapeutically relevant level of hemoglobin can be from about 7 g/dL Hb to about 7.5 g/dL Hb, from about 7.5 g/dL Hb to about 8 g/dL Hb, from about 8 g/dL Hb to about 8.5 g/dL Hb, from about 8.5 g/dL Hb to about 9 g/dL Hb, from about 9 g/dL Hb to about 9.5 g/dL Hb, from about 9.5 g/dL Hb to about 10 g/dL Hb, from about 10 g/dL Hb to about 10.5 g/dL Hb, from about 10.5 g/dL Hb to about 11 g/dL Hb, from about 11 g/dL Hb to about 11.5 g/dL Hb, from about 11.5 g/dL Hb to about 12 g/dL Hb, from about 12 g/dL Hb to about 12.5 g/dL Hb, from about 12.5 g/dL Hb to about 13 g/dL Hb, from about 13 g/dL Hb to about 13.5 g/dL Hb, from about 13.5 g/dL Hb to about 14 g/dL Hb, from about 14 g/dL Hb to about 14.5 g/dL Hb, from about 14.5 g/dL Hb to about 15 g/dL Hb, from about 7 g/dL Hb to about 8 g/dL Hb, from about 8 g/dL Hb to about 9 g/dL Hb, from about 9 g/dL Hb to about 10 g/dL Hb, from about 10 g/dL Hb to about 11 g/dL Hb, from about 11 g/dL Hb to about 12 g/dL Hb, from about 12 g/dL Hb to about 13 g/dL Hb, from about 13 g/dL Hb to about 14 g/dL Hb, from about 14 g/dL Hb to about 15 g/dL Hb, from about 7 g/dL Hb to about 9 g/dL Hb, from about 9 g/dL Hb to about 11 g/dL Hb, from about 11 g/dL Hb to about 13 g/dL Hb, or from about 13 g/dL Hb to about 15 g/dL Hb. In certain embodiments, the therapeutically relevant level of hemoglobin is maintained in the subject for at least 3 days, for at least 1 week, for at least 2 weeks, for at least 1 month, for at least 2 months, for at least 4 months, for at least about 6 months, for at least about 12 months (or 1 year), for at least about 24 months (or 2 years). In certain embodiments, the therapeutically relevant level of hemoglobin is maintained in the subject for up to about 6 months, for up to about 12 months (or 1 year), for up to about 24 months (or 2 years). In certain embodiments, the therapeutically relevant level of hemoglobin is maintained in the subject for about 3 days, for about 1 week, for about 2 weeks, for about 1 month, for about 2 months, for about 4 months, for about 6 months, for about 12 months (or 1 year), for about 24 months (or 2 years). In certain embodiments, the therapeutically relevant level of hemoglobin is maintained in the subject for from about 6 months to about 12 months (e.g., from about 6 months to about 8 months, from about 8 months to about 10 months, from about 10 months to about 12 months), from about 12 months to about 18 months (e.g., from about 12 months to about 14 months, from about 14 months to about 16 months, or from about 16 months to about 18 months), or from about 18 months to about 24 months (e.g., from about 18 months to about 20 months, from about 20 months to about 22 months, or from about 22 months to about 24 months).

In certain embodiments, the transduced cell is autologous to the subject being administered with the cell. In some embodiments, the transduced cell is from the bone marrow or mobilized cells in the peripheral circulation, autologous to the subject being administered with the cell. In some embodiments, the transduced cell is allogeneic to the subject being administered with the cell. In some embodiments, the transduced cell is from the bone marrow autologous to the subject being administered with the cell.

The present disclosure also provides a method of increasing the proportion of red blood cells or erythrocytes compared to white blood cells or leukocytes in a subject. In various embodiments, the method comprises administering an effective amount of the recombinant virions described herein or cells transduced with recombinant virions (e.g., HSCs, CD34+ or CD36 cells, erythroid lineage cells, embryonic stem cells, or iPSCs) to the subject, wherein the proportion of red blood cell progeny cells of the hematopoietic stem cells are increased compared to white blood cell progeny cells of the hematopoietic stem cells in the subject.

The quantity of transduced cells to be administered will vary for the subject and/or the disease being prevented or treated. In some embodiments, from about 1×10⁴ to about 1×10⁵ cells/kg, from about 1×10⁵ to about 1×10⁶ cells/kg, from about 1×10⁶ to about 1×10⁷ cells/kg, from about 1×10⁷ to about 1×10⁸ cells/kg, from about 1×10⁸ to about 1×10⁹ cells/kg, or from about 1×10⁹ to about 1×10¹⁰ cells/kg of the presently disclosed transduced cells are administered to a subject. Depending on the needs, the subject may need multiple doses of the transduced cells. The precise determination of what would be considered an effective dose may be based on factors individual to each subject, including their size, age, sex, weight, and condition of the particular subject. Dosages can be readily ascertained by those skilled in the art from this disclosure and the knowledge in the art.

Without being bound to any particular theory, an important advantage provided by the compositions and methods described herein is an efficient way of treating a subject afflicted with any disease (e.g., a hemoglobinopathy, cystic fibrosis, hemochromatosis) or preventing any disease in a subject, e.g., those at risk of developing such disease. Such at risk subjects can be identified by certain genetic mutations they carry, and/or environmental or physical factors (e.g., sex, age of the subject). The highly efficient and safe gene therapy is achieved by using the compositions and methods described herein (e.g., recombinant virions comprising at least one capsid protein of a protoparvovirus or tetraparvovirus). For example, the targeted integration of the nucleic acid (e.g., therapeutic nucleic acid) to a GSH reduces the chances of deleterious mutation, transformation, or oncogene activation of cellular genes in transduced cells. In addition, the specific tropism of the recombinant virion allows targeting to a specific cell type.

### Compositions and Methods for Producing the Recombinant Virion

In certain aspects, provided herein are methods of producing a recombinant virion described herein. The number of vectors described below may be consolidated by incorporating the structural and/or nonstructural genes into one or more vectors. Certain protoparvovirus or tetraparvovirus genomic sequence may also be integrated into the baculovirus genome to contain the structural (e.g., encoding VP protein(s)) and/or nonstructural genes.

In certain aspects, the methods of producing a recombinant virion comprises: (1) providing at least one vector comprising (i) a nucleotide sequence comprising at least one ITR nucleotide sequence, optionally further comprising a heterologous nucleic acid operably linked to a promoter for expression in a target cell, (ii) a nucleotide sequence comprising at least one gene encoding protoparvovirus or tetraparvovirus capsid proteins VP1 and/or VP2 operably linked to at least one expression control sequence for expression in an insect cell, and (iii) a nucleotide sequence comprising (A) at least one replication protein of an protoparvovirus or tetraparvovirus operably linked to at least one expression control sequence for expression in an insect cell, (B) at least one replication protein of an AAV, optionally wherein the at least one replication protein of an AAV comprises (a) a Rep52 or a Rep40 coding sequence operably linked to at least one expression control sequence for expression in an insect cell, and/or (b) a Rep78 or a Rep68 coding sequence operably linked to at least one expression control sequence for expression in an insect cell, or (C) a combination of (A) and (B), (2) introducing said at least one vector into an insect cell, and (3) maintaining said insect cell under conditions such that a recombinant virion described herein is produced. In preferred embodiments, the vector is an insect cell-compatible vector that comprises a promoter that facilitates the expression of a nucleic acid in insect cells.

In some embodiments, two vectors are provided: (a) a first vector comprising a nucleotide sequence comprising at least one ITR nucleotide sequence, optionally further comprising a heterologous nucleic acid operably linked to a promoter for expression in a target cell, and (b) a second vector comprising (i) a nucleotide sequence comprising at least one gene encoding the protoparvovirus or tetraparvovirus capsid proteins VP1 and/or VP2 operably linked to at least one expression control sequence for expression in an insect cell, and (ii) a nucleotide sequence comprising (A) at least one replication protein of an protoparvovirus or tetraparvovirus operably linked to at least one expression control sequence for expression in an insect cell, (B) at least one replication protein of an AAV, optionally wherein the at least one replication protein of an AAV comprises (a) a Rep52 or a Rep40 coding sequence operably linked to at least one expression control sequence for expression in an insect cell, and/or (b) a Rep78 or a Rep68 coding sequence operably linked to at least one expression control sequence for expression in an insect cell, or (C) a combination of (A) and (B).

In some embodiments, three vectors are provided: (a) a first vector comprising a nucleotide sequence comprising at least one ITR nucleotide sequence, optionally further comprising a heterologous nucleic acid operably linked to a promoter for expression in a target cell, (b) a second vector comprising a nucleotide sequence comprising a gene encoding the protoparvovirus or tetraparvovirus capsid proteins VP1 and/or VP2 operably linked to at least one expression control sequence for expression in an insect cell, and (c) a third vector comprising a nucleotide sequence comprising (A) at least one replication protein of an protoparvovirus or tetraparvovirus operably linked to at least one expression control sequence for expression in an insect cell, (B) at least one replication protein of an AAV, optionally wherein the at least one replication protein of an AAV comprises (a) a Rep52 or a Rep40 coding sequence operably linked to at least one expression control sequence for expression in an insect cell, and/or (b) a Rep78 or a Rep68 coding sequence operably linked to at least one expression control sequence for expression in an insect cell, or (C) a combination of (A) and (B).

In some embodiments, provided herein are methods of producing a recombinant virion described herein in an insect cell, the method comprising: (1) providing an insect cell comprising (i) a nucleotide sequence comprising at least one ITR nucleotide sequence, optionally further comprising a heterologous nucleic acid operably linked to a promoter for expression in a target cell, (ii) a nucleotide sequence comprising at least one gene encoding protoparvovirus or tetraparvovirus capsid proteins VP1 and/or VP2 operably linked to at least one expression control sequence for expression in an insect cell, and (iii) a nucleotide sequence comprising (A) at least one replication protein of an protoparvovirus or tetraparvovirus operably linked to at least one expression control sequence for expression in an insect cell, (B) at least one replication protein of an AAV, optionally wherein the at least one replication protein of an AAV comprises (a) a Rep52 or a Rep40 coding sequence operably linked to at least one expression control sequence for expression in an insect cell, and/or (b) a Rep78 or a Rep68 coding sequence operably linked to at least one expression control sequence for expression in an insect cell, or (C) a combination of (A) and (B), optionally, at least one vector, wherein at least one of (i), (ii), (iii)(A), (iii)(B), and (iii)(C) is/are stably integrated in the insect cell genome, and the at least one vector, when present, comprises the remainder of the (i), (ii), (iii)(A), (iii)(B), and (iii)(C) nucleotide sequences which is/are not stably integrated in the insect cell genome, and (2) maintaining the insect cell under conditions such that the recombinant virion is produced.

In some embodiments, provided herein are methods of producing a recombinant virion having at least one capsid protein of a protoparvovirus or a genotypic variant thereof, wherein the protoparvovirus is of a species selected from Carnivore protoparvovirus, Carnivore protoparvovirus 1, Chiropteran protoparvovirus 1, Eulipotyphla protoparvovirus 1, Primate protoparvovirus 1, Primate protoparvovirus 2, Primate protoparvovirus 3, Primate protoparvovirus 4, Rodent protoparvovirus 1, Rodent protoparvovirus 2, Rodent protoparvovirus 3, Ungulate protoparvovirus 1, and Ungulate protoparvovirus 2. In some embodiments, the protoparvovirus or a genotypic variant thereof is selected from canine parvovirus, feline panelukepenia virus, human bufavirus 1, human bufavirus 2, human bufavirus 3, human tusavirus, human cutavirus, Wuharv parvovirus, porcine parvovirus, minute virus of mice, megabat bufavirus, and a genotypic variant thereof.

In some embodiments, provided herein are methods of producing a recombinant virion having at least one capsid protein of a tetraparvovirus or a genotypic variant thereof, wherein tetraparvovirus is of a species selected from Chiropteran tetraparvovirus 1, Primate tetraparvovirus 1, Ungulate tetraparvovirus 1, Ungulate tetraparvovirus 2, Ungulate tetraparvovirus 3, and Ungulate tetraparvovirus 4. In some embodiments, the tetraparvovirus or a genotypic variant thereof is selected from human parvovirus 4, human parvovirus 4 genotype 1, human parvovirus 4 genotype 2, human parvovirus 4 genotype 3, chimpanzee parvovirus 4, eidolon helvum parvovirus, bovine hokovirus 1, bovine hokovirus 2, porcine hokovirus, porcine cnvirus, yak parvovirus, ovine hokovirus 1, opossum tetraparvovirus, rodent tetraparvovirus, tetraparvovirus sp., and a genotypic variant thereof. In some embodiments, the tetraparvovirus or a genotypic variant thereof is selected from human parvovirus 4, human parvovirus 4 genotype 1, human parvovirus 4 genotype 2, human parvovirus 4 genotype 3, and a genotypic variant thereof.

In some embodiments, the at least one replication protein of a a protoparvovirus is an NS-1 protein of a canine parvovirus, bufavirus, cutavirus, or a genotypic variant thereof.

In some embodiments, the at least one replication protein of a tetravirus is an NS-1 protein of a human parvovirus 4 or a genotypic variant thereof.

In some embodiments, the insect cell is derived from a species of Lepidoptera, e.g., Spodoptera frugiperda, Spodoptera littoralis, Spodoptera exigua, or Trichoplusia ni. In some embodiments, the insect cell is Sf9. In some embodiments, the at least one vector is a baculoviral vector, a viral vector, or a plasmid. In some embodiments, the at least one vector is a baculoviral vector. In some embodiments, subclones of lepidopteran cell lines that demonstrate enhanced vector yield on a per cell or per volume basis are used. In some embodiments, modified lepidopteran cell lines with an integrated copy of NS1, Rep, VP, and/or vector genome, singly or in combinations, are used. The insect cell line, in some embodiments, is “cured” of endogenous or contaminating or adventitious insect viruses such as the Spodoptera rhabdovirus.

In some embodiments, the VP1 comprises an amino acid sequence that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to a sequence selected from SEQ ID NOs: 4, 5, 7, 8, 10, 11, 13, 14, 16, 17, 19, 20, and 22. In some embodiments, the VP2 comprises an amino acid sequence that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to a sequence selected from SEQ ID NOs: 4, 6, 7, 9, 10, 12, 13, 15, 16, 18, 19, 21, and 23. In some embodiments, the capsid protein comprise the structural proteins VP1 and VP2. VP2 may be present in excess of VP1.

In some embodiments, the ITR comprise (a) a dependoparvovirus ITR (b) an AAV ITR, optionally an AAV2 ITR, (c) a protoparvovirus ITR, and/or a tetraparvovirus ITR. In certain embodiments, the ITR is a terminal palindrome with Rep binding elements and trs that is structurally similar to the wild-type ITR. The ITR, in some embodiments, is from AAV1, 2, 3, etc. In certain embodiments, the ITR has the AAV2 RBE and trs. In some embodiments, the ITR is a chimera of different AAVs. In some embodiments, the ITR and the Rep protein are from AAV5. In some embodiments, the ITR is synthetic and is comprised of RBE motifs and trs GGTTGG, AGTTGG, AGTTGA, RRTTRR. The typical T-shaped structure of the terminal palindrome consisting of the B/B′ and C/C′ stems may also be synthetically modified with substitutions and insertions that maintain the overall secondary structure based on folding prediction (available at URL (http) of unafold.ma.albany.edu/?q=mfold/DNA-Folding-Form). The stability of the ITR secondary structure is designated by the Gibbs free energy, delta G, with lower values, i.e., more negative, indicating greater stability. The full-length, 145 nt ITR has a computed ΔG=−69.91 kcal/mol. The B and C stems: GCCCGGGCAAAGCCCGGGCGTCGGGCGACCTTTGGTCGCCCG have ΔG=−22.44 kcal/mol. Substitutions and insertions that result in a structure with ΔG=−15 kcal/mol to −30 kcal/mol are functionally equivalent and not distinct from the wild-type dependoparvovirus ITRs.

In some embodiments, the at least one expression control sequence for expression in an insect cell comprises: (a) a promoter, and/or (b) a Kozak-like expression control sequence. In some embodiments, the promoter comprises: (a) an immediate early promoter of an animal DNA virus, (b) an immediate early promoter of an insect virus, or (c) an insect cell promoter. In some embodiments, the animal DNA virus is cytomegalovirus (CMV), protoparvovirus, tetraparvovirus, or AAV. In some embodiments, the insect virus is a lepidopteran virus or a baculovirus, optionally wherein the baculovirus is Autographa californica multicapsid nucleopolyhedrovirus (AcMNPV). In some embodiments, the promoter is a polyhedrin (polh) or immediately early 1 gene (IE-1) promoter. In some embodiments, the nucleotide sequence comprising at least one replication protein of an AAV (e.g., AAV2) comprises a nucleotide sequence encoding Rep52 and/or Rep78.

In certain aspects, provided herein are insect cells comprising at least one vector, comprising: (i) a nucleotide sequence comprising at least one ITR nucleotide sequence, (ii) a nucleotide sequence comprising at least one gene encoding the protoparvovirus or tetraparvovirus capsid proteins VP1 and/or VP2 operably linked to at least one expression control sequence for expression in an insect cell, and (iii) a nucleotide sequence comprising (A) at least one replication protein of an protoparvovirus or tetraparvovirus operably linked to at least one expression control sequence for expression in an insect cell, (B) at least one replication protein of an AAV, optionally wherein the at least one replication protein of an AAV comprises (a) a Rep52 or a Rep40 coding sequence operably linked to at least one expression control sequence for expression in an insect cell, and/or (b) a Rep78 or a Rep68 coding sequence operably linked to at least one expression control sequence for expression in an insect cell, or (C) a combination of (A) and (B). In some embodiments, the vector is an insect cell-compatible vector that comprises a promoter that facilitates the expression of a nucleic acid in insect cells. In some embodiments, at least one of (i), (ii), (iii)(A), (iii)(B), and (iii)(C) is stably integrated in the insect cell genome.

In some embodiments, the insect cells comprise the at least one gene encoding the protoparvovirus capsid protein(s), wherein the protoparvovirus or a genotypic variant thereof is of a species selected from Carnivore protoparvovirus, Carnivore protoparvovirus 1, Chiropteran protoparvovirus 1, Eulipotyphla protoparvovirus 1, Primate protoparvovirus 1, Primate protoparvovirus 2, Primate protoparvovirus 3, Primate protoparvovirus 4, Rodent protoparvovirus 1, Rodent protoparvovirus 2, Rodent protoparvovirus 3, Ungulate protoparvovirus 1, and Ungulate protoparvovirus 2. In some embodiments, the protoparvovirus or a genotypic variant thereof is selected from canine parvovirus, feline panelukepenia virus, human bufavirus 1, human bufavirus 2, human bufavirus 3, human tusavirus, human cutavirus, Wuharv parvovirus, porcine parvovirus, minute virus of mice, megabat bufavirus, and a genotypic variant thereof. In some embodiments, the insect cells comprise the at least one gene encoding the tetraparvovirus capsid protein(s), wherein the the tetraparvovirus or a genotypic variant thereof is of a species selected from Chiropteran tetraparvovirus 1, Primate tetraparvovirus 1, Ungulate tetraparvovirus 1, Ungulate tetraparvovirus 2, Ungulate tetraparvovirus 3, and Ungulate tetraparvovirus 4. In some embodiments, the tetraparvovirus or a genotypic variant thereof is selected from human parvovirus 4, human parvovirus 4 genotype 1, human parvovirus 4 genotype 2, human parvovirus 4 genotype 3, chimpanzee parvovirus 4, eidolon helvum parvovirus, bovine hokovirus 1, bovine hokovirus 2, porcine hokovirus, porcine cnvirus, yak parvovirus, ovine hokovirus 1, opossum tetraparvovirus, rodent tetraparvovirus, tetraparvovirus sp., and a genotypic variant thereof. In some embodiments, the tetraparvovirus is human parvovirus 4, human parvovirus 4 genotype 1, human parvovirus 4 genotype 2, human parvovirus 4 genotype 3, or a genotypic variant thereof.

In some embodiments, the at least one replication protein of a a protoparvovirus is an NS-1 protein of a canine parvovirus, bufavirus, cutavirus, or a genotypic variant thereof.

In some embodiments, the at least one replication protein of a tetravirus is an NS-1 protein of a human parvovirus 4 or a genotypic variant thereof.

In some embodiments, the insect cell is derived from a species of Lepidoptera, e.g., Spodoptera frugiperda, Spodoptera littoralis, Spodoptera exigua, or Trichoplusia ni. In some embodiments, the insect cell is Sf9. In some embodiments, the at least one vector is a baculoviral vector, a viral vector, or a plasmid. In some embodiments, the at least one vector is a baculoviral vector.

In some embodiments, the VP1 comprises an amino acid sequence that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to a sequence selected from SEQ ID NOs: 4, 5, 7, 8, 10, 11, 13, 14, 16, 17, 19, 20, and 22. In some embodiments, the VP2 comprises an amino acid sequence that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to a sequence selected from SEQ ID NOs: 4, 6, 7, 9, 10, 12, 13, 15, 16, 18, 19, 21, and 23. In some embodiments, the capsid proteins comprise the structural proteins VP1 and VP2. VP2 may be present in excess of VP1.

In some embodiments, the ITR comprise (a) a dependoparvovirus ITR, (b) an AAV ITR, optionally an AAV2 ITR, (c) a protoparvovirus ITR, and/or (d) a tetraparvovirus ITR. In some embodiments, the at least one expression control sequence for expression in an insect cell comprises: (a) a promoter, and/or (b) a Kozak-like expression control sequence. In some embodiments, the promoter comprises: (a) an immediate early promoter of an animal DNA virus, (b) an immediate early promoter of an insect virus, or (c) an insect cell promoter. In some embodiments, the animal DNA virus is cytomegalovirus (CMV), protoparvovirus, tetraparvovirus, or AAV. In some embodiments, the insect virus is a lepidopteran virus or a baculovirus, optionally wherein the baculovirus is Autographa californica multicapsid nucleopolyhedrovirus (AcMNPV). In some embodiments, the promoter is a polyhedrin (polh) or immediately early 1 gene (IE-1) promoter. In some embodiments, the nucleotide sequence comprising at least one replication protein of an AAV (e.g., AAV2) comprises a nucleotide sequence encoding Rep52 and/or Rep78.

While less efficient than the methods described herein, the recombinant virion may also be produced using a mammalian cell, e.g., Grieger et al (2016) Mol Ther 24: 287-297).

### Exemplary Embodiments

Aspects of the present disclosure can also be described as follows:

1. A recombinant virion comprising (1) at least one capsid protein or a variant thereof, of a protoparvovirus or a genotypic variant thereof, and (2) a nucleic acid, wherein the nucleic acid comprises a heterologous nucleic acid.

2. A recombinant virion comprising (1) at least one capsid protein or a variant thereof, of a tetraparvovirus or a genotypic variant thereof, and (2) a nucleic acid, wherein the nucleic acid comprises a heterologous nucleic acid.

3. The recombinant virion of 1, wherein the protoparvovirus or a genotypic variant thereof is of a species selected from Carnivore protoparvovirus, Carnivore protoparvovirus 1, Chiropteran protoparvovirus 1, Eulipotyphla protoparvovirus 1, Primate protoparvovirus 1, Primate protoparvovirus 2, Primate protoparvovirus 3, Primate protoparvovirus 4, Rodent protoparvovirus 1, Rodent protoparvovirus 2, Rodent protoparvovirus 3, Ungulate protoparvovirus 1, and Ungulate protoparvovirus 2.

4. The recombinant virion of 1 or 3, wherein the protoparvovirus or a genotypic variant thereof is selected from canine parvovirus, feline panelukepenia virus, human bufavirus 1, human bufavirus 2, human bufavirus 3, human tusavirus, human cutavirus, Wuharv parvovirus, porcine parvovirus, minute virus of mice, megabat bufavirus, and a genotypic variant thereof.

5. The recombinant virion of 2, wherein the tetraparvovirus or a genotypic variant thereof is of a species selected from Chiropteran tetraparvovirus 1, Primate tetraparvovirus 1, Ungulate tetraparvovirus 1, Ungulate tetraparvovirus 2, Ungulate tetraparvovirus 3, and Ungulate tetraparvovirus 4.

6. The recombinant virion of 2 or 5, wherein the tetraparvovirus or a genotypic variant thereof is selected from human parvovirus 4, human parvovirus 4 genotype 1, human parvovirus 4 genotype 2, human parvovirus 4 genotype 3, chimpanzee parvovirus 4, eidolon helvum parvovirus, bovine hokovirus 1, bovine hokovirus 2, porcine hokovirus, porcine cnvirus, yak parvovirus, ovine hokovirus 1, opossum tetraparvovirus, rodent tetraparvovirus, tetraparvovirus sp., and a genotypic variant thereof.

7. The recombinant virion of 6, wherein the tetraparvovirus is human parvovirus 4, human parvovirus 4 genotype 1, human parvovirus 4 genotype 2, human parvovirus 4 genotype 3, or a genotypic variant thereof.

8. The recombinant virion of any one of 1-7, wherein the virion is icosahedral.

9. The recombinant virion of any one of 1-8, wherein the capsid protein or a variant thereof comprises structural proteins VP1 and/or VP2.

10. The recombinant virion of 9, wherein VP2 is present in excess of VP1.

11. The recombinant virion of 9 or 10, wherein VP1 comprises an amino acid sequence that is at least about 60% identical, about 70% identical, about 80% identical, about 90% identical, about 95% identical, about 96% identical, about 97% identical, about 98% identical, about 99% identical, or 100% identical to a sequence selected from SEQ ID NOs: 4, 5, 7, 8, 10, 11, 13, 14, 16, 17, 19, 20, and 22.

12. The recombinant virion of any one of 9-11, wherein VP2 comprises an amino acid sequence that is at least about 60% identical, about 70% identical, about 80% identical, about 90% identical, about 95% identical, about 96% identical, about 97% identical, about 98% identical, about 99% identical, or 100% identical to a sequence selected from SEQ ID NOs: 4, 6, 7, 9, 10, 12, 13, 15, 16, 18, 19, 21, and 23.

13. The recombinant virion of any one of 1-12, wherein the heterologous nucleic acid comprises a nucleic acid sequence that is at least about 60% identical to a nucleic acid sequence of a target cell.

14. The recombinant virion of any one of 1-13, wherein the heterologous nucleic acid is at least about 60% identical to the nucleic acid of a mammal, preferably wherein the mammal is a human.

15. The recombinant virion of any one of 1-14, wherein the heterologous nucleic acid is not operably linked to a protoparvovirus or tetraparvovirus promoter.

16. The recombinant virion of any one of 1-15, wherein the nucleic acid comprises at least one inverted terminal repeat (ITR).

17. The recombinant virion of 16, wherein the at least one ITR comprises:
(a) a dependoparvovirus ITR,
(b) an AAV ITR, optionally an AAV2 ITR,
(c) a protoparvovirus ITR, or
(d) a tetraparvovirus ITR.

17a. The recombinant virion of 16, wherein the at least one ITR comprises an AAV2 ITR and the AAV2 ITR is in “Flip” conformer, “Flop” conformer, or a combination thereof.

17b. The recombinant virion of 16, wherein the at least one ITR comprises an AAV2 ITR, wherein the AAV2 ITR comprises a nucleic acid sequence that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to the nucleic acid sequence set forth in any one of SEQ ID NOs: 1-3 and SEQ ID NOs: 28-31; or their complementary, reverse, or reverse complementary sequences, optionally any one of SEQ ID NOs: 28-31 or their complementary, reverse, or reverse complementary sequences.

17c. The recombinant virion of 16, wherein the at least one ITR comprises two AAV2 ITRs, wherein the AAV2 ITRs comprise a nucleic acid sequence that is at least about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to the nucleic acid sequence set forth in any combination of SEQ ID NOs: 28-31, e.g.,
(a) SEQ ID NO: 28 (e.g., two Flip conformers; or their complementary or reverse complementary sequences);
(b) SEQ ID NO: 29 (e.g., two Flip conformers; or their complementary or reverse complementary sequences);
(c) SEQ ID NO: 30 (e.g., two Flop conformers; or their complementary or reverse complementary sequences);
(d) SEQ ID NO: 31 (e.g., two Flop conformers; or their complementary or reverse complementary sequences);
(e) SEQ ID NO: 28 and SEQ ID NO: 29 (e.g., two Flip conformers; or their complementary or reverse complementary sequences);
(f) SEQ ID NO: 30 and SEQ ID NO: 31 (e.g., two Flop conformers; or their complementary or reverse complementary sequences);
(g) SEQ ID NO: 28 and SEQ ID NO: 30 (e.g., a Flip conformer and a Flop conformer; or their complementary or reverse complementary sequences);
(h) SEQ ID NO: 28 and SEQ ID NO: 31 (e.g., a Flip conformer and a Flop conformer; or their complementary or reverse complementary sequences);
(i) SEQ ID NO: 29 and SEQ ID NO: 30 (e.g., a Flip conformer and a Flop conformer; or their complementary or reverse complementary sequences); or
(j) SEQ ID NO: 29 and SEQ ID NO: 31 (e.g., a Flip conformer and a Flop conformer; or their complementary, reverse, or reverse complementary sequences);

18. The recombinant virion of 17, wherein the protoparvovirus ITR is selected from the ITRs of canine parvovirus, feline panelukepenia virus, human bufavirus 1, human bufavirus 2, human bufavirus 3, human tusavirus, human cutavirus, Wuharv parvovirus, porcine parvovirus, minute virus of mice, megabat bufavirus, and a genotypic variant thereof.

19. The recombinant virion of 17, wherein the tetraparvovirus ITR is selected from the ITRs of human parvovirus 4, human parvovirus 4 genotype 1, human parvovirus 4 genotype 2, human parvovirus 4 genotype 3, chimpanzee parvovirus 4, eidolon helvum parvovirus, bovine hokovirus 1, bovine hokovirus 2, porcine hokovirus, porcine cnvirus, yak parvovirus, ovine hokovirus 1, opossum tetraparvovirus, rodent tetraparvovirus, tetraparvovirus sp., and a genotypic variant thereof.

20. The recombinant virion of 17 or 19, wherein the tetraparvovirus ITR is selected from the ITRs of human parvovirus 4, human parvovirus 4 genotype 1, human parvovirus 4 genotype 2, human parvovirus 4 genotype 3, and a genotypic variant thereof.

21. The recombinant virion of any one of 1-20, wherein the nucleic acid is deoxyribonucleic acid (DNA).

22. The recombinant virion of 21, wherein the DNA is single-stranded or self-complementary duplex.

23. The recombinant virion of any one of 1-22, wherein the nucleic acid comprises a Rep protein-dependent origin of replication (ori).

24. The recombinant virion of any one of 1-23, wherein the nucleic acid comprises a nucleic acid operably linked to a promoter, optionally placed between two ITRs.

25. The recombinant virion of 24, wherein the promoter is selected from:
(a) a promoter heterologous to the nucleic acid to which it is operably linked;
(b) a promoter that facilitates the tissue-specific expression of the nucleic acid, preferably wherein the promoter facilitates hematopoietic cell-specific expression or erythroid lineage-specific expression;
(c) a promoter that facilitates the constitutive expression of the nucleic acid; and
(d) a promoter that is inducibly expressed, optionally in response to a metabolite or small molecule or chemical entity.

26. The recombinant virion of 24 or 25, wherein the promoter is selected from the CMV promoter, β-globin promoter, CAG promoter, AHSP promoter, MND promoter, Wiskott-Aldrich promoter, and PKLR promoter.

27. The recombinant virion of any one of 1-26, wherein the heterologous nucleic acid encodes a coding RNA and/or a non-coding RNA.

28. The recombinant virion of 25, wherein the heterologous nucleic acid encoding a coding RNA comprises:
(a) a gene encoding a protein or a fragment thereof, preferably a human protein or a fragment thereof;
(b) a nucleic acid encoding a nuclease, optionally a Transcription Activator-Like Effector Nuclease (TALEN), a zinc-finger nuclease (ZFN), a meganuclease, a megaTAL, or a CRISPR endonuclease, (e.g., a Cas9 endonuclease or a variant thereof);
(c) a nucleic acid encoding a reporter, e.g., luciferase or GFP; and/or
(d) a nucleic acid encoding a drug resistance protein, e.g., neomycin resistance.

29. The recombinant virion of 27 or 28, wherein the heterologous nucleic acid encoding a coding RNA is codon-optimized for expression in a target cell.

30. The recombinant virion of any one of 27-29, wherein the heterologous nucleic acid comprises a gene encoding a polypeptide, or a fragment thereof, selected from (HBA1, HBA2, HBB, HBG1, HBG2, HBD, HBE1, and/or HBZ), alpha-hemoglobin stabilizing protein (AHSP), coagulation factor VIII, coagulation factor IX, von Willebrand factor, dystrophin or truncated dystrophin, micro-dystrophin, utrophin or truncated utrophin, micro-utrophin, usherin (USH2A), CEP290, ATPB1, ATPB11, ABCB4, CPS1, ATP7B, KRT5, KRT14, PLEC1, Col7A1, ITGB4, ITGA6, LAMA3, LAMB3, LAMC2, KIND1, INS, F8 or a fragment thereof (e.g., fragment encoding B-domain deleted polypeptide (e.g., VIII SQ, p-VIII)), and cystic fibrosis transmembrane conductance regulator (CFTR).

31. The recombinant virion of 27, wherein the non-coding RNA comprises lncRNA, piRNA, miRNA, shRNA, siRNA, antisense RNA, and/or guide RNA.

32. The recombinant virion of any one of 27-31, wherein the coding RNA, the protein, or the non-coding RNA increases or restores the expression of an endogenous gene of a target cell.

33. The recombinant virion of any one of 27-31, wherein the coding RNA, the protein, or the non-coding RNA decreases or eliminates the expression of an endogenous gene of a target cell.

34. The recombinant virion of any one of 27-33, wherein the recombinant virion comprises a nucleic acid encoding (a) hepcidin or a fragment thereof, and/or homeostatic iron regulator (HFE) or a fragment thereof, (b) at least one non-coding RNA (e.g., piRNA, miRNA, shRNA, siRNA, antisense RNA) that targets DMT-1, ferroportin, and/or an endogenous mutant form of HFE; (c) a CRISPR/Cas system that targets DMT-1, ferroportin, and/or an endogenous mutant form of HFE; and/or (d) any combination of any one of the nucleic acids listed in (a) to (c).

35. The recombinant virion of 34, wherein the recombinant virion (a) increases the expression of HFE and/or hepcidin in the transduced cell; and/or (b) decreases the expression of DMT-1, ferroportin, and/or an endogenous mutant form of HFE in the transduced cell.

36. The recombinant virion of 34 or 35, wherein the recombinant virion prevents or treats hemochromatosis, hereditary hemochromatosis, juvenile hemochromatosis, and/or Wilson's disease.

37. The recombinant virion of any one of 27-33, wherein the recombinant virion comprises a nucleic acid encoding (a) a soluble form of the TNFα receptor, a soluble form of the IL-6 receptor, a soluble form of the IL-12 receptor, and/or a soluble form of the IL-1β receptor; (b) at least one non-coding RNA (e.g., piRNA, miRNA, shRNA, siRNA, antisense RNA) that targets the TNFα receptor, IL-6 receptor, IL-12 receptor, and/or IL-1 receptor; (c) a CRISPR/Cas system that targets the TNFα receptor, IL-6 receptor, IL-12 receptor, and/or IL-1β receptor; and/or (d) any combination of any one of the nucleic acids listed in (a) to (c).

38. The recombinant virion of 37, wherein the recombinant virion (a) increases the expression of a soluble form of the TNFα receptor, a soluble form of the IL-6 receptor, a soluble form of the IL-12 receptor, or a soluble form of the IL-1β receptor in the transduced cell; and/or (b) decreases the expression of the TNFα receptor, IL-6 receptor, IL-12 receptor, or IL-1β receptor in the transduced cell.

39. The recombinant virion of 37 or 38, wherein the recombinant virion prevents or treats rheumatoid arthritis, inflammatory bowel disease, psoriatic arthritis, juvenile chronic arthritis, psoriasis, and/or ankylosing spondylitis.

40. The recombinant virion of any one of 27-33, wherein the recombinant virion comprises a nucleic acid encoding a protein or a fragment thereof selected from IRGM, NOD2, ATG2B, ATG9, ATG5, ATG7, ATG16L1, BECN1, EI24/PIG8, TECPR2, WDR45/WIP14, CHMP2B, CHMP4B, Dynein, EPG5, HspB8, LAMP2, LC3b UVRAG, VCP/p97, ZFYVE26, PARK2/Parkin, PARK6/PINK1, SQSTM1/p62, SMURF, AMPK, and ULK1.

41. The recombinant virion of 40, wherein the recombinant virion increases the expression of said protein or a fragment thereof in the transduced cells.

42. The recombinant virion of 40 or 41, wherein the recombinant virion modulates autophagy.

43. The recombinant virion of any one of 40-42, wherein the recombinant virion prevents or treats an autophagy-related disease.

44. The recombinant virion of 43, wherein the autophagy-related disease is selected from cancer, neurodegenerative disease (e.g., Alzheimer's disease, Parkinson's disease, Huntington's disease, ataxias), inflammatory disease, inflammatory bowel disease, Crohn's disease, rheumatoid arthritis, lupus, multiple sclerosis, chronic obstructive pulmony disease/COPD, pulmonary fibrosis, cystic fibrosis, Sjogren's disease, hyperglycemic disorders, type I diabetes, type II diabetes, insulin resistance, hyperinsulinemia, insulin-resistant diabetes (e.g. Mendenhall's Syndrome, Werner Syndrome, leprechaunism, and lipoatrophic diabetes), dyslipidemia, hyperlipidemia, elevated low-density lipoprotein (LDL), depressed highdensity lipoprotein (HDL), elevated triglycerides, metabolic syndrome, liver disease, renal disease, cardiovascular disease, ischemia, stroke, complications during reperfusion, muscle degeneration, atrophy, symptoms of aging (e.g., muscle atrophy, frailty, metabolic disorders, low grade inflammation, atherosclerosis, stroke, age-associated dementia and sporadic form of Alzheimer's disease, pre-cancerous states, and psychiatric conditions including depression), spinal cord injury, arteriosclerosis, infectious diseases (e.g., bacterial, fungal, viral), AIDS, tuberculosis, defects in embryogenesis, infertility, lysosomal storage diseases, activator deficiency/GM2 gangliosidosis, alpha-mannosidosis, aspartylglucoaminuria, cholesteryl ester storage disease, chronic hexosaminidase A deficiency, cystinosis, Danon disease, Fabry disease, Farber disease, fucosidosis, galactosialidosis, Gaucher Disease (Types I, II and III), GM1 Gangliosidosis, (infantile, late infantile/juvenile and adult/chronic), Hunter syndrome (MPS II), I-Cell disease/Mucolipidosis II, Infantile Free Sialic Acid Storage Disease (ISSD), Juvenile Hexosaminidase A Deficiency, Krabbe disease, Lysosomal acid lipase deficiency, Metachromatic Leukodystrophy, Hurler syndrome, Scheie syndrome, Hurler-Scheie syndrome, Sanfilippo syndrome, Morquio Type A and B, Maroteaux-Lamy, Sly syndrome, mucolipidosis, multiple sulfate deficiency, Niemann-Pick disease, Neuronal ceroid lipofuscinoses, CLN6 disease, Jansky-Bielschowsky disease, Pompe disease, pycnodysostosis, Sandhoff disease, Schindler disease, Tay-Sachs, and Wolman disease.

45. The recombinant virion of any one of 27-33, wherein the recombinant virion comprises a nucleic acid encoding (a) CFTR or a fragment thereof, (b) at least one non-coding RNA (e.g., piRNA, miRNA, shRNA, siRNA, antisense RNA) that targets an endogenous mutant form of CFTR, (c) a CRISPR/Cas system that targets an endogenous mutant form of CFTR; and/or (d) any combination of any one of the nucleic acids listed in (a) to (c).

46. The recombinant virion of 45, wherein the recombinant virion (a) increases the expression of CFTR or fragment thereof, and/or (b) decreases the expression of the endogenous mutant form of CFTR in the transduced cell.

47. The recombinant virion of 45 or 46, wherein the recombinant virion prevents or treats cystic fibrosis.

48. The recombinant virion of any one of 1-47, wherein the nucleic acid comprises a non-coding DNA.

49. The recombinant virion of 48, wherein the non-coding DNA comprises:
(a) a transcription regulatory element (e.g., an enhancer, a transcription termination sequence, an untranslated region (5′ or 3′ UTR), a proximal promoter element, a locus control region, a polyadenylation signal sequence), and/or
(b) a translation regulatory element (e.g., Kozak sequence, woodchuck hepatitis virus post-transcriptional regulatory element).

50. The recombinant virion of 49, wherein the transcription regulatory element is a locus control region, optionally a β-globin LCR or a DNase hypersensitive site (HS) of 3-globin LCR.

51. The recombinant virion of any one of 1-50, wherein the nucleic acid comprises a nucleic acid sequence that is at least about 80% identical to the nucleic acid sequence of a genomic safe harbor (GSH) of the target cell.

52. The recombinant virion of 51, wherein the nucleic acid that is at least about 80% identical to a GSH is placed 5′ and 3′ to the nucleic acid to be integrated, thereby allowing integration to a specific locus in the target genome by homologous recombination.

53. The recombinant virion of 51 or 52, wherein the GSH is AAVS1, ROSA26, CCR5, Kif6, Pax5, an intergenic region of NUPL2, collagen, HTRP, HI 1 (a thymidine kinase encoding nucleic acid at HI 1 locus), beta-2 microglobulin, GAPDH, TCR, RUNX1, KLHL7, mir684, KCNH2, GPNMB, MIR4540, MIR4475, MIR4476, PRL32P21, LOC105376031, LOC105376032, LOC105376030, MELK, EBLN3P, ZCCHC7, or RNF38.

54. The recombinant virion of 53, wherein the GSH is AAVS1, ROSA26, CCR5, Kif6, Pax5, or an intergenic region of NUPL2.

55. The recombinant virion of any one of 1-54, wherein the nucleic acid is integrated into the genome of a target cell upon transduction.

56. The recombinant virion of 55, wherein the nucleic acid is integrated into a GSH of the genome of a target cell upon transduction.

57. The recombinant virion of any one of 1-56, wherein the nucleic acid comprises a nucleic acid sequence encoding at least one replication protein and capsid protein or a variant thereof.

58. The recombinant virion of any one of 1-57, wherein the virion is autonomously replicating.

59. The recombinant virion of any one of 1-58, wherein the virion binds and/or transduces a cancer cell or non-cancerous cell.

60. The recombinant virion of any one of 1-59, wherein the virion binds and/or transduces a stem cell (e.g., hematopoietic stem cell, CD34+ stem cell, CD36+ stem cell, mesenchymal stem cell, cancer stem cell).

61. The recombinant virion of any one of 1, 3, 4, 8-18, and 21-60, wherein the virion binds and/or transduces a cell expressing the transferrin receptor (CD71).

62. The recombinant virion of any one of 1-61, wherein the recombinant virion binds and/or transduces a hematopoietic cell, hematopoietic progenitor cell, hematopoietic stem cell, erythroid lineage cell, megakaryocyte, erythroid progenitor cell (EPC), CD34+ cell, CD36+ cell, mesenchymal stem cell, nerve cell, intestinal cell, intestinal stem cell, gut epithelial cell, endothelial cell, lung cell, enterocyte, liver cell (e.g., hepatocyte, hepatic stellate cells (HSCs), Kupffer cells (KCs), liver sinusoidal endothelial cells (LSECs)), brain microvascular endothelial cell (BMVECs), erythroid progenitor cell, lymphoid progenitor cell, B lymphoblast cell, B cell, T cell, basophilic Endemic Burkitt Lymphoma (EBL), polychromatic erythroblast, epidermal stem cell, P63-positive keratinocyte-derived stem cell, keratinocyte, pancreatic 3-cell, K cell, L cell, and/or orthochromatic erythroblast.

63. The recombinant virion of any one of 1-62, wherein the at least one capsid protein or a variant thereof comprises a VP2 sequence having one or more mutations with respect to canine parvovirus strain N (UniProtKB—P12930) or the amino acid sequence SEQ ID NO: 27.

64. The recombinant virion of 63, wherein said one or more mutations are at a region of VP2 having the amino acid residues (i) 91-95, (ii) 297-301, and/or (iii) 320-324.

65. The recombinant virion of 63 or 64, wherein said one or more mutations comprise a substitution, deletion, and/or insertion.

66. The recombinant virion of any one of 63-65, wherein said one or more mutations alter the affinity and/or specificity of the recombinant virion to at least one cellular receptor involved in internalization of the recombinant virion, optionally wherein the at least one cellular receptor is the transferrin receptor.

67. The recombinant virion of any one of 63-66, wherein said one or more mutations alter:
a) the tropism or affinity of the recombinant virion to a cell;
b) the ability of the recombinant virion to transduce a cell; and/or
c) the ability of the recombinant virion to transcytose across the cell.

68. The recombinant virion of any one of 1-67, wherein the at least one capsid protein or a variant thereof comprises a heterologous peptide tag.

69. The recombinant virion of 68, wherein said heterologous peptide tag allows affinity purification using an antibody, an antigen-binding fragment of an antibody, or a nanobody.

70. The recombinant virion of 68 or 69, wherein said heterologous peptide tag comprises an epitope/tag selected from hemagglutinin, His (e.g., 6×-His), FLAG, E-tag, TK15, Strep-tag II, AU1, AU5, Myc, Glu-Glu, KT3, and IRS.

71. A pharmaceutical composition comprising the recombinant virion of any one of 1-70; and a carrier and/or a diluent.

72. A method of preventing or treating a disease, comprising administering to a subject in need thereof an effective amount of the at least one recombinant virion or pharmaceutical composition of any one of 1-71.

73. A method of preventing or treating a disease, comprising:
(a) obtaining a plurality of cells;
(b) transducing the cells with the at least one recombinant virion or pharmaceutical composition of any one of 1-71, optionally further selecting or screening for the transduced cells; and
(c) administering an effective amount of the transduced cells to a subject in need thereof.

74. The method of 73, wherein the cells are autologous or allogeneic to the subject.

75. The method of any one of 72-74, wherein
(a) the nucleic acid encodes a protein; or
(b) the nucleic acid decreases or eliminates the expression of an endogenous gene.

76. The method of any one of 72-75, wherein the at least one recombinant virion, pharmaceutical composition, or transduced cells are administered via intravascular, intracerebral, parenteral, intraperitoneal, intravenous, epidural, intraspinal, intrasternal, intra-articular, intra-synovial, intrathecal, intra-arterial, intracardiac, intramuscular, intranasal, intrapulmonary, skin graft, or oral administration.

77. The method of any one of 72-76, wherein the disease is selected from endothelial dysfunction, cystic fibrosis, cardiovascular disease, renal disease, cancer, hemoglobinopathy, anemia, hemophilia (e.g., hemophilia A), myeloproliferative disorder, coagulopathy, sickle cell disease, alpha-thalassemia, beta-thalassemia, Fanconi anemia, familial intrahepatic cholestasis, epidermolysis bullosa, Fabry, Gaucher, Nieman-Pick A, Nieman-Pick B, GM1 Gangliosidosis, Mucopolysaccharidosis (MPS) I (Hurler, Scheie, Hurler/Scheie), MPS II (Hunter), MPS VI (Maroteaux-Lamy), hematologic cancer, hemochromatosis, hereditary hemochromatosis, juvenile hemochromatosis, cirrhosis, hepatocellular carcinoma, pancreatitis, diabetes mellitus, cardiomyopathy, arthritis, hypogonadism, heart disease, heart attack, hypothyroidism, glucose intolerance, arthropathy, liver fibrosis, Wilson's disease, ulcerative colitis, Crohn's disease, Tay-Sachs disease, neurodegenerative disorder, Spinal muscular atrophy type 1, Huntington's disease, Canavan's disease, rheumatoid arthritis, inflammatory bowel disease, psoriatic arthritis, juvenile chronic arthritis, psoriasis, and ankylosing spondylitis, and autoimmune disease, neurodegenerative disease (e.g., Alzheimer's disease, Parkinson's disease, Huntington's disease, ataxias), inflammatory disease, inflammatory bowel disease, Crohn's disease, rheumatoid arthritis, lupus, multiple sclerosis, chronic obstructive pulmony disease/COPD, pulmonary fibrosis, Sjogren's disease, hyperglycemic disorders, type I diabetes, type II diabetes, insulin resistance, hyperinsulinemia, insulin-resistant diabetes (e.g. Mendenhall's Syndrome, Werner Syndrome, leprechaunism, and lipoatrophic diabetes), dyslipidemia, hyperlipidemia, elevated low-density lipoprotein (LDL), depressed highdensity lipoprotein (HDL), elevated triglycerides, metabolic syndrome, liver disease, renal disease, cardiovascular disease, ischemia, stroke, complications during reperfusion, muscle degeneration, atrophy, symptoms of aging (e.g., muscle atrophy, frailty, metabolic disorders, low grade inflammation, atherosclerosis, stroke, age-associated dementia and sporadic form of Alzheimer's disease, pre-cancerous states, and psychiatric conditions including depression), spinal cord injury, arteriosclerosis, infectious diseases (e.g., bacterial, fungal, viral), AIDS, tuberculosis, defects in embryogenesis, infertility, lysosomal storage diseases, activator deficiency/GM2 gangliosidosis, alpha-mannosidosis, aspartylglucoaminuria, cholesteryl ester storage disease, chronic hexosaminidase A deficiency, cystinosis, Danon disease, Farber disease, fucosidosis, galactosialidosis, Gaucher Disease (Types I, II and III), GM1 Gangliosidosis, (infantile, late infantile/juvenile and adult/chronic), Hunter syndrome (MPS II), I-Cell disease/Mucolipidosis II, Infantile Free Sialic Acid Storage Disease (ISSD), Juvenile Hexosaminidase A Deficiency, Krabbe disease, Lysosomal acid lipase deficiency, Metachromatic Leukodystrophy, Hurler syndrome, Scheie syndrome, Hurler-Scheie syndrome, Sanfilippo syndrome, Morquio Type A and B, Maroteaux-Lamy, Sly syndrome, mucolipidosis, multiple sulfate deficiency, Neuronal ceroid lipofuscinoses, CLN6 disease, Jansky-Bielschowsky disease, Pompe disease, pycnodysostosis, Sandhoff disease, Schindler disease, and Wolman disease.

78. The method of any one of 72-77, wherein the at least one recombinant virion or pharmaceutical composition comprises at least one capsid protein or variant thereof of a protoparvovirus or a genotypic variant thereof.

79. The method of 78, wherein the at least one recombinant virion, pharmaceutical composition, or transduced cells comprise a nucleic acid encoding (a) hepcidin or a fragment thereof, and/or homeostatic iron regulator (HFE) or a fragment thereof, (b) at least one non-coding RNA (e.g., piRNA, miRNA, shRNA, siRNA, antisense RNA) that targets DMT-1, ferroportin, and/or an endogenous mutant form of HFE; (c) a CRISPR/Cas system that targets DMT-1, ferroportin, and/or an endogenous mutant form of HFE; and/or (d) any combination of any one of the nucleic acids listed in (a) to (c).

80. The method of 78 or 79, wherein the subject is administered with the at least one recombinant virion or pharmaceutical composition comprising a nucleic acid encoding:
a) hepcidin or a fragment thereof, wherein the at least one recombinant virion or pharmaceutical composition is administered to the subject intravenously, optionally wherein (i) said nucleic acid is operably linked to a promotor, and/or (ii) the at least one recombinant virion or pharmaceutical composition transduces a hepatocyte;
b) HFE or a fragment thereof, wherein the at least one recombinant virion or pharmaceutical composition is administered to the subject intravenously, optionally wherein (i) said nucleic acid is operably linked to a promotor, and/or (ii) the at least one recombinant virion or pharmaceutical composition transduces a hepatocyte;
c) HFE or a fragment thereof, wherein the at least one recombinant virion or pharmaceutical composition is administered to the subject orally, optionally wherein (i) said nucleic acid is operably linked to a promotor, and/or (ii) the at least one recombinant virion or pharmaceutical composition transduces an enterocyte;
d) at least one non-coding RNA (e.g., piRNA, miRNA, shRNA, siRNA, antisense RNA) that targets an endogenous mutant form of HFE, wherein the at least one recombinant virion or pharmaceutical composition is administered to the subject intravenously, optionally wherein (i) said nucleic acid is operably linked to a promotor, and/or (ii) the at least one recombinant virion or pharmaceutical composition transduces a hepatocyte;
e) at least one non-coding RNA (e.g., piRNA, miRNA, shRNA, siRNA, antisense RNA) that targets an endogenous mutant form of HFE, wherein the at least one recombinant virion or pharmaceutical composition is administered to the subject orally, optionally wherein (i) said nucleic acid is operably linked to a promotor, and/or (ii) the at least one recombinant virion or pharmaceutical composition transduces an enterocyte;
f) at least one non-coding RNA (e.g., piRNA, miRNA, shRNA, siRNA, antisense RNA) that targets DMT-1, wherein the at least one recombinant virion or pharmaceutical composition is administered to the subject orally, optionally wherein (i) said nucleic acid is operably linked to a promotor, and/or (ii) the at least one recombinant virion or pharmaceutical composition transduces an enterocyte;
g) at least one non-coding RNA (e.g., piRNA, miRNA, shRNA, siRNA, antisense RNA) that targets ferroportin, wherein the at least one recombinant virion or pharmaceutical composition is administered to the subject orally, optionally wherein (i) said nucleic acid is operably linked to a promotor, and/or (ii) the recombinant virion or pharmaceutical composition transduces an enterocyte; or
h) a combination of two or more of any one of a) to g).

81. The method of 80, wherein the combination comprises two or more of any one of b) to e).

82. The method of any one of 78-81, wherein the recombinant virion or pharmaceutical composition a) increases the expression of HFE or a fragment thereof, and/or hepcidin or a fragment thereof in the transduced cell; and/or b) decreases the expression of DMT-1, ferroportin, and/or an endogenous mutant form of HFE in the transduced cell.

83. The method of any one of 78-82, wherein the at least one recombinant virion, pharmaceutical composition, or transduced cells prevent or treat hemochromatosis, hereditary hemochromatosis, juvenile hemochromatosis, and/or Wilson's disease.

84. The method of 78, wherein the at least one recombinant virion, pharmaceutical composition, or transduced cells comprise a nucleic acid encoding (a) a soluble form of the TNFα receptor, a soluble form of the IL-6 receptor, a soluble form of the IL-12 receptor, and/or a soluble form of the IL-1β receptor; (b) at least one non-coding RNA (e.g., piRNA, miRNA, shRNA, siRNA, antisense RNA) that targets the TNFα receptor, IL-6 receptor, IL-12 receptor, and/or IL-1β receptor; (c) a CRISPR/Cas system that targets the TNFα receptor, IL-6 receptor, IL-12 receptor, and/or IL-1β receptor; and/or (d) any combination of any one of the nucleic acids listed in (a) to (c).

85. The method of 84, wherein the at least one recombinant virion or pharmaceutical composition a) increases the expression of a soluble form of the TNFα receptor, a soluble form of the IL-6 receptor, a soluble form of the IL-12 receptor, or a soluble form of the IL-1β receptor in the transduced cell; and/or b) decreases the expression of the TNFα receptor, IL-6 receptor, IL-12 receptor, or IL-1β receptor in the transduced cell.

86. The method of any one of 78, 84, and 85, wherein the at least one recombinant virion, pharmaceutical composition, or transduced cells prevent or treat rheumatoid arthritis, inflammatory bowel disease, psoriatic arthritis, juvenile chronic arthritis, psoriasis, and/or ankylosing spondylitis.

87. The method of 78, wherein the at least one recombinant virion, pharmaceutical composition, or transduced cells comprise a nucleic acid encoding a protein or a fragment thereof selected from IRGM, NOD2, ATG2B, ATG9, ATG5, ATG7, ATG16L1, BECN1, EI24/PIG8, TECPR2, WDR45/WIP14, CHMP2B, CHMP4B, Dynein, EPG5, HspB8, LAMP2, LC3b UVRAG, VCP/p97, ZFYVE26, PARK2/Parkin, PARK6/PINK1, SQSTM1/p62, SMURF, AMPK, and ULK1.

88. The method of 87, wherein the at least one recombinant virion or pharmaceutical composition increases the expression of said protein or a fragment thereof in the transduced cells.

89. The method of any one of 78, 87, and 88, wherein the at least one recombinant virion, pharmaceutical composition, or transduced cells modulate autophagy.

90. The method of any one of 78 and 87-89, wherein the at least one recombinant virion, pharmaceutical composition, or transduced cells prevent or treat an autophagy-related disease.

91. The method of 90, wherein the autophagy-related disease is selected from selected from cancer, neurodegenerative disease (e.g., Alzheimer's disease, Parkinson's disease, Huntington's disease, ataxias), inflammatory disease, inflammatory bowel disease, Crohn's disease, rheumatoid arthritis, lupus, multiple sclerosis, chronic obstructive pulmony disease/COPD, pulmonary fibrosis, cystic fibrosis, Sjogren's disease, hyperglycemic disorders, type I diabetes, type II diabetes, insulin resistance, hyperinsulinemia, insulin-resistant diabetes (e.g. Mendenhall's Syndrome, Werner Syndrome, leprechaunism, and lipoatrophic diabetes), dyslipidemia, hyperlipidemia, elevated low-density lipoprotein (LDL), depressed highdensity lipoprotein (HDL), elevated triglycerides, metabolic syndrome, liver disease, renal disease, cardiovascular disease, ischemia, stroke, complications during reperfusion, muscle degeneration, atrophy, symptoms of aging (e.g., muscle atrophy, frailty, metabolic disorders, low grade inflammation, atherosclerosis, stroke, age-associated dementia and sporadic form of Alzheimer's disease, pre-cancerous states, and psychiatric conditions including depression), spinal cord injury, arteriosclerosis, infectious diseases (e.g., bacterial, fungal, viral), AIDS, tuberculosis, defects in embryogenesis, infertility, lysosomal storage diseases, activator deficiency/GM2 gangliosidosis, alpha-mannosidosis, aspartylglucoaminuria, cholesteryl ester storage disease, chronic hexosaminidase A deficiency, cystinosis, Danon disease, Fabry disease, Farber disease, fucosidosis, galactosialidosis, Gaucher Disease (Types I, II and III), GM1 Gangliosidosis, (infantile, late infantile/juvenile and adult/chronic), Hunter syndrome (MPS II), I-Cell disease/Mucolipidosis II, Infantile Free Sialic Acid Storage Disease (ISSD), Juvenile Hexosaminidase A Deficiency, Krabbe disease, Lysosomal acid lipase deficiency, Metachromatic Leukodystrophy, Hurler syndrome, Scheie syndrome, Hurler-Scheie syndrome, Sanfilippo syndrome, Morquio Type A and B, Maroteaux-Lamy, Sly syndrome, mucolipidosis, multiple sulfate deficiency, Niemann-Pick disease, Neuronal ceroid lipofuscinoses, CLN6 disease, Jansky-Bielschowsky disease, Pompe disease, pycnodysostosis, Sandhoff disease, Schindler disease, Tay-Sachs, and Wolman disease.

92. The method of 78, wherein the protoparvovirus is cutavirus.

93. The method of 92, wherein the at least one recombinant virion or pharmaceutical composition targets a T cell, B cell, and/or a lymphoid progenitor cell.

94. The method of any one of 78, 92, and 93, wherein the at least one recombinant virion, pharmaceutical composition, or transduced cells prevent or treat cancer.

95. The method of 78 or 92, wherein the at least one recombinant virion, pharmaceutical composition, or transduced cells comprise a nucleic acid encoding KRT5, KRT14, PLEC1, Col7A1, ITGB4, ITGA6, LAMA3, LAMB3, LAMC2, and/or KIND 1.

96. The method of 95, wherein the transduced cells are epidermal stem cells, P63-positive keratinocyte-derived stem cells, or keratinocytes.

97. The method of 95 or 96, further comprising grafting the transduced cells on the skin of the subject.

98. The method of any one of 95-97, wherein the at least one recombinant virion, pharmaceutical composition, or transduced cells prevent or treat epidermolysis bullosa.

99. The method of any one of 72-77, wherein the at least one recombinant virion or pharmaceutical composition comprises at least one capsid protein or variant thereof of a tetraparvovirus or a genotypic variant thereof.

100. The method of 99, wherein the at least one recombinant virion or pharmaceutical composition comprises a nucleic acid encoding a protein or a fragment thereof selected from a hemoglobin gene (HBA1, HBA2, HBB, HBG1, HBG2, HBD, HBE1, and/or HBZ), alpha-hemoglobin stabilizing protein (AHSP), coagulation factor VIII, coagulation factor IX, von Willebrand factor, dystrophin or truncated dystrophin, micro-dystrophin, utrophin or truncated utrophin, micro-utrophin, usherin (USH2A), CEP290, ATPB1, ATPB11, ABCB4, CPS1, ATP7B, KRT5, KRT14, PLEC1, Col7A1, ITGB4, ITGA6, LAMA3, LAMB3, LAMC2, KIND1, INS, F8 or a fragment thereof (e.g., fragment encoding B-domain deleted polypeptide (e.g., VIII SQ, p-VIII)), and cystic fibrosis transmembrane conductance regulator (CFTR).

101. The method of 99 or 100, wherein the at least one recombinant virion or pharmaceutical composition transduces (a) a CD34+ stem cell, optionally transduces ex vivo; (b) a mesenchymal stem cell, optionally transduces ex vivo; (c) a liver cell (e.g., hepatocyte), (d) a small intestinal cell, and/or (e) a lung cell.

102. The method of any one of 99-101, wherein the at least one recombinant virion or pharmaceutical composition is delivered to liver via hepatic artery, portal vein, or intravenous administration.

103. The method of any one of 99-101, wherein the at least one recombinant virion or pharmaceutical composition is delivered to small intestine via oral administration.

104. The method of any one of 99-101, wherein the at least one recombinant virion or pharmaceutical composition comprises a nucleic encoding (a) CFTR or a fragment thereof, (b) at least one non-coding RNA (e.g., piRNA, miRNA, shRNA, siRNA, antisense RNA) that targets an endogenous mutant form of CFTR, (c) a CRISPR/Cas system that targets an endogenous mutant form of CFTR; and/or (d) any combination of any one of the nucleic acids listed in (a) to (c).

105. The method of 104, wherein the at least one recombinant virion or pharmaceutical composition is delivered to the lung via an intranasal or intrapulmonary administration.

106. The method of any one of 99-101, 104, and 105, wherein the at least one recombinant virion or pharmaceutical composition (a) increases the expression of CFTR or fragment thereof, and/or (b) decreases the expression of an endogenous mutant form of CFTR in the transduced cell.

107. The method of any one of 99-101 and 104-106, wherein the at least one recombinant virion or pharmaceutical composition prevents or treats cystic fibrosis.

108. The method of any one of 72-107, wherein the method further comprises re-administering at least one additional amount of the virion, pharmaceutical composition, or transduced cells.

109. The method of 108, wherein said re-administering the at least one additional amount is performed after an attenuation in the treatment subsequent to said administering the effective amount of the virion, pharmaceutical composition, or transduced cells.

110. The method of 108 or 109, wherein the at least one additional amount is the same as the said effective amount.

111. The method of 108 or 109, wherein the method further comprises increasing or decreasing the at least one additional amount as compared to the said effective amount.

112. The method of 111, wherein the at least one additional amount is increased or decreased based on the expression of an endogenous gene and/or the nucleic acid of the recombinant virion.

113. The method of any one of 72-112, further comprising administering to the subject or contacting the cells with an agent that modulates the expression of the nucleic acid.

114. The method of 113, wherein the agent is selected from a small molecule, a metabolite, an oligonucleotide, a riboswitch, a peptide, a peptidomimetic, a hormone, a hormone analog, and light.

115, The method of 113 or 114, wherein the agent is selected from tetracycline, cumate, tamoxifen, estrogen, and an antisense oligonucleotide (ASO), rapamycin, FKCsA, blue light, abscisic acid (ABA), and riboswitch.

116. The method of any one of 72-115, further comprising re-administering the agent one or more times at intervals.

117. The method of 116, wherein the re-administration of the agent results in pulsatile expression of the nucleic acid.

118. The method of 116 or 117, wherein the time between the intervals and/or the amount of the agent is increased or decreased based on the serum concentration and/or half-life of the protein expressed from the nucleic acid.

119. A method of modulating (i) gene expression, or (ii) function and/or structure of a protein in a cell, the method comprising transducing the cell with the virion or pharmaceutical composition of any one of 1-71 comprising a nucleic acid that modulates the gene expression, or the function and/or structure of the protein in the cell.

120. The method of 119, wherein the nucleic acid comprises the sequence encoding CRISPRi or CRISPRa agents.

121. The method of 119 or 120, wherein the gene expression, or the function and/or structure of the protein is increased or restored.

122. The method of 119 or 120, wherein the gene expression, or the function and/or structure of the protein is decreased or eliminated.

123. A method of integrating a heterologous nucleic acid into a GSH in a cell, comprising
(a) transducing the cell with one or more virions or pharmaceutical composition according to any one of 1-71 comprising a heterologous nucleic acid flanked at the 5′ end and 3′ end by a donor nucleic acid sequence that is at least about 80% identical to the target GSH nucleic acid; or
(b) transducing the cell with one or more virions or pharmaceutical composition according to any one of 1-71 comprising (i) a heterologous nucleic acid flanked at the 5′ end and 3′ end by a donor nucleic acid sequence that is at least about 80% identical to the target GSH nucleic acid, and (ii) a nucleic acid encoding a nuclease (e.g., Cas9 or a variant thereof, ZFN, TALEN) and/or a guide RNA, wherein the nuclease or the nuclease/gRNA complex makes a DNA break at the GSH, which is repaired using the donor nucleic acid, thereby integrating a heterologous nucleic acid at GSH.

124. The method of 123, wherein (i) the heterologous nucleic acid flanked by a donor nucleic acid that is at least about 80% identical to the target GSH nucleic acid is transduced in one virion, and (ii) the nucleic acid encoding a nuclease and/or the gRNA are transduced in a separate virion.

125. The method of 123 or 124, wherein the GSH is AAVS1, ROSA26, CCR5, Kif6, Pax5, an intergenic region of NUPL2, collagen, HTRP, HI 1 (a thymidine kinase encoding nucleic acid at HI 1 locus), beta-2 microglobulin, GAPDH, TCR, RUNX1, KLHL7, mir684, KCNH2, GPNMB, MIR4540, MIR4475, MIR4476, PRL32P21, LOC105376031, LOC105376032, LOC105376030, MELK, EBLN3P, ZCCHC7, or RNF38.

126. The method of any one of 123-125, wherein the GSH is AAVS1, ROSA26, CCR5, Kif6, Pax5, or an intergenic region of NUPL2.

127. A method of producing a recombinant virion according to any one of 1-70, comprising:
(1) providing at least one vector comprising
   (i) a nucleotide sequence comprising at least one ITR nucleotide sequence, optionally further comprising a heterologous nucleic acid operably linked to a promoter for expression in a target cell,
   (ii) a nucleotide sequence comprising at least one gene encoding the protoparvovirus or tetraparvovirus capsid proteins VP1,VP2, and/or a variant thereof, operably linked to at least one expression control sequence for expression in an insect cell, and
   (iii) a nucleotide sequence comprising
      (A) at least one replication protein of a protoparvovirus or tetraparvovirus operably linked to at least one expression control sequence for expression in an insect cell,
      (B) at least one replication protein of an AAV, optionally wherein the at least one replication protein of an AAV comprises (a) a Rep52 or a Rep40 coding sequence operably linked to at least one expression control sequence for expression in an insect cell, and/or (b) a Rep78 or a Rep68 coding sequence operably linked to at least one expression control sequence for expression in an insect cell, or
      (C) a combination of (A) and (B),
(2) introducing said at least one vector into an insect cell, and
(3) maintaining said insect cell under conditions such that a recombinant virion according to any one of 1-70 is produced.

128. The method of 127, wherein two vectors are provided,
(a) a first vector comprising a nucleotide sequence comprising at least one ITR nucleotide sequence, optionally further comprising a heterologous nucleic acid operably linked to a promoter for expression in a target cell, and
(b) a second vector comprising
   (i) a nucleotide sequence comprising at least one gene encoding the protoparvovirus or tetraparvovirus capsid proteins VP1,VP2, and/or a variant thereof, operably linked to at least one expression control sequence for expression in an insect cell, and
   (ii) a nucleotide sequence comprising
      (A) at least one replication protein of a protoparvovirus or tetraparvovirus operably linked to at least one expression control sequence for expression in an insect cell,
      (B) at least one replication protein of an AAV, optionally wherein the at least one replication protein of an AAV comprises (a) a Rep52 or a Rep40 coding sequence operably linked to at least one expression control sequence for expression in an insect cell, and/or (b) a Rep78 or a Rep68 coding sequence operably linked to at least one expression control sequence for expression in an insect cell, or
      (C) a combination of (A) and (B).

129. The method of 127, wherein three vectors are provided,
(a) a first vector comprising a nucleotide sequence comprising at least one ITR nucleotide sequence, optionally further comprising a heterologous nucleic acid operably linked to a promoter for expression in a target cell,
(b) a second vector comprising a nucleotide sequence comprising a gene encoding the protoparvovirus or tetraparvovirus capsid proteins VP1, VP2, and/or a variant thereof, operably linked to at least one expression control sequence for expression in an insect cell, and
(c) a third vector comprising a nucleotide sequence comprising
   (A) at least one replication protein of a protoparvovirus or tetraparvovirus operably linked to at least one expression control sequence for expression in an insect cell,
   (B) at least one replication protein of an AAV, optionally wherein the at least one replication protein of an AAV comprises (a) a Rep52 or a Rep40 coding sequence operably linked to at least one expression control sequence for expression in an insect cell, and/or (b) a Rep78 or a Rep68 coding sequence operably linked to at least one expression control sequence for expression in an insect cell, or
   (C) a combination of (A) and (B).

130. A method of producing a recombinant virion according to any one of 1-70 in an insect cell, the method comprising:
(1) providing an insect cell comprising
   (i) a nucleotide sequence comprising at least one ITR nucleotide sequence, optionally further comprising a heterologous nucleic acid operably linked to a promoter for expression in a target cell,
   (ii) a nucleotide sequence comprising at least one gene encoding the protoparvovirus or tetraparvovirus capsid proteins VP1, VP2, and/or a variant thereof, operably linked to at least one expression control sequence for expression in an insect cell, and
   (iii) a nucleotide sequence comprising
      (A) at least one replication protein of a protoparvovirus or tetraparvovirus operably linked to at least one expression control sequence for expression in an insect cell,
      (B) at least one replication protein of an AAV, optionally wherein the at least one replication protein of an AAV comprises (a) a Rep52 or a Rep40 coding sequence operably linked to at least one expression control sequence for expression in an insect cell, and/or (b) a Rep78 or a Rep68 coding sequence operably linked to at least one expression control sequence for expression in an insect cell, or
      (C) a combination of (A) and (B),
   wherein at least one of (i), (ii), (iii)(A), (iii)(B), and (iii)(C) is/are stably integrated in the insect cell genome, and at least one vector, if present, comprises the remainder of the (i), (ii), (iii)(A), (iii)(B), and (iii)(C) nucleotide sequences which is/are not stably integrated in the insect cell genome, and
(2) maintaining the insect cell under conditions such that the recombinant virion is produced.

131. The method of any one of 127-130, wherein the protoparvovirus or a genotypic variant thereof is of a species selected from Carnivore protoparvovirus, Carnivore protoparvovirus 1, Chiropteran protoparvovirus 1, Eulipotyphla protoparvovirus 1, Primate protoparvovirus 1, Primate protoparvovirus 2, Primate protoparvovirus 3, Primate protoparvovirus 4, Rodent protoparvovirus 1, Rodent protoparvovirus 2, Rodent protoparvovirus 3, Ungulate protoparvovirus 1, and Ungulate protoparvovirus 2.

132. The method of any one of 127-131, wherein the protoparvovirus or a genotypic variant thereof is selected from canine parvovirus, feline panelukepenia virus, human bufavirus 1, human bufavirus 2, human bufavirus 3, human tusavirus, human cutavirus, Wuharv parvovirus, porcine parvovirus, minute virus of mice, megabat bufavirus, and a genotypic variant thereof.

133. The method of any one of 127-130, wherein the tetraparvovirus or a genotypic variant thereof is of a species selected from Chiropteran tetraparvovirus 1, Primate tetraparvovirus 1, Ungulate tetraparvovirus 1, Ungulate tetraparvovirus 2, Ungulate tetraparvovirus 3, and Ungulate tetraparvovirus 4.

134. The method of any one of 127-130 and 133, wherein the tetraparvovirus or a genotypic variant thereof is selected from human parvovirus 4, human parvovirus 4 genotype 1, human parvovirus 4 genotype 2, human parvovirus 4 genotype 3, chimpanzee parvovirus 4, eidolon helvum parvovirus, bovine hokovirus 1, bovine hokovirus 2, porcine hokovirus, porcine cnvirus, yak parvovirus, ovine hokovirus 1, opossum tetraparvovirus, rodent tetraparvovirus, tetraparvovirus sp., and a genotypic variant thereof.

135. The method of any one of 127-130, 133, and 134, wherein the tetraparvovirus is human parvovirus 4, human parvovirus 4 genotype 1, human parvovirus 4 genotype 2, human parvovirus 4 genotype 3, or a genotypic variant thereof.

136. The method of any one of 127-135, wherein the at least one replication protein of a protoparvovirus is an NS-1 protein of a canine parvovirus, bufavirus, cutavirus, or a genotypic variant thereof.

137. The method of any one of 127-135, wherein the at least one replication protein of a tetravirus is an NS-1 protein of a human parvovirus 4 or a genotypic variant thereof.

138. The method of any one of 127-137, wherein the insect cell is derived from a species of Lepidoptera.

139. The method of 138, wherein the species of Lepidoptera is Spodoptera frugiperda, Spodoptera littoralis, Spodoptera exigua, or Trichoplusia ni.

140. The method of any one of 127-139, wherein the insect cell is Sf9.

141. The method of any one of 127-140, wherein the at least one vector is a baculoviral vector, a viral vector, or a plasmid.

142. The method of any one of 127-141, wherein the at least one vector is a baculoviral vector.

143. The method of any one of 127-142, wherein VP1 comprises an amino acid sequence that is at least about 60% identical to a sequence selected from SEQ ID NOs: 4, 5, 7, 8, 10, 11, 13, 14, 16, 17, 19, 20, and 22.

144. The method of any one of 127-143, wherein VP2 comprises an amino acid sequence that is at least about 60% identical to a sequence selected from SEQ ID NOs: 4, 6, 7, 9, 10, 12, 13, 15, 16, 18, 19, 21, and 23.

145. The method of any one of 127-144, wherein the at least one ITR comprises:
(a) a dependoparvovirus ITR,
(b) an AAV ITR, optionally an AAV2 ITR,
(c) a protoparvovirus ITR, and/or
(d) a tetraparvovirus ITR.

146. The method of any one of 127-145, wherein the at least one expression control sequence for expression in an insect cell comprises:
(a) a promoter, and/or
(b) a Kozak-like expression control sequence.

147. The method of 146, wherein the promoter comprises:
(a) an immediate early promoter of an animal DNA virus,
(b) an immediate early promoter of an insect virus, or
(c) an insect cell promoter.

148. The method of 147, wherein the animal DNA virus is cytomegalovirus (CMV), protoparvovirus, tetraparvovirus, or AAV.

149. The method of 147, wherein the insect virus is a lepidopteran virus or a baculovirus, optionally wherein the baculovirus is Autographa californica multicapsid nucleopolyhedrovirus (AcMNPV).

150. The method of any one of 146-149, wherein the promoter is a polyhedrin (polh) or immediately early 1 gene (IE-1) promoter.

151. The method of any one of 127-150, wherein the nucleotide sequence comprising at least one replication protein of an AAV comprises a nucleotide sequence encoding Rep52 and/or Rep78.

152. The method of any one of 127-151, wherein the AAV is AAV2.

153. An insect cell, comprising at least one vector, comprising:
(i) a nucleotide sequence comprising at least one ITR nucleotide sequence,
(ii) a nucleotide sequence comprising at least one gene encoding the protoparvovirus or tetraparvovirus capsid proteins VP1, VP2, and/or a variant thereof, operably linked to at least one expression control sequence for expression in an insect cell, and
(iii) a nucleotide sequence comprising
   (A) at least one replication protein of a protoparvovirus or tetraparvovirus operably linked to at least one expression control sequence for expression in an insect cell,
   (B) at least one replication protein of an AAV, optionally wherein the at least one replication protein of an AAV comprises (a) a Rep52 or a Rep40 coding sequence operably linked to at least one expression control sequence for expression in an insect cell, and/or (b) a Rep78 or a Rep68 coding sequence operably linked to at least one expression control sequence for expression in an insect cell, or
   (C) a combination of (A) and (B).

154. The insect cell of 153, wherein at least one of (i), (ii), (iii)(A), (iii)(B), and (iii)(C) is stably integrated in the insect cell genome.

155. The insect cell of 153 or 154, wherein the protoparvovirus or a genotypic variant thereof is of a species selected from Carnivore protoparvovirus, Carnivore protoparvovirus 1, Chiropteran protoparvovirus 1, Eulipotyphla protoparvovirus 1, Primate protoparvovirus 1, Primate protoparvovirus 2, Primate protoparvovirus 3, Primate protoparvovirus 4, Rodent protoparvovirus 1, Rodent protoparvovirus 2, Rodent protoparvovirus 3, Ungulate protoparvovirus 1, and Ungulate protoparvovirus 2.

156. The insect cell of any one of 153-155, wherein the protoparvovirus or a genotypic variant thereof is selected from canine parvovirus, feline panelukepenia virus, human bufavirus 1, human bufavirus 2, human bufavirus 3, human tusavirus, human cutavirus, Wuharv parvovirus, porcine parvovirus, minute virus of mice, megabat bufavirus, and a genotypic variant thereof.

157. The insect cell of 153 or 154, wherein the tetraparvovirus or a genotypic variant thereof is of a species selected from Chiropteran tetraparvovirus 1, Primate tetraparvovirus 1, Ungulate tetraparvovirus 1, Ungulate tetraparvovirus 2, Ungulate tetraparvovirus 3, and Ungulate tetraparvovirus 4.

158. The insect cell of any one of 153, 154, and 157, wherein the tetraparvovirus or a genotypic variant thereof is selected from human parvovirus 4, human parvovirus 4 genotype 1, human parvovirus 4 genotype 2, human parvovirus 4 genotype 3, chimpanzee parvovirus 4, eidolon helvum parvovirus, bovine hokovirus 1, bovine hokovirus 2, porcine hokovirus, porcine cnvirus, yak parvovirus, ovine hokovirus 1, opossum tetraparvovirus, rodent tetraparvovirus, tetraparvovirus sp., and a genotypic variant thereof.

159. The insect cell of any one of 153, 154, 157, and 158, wherein the tetraparvovirus is human parvovirus 4, human parvovirus 4 genotype 1, human parvovirus 4 genotype 2, human parvovirus 4 genotype 3, or a genotypic variant thereof.

160. The insect cell of any one of 153-159, wherein the at least one replication protein of a protoparvovirus is an NS-1 protein of a canine parvovirus, bufavirus, cutavirus, or a genotypic variant thereof.

161. The insect cell of any one of 153-159, wherein the at least one replication protein of a tetravirus is an NS-1 protein of a human parvovirus 4, or a genotypic variant thereof.

162. The insect cell of any one of 153-161, wherein the insect cell is derived from a species of Lepidoptera.

163. The insect cell of 162, wherein the species of Lepidoptera is Spodoptera frugiperda, Spodoptera littoralis, Spodoptera exigua, or Trichoplusia ni.

164. The insect cell of any one of 153-163, wherein the insect cell is Sf9.

165. The insect cell of any one of 153-164, wherein the at least one vector is a baculoviral vector, a viral vector, or a plasmid.

166. The insect cell of any one of 153-165, wherein the at least one vector is a baculoviral vector.

167. The insect cell of any one of 153-166, wherein VP1 comprises an amino acid sequence that is at least about 60% a sequence selected from SEQ ID NOs: 4, 5, 7, 8, 10, 11, 13, 14, 16, 17, 19, 20, and 22.

168. The insect cell of any one of 153-167, wherein VP2 comprises an amino acid sequence that is at least about 60% identical to a sequence selected from SEQ ID NOs: 4, 6, 7, 9, 10, 12, 13, 15, 16, 18, 19, 21, and 23.

169. The insect cell of any one of 153-168, wherein the at least one ITR comprises:
(a) a dependoparvovirus ITR,
(b) an AAV ITR, optionally an AAV2 ITR,
(c) a protoparvovirus ITR, and/or
(d) a tetraparvovirus ITR.

170. The insect cell of any one of 153-169, wherein the at least one expression control sequence for expression in an insect cell comprises:
(a) a promoter, and/or
(b) a Kozak-like expression control sequence.

171. The insect cell of 170, wherein the promoter comprises:
(a) an immediate early promoter of an animal DNA virus,
(b) an immediate early promoter of an insect virus, or
(c) an insect cell promoter.

172. The insect cell of 171, wherein the animal DNA virus is cytomegalovirus (CMV), protoparvovirus, tetraparvovirus, or AAV.

173. The insect cell of 171, wherein the insect virus is a lepidopteran virus or a baculovirus, optionally wherein the baculovirus is Autographa californica multicapsid nucleopolyhedrovirus (AcMNPV).

174. The insect cell of any one of 170-173, wherein the promoter is a polyhedrin (polh) or immediately early 1 gene (IE-1) promoter.

175. The insect cell of any one of 153-174, wherein the nucleotide sequence comprising at least one replication protein of an AAV comprises a nucleotide sequence encoding Rep52 and/or Rep78.

176. The insect cell of any one of 153-175, wherein the AAV is AAV2.

### EXAMPLES

### Example 1: Construction of Recombinant Virions Containing Canine Parvovirus or Human Parvovirus 4 Capsid Proteins

### A Nucleic Acid for the Recombinant Virion

A vector genome design consists of inverted terminal repeats (ITRs), e.g., the ITR conformers of the AAV terminal palindrome and an expression or transcription cassette. The generic expression cassettes consist of regulatory elements, typically characterized as enhancer and promoter elements. The region transcribed by the RNA polymerase complex consists of cis acting regulatory elements e.g., TATA—box, and 5′ untranslated exonic sequences, intronic sequences, translated exonic sequences, 3′ untranslated region, poly-adenylation signal sequence. Post-transcriptional elements include a Kozak motif for translational initiation and the woodchuck hepatitis virus post-transcriptional regulatory element. The specific vector is chemically synthesized using a commercial service provider and ligated into a plasmid for propagation in Escherichia coli. The plasmid minimally contains multiple cloning sites, at least one antibiotic resistance gene, a plasmid origin of replication, and sequences to facilitate recombination into a baculovirus genome. Two commonly used approaches are: 1. A bacterial system in which the E. coli harbors a baculovirus genome (bacmid) that uses transposase mediated recombination to transfer the plasmid genes into the bacmid. E. coli with the recombinant bacmid is detectable by growth on agar plates prepared with selective media. The “positive” colonies are expanded in suspension culture medium and the bacmid harvested after about 3 days post-inoculation. Sf9 cells are then transfected with the bacmid which in the permissive insect cell, produce infectious, recombinant baculovirus particles. 2. Alternatively, the vector DNA is inserted into a shuttle plasmid that has several hundred basepairs of baculovirus DNA flanking the insert. Co-transfection of Sf9 cells with the shuttle plasmid and linearized baculovirus subgenomic DNA restores the deleted baculovirus elements producing infectious, recombinant baculovirus. The ≤6 kb vector DNA resides in the baculovirus genome (ca.135 kb) and is propagated as baculovirus unless the Sf9 cell expresses the parvovirus non-structural or Rep proteins. The Rep protein then acts on the ITR allowing resolution of the vector and baculovirus genomes where the vector genome then replicates autonomously of the baculovirus genome (FIG. 1B).

### Nucleic Acid Composed of DNA

DNA can be either single-stranded or self-complimentary (i.e., intramolecular duplex). As illustrated in FIG. 1B, Rep-mediated replication of the vector DNA proceeds through several intermediates. These replicative intermediates are processed into single-stranded virion genomes, however, the fecundity of products may overwhelm processing into single-stranded virion genomes. In this case, the replicative intermediate consisting of an intramolecular duplex molecule, represented as the RFm (FIG. 1B), is packaged into the parvovirus capsid. Packaging of the self-complementary vector genomes occurs despite the presence of functional ITRs.

DNA can have a Rep protein-dependent origin of replication (ori). The on can consist of Rep binding elements (RBEs), and within a terminal palindrome. The terminal palindrome, referred to as the inverted terminal repeats (ITRs), can consist of an overall palindromic sequence with two internal palindromes. The ITR can have cis-acting motifs required for replication and encapsidation in capsids.

RBE represents Rep binding elements canonical GCTC; RBE′ represents non-canonical RBE, unpaired TTT at the tip of the ITR cross-arm; and trs represents terminal resolution site 5′AGTTGG, GGTTGG, etc. The catalytic tyrosine of Rep (Y156) cleaves the trs and forms a covalent link with the scissile, 5′thymidine. Mutation of the trs leads to inefficient or loss of cleavage resulting in self-complimentary DNA. Alternatively, self-complementary virion genomes result from encapsidation of the incomplete processing of the RFm.

### DNA Replication of the Recombinant Virion Comprising a Canine Parvovirus or Human Parvovirus 4 Capsid Proteins

Replication utilizing AAV ITR—Parvovirus DNA replication is referred to as “rolling hairpin” replication. As single-stranded virion DNA, the ITRs form an energetically stable, T-shaped structure (FIG. 1A) that serves as a primer for DNA extension by the host-cell DNA polymerase complex (FIG. 1B). DNA synthesis is leading strand, processive process resulting in a duplex intermediate where the complementary strands are covalently linked through the ITR (FIG. 1B). The p5 Rep protein binds are structurally related to rolling-circle replication (RCR) proteins, bind to the ITR forming a multi-subunit complex. The helicase activity of the Rep proteins unwinds the ITR creating a single-stranded bubble with the terminal resolution site (5′-GGTITGA-3′). The phosphodiester bond between the thymidines is attacked by the hydroxyl group of the Rep protein catalytic tyrosine (AAV2=Y156) forming a tyrosine—thymidine diester with the 5′-thymidine. A cellular DNA polymerase complex extends the newly created 3-OH at the terminal resolution site restoring the terminal sequence to the template strand (FIG. 1B). Resolution of the nucleoprotein complex occurs through an unknown process.

AAV ITRs or cognate ITRs (e.g., parvovirus ITRs) are used to construct the recombinant virions comprising the parvovirus capsid protein(s).

Replication utilizing canine parvovirus (an exemplary protoparvovirus) or human parvovirus 4 (an exemplary tetraparvovirus) terminal repeats and non-structural (NS) protein(s)—protoparvovirus or tetraparvovirus is similar to AAV DNA replication, although the terminal palindromes are unique and require a specific NS protein for processing the replication intermediates. The recombinant canine parvovirus or human parvovirus 4 vector genome may consist of canine parvovirus or human parvovirus 4 termini flanking the transgene cassette. NS1 dependent rolling-hairpin replication process is similar to AAV Rep-dependent replication and capsid contain single stranded genomes of either polarity.

### Encapsidation

Encapsidation or packaging of DNA into an icosahedral virus capsid is an active process requiring a source of energy to overcome the repulsive force created by back-pressure of compressing DNA into a confined volume. The ATPase activities of the NS/Rep proteins translate the stored chemical energy of the trinucleotide by hydrolyzing the gamma phosphate. The backpressure generated determines the length of DNA that can be accommodated in the capsid, i.e., the motive force of the ATPase/helicase can “push” up to 12 pN, for example, which may be reached once 4,800 nucleotides are packaged. AAV p19 Rep proteins are monomeric, non-processive helicases that are necessary for efficient encapsidation. Although there are scant data that support physical interactions between Rep and capsid, the overcoming the backpressure requires that stable interactions form between the packaging helicase(s) and the capsid. The nature of these interactions are unknown and nuclear factors may stabilize or mediate the interactions between the non-structural proteins and capsids. The phylogenetically related protoparvovirus/tetraparvovirus and dependoparvovirus capsids are divergent at the sequence level, therefore, the interactions between NS proteins and capsids of heterologous genera may not result in efficient encapsidation.

To improve the packaging of genomes comprising AAV2 genes into canine parvovirus or human parvovirus 4 capsids, the cognate NS proteins are co-expressed with the AAV Rep proteins in a permissive cell: AAV Rep78 and Rep 52 are required for vector DNA replication and canine parvovirus or human parvovirus 4 NS proteins are involved in packaging.

### Example 2: Production of Components Necessary for Manufacturing the Recombinant Virion Comprising Canine Parvovirus (CPV) Capsid Proteins

Components necessary for manufacturing the recombinant virion comprising CPV capsid proteins were produced using the Baculovirus Expression Vector (BEV) system and Sf9 cells.

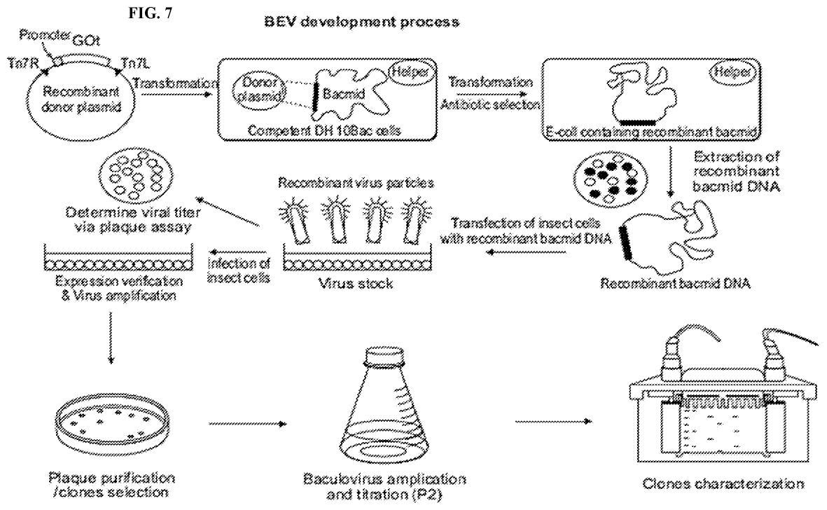

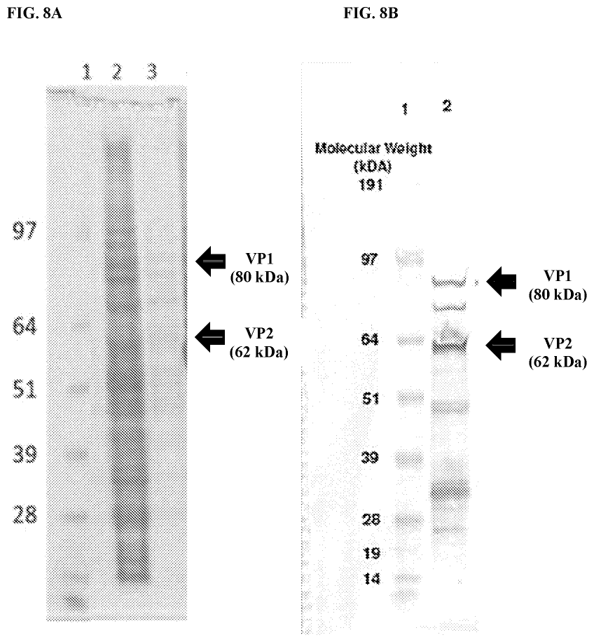

Recombinant CPV capsid proteins were expressed using BEV as illustrated in FIG. 7. The overexpressed CPV capsid proteins (VP1 and VP2) were confirmed by the Coomassie blue-stained SDS-PAGE (FIG. 8A) and Western blot analysis using an anti-CPV VP2 antibody (FIG. 8B). Bands having apparent molecular weights of approximately 80 kDa and 62 kDa were observed, consistent with predicted molecular weights for the capsid proteins, VP1 (80.5 kDa) and VP2 (64.7 kDa). The slight shift in the 64.7 kDa band could be due to charge or hydrophobicity properties of the polypeptide or other factors. In silico design was utilized to identify the VP protein start codon. The expression of the CPV capsid proteins was successfully adapted into the BEV expression system. The expression achieved the desired range of CPV VP1/VP2 ratio where VP2 was in excess of VP1.

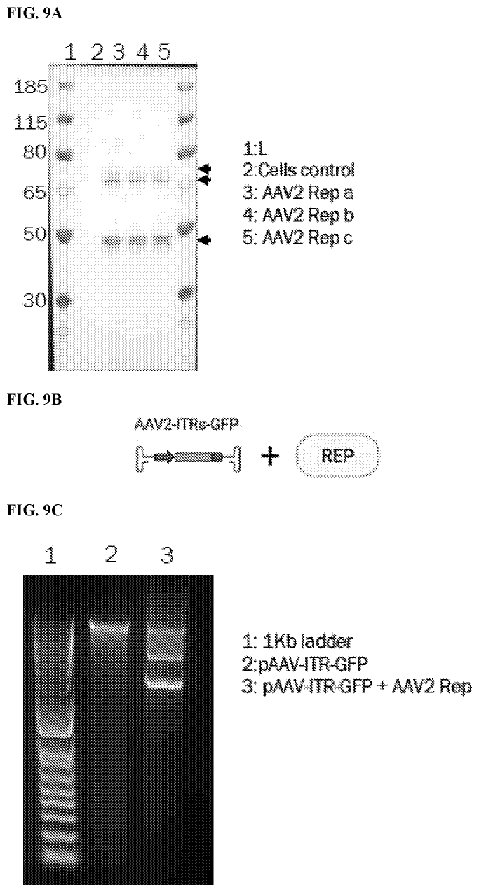

A transgene construct having a GFP reporter gene flanked by AAV2 ITRs was generated (AAV2 ITR-GFP transgene; FIG. 9B). AAV2 Rep proteins were overexpressed using the BEV system and their identity and quantity were confirmed by the Western blot analysis (FIG. 9A). The AAV2 ITR-GFP transgene was successfully amplified using the purified AAV2 Rep proteins, demonstrating the functionality of the AAV2 ITR and Rep system components. FIG. 9C shows successful amplification of AAV2 ITR-GFP transgene and demonstrates the functionality of AAV2 ITR and Rep system components.

### Example 3: Capsid Modification to Decrease the Transcytosis of a Recombinant Virion Containing the Canine Parvovirus Capsid Proteins

Receptor-mediated transcytosis is a type of transcellular transport that mediates the transit of cargo macromolecules across the interior of the cell. This cellular mechanism has been extensively studied to mediate the transport of biomolecules to tissues that are otherwise inaccessible, such as the brain parenchyma. One of the known molecules that mediate cellular transcytosis is the transferrin receptor (TfR). TfR is highly expressed in the brain blood barrier (BBB) and M cells of the intestine. Canine parvovirus (CPV) uses the canid transferrin receptor (TfR) to internalize and infect the target cell (PMCID: PMC2863798). It was also reported that CPV can recognize the murine and human TfR counterparts and internalize in host cells expressing human TfR (PMCID: PMC114880).

The ability of cargo proteins or viral particles to transcytose or remain in the first internalized cell relies on the affinity of the protein cargo/viral particle-TfR interaction (PMCID:PMC6175443, PMCID:PMC3920563). A high affinity of the CPV capsid for TfR leads the viral particle from the early endocytic vesicles to late endosome with increased acidification, promoting endosomal escape and subsequent retrograde transport via the Golgi apparatus to the nucleus where the viral DNA genome is delivered, preferentially avoiding a transcellular transport. On the contrary, a weak CPV particle-TfR interaction promotes transcytosis to secrete the cargo on the opposite side. Discrete amino acids changes within the CPV and FPV VP2 proteins modulate capsid transcytosis, thus providing a novel gene therapy tool with defined tissue tropism. The underlined amino acid residues in SEQ ID NO: 4 or SEQ ID NO: 27 define the receptor binding domains of VP2 that influence the internalization and transcytosis. Mutations of these residues alter the receptor interaction and modulate transcytosis. For example, the amino acid 93 and the adjacent residues of SEQ ID NO: 4 are involved in TfR interaction and host range. The amino acid 300 and the adjacent residues of the CPV VP2 (SEQ ID NO: 4) influence host range (interaction with TfR). In addition, the amino acid 323 and the adjacent residues of SEQ ID NO: 4 are involved in TfR interaction and host range.

### Example 4: Capsid Modification to Increase Viral Vector Affinity and Specificity for the Putative Cellular Receptors

Discrete amino acids changes within the CPV and FPV VP2 proteins modulate capsid affinity for the cellular receptor, TfR, thereby providing a novel gene therapy tool with engineered tissue tropism. The underlined amino acid residues in SEQ ID NO: 4 or SEQ ID NO: 27 define the receptor binding domains that influence the internalization and transcytosis. Mutations of these residues alter the receptor interaction and modulate transcytosis. For example, the amino acid 93 and the adjacent residues of SEQ ID NO: 4 are involved in TfR interaction and host range. The amino acid 300 and the adjacent residues of the CPV VP2 (SEQ ID NO: 4) influence host range (interaction with TfR). In addition, the amino acid 323 and the adjacent residues of SEQ ID NO: 4 are involved in TfR interaction and host range.

### Example 5: Canine Parvovirus VP2 Exemplary Mutations

An exemplary recombinant canine parvovirus VP2 is constructed, which has the amino acid sequence given in SEQ ID NO: 4. The underlined amino acid residues in SEQ ID NO: 4 or SEQ ID NO: 27 define the receptor binding domains that influence the internalization and transcytosis. A plurality of mutations in the underlined residues is made to alter the receptor interaction and modulate transcytosis (see SEQ ID NO: 27 below, and also SEQ ID NO: 4 in Table 4). For example, the amino acid 93 and the adjacent residues (amino acid residues 91-95) of SEQ ID NO: 27 are involved in TfR interaction and host range. The amino acid 300 and the adjacent residues (amino acid residues 297-301) of the CPV VP2 (SEQ ID NO: 27) influence host range (interaction with TfR). In addition, the amino acid 323 and the adjacent residues (amino acid residues 320-324) of SEQ ID NO: 4 are involved in TfR interaction and host range.

```
Canine Parvovirus Strain N VP2
SEQ ID NO: 27
    1 MSDGAVQPDG GQPAVRNERA TGSGNGSGGG GGGGSGGVGI STGTFNNQTE FKFLENGWVE
  61 ITANSSRLVH LNMPESENYR RVVVNNMDKT AVNGNMALDD IHAQIVTPWS LVDANAWGVW
121 FNPGDWQLIV NTMSELHLVS FEQEIFNVVL KTVSESATQP PTKVYNNDLT ASLMVALDSN
181 NTMPFTPAAM RSETLGFYPW KPTIPTPWRY YFQWDRTLIP SHTGTSGTPT NIYHGTDPDD
241 VQFYTIENSV PVHLLRTGDE FATGTFFFDC KPCRLTHTWQ TNRALGLPPF LNSLPQSEGA
301 TNFGDIGVQQ DKRRGVTQMG NTNYITEATI MRPAEVGYSA PYYSFEASTQ GPFKTPIAAG
361 RGGAQTYENQ AADGDPRYAF GRQHGQKTTT TGETPERFTY IAHQDTGRYP EGDWIQNINF
421 NLPVTNDNVL LPTDPIGGKT GINYTNIFNT YGPLTALNNV PPVYPNGQIW DKEFDTDLKP
481 RLHVNAPFVC QNNCPGQLFV KVAPNLTNEY DPDASANMSR IVTYSDFWWK GKLVFKAKLR
541 ASHTWNPIQQ MSINVDNQFN YVPSNIGGMK IVYEKSQLAP RKLY
```

### Example 6: Producing the Canine Parvovirus or Human Parvovirus 4 Recombinant Virions Using Insect Cells

Sf9 cells are grown in serum-free insect cell culture medium (HyClone SFX-Insect Cell Culture Medium) and transferred from an erlenmyer shake flask (Corning) to a Wave single-use bioreactor (GE Healthcare). Cells density density and viability are determined daily using a Cellometer Autor 2000 (Nexelcom). Volume is adjusted to maintain a cell density of 2 to 5 million cells per ml. At the final volume (10L) and density of 2.5 million cells per ml, the baculovirus infected insect cells (BIICs) are added (cryopreserved, 100× concentrated cell “plugs”) 1:10,000 (v:v). The highly diluted BIICs release Rep-VP-Bac, NS-Bac, and vg-Bac that are at very low multiplicity of infection (MOI) and virtually no cells are co-infected during the primary infection. However, subsequent infection cycles release large numbers of each of the requisite baculovirus achieving a very high MOI ensuring that each cell is infected with numerous virus particles. The cells are maintained in culture for four days or until viability drops to ≤30%.

### Example 7: Purification of the Canine Parvovirus or Human Parvovirus 4 Recombinant Virions

The recombinant canine parvovirus or human parvovirus 4 particles are partitioned in both the cellular and extracellular fractions. To recover the maximum number of particles, the entire biomass including cell culture medium is processed. To release the intracellular canine parvovirus or human parvovirus 4 particles, Triton-X 100 is added to the bioreactor with continued agitation for 1 hr. The temperature is increased from 27° C. to 37° C. then Benzonase (EMD Merck) or Turbonuclease (Accelagen, Inc.) is added (2u per ml) to the bioreactor with continued agitation. The biomass is clarified using a staged depth filter, then filter sterilized (0.2 μm) and collected in a sterile bioprocessing bag. The recombinant canine parvovirus or human parvovirus 4 particles are recovered using sequential column chromatography using immune-affinity chromatography medium and Q-Sepharose anion exchange. Chromatograms displaying and recording UV absorption, pH, and conductivity are used to determine completion of the washing and elution steps. Relative efficiency of each step is determined by western blot analysis and quantitatively by ddPCR or qPCR analysis aliquots of the input material (“Load”), the flow-through, the wash, and the elution.

Immune-affinity chromatography uses a “nanobody,” the VhH region of a single-domain immunoglobulin produced in llamas and other camelid species. To produce the nanobody, an antibody provider immunizes llamas with canine parvovirus or human parvovirus 4 virus-like particles, i.e., assembled capsids with no virion genome. The canine parvovirus or human parvovirus 4 VLPs are prepared in Sf9 cells infected with the VP-Bac and purified using using cesium chloride isopycnic gradients, followed by size exclusion chromatography (Superdex 200). Following a prime (1×)/boost (2×) immunization protocol the antibody service provider bleeds the llama and isolates peripheral blood mononuclear cells or mRNA extracted from nucleated blood cells. Reverse transcription using primers specific for the conserved VhH CDR flanking regions (FR1 and FR 4) produces cDNA that is cloned into plasmids used to generate the T7Select 10-3b phage display library (EMD-Millipore). Following several rounds of panning to enrich for phage that interact with canine parvovirus or human parvovirus 4 capsids, phage clones are isolated from plaques. E. coli infected with the recombinant phage are mixed into agarose and applied as an overlay onto LB-agar plates. The E. coli grow to confluency establishing a “lawn” where lysed bacteria and appear as plaques on the plate. To identify phage that bind to canine parvovirus or human parvovirus 4, nitrocellulose filters placed on surface of the agar plates to transfer proteins from the plaques to the filter. The filters are incubated with canine parvovirus or human parvovirus 4 capsids modified with a covalently linked horseradish peroxidase (HRP) (EZLink Plus Activated Peroxidase Kit, ThermoFisher) and washed with phosphate buffered saline. HRP activity can be detected with either a chromogenic (Novex HRP Chromogenic Substrate, ThermoFisher) or chemiluminescent substrate (Pierce ECL Western Blotting Substrate, ThermoFisher). The sequences of the cDNA in the phage are determined and ligated into a bacterial expression plasmid and expressed with a 6×His tag for purification. The chelating column—purified nanobody is covalently linked to chromatography medium, NHS-activated Sepharose 4 Fast Flow (GE Healthcare).

Canine parvovirus or human parvovirus 4 particles are recovered from the clarified Sf9 cell lysate by binding, washing, and eluting from the nanobody-Sepharose column. The efficiency of binding is determined by western blotting the column load and flow through. The wash step is considered complete when the UV280 nm absorbance returns to baseline (i.e., pre-load) values. An acidic pH shift releases the viral particles that are eluted from the nanobody—Sepharose medium. The eluate is collected in 50 nM Tris-Cl, pH 7.2 to neutralize the elution medium.

The concentration of the canine parvovirus or human parvovirus 4 vector particles is determined using canine parvovirus or human parvovirus 4 specific ELISA and qPCR which can be used to estimate the percentage of filled particles, i.e., vector genome-containing.

### Example 8: Purification of CD34+ Cells

CD34+ cells for use in the disclosed methods can be purified according to suitable methods, such as those described in the following articles: Hayakama et al., Busulfan produces efficient human cell engraftment in NOD/LtSz-scid IL2Rγ null mice, Stem Cells 27(1): 175-182 (2009); Ochi et al., Multicolor Staining of Globin Subtypes Reveals Impaired Globin Switching During Erythropoiesis in Human Pluripotent Stem Cells, Stem Cells Translational Medicine 3:792-800 (2014); and McIntosh et al., Nonirradiated NOD,B6.SCID Il2rγ^{-/-} Kit^{W4l/W4l}(NBSGW) Mice Support Multilineage Engraftment of Human Hematopoietic Cells, Stem Cell Reports 4: 171-180 (2015).

### Example 9: In Vitro or Ex Vivo Transduction of Erythroid Progenitor Cells Using the Canine Parvovirus Recombinant Virions

The capacity of the Canine Parvovirus (CPV) is approximately 110% of the wild-type virion 5.3 kb genome, which is about 5,855 nt in length, of which, approximately 300 nt required for the ITRs, leaving 5,555 nt for “cargo.” This represents 1 kb greater capacity than conventional adeno-associated virus vectors.

The recombinant CPV (rCPV) is used to transduce erythroid progenitor cells. The affinity of CPV for CD71 (TfR), provides an improved method to deliver therapeutic transgenes to erythroid progenitor cells and that gene replacement may be accomplished by genomic editing. Transgene expression in genotypically corrected cells facilitates rescue of the phenotype of the differentiated cells and lead to clinical improvement.

Hemaglobinopathies caused by gain of function mutations are inherited as autosomal recessive traits. Heterozygous individuals tend to be either asymptomatic or mildly affected, whereas individuals with mutations in both alleles are severely affected. Thus, correcting or replacing a single allele is clinically beneficial.

Since both beta-thalassemia and sickle cell disease (SCD) are caused by different mutations in the genes that express hemoglobin beta (HbB), a gene replacement strategy benefits patients with either disease. There are clinical studies for SCD using lentivirus vector (LV) that deliver the HbB expression cassette. The b-globin open reading frame (ORF) is regulated by the globin allele locus control region (LCR) and b-globin promoter. In order to fit into the LV, the minimal LCR has been mapped to three DNAse hypersensitive sites (HS) that inhibit DNA methylation and the formation of heterochromatin. Randomly integrating LV may integrate into heterochromatin resulting in shut-off of b-globin expression in the erythrocyte progenitor cells (e.g., erythroblasts), and thus, no phenotypic correction.

The LCR elements, HS, maintain the open, euchromatin structure of LV DNA regardless of integration site. However, the minimized LCR, compared to the b-globin ORF (441 bp and 147 codons) is relatively large limiting the virus vector delivery options.

Inserting the HbB cassette into a genomic safe harbor (GSH) locus. In contrast to transposable elements which constitute approximately 45% of the mammalian genome, heritable integrated parvovirus genomes (or endogenous virus elements, EVEs) occur in very few loci across hundreds of species. The EVEs are genomic markers of sites that tolerate insertion of foreign DNA without affecting embryogenesis, development, maturation, etc. on the short time-line and evolution/speciation on a geologic time-line. Presumably due to the disruptive effects of foreign DNA insertion, there are very few EVE loci that have accumulated in many diverse species over 100 million years. Despite the many species among the highly diverse phylogenetic taxa that harbor EVEs, there appear to be a limited number of genomic loci affected facilitating an empirical analysis of EVEs as GSHs in model systems, e.g., mouse. The conservation of the EVE loci among mammalian species allows us to determine the homologous sites in the human and mouse genomes. However, it is likely that not all GSHs will support long-term, stable expression all tissue types. Using in silico analysis, including RNAseq and ATACseq databases, GSH loci can be mapped to subgenomic regions that are actively expressed in the target tissue. Thus, for beta-globinopathies, erythroblasts are particularly interesting.

Utilizing GSH loci that are actively chromatin regions actively expressed chromatin in erythroblasts, circumvents the necessity of using the LCR elements to ensure euchromatinization where the LV integrated.

The process of homology directed repair (HDR) with a targeting nuclease improves the efficiency and specificity of recombination. “Homology arms” flanking the therapeutic gene, directs the vector DNA to the targeted locus. Recombination either by cellular DNA repair pathway enzymes, or an articificial process, e.g., CRISPR/Cas9 nuclease, integrates the transgene into the GSH.

In addition to b-globin promoter, other promoters have been used for long-term, high-level expression in numerous cell types and also in transgenic mouse strains.

For example, hemoglobin is a heterotetramer composed of 2×HbA and 2×HbB chains. In the absence of HbB, the HbA chain self-associates and form cytotoxic aggregates. The alpha-hemoglobin stabilizing protein (AHSP) is co-expressed in pro-erythrocytes to prevent aggregation of a-globin subunits. The AHSP promoter is highly active in erythrocyte precursors and is well characterized.

As another example, the CAG promoter enhancer is a synthetic promoter engineered from the cytomegalovirus enhancer fused to the chicken beta-globin promoter and exon 1 and intron 1 and splice acceptor of exon 2.

As another example, the MND promoter is active hematopoietic cells

As another example, the Wiskott-Aldrich promoter is active in hematopoietic cells.

As another example, the PKLR promoter is active in hematopoietic cells

Peripheral blood stem cells (PBSCs) are isolated by leukophresis.

Cryopreserved peripheral blood cells in Hemofreeze bags are recovered by rapid thawing in a 37° C. water bath. These thawed cells are suspended in 4% HSA at 4° C. and washed twice by centrifugation at 450 g for 5 min at 4° C. The platelets are removed twice by overlaying on 10% HSA and centrifugation at 450 g for 15 min at 4° C. The erythrocytes are removed by overlaying on Ficoll-Hypaque (FH; 1.077 g/cm3; Pharmacia Fine Chemicals, Piscataway, NJ, USA) and centrifugation at 400 g for 25 min at 4° C. The interface mononuclear cells (Pl−, FH cells) are collected, washed twice in washing solution and resuspended in 4% HSA at 4° C. (MN cells). A nylon-fiber syringe (NF-S) is used to remove adherent cells. Five grams of NF is packed into a 50 ml disposable syringe. The mono nuclear cells were transferred to an additional 50 ml syringe and gently infused into the NF-S, then were incubated at 4° C. for 5 min. The MN cells are then collected into a 50 ml syringe through a plunger of the NF-S, and the cells are pooled in 50 ml of a conical tube. These pooled cells are centrifuged at 400 g for 5 min at 4° C., and resuspended in 4% HSA at 4° C. (NF cells). The cell suspension is then immediately processed for CD34+ selection on the Isolex Magnetic Cell Separation System (Isolex 50; Baxter Healthcare, Immunotherapy Division, Newbury, UK) following the manufacturer's instructions. Briefly, cells are incubated with 9C5 murine immunoglobulin G1 (IgG1) anti-human CD34 antibody (10 m g/1×108 NF cells) for 15 min at 4° C. with slow endover-end rotation. After sensitization, the cells are washed with 4% HSA at 4° C. to remove any excess/unbound antibody. The Dynabeads (Oslo, Norway) are then added to the washed, sensitized cells at a final bead/cell ratio of 1:10. After mixing at 4° C. for 30 min, the cell-bound microspheres and free microspheres become attached to the wall via the magnet (Dynal MPC-1, Dynal, Fort Lee, NJ, USA) and any free cells that do not bind to the microspheres are removed. This washing procedure is repeated twice with 4% HSA at 4° C. The linkage between Dynabeads and CD34+ cells is cleaved by a PR34+ Stem Cell Releasing Agent for 30 min at 4° C. The free Dynabeads are removed from the CD34+ cells via the magnet. D-PBS containing 1% ACD-A and 1% HSA at 25° C. is used for collection of cells. The resulted cell product is controlled by Flow cytometry.

### Example 10: Pulsatile Gene Expression Using the Canine Parvovirus Recombinant Virions or Human Parvovirus 4 Recombinant Virions

Canine parvovirus recombinant virion or human parvovirus 4 comprising a nucleic acid encoding Factor VIII (FVIII), F8 or a fragment encoding a B-domain deleted polypeptide, is used to transduce hepatocytes as a therapy for hemophilia A. FVIII is an essential blood-clotting protein, also known as anti-hemophilic factor (AHF). In humans, factor VIII is encoded by the F8 gene. Defects in this gene result in hemophilia A, a recessive X-linked coagulation disorder. Factor VIII is produced in liver sinusoidal cells and endothelial cells outside the liver throughout the body.

Attempts have been made previously to increase the expression of F8 gene to treat hemophilia A. For example, Valoctocogene Roxaparvovec (also known as BMN270 or), an adenovirus-associated virus (AAV5) vector-mediated gene transfer of human Factor VIII was tested in patients with severe haemophilia A (ClinicalTrials.gov Identifiers: NCT02576795; NCT03370913; NCT03392974; NCT03520712). However, FDA rejected its approval in 2020, requesting long-term safety and efficacy data. The long-term data may be needed to ease the concerns over the increased dosage that may subsequently result in gradual gene expression of the transgene.

FVIII has been a difficult recombinant protein to produce in either microbial or eukaryotic expression systems. The development of the “B-domain” deleted improved expression levels and reduced the size of the open-reading frame, however, FVIII expression levels were substantially lower than other proteins. To overcome these low levels, the clinical dose of Valoctocogene Roxaparvovec viral vector was increased. Patients were treated with 6E+13 vector particles (referred to as vector genomes, or vg) per kg. Based on large animal models, a small minority of hepatocytes were transduced with rAAV5-FVIII. As a result of the large number of vg per cell, the transduced cell expresses relatively large quantities of FVIII. The metabolic demand for FVIII expression likely disrupts the normal requirements for hepatocyte protein expression. The hepatocyte cellular compartments normally involved in protein folding and secretion may become congested with the FVIII. Endothelial cells that produce FVIII production are likely specialized for this activity and produce FVIII from the allele on the single X chromosome under the transcriptional control of the highly regulated native FVIII promoter.

Accordingly, it is hypothesized herein that the perturbations of the hepatocyte homeostasis create cellular stress that induces an inflammatory state. The metabolic and protein folding/export burdens are exacerbated by the use of constitutive, highly active promoters used in the rAAV-FVIII vectors. The inflammation and cytokine production may lead to cell turnover or cell death.

To circumvent this problem and to address the long-felt need for a therapy for hemophilia A, a Canine parvovirus recombinant virion or human parvovirus 4 vector is engineered to comprise (a) the gene F8, or (b) the gene F8 with B-domain deletion. The ability of the Canine parvovirus recombinant virion or human parvovirus 4 viral vector to package a bigger genome size provides an advantage over the rAAV5 currently utilized in the clinical trial, and enables packaging of the full length F8 gene that is not possible with AAV vectors. In addition, in contrast to the constitutive and highly active promoter used in the clinical trial for Valoctocogene Roxaparvovec, the Canine parvovirus recombinant virion or human parvovirus 4 recombinant vector is prepared with an inducible expression system.

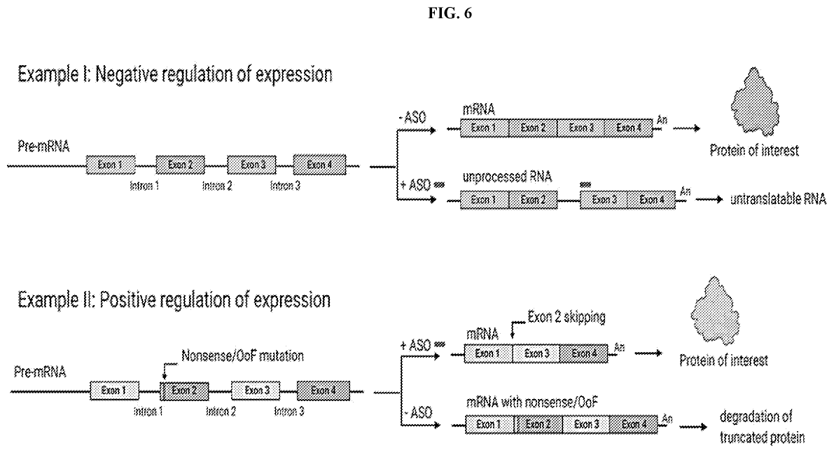

An inducible expression system keeps the F8 gene at the default transcriptionally off state until a reagent turns-on or disinhibits expression (see e.g., FIG. 6). Pulsatile expression spares the hepatocytes from over-expression stress. The timing of the pulses (i.e., the timing of turning on the gene expression) can be determined from the initial serum levels (t0) and the half-life (t½) of FVIII. The t½ is estimated to be 9 to 14 days, thus a 14-day (2 wks) t½ is used, and mild hemophilia is defined as FVIII levels ≥5% normal.

Transgene expression=150%
68 days to decline to 5%
Here, the expression is induced monthly that results in therapeutic levels of FVIII.

A wide range of ASO chemistries (antisense oligo nucleotides ASO or AON) have been developed that increase the t½ in the cell. Here, an ASO chemistry with relatively short t½ is used to achieve a pulse of FVIII expression which diminishes as the ASO is cleared from the cell. The optimal t½ is determined empirically based on among others, the transduced cell number, promoter activity, and kinetics of transcript maturation.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the present invention described herein. Such equivalents are intended to be encompassed by the following claims.

### REFERENCES

Allison, A. B., L. J. Organtini, S. Zhang, S. L. Hafenstein, E. C. Holmes and C. R. Parrish (2016). “Single Mutations in the VP2 300 Loop Region of the Three-Fold Spike of the Carnivore Parvovirus Capsid Can Determine Host Range.” J Virol 90(2): 753-767.
Bacon, B. R., L. W. Powell, P. C. Adams, T. F. Kresina and J. H. Hoofnagle (1999). “Molecular medicine and hemochromatosis: at the crossroads.” Gastroenterology 116(1): 193-207.
Clevers, H. (2013). “The intestinal crypt, a prototype stem cell compartment.” Cell 154(2): 274-284.
de Almeida, S. F. and M. de Sousa (2008). “The unfolded protein response in hereditary haemochromatosis.” J Cell Mol Med 12(2): 421-434.
Ezquer, F., M. T. Nunez, A. Rojas, J. Asenjo and Y. Israel (2006). “Hereditary hemochromatosis: an opportunity for gene therapy.” Biol Res 39(1): 113-124.
Feder, J. N., A. Gnirke, W. Thomas, Z. Tsuchihashi, D. A. Ruddy, A. Basava, F. Dormishian, R. Domingo, Jr., M. C. Ellis, A. Fullan, L. M. Hinton, N. L. Jones, B. E. Kimmel, G. S. Kronmal, P. Lauer, V. K. Lee, D. B. Loeb, F. A. Mapa, E. McClelland, N. C. Meyer, G. A. Mintier, N. Moeller, T. Moore, E. Morikang, C. E. Prass, L. Quintana, S. M. Starnes, R. C. Schatzman, K. J. Brunke, D. T. Drayna, N. J. Risch, B. R. Bacon and R. K. Wolff (1996). “A novel MHC class I-like gene is mutated in patients with hereditary haemochromatosis.” Nat Genet 13(4): 399-408.
Feder, J. N., Z. Tsuchihashi, A. Irrinki, V. K. Lee, F. A. Mapa, E. Morikang, C. E. Prass, S. M. Starnes, R. K. Wolff, S. Parkkila, W. S. Sly and R. C. Schatzman (1997). “The hemochromatosis founder mutation in HLA-H disrupts beta2-microglobulin interaction and cell surface expression.” J Biol Chem 272(22): 14025-14028.
Gochee, P. A., L. W. Powell, D. J. Cullen, D. Du Sart, E. Rossi and J. K. Olynyk (2002). “A population-based study of the biochemical and clinical expression of the H63D hemochromatosis mutation.” Gastroenterology 122(3): 646-651.
Goodman, L. B., S. M. Lyi, N. C. Johnson, J. O. Cifuente, S. L. Hafenstein and C. R. Parrish (2010). “Binding site on the transferrin receptor for the parvovirus capsid and effects of altered affinity on cell uptake and infection.” J Virol 84(10): 4969-4978.
Hendrickson, B. A., R. Gokhale and J. H. Cho (2002). “Clinical aspects and pathophysiology of inflammatory bowel disease.” Clin Microbiol Rev 15(1): 79-94.
Hueffer, K., L. Govindasamy, M. Agbandje-McKenna and C. R. Parrish (2003). “Combinations of two capsid regions controlling canine host range determine canine transferrin receptor binding by canine and feline parvoviruses.” J Virol 77(18): 10099-10105.
Hueffer, K., J. S. Parker, W. S. Weichert, R. E. Geisel, J. Y. Sgro and C. R. Parrish (2003). “The natural host range shift and subsequent evolution of canine parvovirus resulted from virus-specific binding to the canine transferrin receptor.” J Virol 77(3): 1718-1726.
Jones, M. S., A. Kapoor, V. V. Lukashov, P. Simmonds, F. Hecht and E. Delwart (2005). “New DNA viruses identified in patients with acute viral infection syndrome.” J Virol 79(13):8230-8236.
Levine, B., M. Packer and P. Codogno (2015). “Development of autophagy inducers in clinical medicine.” J Clin Invest 125(1): 14-24.
Limanskiy, V., A. Vyas, L. S. Chaturvedi and D. Vyas (2019). “Harnessing the potential of gene editing technology using CRISPR in inflammatory bowel disease.” World J Gastroenterol 25(18): 2177-2187.
Liu, Y., S. Y. Lee, E. Neely, W. Nandar, M. Moyo, Z. Simmons and J. R. Connor (2011). “Mutant HFE H63D protein is associated with prolonged endoplasmic reticulum stress and increased neuronal vulnerability.” J Biol Chem 286(15): 13161-13170.
Matthews, P. C., C. Sharp, P. Simmonds and P. Klenerman (2017). “Human parvovirus 4 ‘PARV4’ remains elusive despite a decade of study.” F1000Res 6: 82.
Navone, S. E., G. Marfia, S. Nava, G. Invernici, S. Cristini, S. Balbi, S. Sangiorgi, E. Ciusani, A. Bosutti, G. Alessandri, M. Slevin and E. A. Parati (2013). “Human and mouse brain-derived endothelial cells require high levels of growth factors medium for their isolation, in vitro maintenance and survival.” Vase Cell 5(1): 10.
Nemeth, E., A. Roetto, G. Garozzo, T. Ganz and C. Camaschella (2005). “Hepcidin is decreased in TFR2 hemochromatosis.” Blood 105(4): 1803-1806.
Nemeth, E., M. S. Tuttle, J. Powelson, M. B. Vaughn, A. Donovan, D. M. Ward, T. Ganz and J. Kaplan (2004). “Hepcidin regulates cellular iron efflux by binding to ferroportin and inducing its internalization.” Science 306(5704): 2090-2093.
Rivera, S., L. Liu, E. Nemeth, V. Gabayan, O. E. Sorensen and T. Ganz (2005). “Hepcidin excess induces the sequestration of iron and exacerbates tumor-associated anemia.” Blood 105(4): 1797-1802.
Roetto, A., G. Papanikolaou, M. Politou, F. Alberti, D. Girelli, J. Christakis, D. Loukopoulos and C. Camaschella (2003). “Mutant antimicrobial peptide hepcidin is associated with severe juvenile hemochromatosis.” Nat Genet 33(1): 21-22.
Sharp, C. P., A. Lail, S. Donfield, R. Simmons, C. Leen, P. Klenerman, E. Delwart, E. D. Gomperts and P. Simmonds (2009). “High frequencies of exposure to the novel human parvovirus PARV4 in hemophiliacs and injection drug users, as detected by a serological assay for PARV4 antibodies.” J Infect Dis 200(7): 1119-1125.
Vaisanen, E., U. Mohanraj, P. M. Kinnunen, P. Jokelainen, H. Al-Hello, A. M. Barakat, M. Sadeghi, F. A. Jalilian, A. Majlesi, M. Masika, D. Mwaengo, O. Anzala, E. Delwart, O. Vapalahti, K. Hedman and M. Soderlund-Venermo (2018). “Global Distribution of Human Protoparvoviruses.” Emerg Infect Dis 24(7): 1292-1299.
Waheed, A., S. Parkkila, X. Y. Zhou, S. Tomatsu, Z. Tsuchihashi, J. N. Feder, R. C. Schatzman, R. S. Britton, B. R. Bacon and W. S. Sly (1997). “Hereditary hemochromatosis: effects of C282Y and H63D mutations on association with beta2-microglobulin, intracellular processing, and cell surface expression of the HFE protein in COS-7 cells.” Proc Natl Acad Sci USA 94(23): 12384-12389.
Wojcik, J. P., M. R. Speechley, A. E. Kertesz, S. Chakrabarti and P. C. Adams (2002). “Natural history of C282Y homozygotes for hemochromatosis.” Can J Gastroenterol 16(5): 297-302.

What is claimed is:

1. A recombinant virion comprising (1) at least one capsid protein or a variant thereof, of a protoparvovirus or a genotypic variant thereof, and (2) a nucleic acid, wherein the nucleic acid comprises a heterologous nucleic acid.

2. A recombinant virion comprising (1) at least one capsid protein or a variant thereof, of a tetraparvovirus or a genotypic variant thereof, and (2) a nucleic acid, wherein the nucleic acid comprises a heterologous nucleic acid.

3. The recombinant virion of claim 1, wherein the protoparvovirus or a genotypic variant thereof is of a species selected from Carnivore protoparvovirus, Carnivore protoparvovirus 1, Chiropteran protoparvovirus 1, Eulipotyphla protoparvovirus 1, Primate protoparvovirus 1, Primate protoparvovirus 2, Primate protoparvovirus 3, Primate protoparvovirus 4, Rodent protoparvovirus 1, Rodent protoparvovirus 2, Rodent protoparvovirus 3, Ungulate protoparvovirus 1, and Ungulate protoparvovirus 2.

4. The recombinant virion of claim 1 or 3, wherein the protoparvovirus or a genotypic variant thereof is selected from canine parvovirus, feline panelukepenia virus, human bufavirus 1, human bufavirus 2, human bufavirus 3, human tusavirus, human cutavirus, Wuharv parvovirus, porcine parvovirus, minute virus of mice, megabat bufavirus, and a genotypic variant thereof.

5. The recombinant virion of claim 2, wherein the tetraparvovirus or a genotypic variant thereof is of a species selected from Chiropteran tetraparvovirus 1, Primate tetraparvovirus 1, Ungulate tetraparvovirus 1, Ungulate tetraparvovirus 2, Ungulate tetraparvovirus 3, and Ungulate tetraparvovirus 4.

6. The recombinant virion of claim 2 or 5, wherein the tetraparvovirus or a genotypic variant thereof is selected from human parvovirus 4, human parvovirus 4 genotype 1, human parvovirus 4 genotype 2, human parvovirus 4 genotype 3, chimpanzee parvovirus 4, eidolon helvum parvovirus, bovine hokovirus 1, bovine hokovirus 2, porcine hokovirus, porcine cnvirus, yak parvovirus, ovine hokovirus 1, opossum tetraparvovirus, rodent tetraparvovirus, tetraparvovirus sp., and a genotypic variant thereof.

7. The recombinant virion of claim 6, wherein the tetraparvovirus is human parvovirus 4, human parvovirus 4 genotype 1, human parvovirus 4 genotype 2, human parvovirus 4 genotype 3, or a genotypic variant thereof.

8. The recombinant virion of any one of the preceding claims, wherein the virion is icosahedral.

9. The recombinant virion of any one of the preceding claims, wherein the capsid protein or a variant thereof comprises structural proteins VP1 and/or VP2.

10. The recombinant virion of claim 9, wherein VP2 is present in excess of VP1.

11. The recombinant virion of claim 9 or 10, wherein VP1 comprises an amino acid sequence that is at least about 60% identical, about 70% identical, about 80% identical, about 90% identical, about 95% identical, about 96% identical, about 97% identical, about 98% identical, about 99% identical, or 100% identical to a sequence selected from SEQ ID NOs: 4, 5, 7, 8, 10, 11, 13, 14, 16, 17, 19, 20, and 22.

12. The recombinant virion of any one of claims 9-11, wherein VP2 comprises an amino acid sequence that is at least about 60% identical, about 70% identical, about 80% identical, about 90% identical, about 95% identical, about 96% identical, about 97% identical, about 98% identical, about 99% identical, or 100% identical to a sequence selected from SEQ ID NOs: 4, 6, 7, 9, 10, 12, 13, 15, 16, 18, 19, 21, and 23.

13. The recombinant virion of any one of preceding claims, wherein the heterologous nucleic acid comprises a nucleic acid sequence that is at least about 60% identical to a nucleic acid sequence of a target cell.

14. The recombinant virion of any one of preceding claims, wherein the heterologous nucleic acid is at least about 60% identical to the nucleic acid of a mammal, preferably wherein the mammal is a human.

15. The recombinant virion of any one of preceding claims, wherein the heterologous nucleic acid is not operably linked to a protoparvovirus or tetraparvovirus promoter.

16. The recombinant virion of any one of preceding claims, wherein the nucleic acid comprises at least one inverted terminal repeat (ITR).

17. The recombinant virion of claim 16, wherein the at least one ITR comprises:
(a) a dependoparvovirus ITR,
(b) an AAV ITR, optionally an AAV2 ITR,
(c) a protoparvovirus ITR, or
(d) a tetraparvovirus ITR.

18. The recombinant virion of claim 17, wherein the protoparvovirus ITR is selected from the ITRs of canine parvovirus, feline panelukepenia virus, human bufavirus 1, human bufavirus 2, human bufavirus 3, human tusavirus, human cutavirus, Wuharv parvovirus, porcine parvovirus, minute virus of mice, megabat bufavirus, and a genotypic variant thereof.

19. The recombinant virion of claim 17, wherein the tetraparvovirus ITR is selected from the ITRs of human parvovirus 4, human parvovirus 4 genotype 1, human parvovirus 4 genotype 2, human parvovirus 4 genotype 3, chimpanzee parvovirus 4, eidolon helvum parvovirus, bovine hokovirus 1, bovine hokovirus 2, porcine hokovirus, porcine cnvirus, yak parvovirus, ovine hokovirus 1, opossum tetraparvovirus, rodent tetraparvovirus, tetraparvovirus sp., and a genotypic variant thereof.

20. The recombinant virion of claim 17 or 19, wherein the tetraparvovirus ITR is selected from the ITRs of human parvovirus 4, human parvovirus 4 genotype 1, human parvovirus 4 genotype 2, human parvovirus 4 genotype 3, and a genotypic variant thereof.

21. The recombinant virion of any one of the preceding claims, wherein the nucleic acid is deoxyribonucleic acid (DNA).

22. The recombinant virion of claim 21, wherein the DNA is single-stranded or self-complementary duplex.

23. The recombinant virion of any one of the preceding claims, wherein the nucleic acid comprises a Rep protein-dependent origin of replication (ori).

24. The recombinant virion of any one of the preceding claims, wherein the nucleic acid comprises a nucleic acid operably linked to a promoter, optionally placed between two ITRs.

25. The recombinant virion of claim 24, wherein the promoter is selected from:
(a) a promoter heterologous to the nucleic acid to which it is operably linked;
(b) a promoter that facilitates the tissue-specific expression of the nucleic acid, preferably wherein the promoter facilitates hematopoietic cell-specific expression or erythroid lineage-specific expression;
(c) a promoter that facilitates the constitutive expression of the nucleic acid; and
(d) a promoter that is inducibly expressed, optionally in response to a metabolite or small molecule or chemical entity.

26. The recombinant virion of claim 24 or 25, wherein the promoter is selected from the CMV promoter, β-globin promoter, CAG promoter, AHSP promoter, MND promoter, Wiskott-Aldrich promoter, and PKLR promoter.

27. The recombinant virion of any one of the preceding claims, wherein the heterologous nucleic acid encodes a coding RNA and/or a non-coding RNA.

28. The recombinant virion of claim 25, wherein the heterologous nucleic acid encoding a coding RNA comprises:
(a) a gene encoding a protein or a fragment thereof, preferably a human protein or a fragment thereof;
(b) a nucleic acid encoding a nuclease, optionally a Transcription Activator-Like Effector Nuclease (TALEN), a zinc-finger nuclease (ZFN), a meganuclease, a megaTAL, or a CRISPR endonuclease, (e.g., a Cas9 endonuclease or a variant thereof);
(c) a nucleic acid encoding a reporter, e.g., luciferase or GFP; and/or
(d) a nucleic acid encoding a drug resistance protein, e.g., neomycin resistance.

29. The recombinant virion of claim 27 or 28, wherein the heterologous nucleic acid encoding a coding RNA is codon-optimized for expression in a target cell.

30. The recombinant virion of any one of claims 27-29, wherein the heterologous nucleic acid comprises a gene encoding a polypeptide, or a fragment thereof, selected from (HBA1, HBA2, HBB, HBG1, HBG2, HBD, HBE1, and/or HBZ), alpha-hemoglobin stabilizing protein (AHSP), coagulation factor VIII, coagulation factor IX, von Willebrand factor, dystrophin or truncated dystrophin, micro-dystrophin, utrophin or truncated utrophin, micro-utrophin, usherin (USH2A), CEP290, ATPB1, ATPB11, ABCB4, CPS1, ATP7B, KRT5, KRT14, PLEC1, Col7A1, ITGB4, ITGA6, LAMA3, LAMB3, LAMC2, KIND1, INS, F8 or a fragment thereof (e.g., fragment encoding B-domain deleted polypeptide (e.g., VIII SQ, p-VIII)), and cystic fibrosis transmembrane conductance regulator (CFTR).

31. The recombinant virion of claim 27, wherein the non-coding RNA comprises lncRNA, piRNA, miRNA, shRNA, siRNA, antisense RNA, and/or guide RNA.

32. The recombinant virion of any one of claims 27-31, wherein the coding RNA, the protein, or the non-coding RNA increases or restores the expression of an endogenous gene of a target cell.

33. The recombinant virion of any one of claims 27-31, wherein the coding RNA, the protein, or the non-coding RNA decreases or eliminates the expression of an endogenous gene of a target cell.

34. The recombinant virion of any one of claims 27-33, wherein the recombinant virion comprises a nucleic acid encoding (a) hepcidin or a fragment thereof, and/or homeostatic iron regulator (HFE) or a fragment thereof; (b) at least one non-coding RNA (e.g., piRNA, miRNA, shRNA, siRNA, antisense RNA) that targets DMT-1, ferroportin, and/or an endogenous mutant form of HFE; (c) a CRISPR/Cas system that targets DMT-1, ferroportin, and/or an endogenous mutant form of HFE; and/or (d) any combination of any one of the nucleic acids listed in (a) to (c).

35. The recombinant virion of claim 34, wherein the recombinant virion (a) increases the expression of HFE and/or hepcidin in the transduced cell; and/or (b) decreases the expression of DMT-1, ferroportin, and/or an endogenous mutant form of HFE in the transduced cell.

36. The recombinant virion of claim 34 or 35, wherein the recombinant virion prevents or treats hemochromatosis, hereditary hemochromatosis, juvenile hemochromatosis, and/or Wilson's disease.

37. The recombinant virion of any one of claims 27-33, wherein the recombinant virion comprises a nucleic acid encoding (a) a soluble form of the TNFα receptor, a soluble form of the IL-6 receptor, a soluble form of the IL-12 receptor, and/or a soluble form of the IL-1β receptor; (b) at least one non-coding RNA (e.g., piRNA, miRNA, shRNA, siRNA, antisense RNA) that targets the TNFα receptor, IL-6 receptor, IL-12 receptor, and/or IL-1 receptor; (c) a CRISPR/Cas system that targets the TNFα receptor, IL-6 receptor, IL-12 receptor, and/or IL-1β receptor; and/or (d) any combination of any one of the nucleic acids listed in (a) to (c).

38. The recombinant virion of claim 37, wherein the recombinant virion (a) increases the expression of a soluble form of the TNFα receptor, a soluble form of the IL-6 receptor, a soluble form of the IL-12 receptor, or a soluble form of the IL-1β receptor in the transduced cell; and/or (b) decreases the expression of the TNFα receptor, IL-6 receptor, IL-12 receptor, or IL-1β receptor in the transduced cell.

39. The recombinant virion of claim 37 or 38, wherein the recombinant virion prevents or treats rheumatoid arthritis, inflammatory bowel disease, psoriatic arthritis, juvenile chronic arthritis, psoriasis, and/or ankylosing spondylitis.

40. The recombinant virion of any one of claims 27-33, wherein the recombinant virion comprises a nucleic acid encoding a protein or a fragment thereof selected from IRGM, NOD2, ATG2B, ATG9, ATG5, ATG7, ATG16L1, BECN1, EI24/PIG8, TECPR2, WDR45/WIP14, CHMP2B, CHMP4B, Dynein, EPG5, HspB8, LAMP2, LC3b UVRAG, VCP/p97, ZFYVE26, PARK2/Parkin, PARK6/PINK1, SQSTM1/p62, SMURF, AMPK, and ULK1.

41. The recombinant virion of claim 40, wherein the recombinant virion increases the expression of said protein or a fragment thereof in the transduced cells.

42. The recombinant virion of claim 40 or 41, wherein the recombinant virion modulates autophagy.

43. The recombinant virion of any one of claims 40-42, wherein the recombinant virion prevents or treats an autophagy-related disease.

44. The recombinant virion of claim 43, wherein the autophagy-related disease is selected from cancer, neurodegenerative disease (e.g., Alzheimer's disease, Parkinson's disease, Huntington's disease, ataxias), inflammatory disease, inflammatory bowel disease, Crohn's disease, rheumatoid arthritis, lupus, multiple sclerosis, chronic obstructive pulmony disease/COPD, pulmonary fibrosis, cystic fibrosis, Sjogren's disease, hyperglycemic disorders, type I diabetes, type II diabetes, insulin resistance, hyperinsulinemia, insulin-resistant diabetes (e.g. Mendenhall's Syndrome, Werner Syndrome, leprechaunism, and lipoatrophic diabetes), dyslipidemia, hyperlipidemia, elevated low-density lipoprotein (LDL), depressed highdensity lipoprotein (HDL), elevated triglycerides, metabolic syndrome, liver disease, renal disease, cardiovascular disease, ischemia, stroke, complications during reperfusion, muscle degeneration, atrophy, symptoms of aging (e.g., muscle atrophy, frailty, metabolic disorders, low grade inflammation, atherosclerosis, stroke, age-associated dementia and sporadic form of Alzheimer's disease, pre-cancerous states, and psychiatric conditions including depression), spinal cord injury, arteriosclerosis, infectious diseases (e.g., bacterial, fungal, viral), AIDS, tuberculosis, defects in embryogenesis, infertility, lysosomal storage diseases, activator deficiency/GM2 gangliosidosis, alpha-mannosidosis, aspartylglucoaminuria, cholesteryl ester storage disease, chronic hexosaminidase A deficiency, cystinosis, Danon disease, Fabry disease, Farber disease, fucosidosis, galactosialidosis, Gaucher Disease (Types I, II and III), GM1 Gangliosidosis, (infantile, late infantile/juvenile and adult/chronic), Hunter syndrome (MPS II), I-Cell disease/Mucolipidosis II, Infantile Free Sialic Acid Storage Disease (ISSD), Juvenile Hexosaminidase A Deficiency, Krabbe disease, Lysosomal acid lipase deficiency, Metachromatic Leukodystrophy, Hurler syndrome, Scheie syndrome, Hurler-Scheie syndrome, Sanfilippo syndrome, Morquio Type A and B, Maroteaux-Lamy, Sly syndrome, mucolipidosis, multiple sulfate deficiency, Niemann-Pick disease, Neuronal ceroid lipofuscinoses, CLN6 disease, Jansky-Bielschowsky disease, Pompe disease, pycnodysostosis, Sandhoff disease, Schindler disease, Tay-Sachs, and Wolman disease.

45. The recombinant virion of any one of claims 27-33, wherein the recombinant virion comprises a nucleic acid encoding (a) CFTR or a fragment thereof, (b) at least one non-coding RNA (e.g., piRNA, miRNA, shRNA, siRNA, antisense RNA) that targets an endogenous mutant form of CFTR, (c) a CRISPR/Cas system that targets an endogenous mutant form of CFTR; and/or (d) any combination of any one of the nucleic acids listed in (a) to (c).

46. The recombinant virion of claim 45, wherein the recombinant virion (a) increases the expression of CFTR or fragment thereof, and/or (b) decreases the expression of the endogenous mutant form of CFTR in the transduced cell.

47. The recombinant virion of claim 45 or 46, wherein the recombinant virion prevents or treats cystic fibrosis.

48. The recombinant virion of any one of the preceding claims, wherein the nucleic acid comprises a non-coding DNA.

49. The recombinant virion of claim 48, wherein the non-coding DNA comprises:
(a) a transcription regulatory element (e.g., an enhancer, a transcription termination sequence, an untranslated region (5′ or 3′ UTR), a proximal promoter element, a locus control region, a polyadenylation signal sequence), and/or
(b) a translation regulatory element (e.g., Kozak sequence, woodchuck hepatitis virus post-transcriptional regulatory element).

50. The recombinant virion of claim 49, wherein the transcription regulatory element is a locus control region, optionally a β-globin LCR or a DNase hypersensitive site (HS) of 3-globin LCR.

51. The recombinant virion of any one of preceding claims, wherein the nucleic acid comprises a nucleic acid sequence that is at least about 80% identical to the nucleic acid sequence of a genomic safe harbor (GSH) of the target cell.

52. The recombinant virion of claim 51, wherein the nucleic acid that is at least about 80% identical to a GSH is placed 5′ and 3′ to the nucleic acid to be integrated, thereby allowing integration to a specific locus in the target genome by homologous recombination.

53. The recombinant virion of claim 51 or 52, wherein the GSH is AAVS1, ROSA26, CCR5, Kif6, Pax5, an intergenic region of NUPL2, collagen, HTRP, HI 1 (a thymidine kinase encoding nucleic acid at HI 1 locus), beta-2 microglobulin, GAPDH, TCR, RUNX1, KLHL7, mir684, KCNH2, GPNMB, MIR4540, MIR4475, MIR4476, PRL32P21, LOC105376031, LOC105376032, LOC105376030, MELK, EBLN3P, ZCCHC7, or RNF38.

54. The recombinant virion of claim 53, wherein the GSH is AAVS1, ROSA26, CCR5, Kif6, Pax5, or an intergenic region of NUPL2.

55. The recombinant virion of any one of the preceding claims, wherein the nucleic acid is integrated into the genome of a target cell upon transduction.

56. The recombinant virion of claim 55, wherein the nucleic acid is integrated into a GSH of the genome of a target cell upon transduction.

57. The recombinant virion of any one of the preceding claims, wherein the nucleic acid comprises a nucleic acid sequence encoding at least one replication protein and capsid protein or a variant thereof.

58. The recombinant virion of any one of the preceding claims, wherein the virion is autonomously replicating.

59. The recombinant virion of any one of the preceding claims, wherein the virion binds and/or transduces a cancer cell or non-cancerous cell.

60. The recombinant virion of any one of preceding claims, wherein the virion binds and/or transduces a stem cell (e.g., hematopoietic stem cell, CD34+ stem cell, CD36+ stem cell, mesenchymal stem cell, cancer stem cell).

61. The recombinant virion of any one of claims 1, 3, 4, 8-18, and 21-60, wherein the virion binds and/or transduces a cell expressing the transferrin receptor (CD71).

62. The recombinant virion of any one of the preceding claims, wherein the recombinant virion binds and/or transduces a hematopoietic cell, hematopoietic progenitor cell, hematopoietic stem cell, erythroid lineage cell, megakaryocyte, erythroid progenitor cell (EPC), CD34+ cell, CD36+ cell, mesenchymal stem cell, nerve cell, intestinal cell, intestinal stem cell, gut epithelial cell, endothelial cell, lung cell, enterocyte, liver cell (e.g., hepatocyte, hepatic stellate cells (HSCs), Kupffer cells (KCs), liver sinusoidal endothelial cells (LSECs)), brain microvascular endothelial cell (BMVECs), erythroid progenitor cell, lymphoid progenitor cell, B lymphoblast cell, B cell, T cell, basophilic Endemic Burkitt Lymphoma (EBL), polychromatic erythroblast, epidermal stem cell, P63-positive keratinocyte-derived stem cell, keratinocyte, pancreatic β-cell, K cell, L cell, and/or orthochromatic erythroblast.

63. The recombinant virion of any one of the preceding claims, wherein the at least one capsid protein or a variant thereof comprises a VP2 sequence having one or more mutations with respect to canine parvovirus strain N (UniProtKB—P12930) or the amino acid sequence SEQ ID NO: 27.

64. The recombinant virion of claim 63, wherein said one or more mutations are at a region of VP2 having the amino acid residues (i) 91-95, (ii) 297-301, and/or (iii) 320-324.

65. The recombinant virion of claim 63 or 64, wherein said one or more mutations comprise a substitution, deletion, and/or insertion.

66. The recombinant virion of any one of claims 63-65, wherein said one or more mutations alter the affinity and/or specificity of the recombinant virion to at least one cellular receptor involved in internalization of the recombinant virion, optionally wherein the at least one cellular receptor is the transferrin receptor.

67. The recombinant virion of any one of claims 63-66, wherein said one or more mutations alter:
a) the tropism or affinity of the recombinant virion to a cell;
b) the ability of the recombinant virion to transduce a cell; and/or
c) the ability of the recombinant virion to transcytose across the cell.

68. The recombinant virion of any one of the preceding claims, wherein the at least one capsid protein or a variant thereof comprises a heterologous peptide tag.

69. The recombinant virion of claim 68, wherein said heterologous peptide tag allows affinity purification using an antibody, an antigen-binding fragment of an antibody, or a nanobody.

70. The recombinant virion of claim 68 or 69, wherein said heterologous peptide tag comprises an epitope/tag selected from hemagglutinin, His (e.g., 6×-His), FLAG, E-tag, TK15, Strep-tag II, AU1, AU5, Myc, Glu-Glu, KT3, and IRS.

71. A pharmaceutical composition comprising the recombinant virion of any one of the preceding claims; and a carrier and/or a diluent.

72. A method of preventing or treating a disease, comprising administering to a subject in need thereof an effective amount of the at least one recombinant virion or pharmaceutical composition of any one of claims 1-71.

73. A method of preventing or treating a disease, comprising:
(a) obtaining a plurality of cells;
(b) transducing the cells with the at least one recombinant virion or pharmaceutical composition of any one of claims 1-71, optionally further selecting or screening for the transduced cells; and
(c) administering an effective amount of the transduced cells to a subject in need thereof.

74. The method of claim 73, wherein the cells are autologous or allogeneic to the subject.

75. The method of any one of claims 72-74, wherein
(a) the nucleic acid encodes a protein; or
(b) the nucleic acid decreases or eliminates the expression of an endogenous gene.

76. The method of any one of claims 72-75, wherein the at least one recombinant virion, pharmaceutical composition, or transduced cells are administered via intravascular, intracerebral, parenteral, intraperitoneal, intravenous, epidural, intraspinal, intrasternal, intra-articular, intra-synovial, intrathecal, intra-arterial, intracardiac, intramuscular, intranasal, intrapulmonary, skin graft, or oral administration.

77. The method of any one of claims 72-76, wherein the disease is selected from endothelial dysfunction, cystic fibrosis, cardiovascular disease, renal disease, cancer, hemoglobinopathy, anemia, hemophilia (e.g., hemophilia A), myeloproliferative disorder, coagulopathy, sickle cell disease, alpha-thalassemia, beta-thalassemia, Fanconi anemia, familial intrahepatic cholestasis, epidermolysis bullosa, Fabry, Gaucher, Nieman-Pick A, Nieman-Pick B, GM1 Gangliosidosis, Mucopolysaccharidosis (MPS) I (Hurler, Scheie, Hurler/Scheie), MPS II (Hunter), MPS VI (Maroteaux-Lamy), hematologic cancer, hemochromatosis, hereditary hemochromatosis, juvenile hemochromatosis, cirrhosis, hepatocellular carcinoma, pancreatitis, diabetes mellitus, cardiomyopathy, arthritis, hypogonadism, heart disease, heart attack, hypothyroidism, glucose intolerance, arthropathy, liver fibrosis, Wilson's disease, ulcerative colitis, Crohn's disease, Tay-Sachs disease, neurodegenerative disorder, Spinal muscular atrophy type 1, Huntington's disease, Canavan's disease, rheumatoid arthritis, inflammatory bowel disease, psoriatic arthritis, juvenile chronic arthritis, psoriasis, and ankylosing spondylitis, and autoimmune disease, neurodegenerative disease (e.g., Alzheimer's disease, Parkinson's disease, Huntington's disease, ataxias), inflammatory disease, inflammatory bowel disease, Crohn's disease, rheumatoid arthritis, lupus, multiple sclerosis, chronic obstructive pulmony disease/COPD, pulmonary fibrosis, Sjogren's disease, hyperglycemic disorders, type I diabetes, type II diabetes, insulin resistance, hyperinsulinemia, insulin-resistant diabetes (e.g. Mendenhall's Syndrome, Werner Syndrome, leprechaunism, and lipoatrophic diabetes), dyslipidemia, hyperlipidemia, elevated low-density lipoprotein (LDL), depressed highdensity lipoprotein (HDL), elevated triglycerides, metabolic syndrome, liver disease, renal disease, cardiovascular disease, ischemia, stroke, complications during reperfusion, muscle degeneration, atrophy, symptoms of aging (e.g., muscle atrophy, frailty, metabolic disorders, low grade inflammation, atherosclerosis, stroke, age-associated dementia and sporadic form of Alzheimer's disease, pre-cancerous states, and psychiatric conditions including depression), spinal cord injury, arteriosclerosis, infectious diseases (e.g., bacterial, fungal, viral), AIDS, tuberculosis, defects in embryogenesis, infertility, lysosomal storage diseases, activator deficiency/GM2 gangliosidosis, alpha-mannosidosis, aspartylglucoaminuria, cholesteryl ester storage disease, chronic hexosaminidase A deficiency, cystinosis, Danon disease, Farber disease, fucosidosis, galactosialidosis, Gaucher Disease (Types I, II and III), GM1 Gangliosidosis, (infantile, late infantile/juvenile and adult/chronic), Hunter syndrome (MPS II), I-Cell disease/Mucolipidosis II, Infantile Free Sialic Acid Storage Disease (ISSD), Juvenile Hexosaminidase A Deficiency, Krabbe disease, Lysosomal acid lipase deficiency, Metachromatic Leukodystrophy, Hurler syndrome, Scheie syndrome, Hurler-Scheie syndrome, Sanfilippo syndrome, Morquio Type A and B, Maroteaux-Lamy, Sly syndrome, mucolipidosis, multiple sulfate deficiency, Neuronal ceroid lipofuscinoses, CLN6 disease, Jansky-Bielschowsky disease, Pompe disease, pycnodysostosis, Sandhoff disease, Schindler disease, and Wolman disease.

78. The method of any one of claims 72-77, wherein the at least one recombinant virion or pharmaceutical composition comprises at least one capsid protein or variant thereof of a protoparvovirus or a genotypic variant thereof.

79. The method of claim 78, wherein the at least one recombinant virion, pharmaceutical composition, or transduced cells comprise a nucleic acid encoding (a) hepcidin or a fragment thereof, and/or homeostatic iron regulator (HFE) or a fragment thereof, (b) at least one non-coding RNA (e.g., piRNA, miRNA, shRNA, siRNA, antisense RNA) that targets DMT-1, ferroportin, and/or an endogenous mutant form of HFE; (c) a CRISPR/Cas system that targets DMT-1, ferroportin, and/or an endogenous mutant form of HFE; and/or (d) any combination of any one of the nucleic acids listed in (a) to (c).

80. The method of claim 78 or 79, wherein the subject is administered with the at least one recombinant virion or pharmaceutical composition comprising a nucleic acid encoding:
a) hepcidin or a fragment thereof, wherein the at least one recombinant virion or pharmaceutical composition is administered to the subject intravenously, optionally wherein (i) said nucleic acid is operably linked to a promotor, and/or (ii) the at least one recombinant virion or pharmaceutical composition transduces a hepatocyte;
b) HFE or a fragment thereof, wherein the at least one recombinant virion or pharmaceutical composition is administered to the subject intravenously, optionally wherein (i) said nucleic acid is operably linked to a promotor, and/or (ii) the at least one recombinant virion or pharmaceutical composition transduces a hepatocyte;
c) HFE or a fragment thereof, wherein the at least one recombinant virion or pharmaceutical composition is administered to the subject orally, optionally wherein (i) said nucleic acid is operably linked to a promotor, and/or (ii) the at least one recombinant virion or pharmaceutical composition transduces an enterocyte;
d) at least one non-coding RNA (e.g., piRNA, miRNA, shRNA, siRNA, antisense RNA) that targets an endogenous mutant form of HFE, wherein the at least one recombinant virion or pharmaceutical composition is administered to the subject intravenously, optionally wherein (i) said nucleic acid is operably linked to a promotor, and/or (ii) the at least one recombinant virion or pharmaceutical composition transduces a hepatocyte;
e) at least one non-coding RNA (e.g., piRNA, miRNA, shRNA, siRNA, antisense RNA) that targets an endogenous mutant form of HFE, wherein the at least one recombinant virion or pharmaceutical composition is administered to the subject orally, optionally wherein (i) said nucleic acid is operably linked to a promotor, and/or (ii) the at least one recombinant virion or pharmaceutical composition transduces an enterocyte;
f) at least one non-coding RNA (e.g., piRNA, miRNA, shRNA, siRNA, antisense RNA) that targets DMT-1, wherein the at least one recombinant virion or pharmaceutical composition is administered to the subject orally, optionally wherein (i) said nucleic acid is operably linked to a promotor, and/or (ii) the at least one recombinant virion or pharmaceutical composition transduces an enterocyte;
g) at least one non-coding RNA (e.g., piRNA, miRNA, shRNA, siRNA, antisense RNA) that targets ferroportin, wherein the at least one recombinant virion or pharmaceutical composition is administered to the subject orally, optionally wherein (i) said nucleic acid is operably linked to a promotor, and/or (ii) the recombinant virion or pharmaceutical composition transduces an enterocyte; or
h) a combination of two or more of any one of a) to g).

81. The method of claim 80, wherein the combination comprises two or more of any one of b) to e).

82. The method of any one of claims 78-81, wherein the recombinant virion or pharmaceutical composition a) increases the expression of HFE or a fragment thereof, and/or hepcidin or a fragment thereof in the transduced cell; and/or b) decreases the expression of DMT-1, ferroportin, and/or an endogenous mutant form of HFE in the transduced cell.

83. The method of any one of claims 78-82, wherein the at least one recombinant virion, pharmaceutical composition, or transduced cells prevent or treat hemochromatosis, hereditary hemochromatosis, juvenile hemochromatosis, and/or Wilson's disease.

84. The method of claim 78, wherein the at least one recombinant virion, pharmaceutical composition, or transduced cells comprise a nucleic acid encoding (a) a soluble form of the TNFα receptor, a soluble form of the IL-6 receptor, a soluble form of the IL-12 receptor, and/or a soluble form of the IL-1β receptor; (b) at least one non-coding RNA (e.g., piRNA, miRNA, shRNA, siRNA, antisense RNA) that targets the TNFα receptor, IL-6 receptor, IL-12 receptor, and/or IL-1β receptor; (c) a CRISPR/Cas system that targets the TNFα receptor, IL-6 receptor, IL-12 receptor, and/or IL-1β receptor; and/or (d) any combination of any one of the nucleic acids listed in (a) to (c).

85. The method of claim 84, wherein the at least one recombinant virion or pharmaceutical composition a) increases the expression of a soluble form of the TNFα receptor, a soluble form of the IL-6 receptor, a soluble form of the IL-12 receptor, or a soluble form of the IL-1β receptor in the transduced cell; and/or b) decreases the expression of the TNFα receptor, IL-6 receptor, IL-12 receptor, or IL-1β receptor in the transduced cell.

86. The method of any one of claims 78, 84, and 85, wherein the at least one recombinant virion, pharmaceutical composition, or transduced cells prevent or treat rheumatoid arthritis, inflammatory bowel disease, psoriatic arthritis, juvenile chronic arthritis, psoriasis, and/or ankylosing spondylitis.

87. The method of claim 78, wherein the at least one recombinant virion, pharmaceutical composition, or transduced cells comprise a nucleic acid encoding a protein or a fragment thereof selected from IRGM, NOD2, ATG2B, ATG9, ATG5, ATG7, ATG16L1, BECN1, E124/PIG8, TECPR2, WDR45/WIP14, CHMP2B, CHMP4B, Dynein, EPG5, HspB8, LAMP2, LC3b UVRAG, VCP/p97, ZFYVE26, PARK2/Parkin, PARK6/PINK1, SQSTM1/p62, SMURF, AMPK, and ULK1.

88. The method of claim 87, wherein the at least one recombinant virion or pharmaceutical composition increases the expression of said protein or a fragment thereof in the transduced cells.

89. The method of any one of claims 78, 87, and 88, wherein the at least one recombinant virion, pharmaceutical composition, or transduced cells modulate autophagy.

90. The method of any one of claims 78 and 87-89, wherein the at least one recombinant virion, pharmaceutical composition, or transduced cells prevent or treat an autophagy-related disease.

91. The method of claim 90, wherein the autophagy-related disease is selected from selected from cancer, neurodegenerative disease (e.g., Alzheimer's disease, Parkinson's disease, Huntington's disease, ataxias), inflammatory disease, inflammatory bowel disease, Crohn's disease, rheumatoid arthritis, lupus, multiple sclerosis, chronic obstructive pulmony disease/COPD, pulmonary fibrosis, cystic fibrosis, Sjogren's disease, hyperglycemic disorders, type I diabetes, type II diabetes, insulin resistance, hyperinsulinemia, insulin-resistant diabetes (e.g. Mendenhall's Syndrome, Werner Syndrome, leprechaunism, and lipoatrophic diabetes), dyslipidemia, hyperlipidemia, elevated low-density lipoprotein (LDL), depressed highdensity lipoprotein (HDL), elevated triglycerides, metabolic syndrome, liver disease, renal disease, cardiovascular disease, ischemia, stroke, complications during reperfusion, muscle degeneration, atrophy, symptoms of aging (e.g., muscle atrophy, frailty, metabolic disorders, low grade inflammation, atherosclerosis, stroke, age-associated dementia and sporadic form of Alzheimer's disease, pre-cancerous states, and psychiatric conditions including depression), spinal cord injury, arteriosclerosis, infectious diseases (e.g., bacterial, fungal, viral), AIDS, tuberculosis, defects in embryogenesis, infertility, lysosomal storage diseases, activator deficiency/GM2 gangliosidosis, alpha-mannosidosis, aspartylglucoaminuria, cholesteryl ester storage disease, chronic hexosaminidase A deficiency, cystinosis, Danon disease, Fabry disease, Farber disease, fucosidosis, galactosialidosis, Gaucher Disease (Types I, II and III), GM1 Gangliosidosis, (infantile, late infantile/juvenile and adult/chronic), Hunter syndrome (MPS II), I-Cell disease/Mucolipidosis II, Infantile Free Sialic Acid Storage Disease (ISSD), Juvenile Hexosaminidase A Deficiency, Krabbe disease, Lysosomal acid lipase deficiency, Metachromatic Leukodystrophy, Hurler syndrome, Scheie syndrome, Hurler-Scheie syndrome, Sanfilippo syndrome, Morquio Type A and B, Maroteaux-Lamy, Sly syndrome, mucolipidosis, multiple sulfate deficiency, Niemann-Pick disease, Neuronal ceroid lipofuscinoses, CLN6 disease, Jansky-Bielschowsky disease, Pompe disease, pycnodysostosis, Sandhoff disease, Schindler disease, Tay-Sachs, and Wolman disease.

92. The method of claim 78, wherein the protoparvovirus is cutavirus.

93. The method of claim 92, wherein the at least one recombinant virion or pharmaceutical composition targets a T cell, B cell, and/or a lymphoid progenitor cell.

94. The method of any one of claims 78, 92, and 93, wherein the at least one recombinant virion, pharmaceutical composition, or transduced cells prevent or treat cancer.

95. The method of claim 78 or 92, wherein the at least one recombinant virion, pharmaceutical composition, or transduced cells comprise a nucleic acid encoding KRT5, KRT14, PLEC1, Col7A1, ITGB4, ITGA6, LAMA3, LAMB3, LAMC2, and/or KIND 1.

96. The method of claim 95, wherein the transduced cells are epidermal stem cells, P63-positive keratinocyte-derived stem cells, or keratinocytes.

97. The method of claim 95 or 96, further comprising grafting the transduced cells on the skin of the subject.

98. The method of any one of claims 95-97, wherein the at least one recombinant virion, pharmaceutical composition, or transduced cells prevent or treat epidermolysis bullosa.

99. The method of any one of claims 72-77, wherein the at least one recombinant virion or pharmaceutical composition comprises at least one capsid protein or variant thereof of a tetraparvovirus or a genotypic variant thereof.

100. The method of claim 99, wherein the at least one recombinant virion or pharmaceutical composition comprises a nucleic acid encoding a protein or a fragment thereof selected from a hemoglobin gene (HBA1, HBA2, HBB, HBG1, HBG2, HBD, HBE1, and/or HBZ), alpha-hemoglobin stabilizing protein (AHSP), coagulation factor VIII, coagulation factor IX, von Willebrand factor, dystrophin or truncated dystrophin, micro-dystrophin, utrophin or truncated utrophin, micro-utrophin, usherin (USH2A), CEP290, ATPB1, ATPB11, ABCB4, CPS1, ATP7B, KRT5, KRT14, PLEC1, Col7A1, ITGB4, ITGA6, LAMA3, LAMB3, LAMC2, KIND1, INS, F8 or a fragment thereof (e.g., fragment encoding B-domain deleted polypeptide (e.g., VIII SQ, p-VIII)), and cystic fibrosis transmembrane conductance regulator (CFTR).

101. The method of claim 99 or 100, wherein the at least one recombinant virion or pharmaceutical composition transduces (a) a CD34+ stem cell, optionally transduces ex vivo; (b) a mesenchymal stem cell, optionally transduces ex vivo; (c) a liver cell (e.g., hepatocyte), (d) a small intestinal cell, and/or (e) a lung cell.

102. The method of any one of claims 99-101, wherein the at least one recombinant virion or pharmaceutical composition is delivered to liver via hepatic artery, portal vein, or intravenous administration.

103. The method of any one of claims 99-101, wherein the at least one recombinant virion or pharmaceutical composition is delivered to small intestine via oral administration.

104. The method of any one of claims 99-101, wherein the at least one recombinant virion or pharmaceutical composition comprises a nucleic encoding (a) CFTR or a fragment thereof, (b) at least one non-coding RNA (e.g., piRNA, miRNA, shRNA, siRNA, antisense RNA) that targets an endogenous mutant form of CFTR, (c) a CRISPR/Cas system that targets an endogenous mutant form of CFTR; and/or (d) any combination of any one of the nucleic acids listed in (a) to (c).

105. The method of claim 104, wherein the at least one recombinant virion or pharmaceutical composition is delivered to the lung via an intranasal or intrapulmonary administration.

106. The method of any one of claims 99-101, 104, and 105, wherein the at least one recombinant virion or pharmaceutical composition (a) increases the expression of CFTR or fragment thereof, and/or (b) decreases the expression of an endogenous mutant form of CFTR in the transduced cell.

107. The method of any one of claims 99-101 and 104-106, wherein the at least one recombinant virion or pharmaceutical composition prevents or treats cystic fibrosis.

108. The method of any one of claims 72-107, wherein the method further comprises re-administering at least one additional amount of the virion, pharmaceutical composition, or transduced cells.

109. The method of claim 108, wherein said re-administering the at least one additional amount is performed after an attenuation in the treatment subsequent to said administering the effective amount of the virion, pharmaceutical composition, or transduced cells.

110. The method of claim 108 or 109, wherein the at least one additional amount is the same as the said effective amount.

111. The method of claim 108 or 109, wherein the method further comprises increasing or decreasing the at least one additional amount as compared to the said effective amount.

112. The method of claim 111, wherein the at least one additional amount is increased or decreased based on the expression of an endogenous gene and/or the nucleic acid of the recombinant virion.

113. The method of any one of claims 72-112, further comprising administering to the subject or contacting the cells with an agent that modulates the expression of the nucleic acid.

114. The method of claim 113, wherein the agent is selected from a small molecule, a metabolite, an oligonucleotide, a riboswitch, a peptide, a peptidomimetic, a hormone, a hormone analog, and light.

115. The method of claim 113 or 114, wherein the agent is selected from tetracycline, cumate, tamoxifen, estrogen, and an antisense oligonucleotide (ASO), rapamycin, FKCsA, blue light, abscisic acid (ABA), and riboswitch.

116. The method of any one of claims 72-115, further comprising re-administering the agent one or more times at intervals.

117. The method of claim 116, wherein the re-administration of the agent results in pulsatile expression of the nucleic acid.

118. The method of claim 116 or 117, wherein the time between the intervals and/or the amount of the agent is increased or decreased based on the serum concentration and/or half-life of the protein expressed from the nucleic acid.

119. A method of modulating (i) gene expression, or (ii) function and/or structure of a protein in a cell, the method comprising transducing the cell with the virion or pharmaceutical composition of any one of claims 1-71 comprising a nucleic acid that modulates the gene expression, or the function and/or structure of the protein in the cell.

120. The method of claim 119, wherein the nucleic acid comprises the sequence encoding CRISPRi or CRISPRa agents.

121. The method of claim 119 or 120, wherein the gene expression, or the function and/or structure of the protein is increased or restored.

122. The method of claim 119 or 120, wherein the gene expression, or the function and/or structure of the protein is decreased or eliminated.

123. A method of integrating a heterologous nucleic acid into a GSH in a cell, comprising
(a) transducing the cell with one or more virions or pharmaceutical composition according to any one of claims 1-71 comprising a heterologous nucleic acid flanked at the 5′ end and 3′ end by a donor nucleic acid sequence that is at least about 80% identical to the target GSH nucleic acid; or
(b) transducing the cell with one or more virions or pharmaceutical composition according to any one of claims 1-71 comprising (i) a heterologous nucleic acid flanked at the 5′ end and 3′ end by a donor nucleic acid sequence that is at least about 80% identical to the target GSH nucleic acid, and (ii) a nucleic acid encoding a nuclease (e.g., Cas9 or a variant thereof, ZFN, TALEN) and/or a guide RNA, wherein the nuclease or the nuclease/gRNA complex makes a DNA break at the GSH, which is repaired using the donor nucleic acid, thereby integrating a heterologous nucleic acid at GSH.

124. The method of claim 123, wherein (i) the heterologous nucleic acid flanked by a donor nucleic acid that is at least about 80% identical to the target GSH nucleic acid is transduced in one virion, and (ii) the nucleic acid encoding a nuclease and/or the gRNA are transduced in a separate virion.

125. The method of claim 123 or 124, wherein the GSH is AAVS1, ROSA26, CCR5, Kif6, Pax5, an intergenic region of NUPL2, collagen, HTRP, HI 1 (a thymidine kinase encoding nucleic acid at HI 1 locus), beta-2 microglobulin, GAPDH, TCR, RUNX1, KLHL7, mir684, KCNH2, GPNMB, MIR4540, MIR4475, MIR4476, PRL32P21, LOC105376031, LOC105376032, LOC105376030, MELK, EBLN3P, ZCCHC7, or RNF38.

126. The method of any one of claims 123-125, wherein the GSH is AAVS1, ROSA26, CCR5, Kif6, Pax5, or an intergenic region of NUPL2.

127. A method of producing a recombinant virion according to any one of claims 1-70, comprising:
(1) providing at least one vector comprising
(i) a nucleotide sequence comprising at least one ITR nucleotide sequence, optionally further comprising a heterologous nucleic acid operably linked to a promoter for expression in a target cell,
(ii) a nucleotide sequence comprising at least one gene encoding the protoparvovirus or tetraparvovirus capsid proteins VP1,VP2, and/or a variant thereof, operably linked to at least one expression control sequence for expression in an insect cell, and
(iii) a nucleotide sequence comprising
(A) at least one replication protein of a protoparvovirus or tetraparvovirus operably linked to at least one expression control sequence for expression in an insect cell,
(B) at least one replication protein of an AAV, optionally wherein the at least one replication protein of an AAV comprises (a) a Rep52 or a Rep40 coding sequence operably linked to at least one expression control sequence for expression in an insect cell, and/or (b) a Rep78 or a Rep68 coding sequence operably linked to at least one expression control sequence for expression in an insect cell, or
(C) a combination of (A) and (B),
(2) introducing said at least one vector into an insect cell, and
(3) maintaining said insect cell under conditions such that a recombinant virion according to any one of claims 1-70 is produced.

128. The method of claim 127, wherein two vectors are provided,
(a) a first vector comprising a nucleotide sequence comprising at least one ITR nucleotide sequence, optionally further comprising a heterologous nucleic acid operably linked to a promoter for expression in a target cell, and
(b) a second vector comprising
(i) a nucleotide sequence comprising at least one gene encoding the protoparvovirus or tetraparvovirus capsid proteins VP1,VP2, and/or a variant thereof, operably linked to at least one expression control sequence for expression in an insect cell, and
(ii) a nucleotide sequence comprising
(A) at least one replication protein of a protoparvovirus or tetraparvovirus operably linked to at least one expression control sequence for expression in an insect cell,
(B) at least one replication protein of an AAV, optionally wherein the at least one replication protein of an AAV comprises (a) a Rep52 or a Rep40 coding sequence operably linked to at least one expression control sequence for expression in an insect cell, and/or (b) a Rep78 or a Rep68 coding sequence operably linked to at least one expression control sequence for expression in an insect cell, or
(C) a combination of (A) and (B).

129. The method of claim 127, wherein three vectors are provided,
(a) a first vector comprising a nucleotide sequence comprising at least one ITR nucleotide sequence, optionally further comprising a heterologous nucleic acid operably linked to a promoter for expression in a target cell,
(b) a second vector comprising a nucleotide sequence comprising a gene encoding the protoparvovirus or tetraparvovirus capsid proteins VP1, VP2, and/or a variant thereof, operably linked to at least one expression control sequence for expression in an insect cell, and
(c) a third vector comprising a nucleotide sequence comprising
(A) at least one replication protein of a protoparvovirus or tetraparvovirus operably linked to at least one expression control sequence for expression in an insect cell,
(B) at least one replication protein of an AAV, optionally wherein the at least one replication protein of an AAV comprises (a) a Rep52 or a Rep40 coding sequence operably linked to at least one expression control sequence for expression in an insect cell, and/or (b) a Rep78 or a Rep68 coding sequence operably linked to at least one expression control sequence for expression in an insect cell, or
(C) a combination of (A) and (B).

130. A method of producing a recombinant virion according to any one of claims 1-70 in an insect cell, the method comprising:
(1) providing an insect cell comprising
(i) a nucleotide sequence comprising at least one ITR nucleotide sequence, optionally further comprising a heterologous nucleic acid operably linked to a promoter for expression in a target cell,
(ii) a nucleotide sequence comprising at least one gene encoding the protoparvovirus or tetraparvovirus capsid proteins VP1, VP2, and/or a variant thereof, operably linked to at least one expression control sequence for expression in an insect cell, and
(iii) a nucleotide sequence comprising
(A) at least one replication protein of a protoparvovirus or tetraparvovirus operably linked to at least one expression control sequence for expression in an insect cell,
(B) at least one replication protein of an AAV, optionally wherein the at least one replication protein of an AAV comprises (a) a Rep52 or a Rep40 coding sequence operably linked to at least one expression control sequence for expression in an insect cell, and/or (b) a Rep78 or a Rep68 coding sequence operably linked to at least one expression control sequence for expression in an insect cell, or
(C) a combination of (A) and (B),
wherein at least one of (i), (ii), (iii)(A), (iii)(B), and (iii)(C) is/are stably integrated in the insect cell genome, and at least one vector, if present, comprises the remainder of the (i), (ii), (iii)(A), (iii)(B), and (iii)(C) nucleotide sequences which is/are not stably integrated in the insect cell genome, and
(2) maintaining the insect cell under conditions such that the recombinant virion is produced.

131. The method of any one of claims 127-130, wherein the protoparvovirus or a genotypic variant thereof is of a species selected from Carnivore protoparvovirus, Carnivore protoparvovirus 1, Chiropteran protoparvovirus 1, Eulipotyphla protoparvovirus 1, Primate protoparvovirus 1, Primate protoparvovirus 2, Primate protoparvovirus 3, Primate protoparvovirus 4, Rodent protoparvovirus 1, Rodent protoparvovirus 2, Rodent protoparvovirus 3, Ungulate protoparvovirus 1, and Ungulate protoparvovirus 2.

132. The method of any one of claims 127-131, wherein the protoparvovirus or a genotypic variant thereof is selected from canine parvovirus, feline panelukepenia virus, human bufavirus 1, human bufavirus 2, human bufavirus 3, human tusavirus, human cutavirus, Wuharv parvovirus, porcine parvovirus, minute virus of mice, megabat bufavirus, and a genotypic variant thereof.

133. The method of any one of claims 127-130, wherein the tetraparvovirus or a genotypic variant thereof is of a species selected from Chiropteran tetraparvovirus 1, Primate tetraparvovirus 1, Ungulate tetraparvovirus 1, Ungulate tetraparvovirus 2, Ungulate tetraparvovirus 3, and Ungulate tetraparvovirus 4.

134. The method of any one of claims 127-130 and 133, wherein the tetraparvovirus or a genotypic variant thereof is selected from human parvovirus 4, human parvovirus 4 genotype 1, human parvovirus 4 genotype 2, human parvovirus 4 genotype 3, chimpanzee parvovirus 4, eidolon helvum parvovirus, bovine hokovirus 1, bovine hokovirus 2, porcine hokovirus, porcine cnvirus, yak parvovirus, ovine hokovirus 1, opossum tetraparvovirus, rodent tetraparvovirus, tetraparvovirus sp., and a genotypic variant thereof.

135. The method of any one of claims 127-130, 133, and 134, wherein the tetraparvovirus is human parvovirus 4, human parvovirus 4 genotype 1, human parvovirus 4 genotype 2, human parvovirus 4 genotype 3, or a genotypic variant thereof.

136. The method of any one of claims 127-135, wherein the at least one replication protein of a protoparvovirus is an NS-1 protein of a canine parvovirus, bufavirus, cutavirus, or a genotypic variant thereof.

137. The method of any one of claims 127-135, wherein the at least one replication protein of a tetravirus is an NS-1 protein of a human parvovirus 4 or a genotypic variant thereof.

138. The method of any one of claims 127-137, wherein the insect cell is derived from a species of Lepidoptera.

139. The method of claim 138, wherein the species of Lepidoptera is Spodoptera frugiperda, Spodoptera littoralis, Spodoptera exigua, or Trichoplusia ni.

140. The method of any one of claims 127-139, wherein the insect cell is Sf9.

141. The method of any one of claims 127-140, wherein the at least one vector is a baculoviral vector, a viral vector, or a plasmid.

142. The method of any one of claims 127-141, wherein the at least one vector is a baculoviral vector.

143. The method of any one of claims 127-142, wherein VP1 comprises an amino acid sequence that is at least about 60% identical to a sequence selected from SEQ ID NOs: 4, 5, 7, 8, 10, 11, 13, 14, 16, 17, 19, 20, and 22.

144. The method of any one of claims 127-143, wherein VP2 comprises an amino acid sequence that is at least about 60% identical to a sequence selected from SEQ ID NOs: 4, 6, 7, 9, 10, 12, 13, 15, 16, 18, 19, 21, and 23.

145. The method of any one of claims 127-144, wherein the at least one ITR comprises:
(a) a dependoparvovirus ITR,
(b) an AAV ITR, optionally an AAV2 ITR,
(c) a protoparvovirus ITR, and/or
(d) a tetraparvovirus ITR.

146. The method of any one of claims 127-145, wherein the at least one expression control sequence for expression in an insect cell comprises:
(a) a promoter, and/or
(b) a Kozak-like expression control sequence.

147. The method of claim 146, wherein the promoter comprises:
(a) an immediate early promoter of an animal DNA virus,
(b) an immediate early promoter of an insect virus, or
(c) an insect cell promoter.

148. The method of claim 147, wherein the animal DNA virus is cytomegalovirus (CMV), protoparvovirus, tetraparvovirus, or AAV.

149. The method of claim 147, wherein the insect virus is a lepidopteran virus or a baculovirus, optionally wherein the baculovirus is Autographa californica multicapsid nucleopolyhedrovirus (AcMNPV).

150. The method of any one of claims 146-149, wherein the promoter is a polyhedrin (polh) or immediately early 1 gene (IE-1) promoter.

151. The method of any one of claims 127-150, wherein the nucleotide sequence comprising at least one replication protein of an AAV comprises a nucleotide sequence encoding Rep52 and/or Rep78.

152. The method of any one of claims 127-151, wherein the AAV is AAV2.

153. An insect cell, comprising at least one vector, comprising:
(i) a nucleotide sequence comprising at least one ITR nucleotide sequence,
(ii) a nucleotide sequence comprising at least one gene encoding the protoparvovirus or tetraparvovirus capsid proteins VP1, VP2, and/or a variant thereof, operably linked to at least one expression control sequence for expression in an insect cell, and
(iii) a nucleotide sequence comprising
(A) at least one replication protein of a protoparvovirus or tetraparvovirus operably linked to at least one expression control sequence for expression in an insect cell,
(B) at least one replication protein of an AAV, optionally wherein the at least one replication protein of an AAV comprises (a) a Rep52 or a Rep40 coding sequence operably linked to at least one expression control sequence for expression in an insect cell, and/or (b) a Rep78 or a Rep68 coding sequence operably linked to at least one expression control sequence for expression in an insect cell, or
(C) a combination of (A) and (B).

154. The insect cell of claim 153, wherein at least one of(i), (ii), (iii)(A), (iii)(B), and (iii)(C) is stably integrated in the insect cell genome.

155. The insect cell of claim 153 or 154, wherein the protoparvovirus or a genotypic variant thereof is of a species selected from Carnivore protoparvovirus, Carnivore protoparvovirus 1, Chiropteran protoparvovirus 1, Eulipotyphla protoparvovirus 1, Primate protoparvovirus 1, Primate protoparvovirus 2, Primate protoparvovirus 3, Primate protoparvovirus 4, Rodent protoparvovirus 1, Rodent protoparvovirus 2, Rodent protoparvovirus 3, Ungulate protoparvovirus 1, and Ungulate protoparvovirus 2.

156. The insect cell of any one of claims 153-155, wherein the protoparvovirus or a genotypic variant thereof is selected from canine parvovirus, feline panelukepenia virus, human bufavirus 1, human bufavirus 2, human bufavirus 3, human tusavirus, human cutavirus, Wuharv parvovirus, porcine parvovirus, minute virus of mice, megabat bufavirus, and a genotypic variant thereof.

157. The insect cell of claim 153 or 154, wherein the tetraparvovirus or a genotypic variant thereof is of a species selected from Chiropteran tetraparvovirus 1, Primate tetraparvovirus 1, Ungulate tetraparvovirus 1, Ungulate tetraparvovirus 2, Ungulate tetraparvovirus 3, and Ungulate tetraparvovirus 4.

158. The insect cell of any one of claims 153, 154, and 157, wherein the tetraparvovirus or a genotypic variant thereof is selected from human parvovirus 4, human parvovirus 4 genotype 1, human parvovirus 4 genotype 2, human parvovirus 4 genotype 3, chimpanzee parvovirus 4, eidolon helvum parvovirus, bovine hokovirus 1, bovine hokovirus 2, porcine hokovirus, porcine cnvirus, yak parvovirus, ovine hokovirus 1, opossum tetraparvovirus, rodent tetraparvovirus, tetraparvovirus sp., and a genotypic variant thereof.

159. The insect cell of any one of claims 153, 154, 157, and 158, wherein the tetraparvovirus is human parvovirus 4, human parvovirus 4 genotype 1, human parvovirus 4 genotype 2, human parvovirus 4 genotype 3, or a genotypic variant thereof.

160. The insect cell of any one of claims 153-159, wherein the at least one replication protein of a protoparvovirus is an NS-1 protein of a canine parvovirus, bufavirus, cutavirus, or a genotypic variant thereof.

161. The insect cell of any one of claims 153-159, wherein the at least one replication protein of a tetravirus is an NS-1 protein of a human parvovirus 4, or a genotypic variant thereof.

162. The insect cell of any one of claims 153-161, wherein the insect cell is derived from a species of Lepidoptera.

163. The insect cell of claim 162, wherein the species of Lepidoptera is Spodoptera frugiperda, Spodoptera littoralis, Spodoptera exigua, or Trichoplusia ni.

164. The insect cell of any one of claims 153-163, wherein the insect cell is Sf9.

165. The insect cell of any one of claims 153-164, wherein the at least one vector is a baculoviral vector, a viral vector, or a plasmid.

166. The insect cell of any one of claims 153-165, wherein the at least one vector is a baculoviral vector.

167. The insect cell of any one of claims 153-166, wherein VP1 comprises an amino acid sequence that is at least about 60% a sequence selected from SEQ ID NOs: 4, 5, 7, 8, 10, 11, 13, 14, 16, 17, 19, 20, and 22.

168. The insect cell of any one of claims 153-167, wherein VP2 comprises an amino acid sequence that is at least about 60% identical to a sequence selected from SEQ ID NOs: 4, 6, 7, 9, 10, 12, 13, 15, 16, 18, 19, 21, and 23.

169. The insect cell of any one of claims 153-168, wherein the at least one ITR comprises:
(a) a dependoparvovirus ITR,
(b) an AAV ITR, optionally an AAV2 ITR,
(c) a protoparvovirus ITR, and/or
(d) a tetraparvovirus ITR.

170. The insect cell of any one of claims 153-169, wherein the at least one expression control sequence for expression in an insect cell comprises:
(a) a promoter, and/or
(b) a Kozak-like expression control sequence.

171. The insect cell of claim 170, wherein the promoter comprises:
(a) an immediate early promoter of an animal DNA virus,
(b) an immediate early promoter of an insect virus, or
(c) an insect cell promoter.

172. The insect cell of claim 171, wherein the animal DNA virus is cytomegalovirus (CMV), protoparvovirus, tetraparvovirus, or AAV.

173. The insect cell of claim 171, wherein the insect virus is a lepidopteran virus or a baculovirus, optionally wherein the baculovirus is Autographa californica multicapsid nucleopolyhedrovirus (AcMNPV).

174. The insect cell of any one of claims 170-173, wherein the promoter is a polyhedrin (polh) or immediately early 1 gene (IE-1) promoter.

175. The insect cell of any one of claims 153-174, wherein the nucleotide sequence comprising at least one replication protein of an AAV comprises a nucleotide sequence encoding Rep52 and/or Rep78.

176. The insect cell of any one of claims 153-175, wherein the AAV is AAV2.
